(19) United States
(12) Patent Application Publication
(10) Pub. No.: US 2009/0169585 A1
(43) Pub. Date: Jul. 2, 2009

(54) Resveratrol-Containing Compositions And Their Use In Modulating Gene Product Concentration Or Activity

(75) Inventor: William F. Sardi, La Verne, CA (US)

Correspondence Address: EDELL, SHAPIRO & FINNAN, LLC, 1901 RESEARCH BOULEVARD, SUITE 400, ROCKVILLE, MD, 20850, US

(73) Assignee: Resveratrol Partners, LLC, Las Vegas, NV (US)

(21) Appl. No.: 12/212,494

(22) Filed: Sep. 17, 2008

Related U.S. Application Data
(63) Continuation-in-part of application No. 10/971,017, filed on Oct. 25, 2004.
(60) Provisional application No. 60/973,817, filed on Sep. 20, 2007.
(60) Provisional application No. 61/023,227, filed on Jan. 24, 2008.
(60) Provisional application No. 61/023,230, filed on Jan. 24, 2008.
(60) Provisional application No. 61/023,234, filed on Jan. 24, 2008.
(60) Provisional application No. 61/048,756, filed on Apr. 29, 2008.
(60) Provisional application No. 61/048,769, filed on Apr. 29, 2008.
(60) Provisional application No. 60/513,225, filed on Oct. 23, 2003.

(51) Int. Cl. A61K 9/00; A61K 31/047; A61K 31/352; A61K 31/728; A61K 31/59

(52) U.S. Cl. 424/400; 514/729; 514/456; 514/54; 514/167

(57) ABSTRACT

The present invention relates to a resveratrol-containing composition capable of modulating gene expression to an extent greater than that observed with resveratrol alone or with calorie restriction. The invention particularly pertains to such resveratrol-containing compositions that comprise resveratrol, a chelator, hyaluronic acid, and/or vitamin D and which upon administration to a recipient, increases the concentration or activity of a survival/longevity gene product and/or decreases the concentration or activity of a gene product that induces or causes cellular damage. Most preferably, the resveratrol-stabilizing composition will comprise the chelator phytic acid (inositol hexaphosphate; IP6), hyaluronic acid, and vitamin D. The invention further pertains to the use of such compositions in the treatment or prevention of cancer, cardiovascular disease, diseases associated with aging, and other conditions and illnesses.

### CROSS-REFERENCE TO RELATED APPLICATIONS

This application claims priority from U.S. patent application Ser. No. 10/971,017 (filed Oct. 25, 2004; pending), which application claims priority to 60/513,225 (filed on Oct. 23, 2003; lapsed), and to U.S. Patent Application Ser. Nos. 60/973,817 (filed on Sep. 20, 2007; pending), 61/023,227 (filed on Jan. 24, 2008; pending), 61/023,230 (filed on Jan. 24, 2008; pending), 61/023,234 (filed on Jan. 24, 2008; pending), 61/048,756 (filed on Apr. 29, 2008; pending) and), 61/048,769 (filed on Apr. 29, 2008; pending) all of which applications are herein incorporated by reference in their entirety.

### FIELD OF THE INVENTION

The present invention relates to a resveratrol-containing composition capable of modulating gene expression to an extent greater than that observed with resveratrol alone or with calorie restriction. The invention particularly pertains to such resveratrol-containing compositions that comprise resveratrol, a chelator, hyaluronic acid, and/or vitamin D and which, upon administration to a recipient, increases the concentration or activity of a survival/longevity gene product and/or decreases the concentration or activity of a gene product that induces or causes cellular damage. The invention particularly relates to the use of such compositions in the treatment or prevention of cancer, cardiovascular disease, diseases associated with aging, and other conditions and illnesses.

### BACKGROUND OF THE INVENTION

Despite a high level of risk factors such as cholesterol, diabetes, hypertension and a high intake of saturated fat, French males display the lowest mortality rate from ischaemic heart disease and cardiovascular diseases in Western industrialized nations (36% lower than the USA and 39% lower than the UK). The so-called ‘French Paradox’ (a low mortality rate specifically from cardiovascular diseases) may be due mainly to the regular consumption of wine (Renaud, S. et al. (1998) “The French Paradox And Wine Drinking,” Novartis Found. Symp. 216:208-222, 152-158).

Resveratrol (3,4′,5-trihydroxy-trans-stilbene) is a naturally occurring phenolic compound found, for example in grape skins, that has been demonstrated to have beneficial properties relating to health of humans (Das, S. et al. (2007) “Resveratrol: A Therapeutic Promise For Cardiovascular Diseases,” Recent Patents Cardiovasc. Drug Discov. 2(2):133-138; Mancuso, C. et al. (2007) “Natural antioxidants in Alzheimer's disease,” Expert Opin. Investig. Drugs. 16(12):1921-1931; Baumann L. (2007) “Botanical Ingredients In Cosmeceuticals,” J. Drugs Dermatol. 6(11):1084-1088; Meeran, S. M. et al. (2008) “Cell Cycle Control As A Basis For Cancer Chemoprevention Through Dietary Agents,” Front. Biosci. 13:2191-2202; de la Lastra, C. A. et al. (2007) “Resveratrol As An Antioxidant And Pro-Oxidant Agent: Mechanisms And Clinical Implications,” Biochem. Soc. Trans. 35(Pt 5):1156-1160; Das, S. et al. (2007) “Anti-Inflammatory Responses Of Resveratrol,” Inflamm. Allergy Drug Targets 6(3):168-173; Cucciolla, V. et al. (Epub 2007 Jul. 31) “Resveratrol: From Basic Science To The Clinic,” Cell Cycle 6(20):2495-2510; Opie, L. H. et al. (Epub 2007 Jun. 7) “The Red Wine Hypothesis: From Concepts To Protective Signaling Molecules,” Eur. Heart J. 28(14):1683-1693; Chen, Y. et al. (2007) “Review: Pro- And Anti-Angiogenesis Effects Of Resveratrol,” In Vivo 21(2):365-370 [Erratum in: In Vivo 21(6):1172 and 21(5):955]; Holme, A. L. et al. (2007) “Resveratrol In Cell Fate Decisions,” J. Bioenerg. Biomembr. 39(1):59-63; Athar, M. et al. (Epub 2007 Jan. 3) “Resveratrol: A Review Of Prectinical Studies For Human Cancer Prevention,” Toxicol. Appl. Pharmacol. 224(3):274-283). In particular, resveratrol is believed to be beneficial to the functioning of the heart and in extending the life of human cells. Resveratrol, when used in dietary supplements, is generally produced as an alcohol extract from plant sources.

Calorie restricted diets have been shown to enhance survival and longevity by up-regulating survival/longevity genes or down-regulating genes whose expression enhances cellular damage (Edwards, M. G. et al. (2007) “Gene Expression Profiling Of Aging Reveals Activation Of A P53-Mediated Transcriptional Program,” BMC Genomics 8:80; Anderson, R. M. et al. (2006) “Calorie Restriction: Progress During Mid-2005-Mid-2006,” Exp. Gerontol. 41(12):1247-1249; Weindruch, R. et al. (2001) “Microarray Profiling of Gene Expression in Aging and Its Alteration by Caloric Restriction in Mice,” J. Nutrition 131:918 S-923S; Lee, K.-C. et al. (2003) “Transcriptional Profiles Associated With Aging And Middle Age-Onset Caloric Restriction In Mouse Hearts,” Proc. Natl. Acad. Sci. (U.S.A.) 99(23): 14988-14993; Weindruch, R. et al. (2002) “Effects Of Caloric Restriction On Gene Expression,” Nestle Nutr. Workshop Ser. Clin. Perform. Programme 6:17-28; 28-32; Mulligan, J. D. et al. (Epub 2007 Nov. 1) “Downregulation Of Plasma Insulin Levels And Hepatic Ppar gamma Expression During The First Week Of Caloric Restriction In Mice,” Exp. Gerontol. 43(3):146-153; Rodgers, J. T. et al. (Epub 2007 Nov. 26) “Metabolic Adaptations Through The PGC-1 Alpha And SIRT1 Pathways,” FEBS Lett. 582(1):46-53; Swindell, W. R. (2007) “Gene Expression Profiling Of Long-Lived Dwarf Mice: Longevity-Associated Genes And Relationships With Diet, Gender And Aging,” BMC Genomics. 8:353; Zhu, M. et al. (Epub 2007 Jun. 6) “Adipogenic Signaling In Rat White Adipose Tissue: Modulation By Aging And Calorie Restriction,” Exp. Gerontol. 42(8):733-744; Chiarpotto, E. et al. (2006) “Molecular Mechanisms Of Calorie Restriction's Protection Against Age-Related Sclerosis,” IUBMB Life. 58(12):695-702; Lu, J. et al. (2007) “Different Gene Expression Of Skin Tissues Between Mice With Weight Controlled By Either Calorie Restriction Or Physical Exercise,” Exp. Biol. Med. (Maywood). 232(4):473-480; Masternak, M. M. et al. (2007) “pPARS In Calorie Restricted And Genetically Long-Lived Mice,” pPAR Res. 2007:28436; Fu, C. et al. (2006) “Tissue Specific And Non-Specific Changes In Gene Expression By Aging And By Early Stage CR,” Mech. Ageing Dev. 127(12):905-916). As indicated by the above-cited references, mice have been used extensively as a model for genetic expression comparisons with humans. Without limitation, the validity of murine models to human gene expression reflects the fact that 98% of human and murine gene are homologous, and that mice and humans have about the same number of genes (e.g., approximately 30,000).

Despite the established benefits of a calorie restricted diet, the severity of the required dietary regime has limited adoption of this approach to increasing longevity. It would therefore be desirable to provide an alternative route to obtaining the benefits of calorie restriction that would avoid the need for dietary regulation and that would be amenable to widespread adoption. The present invention is directed to this and other needs.

### SUMMARY OF THE INVENTION

The present invention relates to a resveratrol-containing composition capable of modulating gene expression to an extent greater than that observed with resveratrol alone or with calorie restriction. The invention particularly pertains to such resveratrol-containing compositions that comprise resveratrol, a chelator, hyaluronic acid, and/or vitamin D and which, upon administration to a recipient, increases the concentration or activity of a survival/longevity gene product and/or decreases the concentration or activity of a gene product that induces or causes cellular damage. Most preferably, the resveratrol-stabilizing composition will comprise the chelator phytic acid (inositol hexaphosphate; IP6), hyaluronic acid, and vitamin D. The invention further pertains to the use of such compositions to up-regulate a survival/longevity gene or down-regulate a gene whose expression enhances cellular damage upon administration to a recipient. The invention particularly relates to the use of such compositions in the treatment or prevention of cancer, cardiovascular disease, diseases associated with aging, and other conditions and illnesses.

In detail, the invention provides a resveratrol-containing composition that, upon administration to a recipient, modulates the concentration or activity, relative to resveratrol alone or calorie restriction, of the product of a survival/longevity gene or the product of a gene whose expression enhances cellular damage. Administration is preferably by oral ingestion.

The invention further provides the embodiments of such compositions wherein the modulation alters:
(A) oxidative phosphorylation;
(B) actin filament length or polymerization;
(C) intracellular transport;
(D) organelle biogenesis;
(E) insulin signaling;
(F) glycolysis;
(G) gluconeogenesis; or
(H) fatty acid metabolism.

The invention further provides the embodiments of such compositions wherein the gene product is a survival/longevity gene product, and especially wherein the gene product is sSirtuin 1, or the forkhead Foxo1 transcription factor.

The invention further provides the embodiments of such compositions wherein the gene product is a gene product that enhances cellular damage, and especially wherein the gene product is encoded by the uncoupling protein 3, Pgc-1, or pyruvate dehydrogenase kinase 4 genes.

The invention further provides the embodiments of such compositions wherein the composition comprises:

(a) trans-resveratrol; and

(b) a metal chelating agent;

wherein the trans-resveratrol is encapsulated to thereby substantially preserve the ability of the composition to modulate the concentration or activity of the product of the survival/longevity gene or the product of the gene whose expression enhances cellular damage, from loss due to exposure of the trans-resveratrol to light or oxygen.

The invention further provides a method of ameliorating a symptom associated with an existing disease of an individual or for preventing the onset of the symptom in an individual prior to the occurrence of the disease in the individual, which comprises administering to the individual, a resveratrol-containing composition that modulates the concentration or activity, relative to resveratrol alone or calorie restriction, of the product of a survival/longevity gene or the product of a gene whose expression enhances cellular damage, wherein the resveratrol is provided in an amount effective to cause a modulation of the concentration or activity of the gene that ameliorates the symptom of the disease, and wherein the disease is selected from the group consisting of: cardiovascular disease, cancer, macular degeneration, a disease associated with aging, and inflammation.

The invention further provides the embodiments of such method wherein the modulation alters:
(A) oxidative phosphorylation;
(B) actin filament length or polymerization;
(C) intracellular transport;
(D) organelle biogenesis;
(E) insulin signaling;
(F) glycolysis;
(G) gluconeogenesis; or
(H) fatty acid metabolism.

The invention further provides the embodiments of such method wherein the survival/longevity gene product is Sirtuin 1 or the forkhead Foxo1 transcription factor. The invention further provides the embodiments of such method wherein the gene whose expression enhances cellular damage encodes uncoupling protein 3 or pyruvate dehydrogenase kinase 4.

The invention further provides the embodiments of such method wherein said composition comprises:

(a) trans-resveratrol; and

(b) a metal chelating agent;

wherein the trans-resveratrol is encapsulated to thereby substantially preserve the ability of the composition to modulate the concentration or activity of the product of the survival/longevity gene or the product of the gene whose expression enhances cellular damage, from loss due to exposure of the trans-resveratrol to light or oxygen.

The invention further provides the embodiments of such method wherein the disease is cancer, or a disease associated with aging (especially a neurodegenerative disease).

The invention further provides the embodiments of such method wherein the composition additionally comprises quercetin, hyaluronic acid and/or vitamin D.

The invention further provides the embodiments of such method wherein the modulation is relative to resveratrol alone or wherein the modulation is relative to calorie restriction.

The invention further provides the embodiments of such methods wherein the gene product is a survival/longevity gene product, and especially wherein the gene product is Sirtuin 1, or the forkhead Foxo1 transcription factor.

The invention further provides the embodiments of such methods wherein the gene product is a gene product that enhances cellular damage, and especially wherein the gene product is encoded by the uncoupling protein 3, Pgc-1, or pyruvate dehydrogenase kinase 4 gene.

### BRIEF DESCRIPTION OF THE FIGURES

FIG. 1 shows the change in body weight of mice administered resveratrol or a composition of the present invention (Longevinex®) relative to control animals and animals maintained on a calorie restricted diet.

FIG. 2 shows the serum insulin level of mice administered resveratrol or a composition of the present invention (Longevinex®) relative to control animals and animals maintained on a calorie restricted diet.

FIG. 3 shows the serum glucose level of mice administered resveratrol (P=0.97) or a composition of the present invention (Longevinex®) (P=0.07) relative to control animals and animals maintained on a calorie restricted diet (P=0.10).

FIG. 4 shows a schematic of a mechanism of action that is consistent with the observed biological activities of the compositions of the present invention.

### DESCRIPTION OF THE PREFERRED EMBODIMENTS

The present invention relates to a resveratrol-containing composition (and especially a resveratrol-containing dietary composition (i.e., a composition amenable for oral ingestion by a recipient)) capable of modulating gene expression to an extent greater than that observed with resveratrol alone or with calorie restriction. The invention particularly pertains to such resveratrol-containing compositions that comprise resveratrol, a chelator, hyaluronic acid, and/or vitamin D and which up-regulate a survival/longevity gene or down-regulate a gene whose expression enhances cellular damage upon administration to a recipient. Most preferably, the resveratrol-stabilizing composition will comprise the chelator phytic acid (inositol hexaphosphate; IP6), hyaluronic acid, and vitamin D. The invention further pertains to the use of such compositions to up-regulate a survival/longevity gene or down-regulate a gene whose expression enhances cellular damage upon administration to a recipient. The mineral chelators of the present invention provide anti-aging effects, as evidence in differentiation of the genome.

A. Resveratrol

As used herein, the term “resveratrol” refers to the phytoalexin: 3,4′,5-trihydroxy-trans-stilbene having the structure:

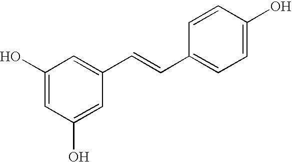

Resveratrol has been ascribed multiple beneficial biological effects (see, U.S. Pat. No. 7,345,178, which listing of disclosed effects is herein incorporated by reference), including preventing or treating cardiovascular disease (see, e.g., Das, S. et al. (2007) “Resveratrol: A Therapeutic Promise For Cardiovascular Diseases,” Recent Patents Cardiovasc. Drug Discov. 2(2):133-138), Opie, L. H. et al. (Epub 2007 Jun. 7) “The Red Wine Hypothesis: From Concepts To Protective Signaling Molecules,” Eur. Heart J. 28(14):1683-1693; Bertelli, A. A. (Epub 2007 May 24) “Wine, Research And Cardiovascular Disease: Instructions For Use,” Atherosclerosis 195(2):242-247; Providência, R. (2006) “Cardiovascular Protection From Alcoholic Drinks: Scientific Basis Of The French Paradox,” Rev. Port. Cardiol. 25(11):1043-1058; Maulik, N. (2006) “Reactive Oxygen Species Drives Myocardial Angiogenesis?” Antioxid. Redox Signal. 8(11-12):2161-2168; Olas, B. et al. (2005) “Resveratrol, A Phenolic Antioxidant With Effects On Blood Platelet Functions,” Platelets 16(5):251-260; Bradamante, S. et al. (2004) “Cardiovascular Protective Effects Of Resveratrol,” Cardiovasc Drug Rev. 22(3):169-188; Hao, H. D. et al. (2004) “Mechanisms Of Cardiovascular Protection By Resveratrol,” J. Med. Food 7(3):290-298), preventing or treating cancer (Jang, M. et al. (1997) “Cancer Chemopreventive Activity Of Resveratrol, A Natural Product Derived From Grapes,” Science 275:218-220; Das, S. et al. (2007) “Anti-Inflammatory Responses Of Resveratrol,” Inflamm. Allergy Drug Targets 6(3):168-173; de la Lastra, C. A. et al. (2007) “Resveratrol As An Antioxidant And Pro-Oxidant Agent: Mechanisms And Clinical Implications,” Biochem. Soc. Trans. 35(Pt 5):1156-1160; Athar, M. et al. (Epub 2007 Jan. 3) “Resveratrol: A Review Of Preclinical Studies For Human Cancer Prevention,” Toxicol. Appl. Pharmacol. 224(3):274-283), Meeran, S. M. et al. (2008) “Cell Cycle Control As A Basis For Cancer Chemoprevention Through Dietary Agents,” Front. Biosci. 13:2191-2202; Shankar, S. et al. (2007) “Chemoprevention By Resveratrol: Molecular Mechanisms And Therapeutic Potential,” Front. Biosci. 12:4839-4854; Delmas, D. et al. (2006) “Resveratrol As A Chemopreventive Agent: A Promising Molecule For Fighting Cancer,” Curr. Drug Targets 7(4):423-442; Signorelli, P. et al. (2005) “Resveratrol As An Anticancer Nutrient: Molecular Basis, Open Questions And Promises,” J. Nutr. Biochem. 16(8):449-466; preventing or treating macular degeneration (U.S. Patent Application Ser. No. 61/023,234; King, R. E. et al. (2005) “Resveratrol reduces oxidation and proliferation of human retinal pigment epithelial cells via extracellular signal-regulated kinase inhibition,” Chem. Biol. Interact. 151(2):143-149; Sparrow, J. R. et al. (2003) “A2E-epoxides damage DNA in retinal pigment epithelial cells. Vitamin E and other antioxidants inhibit A2E-epoxide formation,” J. Biol. Chem. 278(20):18207-18213), attenuating or preventing diseases associated with aging, and other conditions and illnesses, including the incidence or severity of neurodegenerative diseases such as Alzheimer's Disease and Parkinson's Disease (Baxter, R. A. (2008) “Anti-Aging Properties Of Resveratrol: Review And Report Of A Potent New Antioxidant Skin Care Formulation,” J. Cosmet. Dermatol. 7(1):2-7; Engel, N. et al. (2008) “Aging And Anti-Aging: Unexpected Side Effects Of Everyday Medication Through Sirtuin1 Modulation,” Int. J. Mol. Med. 21(2):223-232; Bickenbach, K. A. et al. (Epub 2007 Dec. 21) “Resveratrol Is An Effective Inducer Of Carg-Driven Tnf-Alpha Gene Therapy,” Cancer Gene Ther. 15(3):133-139; Putics, A. et al. (2008) “Resveratrol Induces The Heat-Shock Response And Protects Human Cells From Severe Heat Stress,” Antioxid. Redox Signal. 10(1):65-75; Bass, T. M. et al. (Epub 2007 Aug. 14) “Effects Of Resveratrol On Lifespan In Drosophila melanogaster And Caenorhabditis elegans,” Mech. Ageing Dev. 128(10):546-552; Stefani, M. et al. (Epub 2007 Sep. 5) “The Effect Of Resveratrol On A Cell Model Of Human Aging,” Ann. N.Y. Acad. Sci. 1114:407-418; Heiss, E. H. et al. (Epub 2007 Jul. 11) “Chronic Treatment With Resveratrol Induces Redox Stress- And Ataxia Telangiectasia-Mutated (Atm)-Dependent Senescence In P53-Positive Cancer Cells,” J. Biol. Chem. 282(37):26759-26766; Mayo Clinic (2007) “A Compound In Red Wine Makes Mice Live Longer, Healthier,” Mayo Clin. Health Lett. 25(5):4; Kim, D. et al. (Epub 2007 Jun. 21) “Sirt1 Deacetylase Protects Against Neurodegeneration In Models For Alzheimer's Disease And Amyotrophic Lateral Sclerosis,” EMBO J. 26(13):3169-3179), and anti-inflammatory activity (Das, S. et al. (2007) “Anti-Inflammatory Responses Of Resveratrol,” Inflamm. Allergy Drug Targets 6(3): 168-173).

Reseveratrol may be synthesized chemically (Farina, A. et al. (2006) “An Improved Synthesis Of Resveratrol,” Nat. Prod. Res. 20(3):247-252), or, more preferably, may be extracted from plant sources. Resveratrol is found in at least 72 species of plants distributed among 31 genera and 12 families (see, Counet, C. et al. (2006) “Chocolate And Cocoa: New Sources Of Trans-Resveratrol And Trans-Piceid,” Food Chem. 98:649-657; Jang, M. et al. (1997) “Cancer Chemopreventive Activity Of Resveratrol, A Natural Product Derived From Grapes,” Science 275:218-220; Wang, Y. et al. (2002) “An LC-MS Method For Analyzing Total Resveratrol In Grape Juice, Cranberry Juice, And In Wine,” J. Agricult. Food Chem. 50(3):431-435). All of the families found to contain resveratrol belong to the spermatophytes division: Vitaceae, Myrtaceae, Dipterocarpaceae, Cyperaceae, Gnetaceae, Leguminosae, Pinaceae, Moraceae, Fagaceae, Liliaceae (Langcake, P. et al. (1976) “The Production Of Resveratrol By Vitis Vinifera And Other Members Of The Vitaceae As A Response To Infection Or Injury,” Physiol. Plant Pathol. 9:77-86; Yoshiaki, T. et al. (2002) “Biogenic Reactions On Stilbenetetramers From Vitaceaeous Plants,” Tetrahedron 58:9265-9271). Resveratrol has most often been reported in nonedible plants: vine, eucalyptus, spruce, and the tropical deciduous tree Bauhinia racemosa, Pterolobium Hexapetallum (Cassady, A. et al. (2000) “Isoflavones, Lignans, And Stilbenes-Origins, Metabolism And Potential Importance To Human Health,” J. Science Food Agric. 80:1044-1062; Soleas, G. J. et al. (1997) “Resveratrol: A Molecule Whose Time Has Come? And Gone?,” Clin. Biochem. 30:91-113). Reseveratrol is particularly found in grape skins and Giant Knotweed (see, Burns, J. et al. (2002) “Plant Foods and Herbal Sources of Resveratrol,” J. Agric. Food Chem. 50(11):3337-3340), cocoa and chocolate (Counet, C. et al. (2006) “Chocolate And Cocoa: New Sources Of Trans-Resveratrol And Trans-Piceid,” Food Chem. 98:649-657). Peanut sprouts are also a rich source of resveratrol.

B. Modulation of Gene Product Concentration or Activity

The invention pertains to compositions that, upon administration to a recipient, increase the concentration or activity of a survival/longevity gene product and/or decrease the concentration or activity of a gene product that induces or causes cellular damage. As used herein, such increase (or decrease) in concentration or activity may be accomplished by any mechanism. For example, such increase (or decrease) may reflect a modulation of gene expression resulting in either increased (or decreased) expression of the gene encoding the survival/longevity gene product, or a gene that regulates (e.g., induces or represses) or whose product regulates such expression or activity. Alternatively, or conjunctively, such increase (or decrease) in concentration or activity may reflect a modulation of the recipient's ability to degrade or stabilize any such gene products. Alternatively, or conjunctively, such increase (or decrease) in concentration or activity may reflect a modulation of the recipient's ability to enhance, accelerate, repress or decelerate the activity of any such gene products.

The modulation of concentration or activity discussed above may be a modulation of intracellular, intercellular and/or tissue concentration or activity of such survival/longevity gene products or such gene products that induce or cause cellular damage. Such modulation may be identified by assays of DNA expression, assays of gene product activity, assays of the level of gene product, assays of the rate of gene product turnover, etc. conducted in one or more types of cells, tissues, etc.

An increase in the concentration of a survival/longevity gene product may result from, for example, increased transcription of the gene that encodes the survival/longevity gene product, increased transcription of a gene that induces the expression of the gene that encodes the survival/longevity gene product, decreased transcription of a gene that represses the expression of the gene that encodes the survival/longevity gene product, decreased degradation or enhanced stabilization of expressed molecules of the survival/longevity gene product (leading to the enhanced accumulation of the survival/longevity gene product). Similarly, a decrease in the concentration of a survival/longevity gene product may result from, for example, decreased transcription of the gene that encodes the survival/longevity gene product, decreased transcription of a gene that induces the expression of the gene that encodes the survival/longevity gene product, increased transcription of a gene that represses the expression of the gene that encodes the survival/longevity gene product, increased degradation or decreased stabilization of expressed molecules of the survival/longevity gene product (leading to the enhanced dissipation of the survival/longevity gene product).

One aspect of the present invention thus relates to the use of resveratrol and resveratrol-containing compositions to modulate gene expression, and in particular, to modulate the expression of “survival/longevity” genes and/or “damage inducing” genes. As used herein, a compound is said to “modulate” gene expression if its administration results in a change in expression (relative to a control) of such genes of at least 10%. Modulation may involve an increase in expression (“up-regulation”) or it may involve a decrease in expression (“down-regulation”). The term up-regulate thus denotes an increase of expression of at least 10%, at least 20%, at least 50%, at least 2-fold, at least 5-fold, or most preferably at least 10-fold (relative to a control). The term down-regulate conversely denotes a decrease of expression of at least 10%, at least 20%, at least 50%, at least 2-fold, at least 5-fold, or most preferably at least 10-fold (relative to a control).

A second aspect of the present invention thus relates to the use of resveratrol and resveratrol-containing compositions to modulate the concentration or activity of expressed products of “survival/longevity” genes and/or “damage inducing” genes. As used herein, a compound is said to “modulate” the concentration or activity of such expressed products if its administration results in a change in an intracellular, intercellular or tissue concentration or activity (relative to a control) of such gene products of at least 10%. Modulation may, for example, involve an “enhanced accumulation” or an “enhanced activity” or, for example, it may involve a “diminished accumulation” or a “diminished activity.” The term “enhanced accumulation” (or “enhanced activity”) denotes an increase in concentration (or activity) of at least 10%, at least 20%, at least 50%, at least 2-fold, at least 5-fold, or most preferably at least 10-fold (relative to a control). The term “diminished accumulation” or “diminished activity.” conversely denotes a decrease in concentration (or activity) of at least 10%, at least 20%, at least 50%, at least 2-fold, at least 5-fold, or most preferably at least 10-fold (relative to a control).

As used herein, a “survival/longevity” gene is a gene whose expression contributes to an increase in the survival or longevity of a subject (e.g., a mammal, and particularly a human) expressing such gene. Conversely, a “damage inducing” gene is a gene whose expression contributes to DNA, cellular, or tissue damage in such subject. Such genes are responders to biological stressors, they initiate action in response to stressors such as radiation (e.g., sunlight, gamma rays, UV light, etc.), radiomimetic agents (e.g., vitamin D), heat, near starvation (calorie restriction, or its mimetic, resveratrol) by modulating their expression.

Examples of survival/longevity genes are provided in Table 1. Examples of genes whose expression enhances cellular damage are provided in Table 2. These Tables provide the gene's NCBI “ENTREZGENE” accession number. Most preferably, such genes are human genes. The Sirtuin 1 gene is known to control the rate of aging of living organisms by virtue of its ability to produce DNA repair enzymes and mimics the beneficial effects of calorie restriction. The trans form of resveratrol (but not cis-resveratrol) activates the Sirtuin 1 gene (Alcendor, R. R. (2007) “Sirt1 Regulates Aging And Resistance To Oxidative Stress In The Heart,” Circulation Research 100(10):1512-1521; Howitz, K. T. et al. (2003) “Small Molecule Activators Of Sirtuins Extend Saccharomyces Cerevisiae Lifespan,” Nature 425:191-196). The invention particularly pertains to compositions that increase the concentration of the Sirtuin 1 survival/longevity gene product. The invention further particularly pertains to compositions that increase the concentration of the forkhead Foxo1 (daf-16, dFoxO) transcription factor survival/longevity gene product.

TABLE 1
Exemplary Survival/Longevity Genes

39329
39340
0610007C21Rik
0610007L01Rik
0610010F05Rik
0610037L13Rik
0610037P05Rik
0610040B10Rik
0610042E11Rik
1110001A07Rik
1110002B05Rik
1110003O08Rik
1110005A03Rik
1110007L15Rik
1110007M04Rik
1110008F13Rik
1110008J03Rik
1110008P14Rik
1110014K08Rik
1110018J18Rik
1110019J04Rik
1110020G09Rik
1110028A07Rik
1110028C15Rik
1110032E23Rik
1110033M05Rik
1110036O03Rik
1110038B12Rik
1110038D17Rik
1110054O05Rik
1110058L19Rik
1110059E24Rik
1110059G10Rik
1110067D22Rik
1190017O12Rik
1300010M03Rik
1300012G16Rik
1500002I01Rik
1500002O20Rik
1500005K14Rik
1500011B03Rik
1500011K16Rik
1500031L02Rik
1500034J01Rik
1600012F09Rik
1600015H20Rik
1600027N09Rik
1700001O22Rik
1700011B04Rik
1700017H01Rik
1700020C11Rik
1700021C14Rik
1700021F05Rik
1700023D09Rik
1700029F09Rik
1700029M20Rik
1700030K09Rik
1700040L02Rik
1700051A21Rik
1700113I22Rik
1700127D06Rik
1810007M14Rik
1810011O10Rik
1810012P15Rik
1810013L24Rik
1810015C04Rik
1810020D17Rik
1810021J13Rik
1810022K09Rik
1810026B05Rik
1810029B16Rik
1810030N24Rik
1810034K20Rik
1810035L17Rik
1810044A24Rik
1810049H13Rik
1810058I24Rik
1810059G22Rik
1810063B05Rik
1810073N04Rik
2010106G01Rik
2010109N14Rik
2010111I01Rik
2010200O16Rik
2010305A19Rik
2010309E21Rik
2010315B03Rik
2010320M18Rik
2010321M09Rik
2210010L05Rik
2210020M01Rik
2210408I21Rik
2310001A20Rik
2310002L09Rik
2310007O11Rik
2310011J03Rik
2310014D11Rik
2310014F07Rik
2310016C16Rik
2310026E23Rik
2310030G06Rik
2310033F14Rik
2310036O22Rik
2310038H17Rik
2310042E22Rik
2310043N10Rik
2310044H10Rik
2310046A06Rik
2310047A01Rik
2310047H23Rik
2310047M10Rik
2310061J03Rik
2310067B10Rik
2310076G13Rik
2410001C21Rik
2410002O22Rik
2410003K15Rik
2410004B18Rik
2410005O16Rik
2410012H22Rik
2410017P07Rik
2410017P09Rik
2410018C17Rik
2410018C20Rik
2410019A14Rik
2410022L05Rik
2410042D21Rik
2510003E04Rik
2510042H12Rik
2610001J05Rik
2610008E11Rik
2610019F03Rik
2610024B07Rik
2610028D06Rik
2610029I01Rik
2610030H06Rik
2610101N10Rik
2610200G18Rik
2610209M04Rik
2610301F02Rik
2610507B11Rik
2610528E23Rik
2700029M09Rik
2700038N03Rik
2700097O09Rik
2810004N23Rik
2810008M24Rik
2810410M20Rik
2810422O20Rik
2810423A18Rik
2810430I11Rik
2810455D13Rik
2900002H16Rik
2900006B11Rik
2900008C10Rik
2900011G08Rik
2900024O10Rik
3010003L21Rik
3010027C24Rik
3110003A17Rik
3110031B13Rik
3110043O21Rik
3110073H01Rik
3110080E11Rik
3110082I17Rik
3222402P14Rik
3321401G04Rik
4432414F05Rik
4631424J17Rik
4632404M16Rik
4632411B12Rik
4732416N19Rik
4732418C07Rik
4832420A03Rik
4833408C14Rik
4833439L19Rik
4921506J03Rik
4921509O07Rik
4921513H07Rik
4921517N04Rik
4930402E16Rik
4930426L09Rik
4930429B21Rik
4930432L08Rik
4930432O21Rik
4930448F12Rik
4930453O09Rik
4930455C21Rik
4930466F19Rik
4930486A15Rik
4930505O20Rik
4930513N20Rik
4930523C07Rik
4930524O07Rik
4930544L04Rik
4930551A22Rik
4930554H23Rik
4930557J02Rik
4930570C03Rik
4930570E01Rik
4930573O21Rik
4930579G24Rik
4932442K08Rik
4933402C05Rik
4933403F05Rik
4933404K13Rik
4933407I18Rik
4933411K20Rik
4933413C19Rik
4933421A08Rik
4933426M11Rik
4933428L01Rik
4933429D07Rik
4933433P14Rik
4933434E20Rik
4933440H19Rik
5033414K04Rik
5033421C21Rik
5033423K11Rik
5033430J17Rik
5330423I11Rik
5330439A09Rik
5430402E10Rik
5430402P08Rik
5430407P10Rik
5730470L24Rik
5730507A11Rik
5730536A07Rik
5730601F06Rik
5830404H04Rik
5830415L20Rik
5830428H23Rik
5830432E09Rik
5830436I19Rik
5830457O10Rik
5830469G19Rik
5830487K18Rik
5930434B04Rik
6230429P13Rik
6330403M23Rik
6330407G11Rik
6330409N04Rik
6330415G19Rik
6330417G04Rik
6330503C03Rik
6330564D18Rik
6330569M22Rik
6430548M08Rik
6530404N21Rik
6530413G14Rik
6620401M08Rik
6720462K09Rik
6720475J19Rik
6820401H01Rik
7030402D04Rik
7030407E18Rik
7420416P09Rik
8030463A06Rik
8030475D13Rik
8430436O14Rik
9030411M15Rik
9030418K01Rik
9030425P06Rik
9130011J15Rik
9230110F11Rik
9230114K14Rik
9330109K16Rik
9330120H11Rik
9430010O03Rik
9430013L17Rik
9530018H14Rik
9530018I07Rik
9530097N15Rik
9930024M15Rik
A030007L17Rik
A230046K03Rik
A230051G13Rik
A230062G08Rik
A230067G21Rik
A230091C14Rik
A330043J11Rik
A330076H08Rik
A430005L14Rik
A430102J17Rik
A430110N23Rik
A530082C11Rik
A730008L03Rik
A830018L16Rik
A930001N09Rik
A930006D11Rik
A930018M24Rik
A930026I22Rik
Ahcyl1
Amd1
Ank
Arhgap18
Arhgap20
Arhgap24
Arhgap29
Arhgap4
Arhgap5
Arhgap9
Arhgdia
Arhgef1
Arhgef12
Arhgef17
Arhgef2
B230117O15Rik
B230118H07Rik
B230219D22Rik
B230312A22Rik
B230337E12Rik
B230380D07Rik
B3galnt2
B630005N14Rik
B830007D08Rik
B830028B13Rik
B930093H17Rik
C030002C11Rik
C030007I01Rik
C030044B11Rik
C030046I01Rik
C130057M05Rik
C130065N10Rik
C230091D08Rik
C430003N24Rik
C730025P13Rik
Cdc73
Col10a1
Col19a1
Col1a1
Col1a2
Col23a1
Col27a1
Col4a3bp
Col5a1
Col6a2
D030011O10Rik
D030051N19Rik
D230019N24Rik
D330001F17Rik
D330017J20Rik
D430015B01Rik
D430018E03Rik
D530037H12Rik
D630023B12Rik
D830046C22Rik
D930017J03Rik
D930020B18Rik
E030018N11Rik
E130014J05Rik
E130303B06Rik
E330021D16Rik
E430010N07Rik
E430018J23Rik
EG226654
EG622645
EG633640
ENSMUSG00000050599
ENSMUSG00000071543
ENSMUSG00000074466
ENSMUSG00000074670
ENSMUSG00000075401
Exdl1
G3bp1
Galnt3
Galnt4
Galntl4
Gart
Kcna5
Kcna7
Kcng2
Kcnj3
Kcnj5
Kcnk3
Kcnv2
Kctd10
Kctd2
Kctd7
LOC100044376
LOC100044968
LOC100045002
LOC100045020
LOC100045522
LOC100045629
LOC100046086
LOC100046343
LOC100046855
LOC100047028
LOC100047385
LOC100047539
LOC100047601
LOC100047794
LOC100047915
LOC100048376
LOC100048397
LOC100048439
LOC100048863
LOC640441
LOC668206
LOC675709
LOC677447
Mtm1
OTTMUSG00000001305
OTTMUSG00000016644
P2ry5
P2ry6
Pabpc3
Pah
Paics
Paip1
Paip2
Palm
Papd1
Papd5
Papola
Papolg
Paqr7
Paqr9
Pard3
Pard6g
Parp12
Pbef1
Pbld
Pbrm1
Pcbp2
Pcca
Pcdh7
Pcdh9
Pcgf3
Pcgf6
Pcm1
Pcmt1
Pcnt
Pcnx
Pcp4
Pcp4l1
Pcsk7
Pctk1
Pctk2
Pctk3
Pdcd10
Pdcd4
Pdcd6
Pdcl
Pdcl3
Pde1a
Pde2a
Pde4dip
Pde6a
Pde7a
Pdgfa
Pdha1
Pdia3
Pdia6
Pdk4
Pdlim4
Pdlim5
Pds5b
Pdss1
Pdxk
Pdzd11
Pecam1
Pef1
Per1
Per3
Perp
Pex11c
Pex12
Pex19
Pex5
Pex6
Pex7
Pfdn1
Pfdn4
Pfdn5
Pfkp
Pfn2
Pgam2
Pgbd5
Pggt1b
Pgr
Phc2
Phc3
Phf14
Phf17
Phf20l1
Phf3
Phf6
Phka2
Phkb
Phkg1
Phlda1
Phldb1
Phpt1
Phyh
Pias4
Picalm
Pigz
Pik3ca
Pik3ip1
Pip5k1c
Pir
Pitpna
Pitpnb
Pitpnc1
Pitpnm1
Pkd1
Pkia
Pkm2
Pkp2
Pla2g10
Pla2g2d
Pla2g5
Plcd1
Plce1
Pld3
Pldn
Plec1
Plekhh3
Plekhj1
Plekhm2
Plekhn1
Plod3
Plp2
Pls3
Pltp
Plvap
Plxnb2
Pmm1
Pno1
Pnpla1
Pnpla2
Pnpla6
Pnrc2
Podn
Poldip3
Polg2
Polr2d
Polr2f
Polr2h
Polr2i
Polr2k
Polr3gl
Polr3k
Pot1a
Pou6f1
Ppap2b
Ppard
Pparg
Ppargc1a
Pphln1
Ppic
Ppif
Ppig
Ppil2
Ppl
Ppm1f
Ppme1
Ppp1ca
Ppp1r11
Ppp1r12a
Ppp1r12c
Ppp1r13l
Ppp1r2
Ppp1r3c
Ppp2ca
Ppp2r2d
Ppp2r3c
Ppp2r5c
Ppp4r1l
Ppp5c
Ppp6c
Ppt2
Pqlc1
Prdm4
Prdm5
Prdx2
Prdx3
Preb
Prei4
Prkab2
Prkaca
Prkcbp1
Prkcdbp
Prkch
Prkcn
Prkcsh
Prkcz
Prkrir
Prlr
Prmt7
Prodh
Prosc
Prpf6
Prpsap1
Prr12
Prrc1
Prss12
Prune
Psap
Pscd1
Psd3
Psenen
Pskh1
Psma2
Psma5
Psma6
Psma8
Psmb7
Psmd11
Psmd12
Psmd4
Psmd6
Psmd8
Pstk
Ptdss2
Ptgfrn
Ptms
Ptp4a3
Ptpla
Ptpn1
Ptpn11
Ptpn12
Ptpn20
Ptpn3
Ptpra
Ptprg
Ptprs
Pttg1
Puf60
Pum1
Pus1
Pxmp3
Pxn
Qk
Rab1
Rab11a
Rab11b
Rab2
Rab20
Rab21
Rab24
Rab30
Rab33b
Rab35
Rab3a
Rab3gap1
Rab3gap2
Rab3il1
Rab43
Rab6
Rab8b
Rabep1
Rabgap1l
Rac1
Rad17
Rad23b
Rad54l2
Rag1ap1
Ralgps2
Ramp2
Ranbp10
Ranbp2
Rap1a
Rap1gap
Rap2a
Rap2b
Raph1
Rara
Rarb
Rarg
Rasa1
Rasa3
Rasl2-9
Rassf7
Rb1cc1
Rbbp6
Rbj
Rbm12
Rbm20
Rbm24
Rbm27
Rbm28
Rbm38
Rbm39
Rbms1
Rbms2
Rbmxrt
Rbpms
Rcan2
Rcl1
Rdh13
Reep5
Rem2
Retsat
Rev1
Rfc2
Rfc4
Rfesd
Rfng
Rfwd3
Rfx1
Rgl1
Rgma
Rgs12
Rgs5
Rhbdd2
Rhbdd3
Rhbdf1
Rhd
Rhobtb1
Rhobtb2
Rhoq
Ric8
Ring1
Rlbp1
Rmnd1
Rmnd5b
Rnaset2a
Rnd1
Rnf11
Rnf13
Rnf139
Rnf14
Rnf149
Rnf167
Rnf168
Rnf187
Rnf2
Rnf31
Rnf34
Rnf5
Rnf6
Rock1
Rorc
RP23-136K12.4
rp9
Rpap2
Rpe
Rpl15
Rpl27a
Rpl37
Rpl39
Rpl3l
Rpl7l1
Rpl8
Rplp2
Rpo1-3
Rpo1-4
Rpp30
Rprml
Rps11
Rps26
Rps6
Rps6ka1
Rps6ka4
Rrad
Rragc
Rragd
Rras2
Rrbp1
Rrp1
Rrp9
Rsad1
Rspry1
Rtp3
Rufy1
Rufy3
Rusc1
Rwdd1
Rxrb
Rxrg
Ryk
Ryr2
S3-12
Sae2
Safb
Samd5
Samd8
Samd9l
Saps3
Sar1a
Sars
Sat1
Satb2
Sbds
Sbf2
Sbk1
Sc4mol
Scamp3
Scap
Scara5
Scarb1
Scarb2
Sccpdh
Scfd1
Schip1
Scmh1
Scn4b
Scoc
Scube2
Scyl1
Scyl3
Sdcbp
Sdccag10
Sdha
Sdhd
Sds
Sec11a
Sec14l1
Sec22b
Sec23a
Sec31a
Sec61a1
Sec61a2
Sele
Sema3b
Sephs2
Sepp1
Serbp1
Serinc1
Serpinb6a
Serpinb9
Sertad2
Set
Setd7
Setd8
Setx
Sf3a1
Sf3b1
Sf3b2
Sfrp5
Sfrs1
Sfrs10
Sfrs2ip
Sfrs7
Sfrs9
Sfxn3
Sgca
Sgcg
Sgk2
Sgta
Sh2d3c
Sh2d4a
Sh3bgrl
Sh3bp5l
Sh3d19
Sh3kbp1
Shb
Shmt2
Shroom3
Sirt1
Skil
Skiv2l2
Slain2
Slc10a1
Slc12a4
Slc12a5
Slc16a1
Slc16a4
Slc1a5
Slc1a6
Slc20a1
Slc22a17
Slc22a5
Slc25a11
Slc25a12
Slc25a17
Slc25a22
Slc25a28
Slc25a3
Slc25a32
Slc25a33
Slc25a34
Slc25a36
Slc25a4
Slc25a42
Slc25a46
Slc26a11
Slc27a1
Slc29a1
Slc31a1
Slc35a2
Slc35a3
Slc35b1
Slc35b2
Slc36a2
Slc39a1
Slc39a10
Slc39a8
Slc40a1
Slc44a1
Slc47a1
Slc4a2
Slc4a4
Slc4a7
Slc6a19
Slc6a6
Slc6a9
Slc7a1
Slc7a4
Slc7a7
Slc9a1
Slco1a4
Slco3a1
Slco5a1
Slit3
Slmap
Slmo2
Smarca2
Smarcc2
Smarcd3
Smchd1
Smcr7
Smn1
Smndc1
Smoc2
Smpd1
Smpdl3a
Smtn
Smtnl2
Smu1
Smurf1
Smyd1
Smyd4
Snapap
Snapc1
Snf1lk2
Snora65
Snrk
Snrp70
Snrpb2
Snrpd3
Snx12
Snx13
Snx16
Socs3
Socs4
Sorbs1
Sorcs2
Sort1
Sost
Sox17
Sox4
Sox9
Sp3
Spag9
Spcs1
Speer7-ps1
Spg3a
Spg7
Spin1
Spink10
Spna2
Spnb1
Spnb2
Spop
Spry2
Sqstm1
Srd5a2l2
Srebf1
Srebf2
Sri
Srl
Srp19
Srpr
Ssbp3
Ssbp4
Ssh1
Ssr3
Sstr5
Ssu72
St13
St3gal6
St6galnac6
St7
St8sia2
St8sia4
Stab1
Stard10
Stat6
Stbd1
Stch
Stip1
Stk11
Stk19
Stk38
Stk39
Stom
Strn3
Stx6
Stxbp2
Styx
Suhw3
Sulf2
Supt5h
Supv3l1
Surf4
Svep1
Sybl1
Syk
Syn2
Syngr2
Synj2bp
Synpo
Sypl
Taf2
Taf6
Tanc1
Taok2
Taok3
Tap1
Tap2
Tapt1
Tardbp
Tatdn3
Tbc1d10b
Tbc1d15
Tbc1d19
Tbc1d20
Tbc1d2b
Tbc1d5
Tbc1d7
Tbcb
Tbcc
Tbce
Tbcel
Tbkbp1
Tbpl1
Tbx19
Tbx20
Tcap
Tcea1
Tcea3
Tcf15
Tcf20
Tcf25
Tcfe2a
Tcof1
Tcp1
Tcp11l2
Tcta
Tead4
Tef
Tesk1
Tex2
Tex261
Tfam
Tfb2m
Tfpi
Tfrc
Tgds
Tgfbr1
Thbs4
Thnsl2
Thoc1
Thoc4
Thrb
Tie1
Tigd2
Timm22
Timm50
Timp3
Timp4
Tinagl
Tjap1
Tk2
Tle6
Tlk2
Tln1
Tloc1
Tm2d1
Tm2d2
Tm2d3
Tm4sf1
Tm6sf2
Tm9sf2
Tm9sf3
Tmc1
Tmcc1
Tmcc3
Tmco1
Tmed7
Tmem103
Tmem109
Tmem110
Tmem112b
Tmem115
Tmem119
Tmem123
Tmem126b
Tmem132a
Tmem142a
Tmem142c
Tmem147
Tmem14c
Tmem157
Tmem159
Tmem167
Tmem168
Tmem16f
Tmem176a
Tmem176b
Tmem182
Tmem188
Tmem19
Tmem30a
Tmem37
Tmem38a
Tmem38b
Tmem41a
Tmem41b
Tmem46
Tmem50a
Tmem55b
Tmem57
Tmem64
Tmem69
Tmem70
Tmem77
Tmem85
Tmem86a
Tmem93
Tmem9b
Tmlhe
Tmod1
Tmod4
Tmub1
Tmub2
Tnfaip1
Tnfaip8l1
Tnfrsf11a
Tnfrsf18
Tnfrsf1a
Tnfsf5ip1
Tnip1
Tnks1bp1
Tnni3
Tnni3k
Tnnt2
Tnpo1
Tnpo2
Tnrc6a
Tns4
Tnxb
Toe1
Tollip
Tomm22
Tomm34
Tomm40
Tomm70a
Top1
Top2b
Topors
Tor1aip2
Tpcn1
Tpm1
Tpm3
Tpm4
Tpp1
Tpp2
Tppp3
Tpr
Tprkb
Tpst1
Traf3ip2
Traip
Trak2
Trappc2
Trappc2l
Trem3
Trex1
Trim11
Trim12
Trim23
Trim26
Trim29
Trim3
Triobp
Trip4
Trmt11
Tro
Troap
Trpc1
Trpc4ap
Trpm4
Tsc22d1
Tsc22d4
Tsfm
Tsga10
Tsnax
Tspan13
Tspan18
Tspan4
Tspan7
Tssc4
Tsta3
Ttc1
Ttc28
Ttc32
Ttc33
Ttc35
Ttc9c
Tub
Tuba4a
Tuba8
Tubb2c
Tubb5
Tufm
Tug1
Tulp4
Twsg1
Txlna
Txlnb
Txndc1
Txndc10
Txndc12
Txndc14
Txndc4
Txnip
Txnl1
Txnl4
Txnl4b
Tyk2
Uaca
Ubac1
Ubap1
Ubash3a
Ubd
Ube1c
Ube1l2
Ube1x
Ube2b
Ube2d2
Ube2d3
Ube2e1
Ube2f
Ube2h
Ube2n
Ube2o
Ube2q2
Ube2v1
Ube2v2
Ube2w
Ube3a
Ubl4
Ubl7
Ublcp1
Ubtf
Ubxd7
Uchl5
Ucp2
Ucp3
Ufm1
Ugcgl2
Ugp2
Umps
Ung
Unk
Uqcc
Uqcrc1
Uqcrfs1
Usp11
Usp19
Usp2
Usp21
Usp22
Usp34
Usp36
Usp45
Usp47
Usp52
Usp54
Usp9y
Utp6
Utrn
Uvrag
Uxt
V1ra5
Vamp4
Vasn
Vbp1
Vdac1
Vdac2
Vdac3
Vdp
Vegfa
Vegfb
Vegfc
Vezf1
Vkorc1l1
Vldlr
Vps16
Vps18
Vps29
Vps35
Vps36
Vps37b
Vps4b
Vps54
Vwf
Wac
Wapal
Was
Wbp4
Wdfy1
Wdr13
Wdr21
Wdr22
Wdr23
Wdr3
Wdr47
Wdr5b
Wdr92
Wdsof1
Wfdc3
Wipi2
Wnk1
Wtap
Wwp2
Xbp1
Xdh
Xlr5a
Xpnpep1
Xpr1
Xrcc1
Xrcc6
Yap1
Yeats2
Yif1a
Yipf3
Yipf4
Yipf7
Ypel2
Ypel3
Ywhaq
Zadh1
Zbed3
Zbtb43
Zbtb5
Zc3h11a
Zc3h12c
Zc3h15
Zc3h6
Zc3h8
Zcchc6
Zdhhc13
Zdhhc3
Zeb1
Zfand5
Zfml
Zfp106
Zfp110
Zfp187
Zfp191
Zfp213
Zfp236
Zfp238
Zfp26
Zfp260
Zfp277
Zfp289
Zfp30
Zfp313
Zfp319
Zfp322a
Zfp335
Zfp341
Zfp35
Zfp383
Zfp384
Zfp414
Zfp422
Zfp422-rs1
Zfp512
Zfp516
Zfp560
Zfp568
Zfp579
Zfp597
Zfp608
Zfp628
Zfp629
Zfp639
Zfp644
Zfp650
Zfp651
Zfp667
Zfp672
Zfp68
Zfp703
Zfp715
Zfp719
Zfp740
Zfp758
Zfp817
Zfp82
Zfyve21
Zhx2
Zhx3
Zic2
Zkscan17
Zmat2
Zmat5
Zmym4
Zmynd10
Znrf1
Zrsr1
Zscan12
Zswim6
Zyg11b
Zyx
Zzef1

TABLE 2
Exemplary Genes Whose Expression Enhances Cellular Damage

AA407175
AA415038
AA987161
Aadacl1
Aars
Aasdhppt
AB182283
Abca4
Abca7
Abcb4
Abcb7
Abcd1
Abce1
Abhd1
Abhd12
Abhd4
Abi1
Abi2
Ablim2
Abra
Abtb1
Acaa2
Acad11
Acad9
Acadl
Acads
Acadvl
Acbd3
Acbd5
Acbd6
Ace
Aco2
Acot5
Acox3
Acsl1
Acss2
Acta1
Actb
Actn1
Actn2
Actn4
Actr1b
Actr2
Acvr1b
Acvr2a
Acvrl1
Acyp1
Acyp2
Adal
Adam10
Adam15
Adam21
Adamts10
Adamts2
Adamts7
Adamts9
Adar
Adcy1
Adcy2
Adcy3
Adcy6
Add1
Adh5
Adra1b
Adrbk1
Aebp1
Aes
Afap1l1
Aff4
Afg3l1
Aga
Agbl5
Agpat1
Agpat5
Agrn
Agtr1a
Agxt2l2
Ahdc1
Ahr
Ahsa1
AI118078
AI225934
AI413194
AI428479
AI429363
AI462493
AI480535
AI506816
AI597468
AI662270
AI662476
AI747699
AI790298
AI837181
AI848100
AI852064
AI987944
Ak7
Akap13
Akap2
Akp2
Akr1a4
Akr1b8
Akr7a5
Akt1s1
Alas1
Aldh1a3
Aldh2
Aldh4a1
Aldh7a1
Aldh9a1
Alg12
Alg13
Alg5
Alkbh6
Alkbh8
Als2cr2
Anapc10
Anapc2
Angptl2
Ank1
Ankhd1
Ankrd1
Ankrd10
Ankrd13a
Ankrd13c
Ankrd13d
Ankrd25
Ankrd28
Ankrd32
Ankrd37
Ankrd38
Ankrd9
Anp32b
Anxa3
Anxa6
Aoc3
Ap1s2
Ap2a2
Ap2b1
Ap2m1
Ap4m1
Ap4s1
Apbb1
Aplp2
Apobec2
Apod
Apoe
Apool
Appl1
Appl2
Arf1
Arf3
Arg2
Arid4b
Arl1
Arl2bp
Arl3
Arl4a
Arl5b
Arpc1a
Arpc1b
Arpc2
Arpc4
Arrb1
Art5
Asb1
Asb14
Asb5
Ascc3l1
Asnsd1
Asph
Atad2b
Atf3
Atg10
Atg3
Atg4d
Atg5
Atp11b
Atp13a1
Atp1a2
Atp5h
Atp5s
Atp6ap2
Atp6v0a2
Atp6v0d1
Atp6v1b2
Atp6v1f
Atp9a
Atp9b
Atpaf1
Atpbd1c
Atpif1
Atr
Atxn2
Atxn7l1
AU020772
AU041133
Aup1
AV009015
AV024533
AV025504
Avpr1a
AW046287
AW112010
AW209491
AW555464
AW556556
AW742931
Azi2
Azin1
Bach1
Bag4
Bambi
Banp
Bat1a
Bat2
Baz1a
Baz1b
BB217526
Bbc3
Bbs10
BC003331
BC003885
BC003965
BC010304
BC010981
BC011248
BC013529
BC016495
BC019943
BC020077
BC021395
BC023882
BC024659
BC024814
BC025076
BC028440
BC028528
BC030183
BC030308
BC030336
BC031353
BC031781
BC032203
BC034069
BC037034
BC037112
BC038479
BC039210
BC043098
BC043476
BC048679
BC049349
BC057893
Bcam
Bcat2
Bckdha
Bckdk
Bcl2l13
Bcl6b
Bclaf1
Bdp1
Bet1
Bgn
Bhlhb2
Bhlhb3
Bicd2
Birc4
Blvra
Bmi1
Bmp6
Bmpr1a
Bnip3
Brd3
Btaf1
Btbd14b
Btbd2
Btbd3
Btbd6
Btf3l4
Btnl9
Bxdc2
C130094E24
C2
C77058
C78441
C78651
C79741
C86942
C87259
Cab39
Cabin1
Cacna1g
Cacna1h
Cacybp
Cadm4
Calm1
Calr
Calr3
Caml
Camsap1
Cand2
Canx
Capg
Capn1
Capns1
Caprin1
Card10
Caskin2
Casp8ap2
Casp9
Casq1
Cav1
Cav2
Cbfb
Cblb
Cbr1
Cbx3
Ccar1
Ccdc12
Ccdc122
Ccdc125
Ccdc127
Ccdc3
Ccdc34
Ccdc47
Ccdc58
Ccdc69
Ccdc7
Ccdc72
Ccdc85b
Ccdc88a
Ccdc90a
Ccdc90b
Ccl9
Ccm2
Ccnd3
Ccng1
Ccnh
Ccni
Ccnl2
Ccnt2
Ccr1
Ccr1l1
Ccr5
Ccs
Cct5
Cct7
Cd151
Cd163
Cd200
Cd207
Cd36
Cd38
Cd74
Cd83
Cd93
Cd97
Cdadc1
Cdc27
Cdc2l5
Cdc2l6
Cdc37
Cdc42ep3
Cdgap
Cdh13
Cdipt
Cdk5rap3
Cdk7
Cdv3
Cebpz
Cenpa
Cenpq
Centa1
Centa2
Centb2
Centd1
Centd2
Centg2
Cetn3
Cfl1
Cflar
Cgnl1
Cgrrf1
Chac1
Chac2
Chchd4
Chd1
Chd2
Chd4
Chmp1b
Chmp2b
Chordc1
Chrac1
Chrd
Chrng
Chst14
Chuk
Churc1
Ciao1
Cib1
Cic
Cilp2
Cisd2
Cish
Ckm
Ckmt2
Clcn5
Cldnd1
Clec2d
Clic1
Clic4
Clint1
Clk3
Cln5
Clock
Clptm1
Clstn1
Cltc
Cmpk
Cmya5
Cndp2
Cnot6l
Cnot7
Cntfr
Cntn4
Commd1
Commd3
Commd4
Commd5
Comp
Cope
Copg
Cops2
Cops7a
Coq10b
Coq9
Coro1b
Cox11
Cox4i2
Cox5a
Cox8a
Cp
Cpeb4
Cpm
Cpsf1
Cpsf3
Cpt1a
Cpt1b
Cramp1l
Crat
Crbn
Creb1
Creb3l1
Crebbp
Crebzf
Creg1
Crip1
Crip2
Cript
Crnkl1
Crot
Cry1
Cryab
Crybb1
Cryz
Csdc2
Cse1l
Csf2ra
Csl
Csnk1a1
Csnk1d
Csnk2a1
Cst3
Cst8
Cstf2
Ctage5
Ctcf
Ctgf
Ctps
Ctsb
Ctsf
Ctss
Ctsz
Cttnbp2nl
Cul1
Cul3
Cxcl12
Cxcl14
Cxxc1
Cxxc5
Cyb561
Cyb5b
Cyb5r3
Cyb5r4
Cybasc3
Cyc1
Cyfip1
Cyp1b1
Cyp27a1
Cyp2f2
Cys1
D030063E12
D0H4S114
D10Ertd641e
D13Ertd787e
D14Ertd16e
D14Ertd581e
D15Ertd50e
D16H22S680E
D19Ertd721e
D19Ertd737e
D19Wsu162e
D1Bwg1363e
D2Ertd391e
D3Ertd254e
D3Wsu106e
D4Ertd429e
D4Ertd571e
D6Wsu176e
D8Ertd457e
D8Ertd54e
D8Ertd620e
D8Ertd82e
D9Ertd402e
Daam1
Dad1
Dap
Dapk2
Daxx
Dbh
Dcn
Dctn1
Dctn2
Dcun1d1
Dcun1d2
Dcun1d5
Ddah2
Ddb1
Ddb2
Ddit3
Ddr1
Ddr2
Ddx1
Ddx17
Ddx39
Ddx51
Ddx54
Ddx58
Ddx6
Deb1
Dedd
Defb1
Defb5
Defcr15
Depdc7
Derl2
Des
Dfna5h
Dgat2
Dgcr2
Dgka
Dgke
Dguok
Dhodh
Dhrs1
Dhrs7
Dhx30
Dhx32
Dhx34
Dhx8
Dhx9
Diablo
Diap1
Diras1
Dirc2
Dkk3
Dld
Dll4
Dlst
Dmd
Dmpk
Dmtf1
Dmwd
Dmxl2
Dnahc9
Dnaja3
Dnajb1
Dnajb4
Dnajb9
Dnajc12
Dnajc3a
Dnajc7
Dnm2
Dock11
Dock6
Dom3z
Dopey1
Dot1l
Dpagt1
Dph3
Dpp8
Dpp9
Dpysl2
Dpysl3
Dr1
Drg1
Dstn
Dtnbp1
Dus3l
Dusp1
Dusp6
Dusp8
Dvl2
Dync1h1
Dync1li2
Dyrk1a
E2f6
Eaf1
Eapp
Ears2
Ebag9
Ece1
Ecm1
Ecm2
Edaradd
Edg3
Eea1
Eef1a1
Eef1b2
Eef1e1
Eef2
Efcab2
Efemp2
Efnb3
Egf
Egfl7
Egflam
Egfr
Egln1
Egln3
Egr1
Ehbp1l1
Ehd4
Ei24
Eif1ay
Eif2ak1
Eif2s2
Eif3e
Eif4a1
Eif4a2
Eif4b
Eif4e2
Eif4ebp1
Eif4g3
Eif5
Eif5b
Elac2
Elf2
Elk3
Ell
Ell2
Elovl5
Elp3
Elp4
Eltd1
Emb
Emd
Eme2
Emg1
Emilin1
Eml2
Enc1
Eng
Eno3
Enpep
Enpp5
Entpd5
Entpd6
Ep300
Epb4.1l3
Epha4
Ephb1
Ephb4
Epm2aip1
Epn1
Eps15l1
Erc1
Ergic3
Erlin1
Ero1lb
Errfi1
Esco1
Esd
Esf1
Esrrg
Etfa
Etnk1
Ets2
Ewsr1
Exoc5
Exosc1
Exosc10
Exosc7
Exosc9
Ext1
Eya3
F11r
F13b
F5
Fads3
Fahd2a
Fam18b
Fancg
Fap
Fas
Fastkd1
Fastkd2
Fbln1
Fbln2
Fbp2
Fbxl2
Fbxl6
Fbxo3
Fbxo30
Fbxw4
Fbxw5
Fcer2a
Fcgr4
Fdx1
Fem1c
Fert2
Fgfr1op
Filip1
Fkbp10
Fkbp5
Fkbp8
Flcn
Flii
Flot1
Flot2
Flywch1
Fmn1
Fmo2
Fmr1
Fnbp1l
Fnip1
Foxa3
Foxj2
Foxk1
Foxk2
Foxo1
Foxp1
Frag1
Frap1
Frmd4b
Frmd5
Fscn1
Fth1
Ftl1
Fuca2
Fundc2
Furin
Fus
Fxc1
Fxyd1
Fxyd5
Fzd10
Fzd2
Fzd9
G0s2
G6pc2
Gaa
Gab1
Gabpa
Gadd45b
Gadd45g
Gale
Galk1
Gapdh
Gapvd1
Garnl1
Gas6
Gata4
Gba2
Gbas
Gbe1
Gbf1
Gcdh
Gdi1
Gdi2
Gdpd1
Gdpd5
Gemin5
Ggta1
Ghitm
Gimap4
Gimap8
Git1
Gja3
Gle1l
Glg1
Gli1
Glo1
Glod4
Gls
Gltscr2
Glud1
Glul
Gm104
Gm561
Gmeb1
Gmfb
Gmppa
Gna-rs1
Gnb2
Gnb4
Gne
Gng10
Gnl3
Gnpda1
Golga2
Golga7
Golgb1
Got1
Got2
Gpaa1
Gpam
Gpatch1
Gpbp1
Gpbp1l1
Gpc1
Gpc6
Gpd1l
Gper
Gpkow
Gpr115
Gpr137
Gpr175
Gpr22
Gpr4
Gpr98
Gpsn2
Gpt2
Gpx3
Gramd1a
Grb14
Grina
Grk1
Grk5
Grlf1
Grm8
Grn
Grpel2
Gsdmdc1
Gsn
Gsta4
Gstcd
Gstm1
Gstm2
Gstm5
Gstm7
Gstp1
Gstt1
Gtf2a1
Gtf2a2
Gtf2e1
Gtf2e2
Gtf2h3
Gtf2h4
Gtf3c1
Gtf3c4
Gtpbp1
Gtpbp2
Gulo
Gyg
Gyk
Gys1
H2afv
H2afy
H2-Bl
H2-Oa
H2-T24
H6pd
Hadh
Hadha
Hadhb
Hand2
Hars
Hars2
Hat1
Hax1
Hccs
Hcfc1r1
Hcfc2
Hdac2
Hdac4
Hdac7a
Hdhd2
Hdlbp
Heatr5b
Heatr6
Hectd1
Heph
Herpud1
Heyl
Hfe2
Hgs
Hhatl
Hiat1
Hiatl1
Hibadh
Hif1a
Higd1b
Hint3
Hirip3
Hivep2
Hk3
Hlf
Hmcn1
Hmg20b
Hmgb1
Hmgb3
Hmgcl
Hmgcs1
Hnrpab
Hnrph3
Hnrpk
Hnrpl
Hnrpll
Hnrpr
Hnrpul1
Hoxd11
Hrasls
Hrc
Hs2st1
Hsd17b11
Hsd17b13
Hsd17b4
Hsdl2
Hsp110
Hspa1b
Hspa5
Hspb2
Hspb3
Hspb6
Hspb7
Hspe1
Htra1
Htra3
Hus1
Hyal4
lah1
lbrdc2
ld1
Ide
Idh3a
Idh3b
Ifi30
Ifit3
Ifnar1
Ifnar2
Ifngr1
Ifngr2
Ift122
Ift57
Igfbp4
Igfbp6
Igsf11
Igsf3
Igsf8
Ihpk1
Ikbkap
Il10rb
Il13ra1
Il18bp
Il6st
Ilk
Ilvbl
Immp1l
Immp2l
Immt
Imp3
Impa2
Impad1
Ints8
Ipmk
Ipo13
Ipo7
Ipo8
Iqsec1
Iqwd1
Irf4
Irs1
Isca2
Isg20
Isyna1
Itfg3
Itgb1bp1
Itgb1bp2
Itgb1bp3
Itgb2
Itgb5
Itih3
Itk
Itm2b
Itm2c
Itpr3
Ivns1abp
Jam2
Jmjd1c
Jmjd2a
Jmjd6
Josd2
Jtv1
Jun
Kbtbd10
Kbtbd5
Kcnip2
Khk
Kif1b
Kif1c
Kif21a
Kif2a
Kif3a
Kif5b
Klc3
Klf11
Klf13
Klf15
Klf16
Klf4
Klf7
Klhdc1
Klhdc3
Klhl13
Klhl22
Klhl23
Klhl24
Klhl4
Klhl9
Klk1b24
Kpna1
Kpna4
Krr1
Kti12
Ktn1
L1cam
l7Rn6
Lace1
Lactb
Lama2
Lamb2
Laptm4a
Larp1
Larp2
Larp4
Larp5
Lcmt1
Lcmt2
Ldb1
Ldb3
Ldhb
Ldhd
Leo1
Lgals3bp
Lgals7
Lgmn
Lgr4
Lgr6
Lias
Limd1
Lims2
Lipe
Lix1l
Llgl1
Lmbrd1
Lmln
Lmna
Lmo4
Lmtk2
LOC100040515
LOC100043489
LOC100046468
LOC100046982
LOC552902
Lonp1
Lor
Lpgat1
Lphn1
Lrch1
Lrch4
Lrp10
Lrp2bp
Lrp6
Lrpap1
Lrrc1
Lrrc20
Lrrc39
Lrrc3b
Lrrc40
Lrrc44
Lsm14a
Lsm14b
Lsm3
Ltb4dh
Ltbp3
Ltbp4
Ly6a
Lypla1
Lypla2
Lyrm4
Lyrm5
Lysmd2
Lysmd3
Lztfl1
Lztr1
M6prbp1
Macf1
Macrod1
Maf1
Magea5
Magee1
Magi3
Mall
Man2b1
Maob
Map1lc3a
Map1lc3b
Map2k1ip1
Map2k2
Map3k1
Map3k12
Map3k2
Map3k7
Map3k7ip1
Map4k5
Mapbpip
Mapk14
Mapk6
Mapkapk2
Mapre1
Marcks
Mat2a
Mat2b
Matn4
Maz
Mbc2
Mbd2
Mbd3
Mbd5
Mboat5
Mbtps1
Mbtps2
Mcf2l
Mctp2
Mdfic
Mdh2
Med13
Med16
Med19
Med25
Med30
Med7
Mef2b
Mef2c
Mef2d
Megf11
Megf8
Mel13
Mertk
Mesdc2
Metrnl
Mett10d
Mex3c
Mfap4
Mfge8
Mfn1
Mfsd8
Mgam
Mgat1
Mgat4b
Mgp
Mgrn1
Mif4gd
Mkl1
Mknk1
Mlf1
Mlkl
Mll2
Mllt1
Mllt6
Mlx
Mlxip
Mlycd
Mme
Mmp15
Mmp1b
Mmp2
Mmrn2
Mobkl3
Mocos
Mocs2
Morc2a
Mosc2
Mospd1
Mpa2l
Mpp6
Mpv17
Mrfap1
Mrgprf
Mrpl1
Mrpl15
Mrpl17
Mrpl19
Mrpl28
Mrpl30
Mrpl32
Mrpl36
Mrpl38
Mrpl4
Mrpl41
Mrps17
Mrps18c
Mrps22
Mrps5
Mrs2l
Msl31
Msra
Msrb2
Msrb3
Msx1
Mtap4
Mtap7d1
Mtbp
Mtch2
Mterf
Mterfd1
Mterfd2
Mterfd3
Mtif3
Mtmr1
Mtmr3
Mtrr
Mustn1
Mxd4
Mxi1
Mxra8
Mybbp1a
Mybpc3
Myc
Mycbp
Myct1
Myd116
Myd88
Myef2
Myh14
Myh6
Myl4
Myl7
Mylip
Myo10
Myo1c
Myo9b
Myocd
Myom1
Mypn
N6amt1
N6amt2
Naca
Nagpa
Nars
Nat5
Nbeal1
Nckap1l
Ndst4
Ndufa5
Ndufab1
Ndufaf1
Ndufb3
Ndufb7
Ndufb8
Ndufc1
Ndufc2
Ndufs1
Ndufs2
Ndufs3
Ndufs5
Ndufs7
Ndufs8
Ndufv1
Ndufv2
Nedd4
Neil1
Nek3
Nf2
Nfu1
Ngly1
Ngrn
Nid1
Nif3l1
Ninj1
Nipbl
Nkiras1
Nkiras2
Nlgn2
Nmnat1
Npc1
Nppb
Nras
Nrd1
Nrp1
Nrp2
Nrtn
Nsmaf
Nt5c2
Nt5c3
Nub1
Nwd1
Oasl2
Ogg1
Oplah
Pfkfb2
Pfkfb4
Pgc1
Pgls
Pygb
Rars
Rars2
Rere
Rnpepl1
RP23-233B9.8
Rsrc2
Smad1
Smad3
Smad6
Ucp3
X83328
Xk
Xpo4
Xpo6
Xpot
Xrn1

C. Preferred Compositions of the Present Invention

The invention particularly pertains to resveratrol-containing compositions in which the specific activity of the resveratrol has been stabilized or enhanced. As used herein, the term “specific activity” refers to the ratio of the extent of gene modulation (relative to control) per amount (mass) of administered resveratrol.

Preferably, such compositions will comprise a chelator, hyaluronic acid, and/or vitamin D. The invention particularly pertains to such compositions that comprise resveratrol (preferably, the compositions of the present invention will provide a composition dosage of from about 10 mg to about 2 g, more preferably from about 100 mg to about 500 mg), and at least one compound selected from the group consisting of an antioxidant (chelator), hyaluronic acid, and vitamin D. Preferably, the compositions of the present invention will contain resveratrol, an antioxidant, hyaluronic acid, and vitamin D.

As used herein the term “chelator” refers to an organic compound that bonds with and removes free metal ions from solution. Examples of suitable chelators include ethylenediaminetetraacetic acid (EDTA), histidine, antibiotic drugs of the tetracycline family, pyridoxal 2-chlorobenzoyl hydrazone, desferrioxamine, dexrazoxane, deferasirox, pyoverdine, pseudan, citrate, NDGA (nordihydroguairetic acid: 1,4-bis[3,4-dihydroxyphenyl]2,3-dimethylbutane), ferulic acid and phytic acid. Preferably, the compositions of the present invention will provide a composition dosage of chelator of from about 1 g to about 15 g, more preferably from about 2 g to about 12 g.

Phytic acid is a particularly preferred chelator for the purposes of the present invention. As used herein, the term “phytic acid” refers to inositol hexaphosphate ((2,3,4,5,6-pentaphosphonooxycyclohexyl) dihydrogen phosphate; also known as “IP6”;) (see, Thorne Research, Inc. (2002) “Inositol Hexaphosphate. Monograph,” Altem. Med. Rev. 7(3):244-248; Vucenik, I. et al. (2006) “Protection Against Cancer By Dietary IP6 And Inositol,” Nutr. Cancer. 55(2):109-125; López, M. A. et al. (2004) “Iron Availability: An Updated Review,” Int. J. Food Sci. Nutr. 55(8):597-606; Singh, R. P. et al. (2005) “Prostate Cancer And Inositol Hexaphosphate: Efficacy And Mechanisms,” Anticancer Res. 25(4):2891-2903; Vucenik, I. et al. (2006) “Cancer Inhibition By Inositol Hexaphosphate (IP6) And Inositol: From Laboratory To Clinic,” J. Nutr. 133(11 Suppl 1):3778S-3784S; Raboy, V. (2003) “Myo-Inositol-1,2,3,4,5,6-Hexakisphosphate,” Phytochemistry. 64(6):1033-19043; Vohra, A. et al. (2003) “Phytases: Microbial Sources, Production, Purification, And Potential Biotechnological Applications,” Crit. Rev. Biotechnol. 23(1):29-60; Fox, C. H. et al. (2002) “Phytic Acid (IP6), Novel Broad Spectrum Anti-Neoplastic Agent: A Systematic Review,” Complement Ther. Med. 10(4):229-234; Grases, F. et al. (1999) “Phytate (IP6) Is A Powerful Agent For Preventing Calcifications In Biological Fluids: Usefulness In Renal Lithiasis Treatment,” Anticancer Res. 19(5a):3717-3722; Jariwalla, R. J. (1999) “Inositol Hexaphosphate (IP6) As An Anti-Neoplastic And Lipid-Lowering Agent,” Anticancer Res. 19(5a):3699-3702; Katayama T. (1999) “Hypolipidemic Action Of Phytic Acid (IP6): Prevention Of Fatty Liver,” Anticancer Res. 19(5a):3695-3698). The structure of phytic acid is provided below:

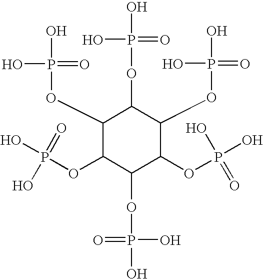

Phytic acid is found in substantial amounts in whole grains, cereals, legumes, nuts, and seeds, and is the primary energy source for the germinating plant (Graf, E. (1983) “Applications of Phytic Acid,” J. Am. Oil. Chem. Soc 60:1861-1867). Phytic acid and its lower phosphorylated forms (such as IP3) are also found in most mammalian cells, where they assist in regulating a variety of important cellular functions (Szwergold, B. S. et al. (1987) “Observation Of Inositol Pentakis- And Hexakisphosphates In Mammaian Tissues By ³¹P NMR,” Biochem. Biophys. Res. Commun. 264:874-881). Phytic Acid is preferably provided in the form of rice bran (Srinivasan, M. (2007) “Ferulic Acid: Therapeutic Potential Through Its Antioxidant Property,” J. Clin. Biochem. Nutr. (2007) 40(2):92-100; Kim, M. J. et al. (2007) “Ferulic Acid Supplementation Prevents Trimethyltin-Induced Cognitive Deficits in Mice,” Biosci. Biotechnol. Biochem. (2007) 71(4):1063-1068). Phytic acid is reported to function as an antioxidant by chelating divalent cations such as copper and iron, thereby preventing the generation of reactive oxygen species responsible for cell injury and carcinogenesis (Harland, B. F. et al. (1987) “Phytate In Foods,” World Rev. Nutr. Diet 52:235-259). The preferred composition dosage of phytic acid (for example, as rice bran) is in the range of 2000-12,000 mg.

As used herein, the term “hyaluronic acid” (also known as hyaluronan) refers to linear polymer composed of repeating disaccharides of D-glucuronic acid and D-N-acetylglucosamine, linked together via alternating β-1,4 and β-1,3 glycosidic bonds ([-β(1,4)-GlcUA-β(1,3)-GlcNAc-]ₙ). Hyaluronic acid can be 25,000 disaccharide repeats (n) in length:

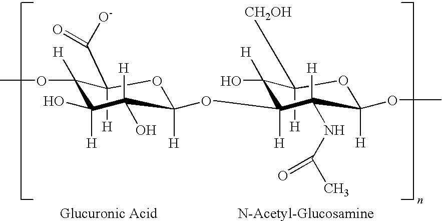

Hyaluronic acid is a water-retaining molecule that is generated naturally in the human body but in decreasing amounts as the body ages. Hyaluronic acid is a multifunctional glycosaminoglycan that forms the basis of the pericellular matrix of cells. Hyaluronic acid is synthesized by 3 different but related enzymes (hyaluronan synthases: HAS1, HAS2 and HAS3 (Weigel, P. H. et al. (1997) “Hyaluronan Synthases,” J. Biol. Chem. 272:13997-14000; Tammi, M. I. et al. (2002) “Hyaluronan And Homeostasis: A Balancing Act,” J. Biol. Chem. 277:4581-4584; Kakehi, K. et al. (2003) “Hyaluronic Acid: Separation And Biological Implications,” J. Chromatogr. B Analyt. Technol. Biomed. Life Sci. 797(1-2):347-355; Radaeva, I. F. et al. (1997) “Hyaluronic Acid: Biological Role, Structure, Synthesis, Isolation, Purification, And Application (Review)” Prikl. Biokhim. Mikrobiol. 33(2):133-137; Stoolmiller, A. C. et al. (1970) “The Biosynthesis Of Hyaluronic Acid By Group A Streptococcus,” Expos. Annu. Biochim. Med. 30:65-78). U.S. Patent Application Publication 2004/0234497 discloses the use of hyaluronic acid for cancer drug delivery. The entire disclosure of that publication is incorporated herein by reference.

Hyaluronic acid has been traditionally extracted from rooster combs, from bovine or fish vitreous humor, from microbial production or from other sources (Rangaswamy, V. et al. (Epub 2007 Oct. 24) “An Efficient Process For Production And Purification Of Hyaluronic Acid From Streptococcus Equi Subsp. Zooepidemicus,” Biotechnol. Lett. 30(3):493-496; Gao, F. et al. (2006) “Preparation And Characterization Of Hyaluronan Oligosaccharides For Angiogenesis Study,” J. Biomed. Mater. Res. B Appl. Biomater. 78(2):385-392; Blank, L. M. et al. (2005) “Stable Production Of Hyaluronic Acid In Streptococcus Zooepidemicus Chemostats Operated At High Dilution Rate,” Biotechnol. Bioeng. 90(6):685-693; Kakehi, K. et al. (2003) “Hyaluronic Acid: Separation And Biological Implications,” J. Chromatogr. B Analyt. Technol. Biomed Life Sci. 797(1-2):347-355; Volpi, N. et al. (2003) “Purification And Characterization Of Hyaluronic Acid From The Mollusc Bivalve Mytilus Galloprovincialis,” Biochimie 85(6):619-625; Tawada, A. et al. (2002) “Large-Scale Preparation, Purification, And Characterization Of Hyaluronan Oligosaccharides From 4-Mers To 52-Mers,” Glycobiology. 2002 July; 12(7):421-426; Mahoney, D. J. et al. (2001) “Novel Methods For The Preparation And Characterization Of Hyaluronan Oligosaccharides Of Defined Length,” Glycobiology. 11(12):1025-1033; Mcdonald, J. et al. (2002) “Hyaluronan Minireview Series,” J. Biol. Chem. 277(7):4575-4579; Radaeva, I. F. et al. (1997) “Hyaluronic Acid: Biological Role, Structure, Synthesis, Isolation, Purification, And Application (Review)” Prikl. Biokhim. Mikrobiol. 33(2): 133-137). Most preferably, the hyaluronic acid of the present invention is obtained from rooster combs. Hyaluronic acid is widely available commercially, and such preparations are suitable for the purposes of the present invention. Preferably, the compositions of the present invention will provide a composition dosage of hyaluronic acid of from about 1 mg to about 400 mg, more preferably from about 50 mg to about 200 mg.

As used herein, the term “Vitamin D” refers to a fat-soluble prohormone. Two major forms of vitamin D are vitamin D₂ (ergocalciferol) and vitamin D₃ (cholecalciferol) (DeLuca, H. F. et al. (1998) “Mechanisms And Functions Of Vitamin D,” Nutr. Rev. 56:S4-S10):

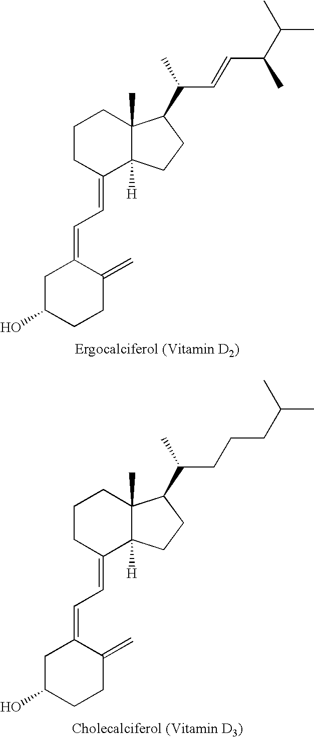

Vitamin D exhibits many biological actions. While vitamin D is widely known for its ability to stave off bone disease (rickets in growing children, osteoporosis in senior adults), it is becoming a central player in the battle against cancer. Regarding the role of vitamin D in immunity and cancer, vitamin D improves the chemotactic (affinity for) neutrophils to mobilize and migrate. Patients with rickets due to vitamin D deficiency are observed to have sluggish neutrophils that cannot migrate properly. Vitamin D stimulates the maturation of monocytes to macrophages. This results in an enlarged army of immune fighting cells to mount against tumors. Vitamin D is widely available commercially, and such preparations are suitable for the purposes of the present invention.

Vitamin D is essential for optimal muscle, bone, brain, immune and cardiovascular health and is undergoing re-discovery by aging researchers worldwide. Vitamin D supplementation up to 2000 IU has been shown to significantly reduce mortality rates, thus adding vitamin D to the lineup of molecules now considered to be true longevity factors (Autier, P. et al. (2007) “Vitamin D Supplementation And Total Mortality: A Meta-Analysis Of Randomized Controlled Trials,” Arch Intern Med. 167(16): 1730-1737). Its anti-calcifying properties (Zittermann, A. et al. (2007) “Vitamin D And Vascular Calcification,” Curr. Opin. Lipidology 18(1):41-46) qualify vitamin D as another powerful agent that inhibits progressive overmineralization in the human body with advancing age and parallels the action of other mineral chelators in the compositions of the present invention. While the 1200 IU dose is three times more than the Recommended Daily Allowance, it is well within the Safe Upper Limit established by the National Academy of Sciences (2000 IU) and corresponds with a supplemental dosage recently found to be beneficial in a human clinical trial (Lappe, J. M. et al. (2007) “Vitamin D and calcium supplementation reduces cancer risk: results of a randomized trial,” Amer. J. Clin. Nutr. 85(6):1586-1591). A 2,000 IU dosage is roughly equivalent the natural vitamin D3 produced by 15-30 minutes of total-body summer sun exposure at noontime at a southern latitude, for which no side effects have been reported. Preferably, the compositions of the present invention will provide a composition dosage of vitamin D of from about 100 IU to about 100,000 IU, more preferably from about 1,000 IU to about 50,000 IU.

The compositions of the present invention may contain additional components, including additional active components that act to enhance resveratrol biological activity and inactive compounds (e.g., flavorants, sweeteners, dyes, vitamins, amino acids (e.g., lysine, proline, etc.), minerals, nutrients, etc.).

In particular, quercetin (3,3′,4′,5,7-pentahydroxy-2-phenylchromen-4-one)

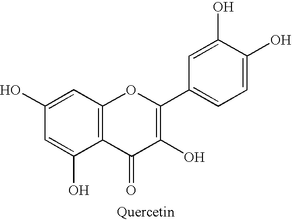

butein, fisetin, myricetin, kaempferol, cis-resveratrol or piceatannol may be added to the compositions.

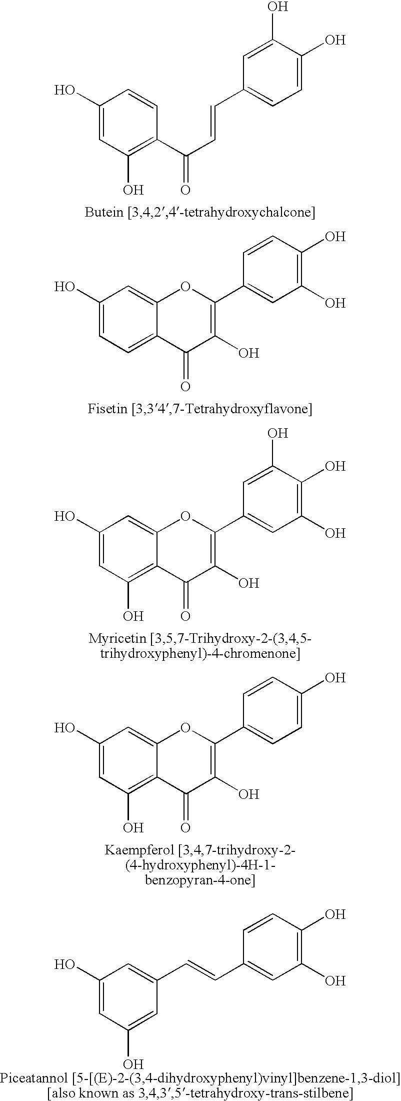

Such compounds are described, for example by Stecher, G. et al. (2001) (“Determination Of Flavonoids And Stilbenes In Red Wine And Related Biological Products By HPLC And HPLC-ESI-MS-MS,” Fresenius J. Anal Chem. 2001 September; 371(1):73-80).

Resveratrol is glucuronated in the human liver, which may reduce its bioavailability. Flavonoids, such as quercetin, inhibit resveratrol glucuronidation and thus may act to improve resveratrol bioavailability (see, de Santi, C. et al. (2000) “Glucuronidation Of Resveratrol, A Natural Product Present In Grape And Wine, In The Human Liver,” Xenobiotica 30(11):1047-1054); De Santi, C. et al. (2000) “Sulphation Of Resveratrol, A Natural Compound Present In Wine, And Its Inhibition By Natural Flavonoids,” Xenobiotica 30(9):857-866; De Santi, C. et al. (2000) “Sulphation Of Resveratrol, A Natural Product Present In Grapes And Wine, In The Human Liver And Duodenum,” Xenobiotica 30(6):609-617). Quercetin may also act synergistically with resveratrol or independently of resveratrol to provide beneficial function (Kampkötter, A. et al. (Epub 2007 Oct. 16) “Increase Of Stress Resistance And Lifespan Of Caenorhabditis Elegans By Quercetin,” Comp. Biochem. Physiol. B Biochem. Mol. Biol. 149(2):314-323). (See also, Kaindl, U. et al. (2008) “The Dietary Antioxidants Resveratrol And Quercetin Protect Cells From Exogenous Pro-Oxidative Damage,” Food Chem. Toxicol. 46(4):1320-1326; Melzig, M. F et al. (2002) “Induction Of Neutral Endopeptidase And Angiotensin-Converting Enzyme Activity Of Sk-N-Sh Cells In Vitro By Quercetin And Resveratrol,” Pharmazie. 57(8):556-558; Hsu, C. L. et al. (2006) “Induction Of Cell Apoptosis In 3T3-LJ Pre-Adipocytes By Flavonoids Is Associated With Their Antioxidant Activity,” Molec. Nutr. Food Res. 50(11):1072-1079; Chan, M. M. et al. (2000) “Synergy Between Ethanol And Grape Polyphenols, Quercetin, And Resveratrol, In The Inhibition Of The Inducible Nitric Oxide Synthase Pathway,” Biochem. Pharmacol. 60(10): 1539-1548; Nicholson S. K. et al. (2008) “Effects Of Dietary Polyphenols On Gene Expression In Human Vascular Endothelial Cells,” Proc. Nutr. Soc. 67(1):42-47; Lemos, C. et al. (2007) “Modulation Of Folate Uptake In Cultured Human Colon Adenocarcinoma Caco-2 Cells By Dietary Compounds,” Eur. J. Nutr 46(6):329-336).

Emulsifiers, fillers, binding agents, and the like may also be included in the compositions of the present invention.

In one embodiment, the compositions of the present invention comprise a novel combination of: small molecules such as quercetin and resveratrol with widespread preventive and therapeutic health properties; and/or collagen-building nutrients (such as vitamin C-ascorbate, lysine, proline, etc.); and a glycosaminoglycan such as a shortened (low molecular weight) chain of hyaluronic acid (HA) or its singular components (glucosamine, glucuronate) or chondroitin sulfate, which are linear disaccharides (sugar-like molecules) that serve as structural components of cartilage, but in this combination serve as synergistic co-healing agents in non-cellular (connective) tissue that surrounds living cells. The combination of glycosaminoglycans (hyaluronan, glucuronate or chondroitin) and/or ascorbate, lysine or proline, to encourage the generation of collagen, and small molecules, that operate on intra-cellular basis, comprise therapeutic or preventive action that encompasses the total tissue matrix. The combination of the present invention is intended for human or animal oral intake as a dietary supplement. In a sub-embodiment thereof, such compositions may comprise a combination of resveratrol and hyaluronan in a dietary supplement that serves to heal a variety of illnesses including some cancers. Resveratrol is known to be an anti-cancer molecule and to have other healing and longevity enhancing properties. Hyaluronan (hyaluronic acid, HA) is taken as an oral supplement or can be given intravenously to target cancer cells. When combined with or attached to other molecules, hyaluronan will deliver other anti-cancer and healing agents such as resveratrol to tumor sites. The combination may or may not include a chelating agent, an antioxidant and/or an emulsifier as described in my above-referenced co-pending patent application. When encapsulated or otherwise applied together, with or without those additives, resveratrol and HA have powerful healing properties for animals and humans.

Most preferably, the compositions of the present invention stabilize resveratrol specific activity such that the resveratrol of the compositions has a specific activity that is greater than that of resveratrol maintained in the presence of oxygen gas, or maintained in the absence of a chelator, hyaluronic acid, or vitamin D. Preferably, the amounts of the non-resveratrol constituents of the compositions will stabilize the composition's resveratrol so that it exhibits at least 10% more activity, at least 20% more activity, at least 50% more activity, at least 2-times the activity, at least 5-times the activity, or at least 10-times the activity of resveratrol maintained in the presence of oxygen gas, or maintained in the absence of a chelator, hyaluronic acid, or vitamin D and so that it remains capable of exhibiting such specific activity over extended periods (for example, 1, 2, 4, 6, 10, 12, 18, 24, or 36 months or longer) at ambient conditions of temperature and humidity (i.e., without need for special precautions as to temperature or humidity).

D. Packaging of the Preferred Compositions of the Present Invention

Resveratrol is typically unstable to light and oxidation (Shaanxi University of Science & Technology, Xianyang China (2007) “Study On The Stability Of Resveratrol In Rhizoma Polygoni cuspidate,” Zhong Yao Cai. 30(7):805-80). The resveratrol of the present invention is preferably prepared, packaged and/or stored in a manner that maximizes its specific activity. It is preferred to prepare, package and/or store resveratrol in low light (or in the dark) and/or in low oxygen, so as to minimize light-induced degradation (e.g., photo-isomerization) or oxygen-induced degradation. The preferred compositions of the present invention are formulated as dietary supplements for oral ingestion in the form of a pill, lozenge, capsule, elixir, syrup, etc. Other modalities of administration may alternatively be employed (e.g., intranasal, parenteral, intravenous, intraarterial, topical, etc.).

In a first example of such preferred packaging, the compositions of the present invention are formulated as air-tight capsules in which encapsulation is conducted so as to prevent or minimize exposure to oxygen. In one embodiment, such encapsulation is conducted in an oxygen-free environment. For example, the components of the compositions of the present invention may be inserted into a capsule in an inert gas (e.g., nitrogen, argon, etc.) environment. Preferably, a nitrogen bubble (e.g., 5-20% of the capsule volume) may be introduced into the capsule to further stabilize and protect the components against oxidation (see, PCT Publication No. WO 01/08631, herein incorporated by reference). That international application has a corresponding U.S. patent application. Suitable capsules useful in the encapsulation of resveratrol and other oxidation prone ingredients of dietary supplements include Licaps® (Capsugel), an air-tight gelatin capsule. The presence of phytic acid, which has the ability to protect the components from metal-induced oxidation, augments such anti-oxidation precautions. A particularly preferred example of such a resveratrol-containing composition is Longevinex® (Resveratrol Partners, LLC, San Dimas, Calif.), which comprises resveratrol and phytic acid. Longevinex® contains as active ingredients (per capsule): 5 mg Vitamin E (as mixed tocopherols), 215 mg total resveratrol (obtained from French red wine and giant knotwood (Polygonum cuspidatum), and providing 100 mg of trans-resveratrol), 25 mg quercetin dihydrate, 75 mg phytic acid (rice bran extract), 380 mg rice bran oil, 55 mg sunflower lecithin.

Once a composition has been sealed into an air-tight capsule, it is important to maintain a low or no-oxygen environment in the packaging surrounding the capsules in order to protect the composition from oxidation should a break or leak occur in the sealed capsule. Therefore, an oxygen absorbing packette is preferably employed to reduce the presence of free oxygen. Vacuum or nitrogen-flushed packaging (bottles, pill cases, etc.) in air-tight materials is desirable.

In an alternative embodiment, the components and compositions of the present invention may be prepared as a microencapsulated process (see, generally, Rubiana, M. et al. (2004) “Drug Delivery Systems: Past, Present, and Future,” Current Drug Targets, 5(5):449-455). Micro-encapsulation is a process by which tiny particles or droplets (ranging in size from a few nanometers to one micron) are coated with a protective layer to create small capsules with controlled properties. Suitable micron-sized, encapsulated, preparations can be obtained using the microencapsulation processes of Maxx Performance Inc. (Chester, N.Y.), Blue California (Rancho Santa Margarita, Calif.), Southwest Research Institute (San Antonio, Tex.), Coating Place, Inc. (Verona, Wis.), Microtek Laboratories (Dayton, Ohio), Particle Sciences, Inc. (Bethlehem, Pa.), etc. 3^{rd}-generation Longevinex® (“Longevinex-3®”) (Resveratrol Partners, LLC), which contains Vitamin D3, Vitamin E, Resveratrol, Quercetin, and Phytic Acid is a particularly preferred microencapsulated form of the compositions of the present invention.

The present invention further comprises a practical method of stabilizing quercetin and other easily oxidized dietary supplement ingredients which may come in contact with oxidizing metals.

E. Utility of the Compositions of the Present Invention

The compositions of the present invention enhance resveratrol's specific activity. The compositions of the present invention therefore find utility in the treatment of diseases (or in the amelioration of the symptoms of diseases) such as cardiovascular disease, cancer, macular degeneration, aging, neurodegenerative diseases (e.g., Alzheimer's Disease, Parkinson's Disease, etc.) and inflammation in which the modulation of expression of “survival/longevity” genes and/or “damage inducing” genes is desired. Over time, as minerals such as calcium and iron accumulate in the human body, genes respond in deleterious ways. Liu, Y. et al. (2005) “Global Genomic Approaches To The Iron-Regulated Proteome,” Ann. Clin. Lab. Sci. 35(3):230-239; Templeton, D. M. et al. (2003) “Genetic Regulation Of Cell Function In Response To Iron Overload Or Chelation,” Biochim. Biophys. Acta. 1619(2): 113-124; Ikeda, H. et al. (1992) “Evidence That An Iron Chelator Regulates Collagen Synthesis By Decreasing The Stability Of Procollagen mRNA,” Hepatology 15(2):282-287. The present invention has particular utility in the treatment of macular degeneration, cancer and the conditions of aging.

1. Macular Degeneration

The prolongation of the human lifespan over the past few decades in the US has spawned the proliferation of macular degeneration, an age-related eye disease. While not resulting in total vision loss, the disease robs older adults of their central vision used for reading as well as color vision. Macular degeneration affects the visual center of the eye, called the macula. The macula is part of the retina where color-vision cells (cones) are located.

Macular degeneration is a progressive, age-related disease that can be broken down into four stages:
1. Beginning in about the third decade of life, the inability of the “garbage cleaning” cells, called the retinal pigment epithelia (RPE), to engulf and remove cellular debris from the back of the eyes, results in the formation of small microscopic deposits called lipofuscin (ly-poh-fus-kin). Lipofuscin is from by iron and copper-induced oxidation of cellular debris and its accumulation correlates with premature aging and shortened lifespan of organisms. The prevalence of macular degeneration is greater in Caucasians than persons with darkly-pigmented skin and Caucasians have more lipofuscin deposits in their retinas. Some of this cellular debris in the retina is comprised of used-up vitamin A that is shed from night-vision (rod) cells each morning in the human eye. The failure of the RPE cells to function results from accumulation of iron and calcium within the RPE.
2. Later, in about the fifth decade of life, there is progressive calcification of an underlying cellophane-thin retinal layer called Bruch's membrane, which resides between the RPE and the blood supply layer (choroid). While drusen that forms within the retina is partially composed of cholesterol, this lipid does not originate from the blood circulation or the liver where most cholesterol is produced. Calcifications within Bruch's membrane further impairs the exit of lipids (fats), protein, and cellular debris, from the photoreceptor layer, which results in the formation of yellow spots called drusen on the retina. Drusen can be observed during an eye examination using an opthalmoscope. There is currently no method of removing drusen.
3. The death of the RPE cells is the third stage of this progressive disease. This is sometimes called RPE dropout. As the RPE cells are either impaired or have died, and Bruch's membrane is clogged with calcium, the photoreceptors then cannot be nourished and also begin to die off. There is currently no treatment for stages 1-3 of macular degeneration. Stage 1-3 is called the “dry” form of macular degeneration because it has not resulted in hemorrhage or edema or new blood vessel formation. About 85% of macular degeneration patients have the “dry” form of this disease.
4. As breaks in Bruch's membrane occur, or Bruch's membrane becomes totally calcified, the photoreceptor layer is deprived of oxygen and new blood vessels form (called neovascularization) which can invade the photoreceptor layer in the macula and impair vision; or there may be leakage of blood serum or frank release of red blood cells, which results in edema or hemorrhage. This is the more advanced and sight-threatening form of macular degeneration, often called “wet” macular degeneration because of the presence of the leakage of blood serum or red blood cells into the photoreceptor layer. This stage of the disease, if caught early, can be treated with laser beams, which can seal up leaky blood vessels. However, this treatment is only effective in delaying the progression of the disease, not curing it.

The cell cleansing process facilitated by the lysosomes cannot keep up with the accumulation of metabolic waste over a lifetime. The parafoveal ring, where rod cell density is highest, and therefore more discs of used-up vitamin A are shed, is where macular degeneration begins, and where the highest concentration of lipofuscin is observed in the retina. Eventually, the RPE cells die off with advancing age, which increases the burden on the remaining RPE cells to maintain a healthy retina.

In the past, lipofuscin has been considered a harmless wear-and-tear byproduct of cellular metabolism. One aspect of the present invention relates to the recognition that lipofuscin, which forms from iron and copper-induced oxidation, and hardens within lysosomal bodies within retinal pigment epithelial cells, sensitizes the retina to damage by mild amounts of radiation and oxidation. The retina becomes increasingly sensitive to blue-light damage with advancing age. Drusen formation within the retina is associated with RPE cell inability to produce superoxide dismutase, an endogenous antioxidant enzyme. Mice deficient in superoxide dismutase develop features that are typical of age-related macular degeneration in humans. Superoxide dismutase protects retinal cells against unbound (free) iron. High iron diets and cellular environments have been shown to reduce superoxide dismutase activity.

Retinal photoreceptors and retinal pigment epithelial cells are believed to be especially vulnerable to damage by low-molecular weight complexes of iron. Since antioxidants in the blood circulation may not always be able to cross the blood-retinal barrier, the retina produces its own protective antioxidants that bind iron. Iron chelators inhibit the adverse effects of unbound (free) iron (not bound to proteins). Heme oxygenase also serves in a similar manner to iron chelators to prevent retinal damage induced by loose iron.

Numerous agents have been used experimentally to clear up lipofuscin and drusen. Statin drugs, commonly used to reduce blood serum levels of cholesterol, have also been tested to prevent lipofuscin deposits in animals. Statin drugs reduced lipofuscin formation but were toxic to the liver and brought about the early death of these animals. Piracetam, a derivative of the neurotransmitter GABA, now available as a dietary supplement, has been used successfully to reduce lipofuscin formation in brain tissues. Sorbinil is an enzyme inhibiting drug (aklose reductase inhibitor) that underwent unsuccessful human trials in the 1990s to prevent retinal problems associated with diabetes. Sorbinil has been shown to partially reduce lipofuscin deposits in the retinal pigment epithelium cells of rodents. Hydergine is a drug used to treat senile dementia. In a rodent study, hydergine was reported to have reduced brain lipofuscin levels, but also led to the early demise of the animals. The East Indian spice turmeric contains an antioxidant molecule called curcumin. Curcumin has been used in an experimental mouse study to reduce lipofuscin in the brain. Purslane is a flowering plant rich in magnesium, beta carotene and omega-3 oil. The provision of purslane to mice has been shown to reduce lipofuscin deposition in the brain of mice.

In a lab dish study, sulforaphane, an antioxidant molecule found in Brussels sprouts and broccoli in 1992, has been used successfully to reduce lipofuscin deposits in RPE cells exposed to blue light.

Intraperitoneal administration of lipoic acid to aged rats leads to a reduction and elevation in lipofuscin and enzyme activity, respectively, in the cortex, cerebellum, striatum, hippocampus, and hypothalamus of the brain. These results suggest that lipoic acid, a natural metabolic antioxidant, should be useful as a therapeutic tool in preventing neuronal dysfunction in aged individuals. Lipoic acid, a natural antioxidant produced within living tissues, and also available as a dietary supplement, has been shown to protect RPE cells from oxidative damage in lab dish studies.

Lipofuscin formation dramatically increases in brain tissues following alcohol consumption. Supplementation with high-dose grape seed flavonols prevents increase lipofuscin formation. Lipofuscin is an end-product of lipid peroxidation which dramatically increases following ethanol consumption.

Oolong and green tea drinks reverse the cognitive impairment and lipofuscin formation in mice. Epigallocatechin-3-gallate (EGCG), the major constituent of green tea, upregulates the activity of heme oxygenase in lab dish studies. Heme oxygenase is a protective enzyme against iron-induced oxidation, which occurs in the retina.

It has been shown that the provision of supplemental estrogen decreases lipofuscin deposition in brain tissues. In a lab dish study, the provision of lutein and zeaxanthin to RPE cells reduced lipofuscin formation. In rodents given supplemental acetyl-L-camitine, a decline in lipofuscin deposits has been measured in brain cells.

U.S. Pat. No. 5,747,536 describes the combined therapeutic use of L-camitine, lower alkanoyl L-camitines or the pharmacologically acceptable salts thereof, with resveratrol, resveratrol derivatives or resveratrol-containing natural products, for producing a medicament for the prophylaxis and treatment of cardiovascular disorders, peripheral vascular diseases and peripheral diabetic neuropathy.

Melanin is an iron-binding antioxidant in the retina. As melanin levels decline in the retina with advancing age, there is a greater accumulation of lipofuscin.

In one embodiment, the present invention relates to a composition comprising a combination of:
(a) A chelator such as inositol hexaphosphate (IP6), trans resveratrol, quercetin, or any polyphenol or bioflavonoid for metal(s) such as iron, copper, heavy metals;
(b) A calcium chelator, such as inositol hexaphosphate (IP6);
(c) A heme oxygenase activator, such as trans resveratrol, piceatannol, or any of resveratrol's natural analogs, or similar small molecules such as fisetin, myricetin, quercetin or other bioflavonoids;
(d) An agent that lowers the affinity of oxygen for red blood cells, such as inositol hexaphosphate (IP6); and, optionally
(e) Other antioxidants such as vitamin E, lutein/zeaxanthin, alpha lipoic acid.

The formulation functions to:
(1) Limit oxidation in retinal tissues (photoreceptors, retinal pigment epithelial cells (RPE), choroid, specifically mitochondria and lysosomes in RPE cells);
(2) Inhibit accumulation of lipofuscin deposits;
(3) Inhibit formation of drusen; and
(4) Limit calcifications to retinal tissues, especially Bruch's membrane.

2. Cancer

A major challenge in cancer therapy is to selectively target cytotoxic agents to tumor cells (Luo, Y. et al. (2000) “A Hyaluronic Acid-Taxol Antitumor Bioconjugate Targeted To Cancer Cells,” Biomacromolecules 1(2):208-218). To decrease undesirable side effects of small molecule anticancer agents, many targeting approaches have been examined. One of the most promising methods involves the combination or covalent attachment of the cytotoxin with a macromolecular carrier, and in particular with hyaluronic acid (Luo, Y. et al. (1999) “Synthesis And Selective Cytotoxicity Of A Hyaluronic Acid-Antitumor Bioconjugate,” Bioconjug. Chem. 10(5):755-763; Luo, Y. et al. (2000) “A Hyaluronic Acid-Taxol Antitumor Bioconjugate Targeted To Cancer Cells,” Biomacromolecules 1(2):208-218; Luo, Y. et al. (1999) “Hyaluronic Acid-N-Hydroxysuccinimide: A Useful Intermediate For Bioconjugation,” Bioconjug. Chem. 12(6):1085-1088; Luo, Y. et al. (2002) “Targeted Delivery Of Doxorubicin By HPMA Copolymer-Hyaluronan Bioconjugates,” Pharm. Res. 19(4):396-402).

In one embodiment, the present invention relates to a resveratrol- and hyaluronic acid-containing composition for the treatment of cancer comprising: resveratrol, hyaluronan, and optionally vitamin D and/or IP6. It is believed that these components act synergistically with one another to mediate an effect in curing and/or in preventing cancer in humans and/or in improving immunity (e.g., immune system response) in patients threatened by tumors. This aspect of the present invention is based in part upon the recognition that natural molecules can boost cancer immunity, possibly in a manner similar to that observed in cancer-proof mice.

Upon provision with such composition, the sentinels of the innate immune system, dendritic cells, can be alerted and neutrophils, macrophages and natural killer cell activity can be significantly enhanced. The enhancement of vitamin D receptors via resveratrol is yet another major advantage of a combination approach to treat or prevent cancer. This approach appears to be more appropriate for senior adults, the highest risk group for cancer, who are often immune-compromised due to poor nutrition or lack of nutrient absorption. The fact that this therapy can now be immediately measured for effectiveness by non-invasive cancer cell counting technology means that expensive and equivocal tests on animals may not be required to prove efficacy.

Vitamin D exhibits many biological actions. While vitamin D is widely known for its ability to stave off bone disease (rickets in growing children, osteoporosis in senior adults), it is becoming a central player in the battle against cancer. Only recently is it also gaining attention as an antibiotic. Vitamin D-deficient mice exhibit a defective response from phagocyte cells in the face of infection or inflammation. Vitamin D deficiency is frequently associated with recurrent infections. Only about half of the macrophage cells accumulate at the site of inflammation in vitamin D-deficient animals compared to animals whose vitamin D levels are adequate.

To delve deeper into the role of vitamin D in immunity and cancer, vitamin D improves the chemotactic (affinity for) neutrophils to mobilize and migrate. Patients with rickets due to vitamin D deficiency are observed to have sluggish neutrophils that cannot migrate properly. Vitamin D stimulates the maturation of monocytes to macrophages. This results in an enlarged army of immune fighting cells to mount against tumors. Greater attention is now being given to vitamin D as an anti-cancer weapon because of studies which show supplemental vitamin D drastically reduces the risk for all types of cancer. A study that employed 1100 IU of vitamin D3 produced a 60-77% reduction in cancer risk among women in Nebraska in just a 4-year period.

Even though cancer risk is lowest in sunnier and Equatorial areas geographically, where vitamin D levels are higher in sun-exposed populations, the protective effect of vitamin D against cancer has been repeatedly dismissed or discounted. The consumption of vitamin D orally eliminates the concern of skin cancer emanating from overexposure to unfiltered sun rays. One of the latest analyses shows that the risk of colon cancer can be halved by taking 2000 IU of vitamin D per day and that the risk for breast cancer can be halved by taking 3500 IU of vitamin D per day. The median dietary intake of vitamin D is only about 230 IU per day, so the prospect of food fortification or supplementation to prevent or treat cancer now becomes real.

In order for tissues to utilize and benefit from vitamin D they must have proteins in their outer coat (cell membrane) that are designed to receive and bind to vitamin D. For example, about 80% of human breast tumors produce vitamin D cell receptors, though gene expression (production) of vitamin D receptor is at low levels.

Vitamin D's ability to inhibit cancer may be heightened when it is aided by weak estrogen-like molecules in the diet. Resveratrol, an estrogen-like molecule commonly found in red wine, upregulates the vitamin D receptor in breast cancer cells without increasing cancer growth. Resveratrol, in effect, can sensitize breast cancer cells to the anti-cancer properties of vitamin D.

Laboratory experiments show that low-dose vitamin D3 does not reduce breast tumor cell growth but when combined with resveratrol, tumor cell numbers declines by 40%. At higher concentrations vitamin D3 reduces the number of breast cancer cells in a lab dish by about 25%, and this decline improves to 50% when combined with resveratrol. Whereas estrogen increases vitamin D receptor gene expression, it also stimulates breast tumor growth. Resveratrol does not have this drawback. Resveratrol potentiates or “weaponizes” the cancer-inhibiting effect of vitamin D.

Furthermore, resveratrol by itself has been shown to calm the response of phagocytes to foreign invaders like germs and tumor cells. Resveratrol dampens production of reactive oxygen species (free radicals) and normalizes particle ingestion in macrophage cells. Therefore, resveratrol prevents the over-response of immune cells that can produce autoimmunity.

Resveratrol blocks cancer in so many ways that it is difficult to find a pathway for cancer that is not obstructed by resveratrol. Resveratrol induces the cell energy compartments in tumor cells, called mitochondria, to release an enzyme called cytochrome C oxidase that usually leads to a cascade of other enzymes that induce programmed cell death, called apoptosis. But a recent experiment also shows that resveratrol releases cytochrome C from ovarian tumor cells that leads to rapid cell death via a process called autophagy, a process where enzymes produced inside the tumor cell actually digest its innards (kind of a form of intracellular cannibalism). This is a form of cell suicide that resveratrol activates in tumor cells, but not healthy cells.

The contribution of innate immunity in surveillance of tumors is comparatively neglected in cancer biology. Phagocytosis, or “cell eating” is the cornerstone of the innate immune response. Focus has been directed to dendritic cells which are believed to be sentinels of the innate immune response. A limited number of immune-boosting agents have been investigated.

Skepticism surrounds interest in innate immune approaches to cancer treatment. For example, patients taking immune-suppressing don't necessarily develop cancer with more frequency. However, this may be misunderstood. An over-responsive immune system may lead to more tissue and organ damage that can be mortal to cancer patients. Most of the drugs used for breast cancer therapy induce immune suppression.

Nature's most potent iron chelator is inositol hexaphosphate (IP6), which is found in seeds and the bran fraction of whole grains. A low dosage of IP6 has been found to suppress the growth of rhabdomyosarcoma cells by 50%. Removal of IP6 allows these tumor cells to recover and grow once again. IP6-treated mice with injected tumors exhibit tumors that are 50 times smaller than non-treated mice. IP6 has also been shown to reduce the growth of injected fibrosarcoma cells in mice and prolong their survival.

In examining the immune enhancing properties of IP6 it has been shown that it boosts production of free radicals (superoxide) and the cell digesting action of neutrophils in the presence of bacteria. IP6 increases the release of interleukin-8.

The action of natural killer cells, which are involved in tumor cell destruction, is enhanced by IP6.

In one embodiment, the hyaluronic acid of such composition is conjugated to a chemotherapeutic agent. The invention particularly pertains to such compositions in which the chemotherapeutic agent is taxol. The invention particularly pertains to such compositions that additionally and preferably comprise a chelator, and/or vitamin D. Most malignant solid tumors contain elevated levels of Hyaluronic Acid (Rooney, P. et al. (1995) “The Role Of Hyaluronan In Tumour Neovascularization (Review),” Int. J. Cancer 60(5):632-636) and these high levels of HA production provide a matrix that facilitates invasion (Hua, Q. et al. (1993) “Internalization Of Hyaluronan By Chondrocytes Occurs Via Receptor-Mediated Endocytosis,” J. Cell. Sci. 106(Pt 1):365-375; Luo, Y. et al. (2000) “A Hyaluronic Acid-Taxol Antitumor Bioconjugate Targeted To Cancer Cells,” Biomacromolecules 1(2):208-218). Thus chemotherapeutic agents that are conjugated to Hyaluronic Acid target tumor cells, and can provide an effective anti-tumor dosage at lower overall concentration.

In brief, a preferred method of conjugation entails forming an NHS (N-hydroxy-succinimide derivative of the chemotherapeutic agent. Such a derivative can be made by adding a molar excess of dry pyridine to a stirred solution of Taxol and succinic anhydride in CH₂Cl₂ at room temperature. The reaction mixture is then stirred for several days at room temperature and then concentrated in vacuo. The residue is dissolved in 5 ml of CH₂Cl₂ and the produced Taxol-2′-hemisuccinate can be purified on silica gel (washed with hexane; eluted with ethyl acetate) to give the desired product (Luo, Y. et al. (1999) “Synthesis And Selective Cytotoxicity Of A Hyaluronic Acid-Antitumor Bioconjugate,” Bioconjug. Chem. 10(5):755-763).

The N-hydroxy-succinimide derivative of the chemotherapeutic agent is then conjugated to adipic dihydrazido-functionalized hyaluronic acid. Adipic dihydrazido-functionalized hyaluronic acid is preferably prepared as described by Pouyani, T. et al. (1994) (“Functionalized Derivatives Of Hyaluronic Acid Oligosaccharides—Drug Carriers And Novel Biomaterials,” Bioconjugate Chem. 5:339-347); Pouyani, T. et al. (1994) (“Novel Hydrogels Of Hyaluronic Acid Synthesis, Surface Morphology, And Solid-State NMR,” J. Am. Chem. Soc. 116:7515-7522); Vercruysse, K. P. et al. (1997) (“Synthesis And In Vitro Degradation Of New Polyvalent Hydrazide Cross-Linked Hydrogels Of Hyaluronic Acid,” Bioconjugate Chem. 8:686-694). Thus, hyaluronic acid is preferably dissolved in water and an excess of adipic dihydrazide (ADH). The pH of the reaction mixture is adjusted to 4.75 by addition acid. Next, 1 equivalent of 1-Ethyl-3-[3-(dimethylamino)-propyl]carbodiimide (EDCI) is added in solid form. The pH of the reaction mixture is maintained at 4.75 by addition of acid. The reaction is quenched by addition of 0.1 N NaOH to adjust the pH of reaction mixture to 7.0. The reaction mixture is then transferred to pretreated dialysis tubing (Mw cutoff 3,500) and dialyzed exhaustively against 100 mM NaCl, then 25% EtOH/H2O and finally water. The solution is then filtered through 0.2 m cellulose acetate membrane, flash frozen, and lyophilized (Luo, Y. et al. (1999) “Synthesis And Selective Cytotoxicity Of A Hyaluronic Acid-Antitumor Bioconjugate,” Bioconjug. Chem. 10(5):755-763).

3. Aging

Calcification and rusting are major accelerators of aging. The human body is composed of cells that must continually be replaced or renewed from within, and a gooey substance that fills space between cells called collagen or connective tissue which also must be continually regenerated. As the human body ages at the cellular level, there is a slow accumulation of cellular debris called lipofuscin. The formation of lipofuscin is facilitated by the progressive accumulation of iron and calcium within cell bodies called lysosomes and mitochondria. A cell cleansing and renewal process called autophagy prevents the accumulation of lipofuscin. Progressive inability to remove cellular debris results in declining cell function and then premature death of the cell. A young cell efficiently removes debris from within. An old cell cannot efficiently remove debris and accumulates lipofuscin.

Calcification and rusting of cells impairs the cleansing of cellular debris (lipofuscin) from cells by enzymes produced by lysosomes, and results in impairment of cellular energy (ATP) produced by the mitochondria within cells. The compositions of the present invention inhibit and/or reverse cellular aging and/or connective tissue aging, and in particular, inhibit and/or reverse cellular aging and/or connective tissue aging caused by an accumulation of major minerals (e.g., iron, calcium, etc.). As a consequence, recipients of the compositions of the present invention exhibit enhanced longevity and enhanced cellular and connective tissue health and structure.

The human body ages at the cellular level by the slow accumulation of cellular debris called lipofuscin, which is facilitated by the progressive accumulation of iron and calcium within cell bodies called lysosomes and mitochondria. A cell cleansing and renewal process called autophagy prevents the accumulation of lipofuscin during the years of youthful growth, but this lysosomal mechanism declines once full growth is achieved due to accumulation of intracellular iron and calcium. Progressive inability to remove cellular debris results declining cell function and then premature death of the cell. A young cell efficiently removes debris from within. An old cell cannot efficiently remove debris and accumulates lipofuscin. The mitochondria, which provides cellular energy for lysosomal bodies to perform their cell cleansing activity, also becomes progressively calcified and ironized once childhood growth ceases. Only about 5% of mitochondria are functioning by age 80. Iron and calcium chelators are proposed to remedy mitochondrial aging which impacts cellular functions such as lysosomal enzymatic activity

The human body ages within connective tissue by failure of cells called fibroblasts to regenerate collagen and hyaluronic acid, the latter being a space-filling, water-holding molecule. Collagen formation is facilitated by vitamins and amino acids in the diet (vitamin C, lysine, proline). Fibroblasts can be stimulated to produce hyaluronic acid by estrogen, made naturally in the body, and by estrogen-like molecules found in plants, called phytoestrogens, provided in the diet of by hyaluronic acid itself. Young females, by virtue of the ability to produce estrogen, exhibit thicker hair, smoother skin and more flexible joints, due to the abundance of hyaluronic acid. All of these being attributes of youthfulness.

The inability to regenerate hyaluronic acid results in tissues losing their physical integrity by virtue of loss of the space-filling properties of hyaluronic acid. Without adequate hyaluronic acid, a dehydrated state results and tissues shrink and shrivel up. For example, skin that is lacking hyaluronic acid will appear wrinkled and dry. Joint spaces will lack the cushioning and space-filling needed to prevent bone from rubbing on bone. The eyes will begin to shrink in size. Hair will thin due to the lack of hydration. These are the most prominent visible or cosmetic signs of aging.

In one embodiment, the present invention invention addresses both cellular and extracellular (connective tissue) aging, thus (a) preserving youthful function of living cells by removal of excess minerals, largely calcium and iron, from cells, this facilitating autophagy (cleanup of cellular debris, such as lipofuscin, via lysosomal enzymes) and (b) invigorating and preserving production of hyaluronan by stimulation of fibroblasts by HA, phytoestrogens (resveratrol, quercetin, genistein, are a few), to inhibition of degradation of HA by provision of metal chelators, such as phytic acid, ferulate, quercetin, resveratrol, etc.

In one embodiment, the present invention is a dietary supplement that addresses both cellular and non-cellular aging by its ability to:
(a) stimulate renewal of living cells from within via enzymatic degradation of cellular debris by intracellular lysosomal bodies. This is facilitated by the inclusion of metal (iron, copper, heavy metal) and calcium chelating molecules within the formula. Lysosomes lose their ability to enzymatically digest cellular debris with the progressive accumulation of iron, copper and other metals, and the crystallization of calcium.
(b) stimulate fibroblasts to produce hyaluronic acid at youthful levels again. This is accomplished by provision of orally-consumed molecules that stimulate fibroblasts to produce hyaluronic acid.
(c) metal chelating molecules that help maintain youthful lysosomal function are identified as antioxidants, like vitamin E or vitamin C, lipoic acid, metal chelators like IP6 phytate, quercetin, bioflavonoids or polyphenols, resveratrol. Resveratrol works by its ability to stimulate production of heme oxygenase, an enzyme that helps to control iron.
(d) molecules that inhibit crystallization of calcium are magnesium and IP6 phytate.
(e) orally consumed molecules that stimulate fibroblasts to produce hyaluronic acid are hyaluronic acid, glucosamine, chondroitin, or estrogen-like molecules such as genistein, lignans, hydroxytyrosol, or other molecules configured like estrogen. Orally consumed HA stimulates greater HA and chondroitin synthesis. Similarly, glucosamine stimulate fibroblasts to produce HA. Alternatively, or additionally, glucosamine stimulates synovial production of hyaluronic acid, which is primarily responsible for the lubricating and shock-absorbing properties of synovial fluid” (McCarty, M. F. (1998) “Enhanced Synovial Production Of Hyaluronic Acid May Explain Rapid Clinical Response To High-Dose Glucosamine In Osteoarthritis,” Medical Hypotheses 50:507-510, 1998).
(f) orally consumed molecules that stimulate production of collagen are vitamin C, proline and lysine.

In such embodiment, the present invention relates to a resveratrol and hyaluronic acid-containing dietary supplement that restores youthful function and appearance to human cells and tissue. The invention particularly pertains to such compositions that additionally comprise a chelator, and/or vitamin D. Most preferably, the composition will comprise the chelator phytic acid (inositol hexaphosphate; IP6). The compositions of the present invention synergistically enhance the specific activity of the resveratrol and/or hyaluronic acid, and thus the compositions of the present invention provide an enhancement of activity above and beyond that obtained with the components administered individually. In such embodiment, the invention relates to a method for restoring youthful function and appearance to human cells and tissues comprising the following steps:
(a) stimulating renewal of living cells from within via enzymatic degradation of cellular debris by intracellular lysosomal bodies (preferably by providing a metal chelating molecule that helps maintain youthful lysosomal function, such molecules comprising antioxidants, such as vitamin E or vitamin C, lipoic acid, metal chelators like IP6 phytate, quercetin, bioflavonoids or polyphenols, and/or resveratrol); and
(b) stimulating fibroblasts to produce hyaluronic acid (comprises providing orally consumed molecules that stimulate fibroblasts to produce hyaluronic acid, such orally consumed molecules comprising, for example, hyaluronic acid, glucosamine, chondroitin, and/or estrogen-like molecules such as genistein, lignans, hydroxytyrosol, or other molecules configured like estrogen).
   Preferably, such stimulation is achieved by the dietary administration of a composition comprising the stated compounds, more preferably in combination with an orally consumable molecule that stimulates production of collagen, such molecules comprising, for example, vitamin C, proline and/or lysine.

Without intending to be bound by any mechanism, when administered together, the four preferred components are believed to act in the following manner:
(a) Vitamin D: Vitamin D3 works as an agent that mimics the response to a biological stressor, solar radiation. In particular, vitamin D3 upregulates protective genes involved in activation of the immune system, particularly neutrophil count and motility, and aids in overcoming the decline in endogenous vitamin D3 production with advancing age due to thickening of the skin, which reduces sun/skin production of vitamin D. Furthermore, vitamin D3 works synergistically to breakdown IP6 to IP3, thought to be a major active molecule. Resveratrol also works synergistically to sensitize cells to vitamin D3 (sensitizes the vitamin D receptor on the cell surface). Vitamin D serves to break down IP6 to IP3, which is its primary active form. Vitamin D is also believed to act as an immune system enhancing agent, boosting innate immunity in humans. In this capacity, vitamin D has been shown experimentally to have important cancer-preventive and cancer-curing properties.
(b) Resveratrol: Resveratrol increases the sensitivity of the vitamin D receptor on the surface of cells, and thus is believed to act as an enhancing agent for vitamin D and as an anti-cancer agent. Resveratrol up-regulates the vitamin D receptor on the surface of cancer cells, and sensitizes cancer cells to vitamin D (Wietzke, J. A. et al. (2003) “Phytoestrogen Regulation Of A Vitamin D3 Receptor Promoter And 1,25-Dihydroxyvitamin D3 Actions In Human Breast Cancer Cells,” J. Steroid Biochem. Molec. Biol. 84(2-3):149-157; Wietzke, J. A. et al. (2005) “Regulation Of The Human Vitamin D3 Receptor Promoter In Breast Cancer Cells Is Mediated Through Sp1 Sites,” Molec. Cell. Endocrinol. 230(1-2): 59-68). Resveratrol is also believed to be a monoamine oxidase inhibitor (MAO Inhibitor).
(c) Hyaluronic acid: Hyaluronic acid is the water gelling molecule of the human body which serves as its scaffolding and hydrating agent. As aging progresses, less Hyaluronic acid is produced, resulting in wrinkled skin, thinning hair, unlubricated joints. The chelators of the present composition also help to preserve hyaluronic acid in the body. The hyaluronic acid component and the mineral chelating components (e.g., resveratrol, quercetin, phytic acid IP6, ferulate) work as a total anti-aging strategy to maintain youthful function within cells and connective tissues. Hyaluronic acid is believed to have an affinity to cancer cells. It is believed to serve as a delivery and targeting (drug delivery agent) molecule in blood circulation and to address aging of the connective tissue. The collapse and loss of integrity of connective tissue between cells provides the signs of aging (e.g., skin wrinkling, hair thinning, joint stiffness, loss of stature, etc.). The addition of hyaluronic acid to the compositions of the present invention is believed to activate fibroblast cells in the human body to produce additional hyaluronic acid, thus serving to preserve connective tissue (collagen) in a youthful state (Yadav, A. K. et al. (2008) “An Insight On Hyaluronic Acid In Drug Targeting And Drug Delivery,” J. Drug Target. 16(2):91-107; Liao, Y. H. et al. (2005) “Hyaluronan: Pharmaceutical Characterization And Drug Delivery,” Drug Deliv. 12(6):327-342; Joddar, B. et al. (2006) “Elastogenic Effects Of Exogenous Hyaluronan Oligosaccharides On Vascular Smooth Muscle Cells,” Biomaterials 27(33):5698-5707; Girish, K. S. et al. (2007) “The Magic Glue Hyaluronan And Its Eraser Hyaluronidase: A Biological Overview,” Life Sci. 80(21):1921-1943).
(d) Phytic Acid: Phytic Acid, preferably in the form of rice bran, is believed to act as an iron and copper chelator and as an inhibitor of calcium crystallization. Phytic Acid also is believed to reduce the availability of metallic minerals that serve as growth factors in tumor cells. It is also believed to serve as a neutrophil priming and motility agent. Additionally, phytic acid has been found to be neuroprotective, and thus to attenuate the severity of conditions associated with neurodegenerative diseases (especially Parkinson's Disease, camptocormia, and Alzheimer's Disease) (Xu, Q. et al. (Epub 2007 Dec. 27) “Neuroprotective Effect Of The Natural Iron Chelator, Phytic Acid In A Cell Culture Model Of Parkinson's Disease,” Toxicology 245(1-2):101-108). The components of the compositions of the present invention are believed to enhance such neuroprotection.

The iron chelator, quercetin, if present, is believed to serve to increase immediate bioavailability of resveratrol by permitting more passes through the liver before it is metabolized.

The individual components of the composition are believed to act synergistically to enhance the effect of, for example, resveratrol. Without intending to be limited thereby, it is proposed that the body's control or chelation of iron and calcium regulates the rate of aging after full growth has been achieved. During childhood growth all the iron and calcium are directed towards production of new bone and new red blood cells (hemoglobin). The cessation of childhood growth results in excess iron, copper and calcium, which then progressively (a) calcifies and (b) rusts tissues. The lysosomes begin to accumulate iron and calcium, which results in their dysfunction. The mitochondria begin to malfunction as they also progressively rust and calcify. The compositions of the present invention are believed to be capable of limiting or slowing the progressive rusting and calcification of cells and cellular organelles to thereby facilitate a slowing or reversal of the aging process. The chelation is what controls the genes. Genes are then favorably upregulated or downregulated. Resveratrol and a copper chelator are believed to act: (1) as controllers of calcium concentration via upregulation of osteocalcin, the hormone that helps retain calcium in bones and (2) as controllers of iron concentration via heme oxygenase, an antioxidant enzyme.

MAO inhibitors and iron chelators have been proposed as treatments for Parkinson's disease (Youdim, M. B. et al. (2004) “Novel Bifunctional Drugs Targeting Monoamine Oxidase Inhibition And Iron Chelation As An Approach To Neuroprotection In Parkinson's Disease And Other Neurodegenerative Diseases,” J. Neural. Transm. 111(10-11):1455-1471; Yáñez, M. et al. (2006) “(−)-Trans-Epsilon-Viniferin, A Polyphenol Present In Wines, Is An Inhibitor Of Noradrenaline And 5-Hydroxytryptamine Uptake And Of Monoamine Oxidase Activity,” Eur. J. Pharmacol. 542(1-3):54-60; Bureau, G. et al. (2008) “Resveratrol And Quercetin, Two Natural Polyphenols, Reduce Apoptotic Neuronal Cell Death Induced By Neuroinflammation,” J. Neurosci. Res. 86(2):403-410; Singh, A. et al. (2003) “Quercetin Potentiates L-Dopa Reversal Of Drug-Induced Catalepsy In Rats: Possible COMT/MAO Inhibition,” Pharmacol. 68(2):81-88; Gao, X. et al. (2007) “Prospective Study Of Dietary Pattern And Risk Of Parkinson Disease,” Am. J. Clin. Nutr. 86(5):1486-1494; Johnson, S. (2001) “Is Parkinson's Disease The Heterozygote Form Of Wilson's Disease: PD=½ WD?,” Med. Hypotheses 56(2):171-173). The compositions of the present invention which contain the MAO inhibitor and copper chelator, resveratrol, the iron chelator and MAO inhibitor, quercetin, and the broad metal chelator, phytic acid are particularly preferred for the treatment of neurodegenerative diseases (especially Parkinson's Disease, camptocormia, and Alzheimer's Disease) or in the amelioration of the symptoms of such diseases.

Having now generally described the invention, the same will be more readily under-stood through reference to the following examples, which are provided by way of illustration and are not intended to be limiting of the present invention unless specified.

### Example 1

### Comparative Effects of Resveratrol and the Compositions of the Present Invention

In order to determine if the compositions of the present invention were more effective than resveratrol alone in mediating a resveratrol biological activity, an analysis of gene expression was conducted, comparing the modulation of gene expression achieved by calorie restriction to the modulation of gene expression achieved by the compositions of the present invention.

Accordingly, the ability of resveratrol alone and the resveratrol-containing compositions of the present invention to up-regulate survival/longevity genes or down-regulate genes whose expression enhances cellular damage was compared using the expression profile of a calorie restricted (“CR”) animal as a positive control and the expression profile of a normally fed animal as a negative control. Male B6CHF1 mice (2 months of age) were thus either placed on a 40% calorie restricted diet, provided commercially obtained trans-resveratrol (Sigma Chemical; 1.25 mg/kg per day), provided a resveratrol-containing composition of the present invention (Longevinex®; Resveratrol Associates, LLC; 100 mg trans-resveratrol containing capsule per 80 kg human per day (i.e., 2.5 mg/kg per day of resveratrol (1.25 mg/kg per day trans-resveratrol) 0.31 mg/kg per day quercetin dihydrate, 0.94 mg/kg per day rice bran extract, 4.75 mg/kg per day rice bran oil and 0.70 mg/kg per day sunflower lecithin)). The mice were monitored until they had reached five months of age.

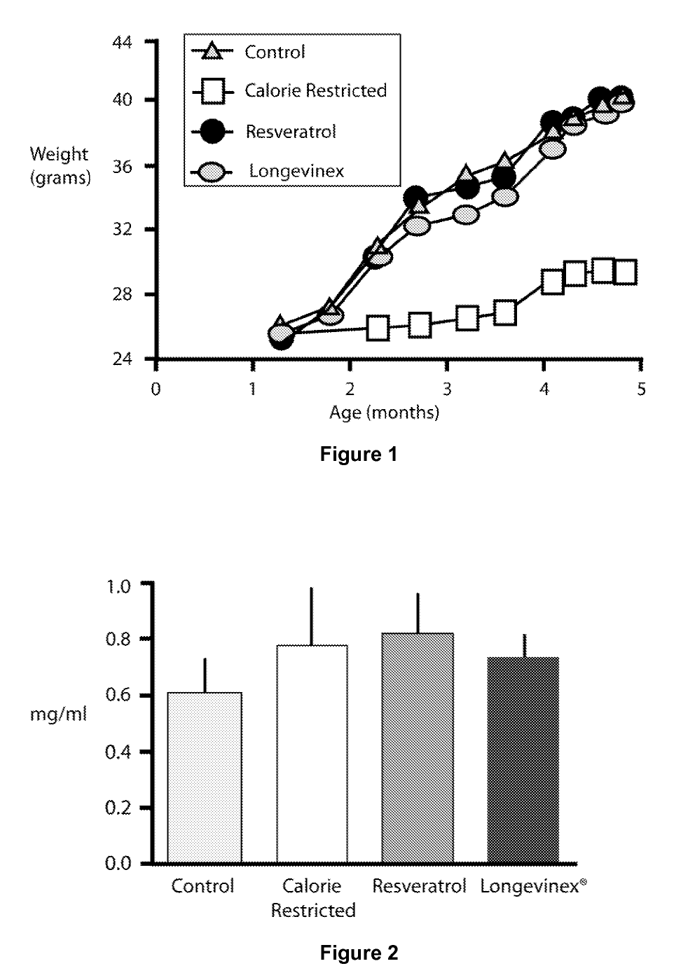

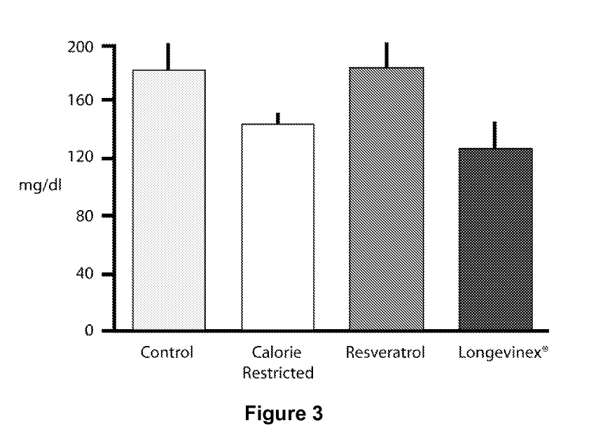

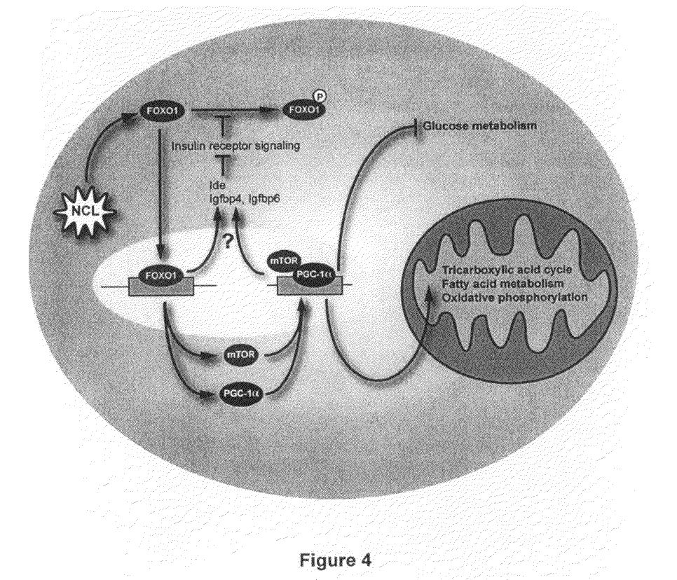

Body weight, serum glucose levels, serum insulin levels and lipid peroxidation in brain and muscle tissue were measured. The results showed that Longevinex® did not result in an increase in weight that was distinguishable from control animals (FIG. 1). Serum insulin levels were found to be approximately the same as that observed in the calorie restricted animals (FIG. 2). Serum glucose levels were found to be lower than that observed in the calorie restricted animals (FIG. 3).

### Example 2

### Comparative Effects of Resveratrol and the Compositions of the Present Invention on Gene Expression in Cardiac Tissue

The profile of expressed genes in the cardiac tissue of mice receiving resveratrol or a composition of the present invention (Longevinex®) was compared to that of mice placed on a calorie restricted diet and control mice. Gene expression was monitored using an Affymetrix MG430 2.0 Array, containing 45,101 probe sets per array. In cases in which the array represented the same gene with multiple probes, the probe set with the highest signal intensity was employed. Unknown genes (including uncharacterized ESTs and cDNA sequences were not analyzed. Thus, the array provided a means for analyzing 20,341 genes having a single Entrez Gene ID. Analysis was conducted substantially as described by Lee, C.-K. et al. (2002) “Transcriptional Profiles Associated With Aging And Middle Age-Onset Caloric Restriction In Mouse Hearts,” Proc. Natl. Acad. Sci. (U.S.A.) 99:14988-14993, herein incorporated by reference. The mean of all arrays in a group were calculated. The means of treated groups were compared to the mean of the control group, and the statistical significance of any differences were determined using two-tailed t-tests (P<0.01). The results of the analysis are presented in Table 3 (CO, control; CR, calorie restricted; RES, resveratrol; LGX, Longevinex®; FC, fold change. FC is calculated as the mean of the treated group divided by the mean of the control group, and this value is then log-transformed (base 2) for statistical purposes. As an example, a gene that is expressed at 100 in the control and 200 in a treated group would be have an Fc of 2 (i.e., a twofold increase in expression); a gene that is expressed at 100 in the control and 50 in the treated group, would have an Fc of −2 (i.e., a twofold decrease in expression).

Treatment of human umbilical vein epithelial cells with ferulic acid, quercetin or resveratrol has been reported to result in changes to gene expression of greater than 2-fold down-regulation of 363 genes, and greater than 2-fold up-regulation of 233 genes of 10,000 genes probed Nicholson, S. K. et al. (2008) “Effects Of Dietary Polyphenols On Gene Expression In Human Vascular Endothelial Cells,” Proc. Nutr. Soc. 67(1):42-47). In contrast, Table 3 shows that 2,829 genes were found to exhibit a statistically significant change in expression in treated vs. control mice. Of these genes, 7% were found to exhibit altered expression in mice that had been subjected to only calorie restriction; 8% were found to exhibit altered expression in mice subjected only to resveratrol. Combining calorie restriction with resveratrol administration failed to alter the expression of any additional genes. In contrast, administration of Longevinex® was found to alter the expression of 61% of the 2,829 genes. Administration of Longevinex® to calorie restricted mice was found to alter the expression of an additional 2% of the genes. Administration of Longevinex® to mice receiving resveratrol was found to alter the expression of an additional 21% of the genes. Thus, Longevinex® alone or in combination with other regimens was found to affect 85% (2,406) of the total genes showing altered expression.

TABLE 3

| | | CO | CR | RES | LGX | FC | FC | FC | |
|---|---|---|---|---|---|---|---|---|---|
| Probe Set ID | Treatment | mean | mean | mean | mean | CR | Res | Lgx | Entrez Info |
| 1415670_at | Lgx only | 987 | 1139 | 1187 | 1378 | 1.15 | 1.20 | 1.40 | Copg |
| 1415671_at | Res & Lgx | 2454 | 2539 | 2030 | 2071 | 1.03 | −1.21 | −1.19 | Atp6v0d1 |
| 1415672_at | Res & Lgx | 3213 | 2819 | 2637 | 2262 | −1.14 | −1.22 | −1.42 | Golga7 |
| 1415677_at | Res & Lgx | 706 | 747 | 933 | 934 | 1.06 | 1.32 | 1.32 | Dhrs1 |
| 1415679_at | Lgx only | 4825 | 4129 | 3776 | 3154 | −1.17 | −1.28 | −1.53 | Psenen |
| 1415684_at | Lgx only | 638 | 573 | 490 | 381 | −1.11 | −1.30 | −1.67 | Atg5 |
| 1415696_at | Res only | 6629 | 7063 | 5491 | 5988 | 1.07 | −1.21 | −1.11 | Sar1a |
| 1415700_a_at | Res & Lgx | 5461 | 5179 | 3791 | 2603 | −1.05 | −1.44 | −2.10 | Ssr3 |
| 1415704_a_at | Lgx only | 5010 | 4472 | 3850 | 3285 | −1.12 | −1.30 | −1.52 | Cdv3 |
| 1415707_at | Lgx only | 1111 | 1317 | 1468 | 1535 | 1.19 | 1.32 | 1.38 | Anapc2 |
| 1415714_a_at | Res & Lgx | 4941 | 4632 | 2720 | 2379 | −1.07 | −1.82 | −2.08 | 2610209M04Rik |
| 1415723_at | Res & Lgx | 4272 | 3466 | 3385 | 3062 | −1.23 | −1.26 | −1.40 | Eif5 |
| 1415733_a_at | Lgx only | 13615 | 13963 | 9719 | 9991 | 1.03 | −1.40 | −1.36 | 1110019J04Rik |
| 1415735_at | Lgx only | 2025 | 2617 | 3355 | 3642 | 1.29 | 1.66 | 1.80 | Ddb1 |
| 1415736_at | Res & Lgx | 4957 | 3953 | 2536 | 1805 | −1.25 | −1.95 | −2.75 | Pfdn5 |
| 1415738_at | Res & Lgx | 1914 | 1941 | 1575 | 1395 | 1.01 | −1.22 | −1.37 | Txndc12 |
| 1415742_at | Lgx only | 749 | 837 | 856 | 987 | 1.12 | 1.14 | 1.32 | Aup1 |
| 1415746_at | Lgx only | 726 | 949 | 1075 | 1501 | 1.31 | 1.48 | 2.07 | Cic |
| 1415749_a_at | Lgx only | 2368 | 2397 | 2111 | 1912 | 1.01 | −1.12 | −1.24 | Rragc |
| 1415754_at | Lgx only | 3512 | 3195 | 3099 | 2411 | −1.10 | −1.13 | −1.46 | Polr2f |
| 1415755_a_at | Lgx only | 2983 | 3602 | 3890 | 4068 | 1.21 | 1.30 | 1.36 | Ube2v1 |
| 1415756_a_at | Res & Lgx | 3581 | 3290 | 2710 | 2706 | −1.09 | −1.32 | −1.32 | Snapap |
| 1415757_at | Lgx only | 901 | 1073 | 1189 | 1337 | 1.19 | 1.32 | 1.48 | Gbf1 |
| 1415764_at | Lgx only | 3381 | 3218 | 2545 | 2419 | −1.05 | −1.33 | −1.40 | Zc3h11a |
| 1415783_at | Lgx only | 4869 | 4728 | 4237 | 3960 | −1.03 | −1.15 | −1.23 | Vps35 |
| 1415788_at | Lgx only | 1490 | 1572 | 1169 | 1046 | 1.05 | −1.27 | −1.42 | Ublcp1 |
| 1415791_at | Res & Lgx | 2983 | 2870 | 2364 | 1805 | −1.04 | −1.26 | −1.65 | Rnf34 |
| 1415797_at | Lgx only | 319 | 488 | 522 | 805 | 1.53 | 1.64 | 2.53 | Ddr1 |
| 1415802_at | Res & Lgx | 8566 | 8379 | 4763 | 4548 | −1.02 | −1.80 | −1.88 | Slc16a1 |
| 1415812_at | Lgx only | 31926 | 36029 | 41778 | 58920 | 1.13 | 1.31 | 1.85 | Gsn |
| 1415814_at | CR only | 1760 | 2239 | 1514 | 1433 | 1.27 | −1.16 | −1.23 | Atp6v1b2 |
| 1415816_at | Res only | 5617 | 5279 | 4428 | 5553 | −1.06 | −1.27 | −1.01 | Cct7 |
| 1415818_at | Lgx only | 2554 | 2785 | 2838 | 3335 | 1.09 | 1.11 | 1.31 | Anxa6 |
| 1415830_at | Lgx only | 586 | 494 | 424 | 318 | −1.19 | −1.38 | −1.84 | Orc5l |
| 1415834_at | Res only | 579 | 703 | 419 | 430 | 1.21 | −1.38 | −1.35 | Dusp6 |
| 1415840_at | Res & Lgx | 1636 | 1461 | 974 | 856 | −1.12 | −1.68 | −1.91 | Elovl5 |
| 1415850_at | Res & Lgx | 824 | 880 | 1129 | 1093 | 1.07 | 1.37 | 1.33 | Rasa3 |
| 1415856_at | Lgx only | 148 | 168 | 172 | 242 | 1.14 | 1.16 | 1.63 | Emb |
| 1415875_at | Res only | 83 | 38 | 34 | 51 | −2.19 | −2.43 | −1.63 | 3010003L21Rik |
| 1415876_a_at | Lgx only | 13888 | 14344 | 16149 | 18965 | 1.03 | 1.16 | 1.37 | Rps26 |
| 1415879_a_at | Lgx only | 33385 | 37008 | 41034 | 49898 | 1.11 | 1.23 | 1.49 | Rplp2 |
| 1415882_at | Res only | 20588 | 18774 | 17207 | 17741 | −1.10 | −1.20 | −1.16 | Ghitm |
| 1415886_at | All | 637 | 966 | 1017 | 1132 | 1.51 | 1.60 | 1.78 | Sh2d3c |
| 1415901_at | Lgx only | 1435 | 1699 | 1682 | 1903 | 1.18 | 1.17 | 1.33 | Plod3 |
| 1415907_at | Lgx only | 1656 | 1817 | 2329 | 2446 | 1.10 | 1.41 | 1.48 | Ccnd3 |
| 1415909_at | Res & Lgx | 1788 | 1949 | 1414 | 1451 | 1.09 | −1.26 | −1.23 | Stip1 |
| 1415915_at | Res & Lgx | 2843 | 3059 | 3559 | 3668 | 1.08 | 1.25 | 1.29 | Ddx1 |
| 1415930_a_at | Lgx only | 14546 | 13685 | 14019 | 11677 | −1.06 | −1.04 | −1.25 | Map1lc3b |
| 1415935_at | Res & Lgx | 1687 | 1988 | 2011 | 2446 | 1.18 | 1.19 | 1.45 | Smoc2 |
| 1415947_at | CR only | 7499 | 6219 | 8373 | 7163 | −1.21 | 1.12 | −1.05 | Creg1 |
| 1415951_at | Lgx only | 496 | 588 | 587 | 717 | 1.19 | 1.18 | 1.45 | Fkbp10 |
| 1415961_at | Lgx only | 2152 | 2177 | 2503 | 3192 | 1.01 | 1.16 | 1.48 | Itm2c |
| 1415966_a_at | Lgx only | 29789 | 33932 | 35207 | 35979 | 1.14 | 1.18 | 1.21 | Ndufv1 |
| 1415971_at | CR only | 1825 | 1273 | 1649 | 1440 | −1.43 | −1.11 | −1.27 | Marcks |
| 1415974_at | Lgx only | 1694 | 2075 | 2211 | 2392 | 1.22 | 1.31 | 1.41 | Map2k2 |
| 1415977_at | Lgx only | 444 | 603 | 658 | 715 | 1.36 | 1.48 | 1.61 | Isyna1 |
| 1415987_at | Lgx only | 8838 | 9568 | 10218 | 11109 | 1.08 | 1.16 | 1.26 | Hdlbp |
| 1415990_at | CR & Lgx | 27024 | 32941 | 29714 | 35847 | 1.22 | 1.10 | 1.33 | Vdac2 |
| 1415991_a_at | Res & Lgx | 3051 | 3384 | 4293 | 4892 | 1.11 | 1.41 | 1.60 | Klhdc3 |
| 1415996_at | All | 10650 | 22628 | 23188 | 27989 | 2.12 | 2.18 | 2.63 | Txnip |
| 1415998_at | Lgx only | 33371 | 40497 | 35239 | 46095 | 1.21 | 1.06 | 1.38 | Vdac1 |
| 1416013_at | Res & Lgx | 926 | 1171 | 1314 | 1772 | 1.26 | 1.42 | 1.91 | Pld3 |
| 1416014_at | All | 1518 | 981 | 801 | 662 | −1.55 | −1.90 | −2.29 | Abce1 |
| 1416016_at | Res & Lgx | 232 | 236 | 387 | 358 | 1.02 | 1.67 | 1.55 | Tap1 |
| 1416019_at | Res only | 336 | 291 | 275 | 269 | −1.15 | −1.22 | −1.25 | Dr1 |
| 1416027_at | Res & Lgx | 2414 | 2298 | 1834 | 1569 | −1.05 | −1.32 | −1.54 | Pdcd6 |
| 1416032_at | Lgx only | 1053 | 1425 | 1776 | 1638 | 1.35 | 1.69 | 1.56 | Tmem109 |
| 1416046_a_at | Lgx only | 4982 | 5500 | 3916 | 3426 | 1.10 | −1.27 | −1.45 | Fuca2 |
| 1416048_at | Res & Lgx | 1089 | 1174 | 1589 | 1635 | 1.08 | 1.46 | 1.50 | Phc2 |
| 1416050_a_at | Lgx only | 903 | 1023 | 1209 | 1283 | 1.13 | 1.34 | 1.42 | Scarb1 |
| 1416051_at | Lgx only | 128 | 165 | 178 | 252 | 1.28 | 1.39 | 1.97 | C2 |
| 1416061_at | Lgx only | 1318 | 1255 | 1052 | 964 | −1.05 | −1.25 | −1.37 | Tbc1d15 |
| 1416064_a_at | Res only | 12341 | 12004 | 9273 | 10433 | −1.03 | −1.33 | −1.18 | Hspa5 |
| 1416069_at | CR only | 1055 | 1396 | 1197 | 1313 | 1.32 | 1.13 | 1.24 | Pfkp |
| 1416079_a_at | Lgx only | 1323 | 1494 | 1456 | 1611 | 1.13 | 1.10 | 1.22 | Arpc1a |
| 1416082_at | Res & Lgx | 13983 | 13415 | 11321 | 10270 | −1.04 | −1.24 | −1.36 | Rab1 |
| 1416091_at | Lgx only | 1996 | 2372 | 2622 | 3315 | 1.19 | 1.31 | 1.66 | Mtap4 |
| 1416106_at | Lgx only | 1108 | 939 | 1035 | 775 | −1.18 | −1.07 | −1.43 | Kti12 |
| 1416111_at | CR & Lgx | 368 | 212 | 288 | 138 | −1.74 | −1.28 | −2.68 | Cd83 |
| 1416112_at | Lgx only | 7587 | 9334 | 8583 | 12194 | 1.23 | 1.13 | 1.61 | Cox8a |
| 1416113_at | Lgx only | 1810 | 1965 | 2224 | 2559 | 1.09 | 1.23 | 1.41 | Fkbp8 |
| 1416125_at | Lgx only | 550 | 764 | 772 | 809 | 1.39 | 1.40 | 1.47 | Fkbp5 |
| 1416129_at | Lgx only | 1270 | 1118 | 1078 | 734 | −1.14 | −1.18 | −1.73 | Errfi1 |
| 1416140_a_at | Lgx only | 1672 | 1904 | 1977 | 1952 | 1.14 | 1.18 | 1.17 | Dhx30 |
| 1416142_at | Lgx only | 614 | 690 | 511 | 333 | 1.12 | −1.20 | −1.85 | Rps6 |
| 1416155_at | Res & Lgx | 3723 | 3890 | 2824 | 2551 | 1.04 | −1.32 | −1.46 | Hmgb3 |
| 1416175_a_at | Res & Lgx | 21409 | 19416 | 15845 | 13003 | −1.10 | −1.35 | −1.65 | Vdac3 |
| 1416176_at | Lgx only | 2064 | 1801 | 1869 | 1358 | −1.15 | −1.10 | −1.52 | Hmgb1 |
| 1416177_at | Res & Lgx | 884 | 734 | 669 | 666 | −1.20 | −1.32 | −1.33 | Rbmxrt |
| 1416181_at | Res only | 1982 | 1958 | 1504 | 1705 | −1.01 | −1.32 | −1.16 | Mesdc2 |
| 1416183_a_at | Lgx only | 40384 | 48786 | 54318 | 64528 | 1.21 | 1.35 | 1.60 | Ldhb |
| 1416185_a_at | Lgx only | 4447 | 4224 | 3331 | 2724 | −1.05 | −1.33 | −1.63 | Adh5 |
| 1416186_at | Lgx only | 404 | 340 | 342 | 213 | −1.19 | −1.18 | −1.90 | Pnrc2 |
| 1416195_at | Lgx only | 1575 | 1576 | 1933 | 2022 | 1.00 | 1.23 | 1.28 | RP23-136K12.4 |
| 1416209_at | CR & Lgx | 3673 | 4359 | 4108 | 4135 | 1.19 | 1.12 | 1.13 | Glud1 |
| 1416210_at | Lgx only | 2605 | 2387 | 2225 | 2041 | −1.09 | −1.17 | −1.28 | Imp3 |
| 1416223_at | Lgx only | 629 | 633 | 838 | 882 | 1.01 | 1.33 | 1.40 | Sh3bp5l |
| 1416226_at | Lgx only | 1514 | 1558 | 2186 | 2127 | 1.03 | 1.44 | 1.40 | Arpc1b |
| 1416238_at | Lgx only | 982 | 1045 | 1322 | 1446 | 1.06 | 1.35 | 1.47 | Tie1 |
| 1416240_at | Lgx only | 22627 | 18896 | 18958 | 17768 | −1.20 | −1.19 | −1.27 | Psmb7 |
| 1416252_at | Lgx only | 1031 | 1322 | 1235 | 1464 | 1.28 | 1.20 | 1.42 | Stk38 |
| 1416254_a_at | Lgx only | 432 | 478 | 497 | 673 | 1.11 | 1.15 | 1.56 | Vps16 |
| 1416256_a_at | CR only | 4550 | 5806 | 4704 | 5339 | 1.28 | 1.03 | 1.17 | Tubb5 |
| 1416259_at | CR & Lgx | 573 | 393 | 417 | 336 | −1.46 | −1.37 | −1.71 | Pex12 |
| 1416261_at | Lgx only | 328 | 394 | 350 | 470 | 1.20 | 1.07 | 1.43 | Tmem19 |
| 1416268_at | Lgx only | 4666 | 4083 | 4613 | 3784 | −1.14 | −1.01 | −1.23 | Ets2 |
| 1416271_at | CR & Lgx | 3787 | 4661 | 4246 | 5035 | 1.23 | 1.12 | 1.33 | Perp |
| 1416272_at | Lgx only | 4874 | 4932 | 3921 | 3693 | 1.01 | −1.24 | −1.32 | Map2k1ip1 |
| 1416280_at | Lgx only | 1640 | 1595 | 1340 | 1270 | −1.03 | −1.22 | −1.29 | Sae2 |
| 1416283_at | Res & Lgx | 1026 | 1225 | 1415 | 1355 | 1.19 | 1.38 | 1.32 | Gart |
| 1416284_at | Lgx only | 5006 | 5498 | 6000 | 6739 | 1.10 | 1.20 | 1.35 | Mrpl28 |
| 1416292_at | Res & Lgx | 13464 | 12624 | 11145 | 11453 | −1.07 | −1.21 | −1.18 | Prdx3 |
| 1416294_at | Lgx only | 1274 | 1556 | 1654 | 2003 | 1.22 | 1.30 | 1.57 | Scamp3 |
| 1416300_a_at | Lgx only | 81172 | 93900 | 86598 | 106206 | 1.16 | 1.07 | 1.31 | Slc25a3 |
| 1416312_at | Res only | 1570 | 1597 | 1351 | 1503 | 1.02 | −1.16 | −1.04 | Rars |
| 1416315_at | Lgx only | 1812 | 2063 | 2221 | 2496 | 1.14 | 1.23 | 1.38 | Abhd4 |
| 1416326_at | Lgx only | 8540 | 9522 | 10268 | 12292 | 1.11 | 1.20 | 1.44 | Crip1 |
| 1416329_at | CR only | 1231 | 933 | 1064 | 976 | −1.32 | −1.16 | −1.26 | Cyfip1 |
| 1416331_a_at | Lgx only | 4998 | 5244 | 6828 | 7398 | 1.05 | 1.37 | 1.48 | Nfe2l1 |
| 1416339_a_at | Lgx only | 1730 | 1851 | 1929 | 2312 | 1.07 | 1.12 | 1.34 | Prkcsh |
| 1416340_a_at | Lgx only | 1678 | 1836 | 2208 | 2386 | 1.09 | 1.32 | 1.42 | Man2b1 |
| 1416350_at | Lgx only | 209 | 134 | 177 | 85 | −1.56 | −1.18 | −2.46 | Klf16 |
| 1416366_at | Lgx only | 27546 | 25654 | 21110 | 19836 | −1.07 | −1.30 | −1.39 | Ndufc2 |
| 1416368_at | Lgx only | 3559 | 3865 | 3913 | 4425 | 1.09 | 1.10 | 1.24 | Gsta4 |
| 1416369_at | Lgx only | 728 | 542 | 558 | 430 | −1.34 | −1.30 | −1.69 | Hiatl1 |
| 1416371_at | Lgx only | 934 | 773 | 973 | 1314 | −1.21 | 1.04 | 1.41 | Apod |
| 1416384_a_at | Lgx only | 3771 | 3990 | 4230 | 4569 | 1.06 | 1.12 | 1.21 | Cope |
| 1416393_at | Lgx only | 5020 | 5227 | 4128 | 2942 | 1.04 | −1.22 | −1.71 | Emg1 |
| 1416405_at | Lgx only | 5254 | 5912 | 7260 | 10205 | 1.13 | 1.38 | 1.94 | Bgn |
| 1416411_at | Lgx only | 3752 | 3573 | 3504 | 3347 | −1.05 | −1.07 | −1.12 | Gstm2 |
| 1416412_at | Res & Lgx | 716 | 471 | 369 | 274 | −1.52 | −1.94 | −2.61 | Nsmaf |
| 1416414_at | Lgx only | 461 | 561 | 703 | 739 | 1.22 | 1.52 | 1.60 | Emilin1 |
| 1416417_a_at | Lgx only | 29794 | 33340 | 35044 | 38096 | 1.12 | 1.18 | 1.28 | Ndufb7 |
| 1416424_at | Lgx only | 3969 | 4305 | 4914 | 5377 | 1.08 | 1.24 | 1.35 | M6prbp1 |
| 1416425_at | Lgx only | 3908 | 4090 | 4560 | 4505 | 1.05 | 1.17 | 1.15 | Pex19 |
| 1416427_at | Lgx only | 14038 | 14608 | 14480 | 17881 | 1.04 | 1.03 | 1.27 | Ccni |
| 1416436_a_at | Res & Lgx | 3463 | 3513 | 2761 | 2608 | 1.01 | −1.25 | −1.33 | Uqcc |
| 1416438_at | Lgx only | 1704 | 1946 | 2041 | 2300 | 1.14 | 1.20 | 1.35 | Puf60 |
| 1416452_at | Lgx only | 5918 | 6059 | 6368 | 7090 | 1.02 | 1.08 | 1.20 | Oat |
| 1416455_a_at | Lgx only | 49845 | 53641 | 55445 | 64019 | 1.08 | 1.11 | 1.28 | Cryab |
| 1416457_at | Lgx only | 1208 | 1356 | 1592 | 1930 | 1.12 | 1.32 | 1.60 | Ddah2 |
| 1416462_at | Res & Lgx | 3796 | 3321 | 2539 | 2621 | −1.14 | −1.49 | −1.45 | Caprin1 |
| 1416478_a_at | Lgx only | 49370 | 54196 | 49980 | 56965 | 1.10 | 1.01 | 1.15 | Mdh2 |
| 1416479_a_at | Lgx only | 3314 | 4024 | 3824 | 5065 | 1.21 | 1.15 | 1.53 | Tmem14c |
| 1416494_at | Lgx only | 24034 | 24945 | 28712 | 32059 | 1.04 | 1.19 | 1.33 | Ndufs5 |
| 1416496_at | CR & Lgx | 9192 | 10747 | 9867 | 10827 | 1.17 | 1.07 | 1.18 | Mrfap1 |
| 1416498_at | CR & Lgx | 1234 | 1020 | 1151 | 968 | −1.21 | −1.07 | −1.28 | Ppic |
| 1416502_a_at | Res & Lgx | 1327 | 1624 | 1659 | 1918 | 1.22 | 1.25 | 1.45 | Preb |
| 1416506_at | Lgx only | 5718 | 5208 | 4876 | 4228 | −1.10 | −1.17 | −1.35 | Psma6 |
| 1416510_at | Lgx only | 5555 | 6339 | 5912 | 6679 | 1.14 | 1.06 | 1.20 | Mrpl4 |
| 1416513_at | Lgx only | 1582 | 2144 | 2762 | 2762 | 1.35 | 1.75 | 1.75 | Lamb2 |
| 1416514_a_at | Lgx only | 888 | 805 | 648 | 439 | −1.10 | −1.37 | −2.02 | Fscn1 |
| 1416517_at | Lgx only | 411 | 441 | 486 | 580 | 1.07 | 1.18 | 1.41 | Pnpla6 |
| 1416524_at | Lgx only | 4034 | 4167 | 4527 | 5256 | 1.03 | 1.12 | 1.30 | Spop |
| 1416540_at | Lgx only | 1017 | 1340 | 1386 | 1769 | 1.32 | 1.36 | 1.74 | Hgs |
| 1416547_at | Lgx only | 12925 | 12431 | 11457 | 10620 | −1.04 | −1.13 | −1.22 | Ndufb3 |
| 1416555_at | Res & Lgx | 2837 | 2868 | 2221 | 2008 | 1.01 | −1.28 | −1.41 | Ei24 |
| 1416563_at | Lgx only | 1374 | 1532 | 1043 | 788 | 1.11 | −1.32 | −1.74 | Ctps |
| 1416576_at | Lgx only | 230 | 236 | 432 | 441 | 1.02 | 1.88 | 1.92 | Socs3 |
| 1416587_a_at | Res & Lgx | 372 | 605 | 623 | 822 | 1.63 | 1.68 | 2.21 | Xrcc1 |
| 1416595_at | Lgx only | 1722 | 1750 | 1272 | 1269 | 1.02 | −1.35 | −1.36 | Mrps22 |
| 1416604_at | Lgx only | 55121 | 62820 | 59589 | 71198 | 1.14 | 1.08 | 1.29 | Cyc1 |
| 1416612_at | Lgx only | 424 | 520 | 635 | 826 | 1.23 | 1.50 | 1.95 | Cyp1b1 |
| 1416621_at | Res & Lgx | 869 | 1007 | 1063 | 1287 | 1.16 | 1.22 | 1.48 | Llgl1 |
| 1416629_at | Lgx only | 412 | 524 | 548 | 620 | 1.27 | 1.33 | 1.50 | Slc1a5 |
| 1416634_at | Res & Lgx | 3167 | 2719 | 1899 | 1420 | −1.16 | −1.67 | −2.23 | 5730536A07Rik |
| 1416635_at | Res & Lgx | 4444 | 3643 | 3384 | 2684 | −1.22 | −1.31 | −1.66 | Smpdl3a |
| 1416637_at | Lgx only | 525 | 603 | 661 | 780 | 1.15 | 1.26 | 1.49 | Slc4a2 |
| 1416647_at | Lgx only | 5775 | 6096 | 7099 | 7557 | 1.06 | 1.23 | 1.31 | Bckdha |
| 1416648_at | Lgx only | 2599 | 3174 | 3890 | 4330 | 1.22 | 1.50 | 1.67 | Dync1h1 |
| 1416656_at | Lgx only | 1412 | 1659 | 1596 | 1864 | 1.18 | 1.13 | 1.32 | Clic1 |
| 1416668_at | Lgx only | 10958 | 10417 | 8267 | 7237 | −1.05 | −1.33 | −1.51 | Ttc35 |
| 1416680_at | Res & Lgx | 2599 | 1810 | 1455 | 813 | −1.44 | −1.79 | −3.20 | Ube3a |
| 1416683_at | Lgx only | 1687 | 2209 | 2493 | 2585 | 1.31 | 1.48 | 1.53 | Plxnb2 |
| 1416690_at | Res & Lgx | 1379 | 1680 | 1929 | 1834 | 1.22 | 1.40 | 1.33 | Gtpbp2 |
| 1416699_at | Res only | 6446 | 6175 | 4968 | 5208 | −1.04 | −1.30 | −1.24 | 1110008F13Rik |
| 1416703_at | Lgx only | 2541 | 2311 | 2251 | 1680 | −1.10 | −1.13 | −1.51 | Mapk14 |
| 1416706_at | Res only | 408 | 412 | 258 | 254 | 1.01 | −1.58 | −1.61 | Rpe |
| 1416708_a_at | Lgx only | 807 | 815 | 975 | 1044 | 1.01 | 1.21 | 1.29 | Gramd1a |
| 1416709_a_at | Res & Lgx | 1287 | 1148 | 920 | 920 | −1.12 | −1.40 | −1.40 | Ngrn |
| 1416713_at | Lgx only | 920 | 1162 | 1424 | 2002 | 1.26 | 1.55 | 2.18 | Tppp3 |
| 1416730_at | Lgx only | 436 | 492 | 540 | 650 | 1.13 | 1.24 | 1.49 | Rcl1 |
| 1416731_at | Lgx only | 1678 | 1349 | 1389 | 1118 | −1.24 | −1.21 | −1.50 | Top2b |
| 1416737_at | Lgx only | 1600 | 2387 | 2293 | 2936 | 1.49 | 1.43 | 1.84 | Gys1 |
| 1416740_at | Lgx only | 1068 | 1202 | 1516 | 1606 | 1.13 | 1.42 | 1.50 | Col5a1 |
| 1416749_at | CR only | 4558 | 5528 | 3766 | 3810 | 1.21 | −1.21 | −1.20 | Htra1 |
| 1416752_at | Lgx only | 18842 | 22289 | 23077 | 26541 | 1.18 | 1.22 | 1.41 | Ldb3 |
| 1416755_at | CR & Lgx | 1571 | 1157 | 1295 | 897 | −1.36 | −1.21 | −1.75 | Dnajb1 |
| 1416766_at | Lgx only | 2214 | 2261 | 2504 | 2834 | 1.02 | 1.13 | 1.28 | Mosc2 |
| 1416791_a_at | Lgx only | 1176 | 1162 | 1174 | 1630 | −1.01 | −1.00 | 1.39 | Nxf1 |
| 1416805_at | Lgx only | 1361 | 1327 | 1132 | 959 | −1.03 | −1.20 | −1.42 | 1110032E23Rik |
| 1416808_at | Res & Lgx | 2839 | 2486 | 1876 | 1518 | −1.14 | −1.51 | −1.87 | Nid1 |
| 1416819_at | CR only | 7318 | 8511 | 7739 | 8109 | 1.16 | 1.06 | 1.11 | Cdc37 |
| 1416824_at | Res & Lgx | 6820 | 5799 | 4916 | 4447 | −1.18 | −1.39 | −1.53 | B230118H07Rik |
| 1416832_at | Lgx only | 179 | 250 | 238 | 315 | 1.40 | 1.33 | 1.76 | Slc39a8 |
| 1416836_at | Lgx only | 2043 | 2339 | 2345 | 2519 | 1.15 | 1.15 | 1.23 | Lrp10 |
| 1416841_at | Lgx only | 761 | 651 | 582 | 494 | −1.17 | −1.31 | −1.54 | 1110059E24Rik |
| 1416842_at | Lgx only | 5799 | 5337 | 5053 | 4625 | −1.09 | −1.15 | −1.25 | Gstm5 |
| 1416845_at | Res & Lgx | 253 | 365 | 392 | 412 | 1.44 | 1.55 | 1.63 | Tmem132a |
| 1416867_at | Res only | 539 | 510 | 644 | 503 | −1.06 | 1.19 | −1.07 | Bet1 |
| 1416883_at | Res & Lgx | 3457 | 4227 | 5144 | 5330 | 1.22 | 1.49 | 1.54 | Clptm1 |
| 1416884_at | Lgx only | 1755 | 1451 | 1259 | 1064 | −1.21 | −1.39 | −1.65 | Cbx3 |
| 1416896_at | Res only | 354 | 336 | 521 | 404 | −1.05 | 1.47 | 1.14 | Rps6ka1 |
| 1416903_at | Lgx only | 2268 | 2513 | 2807 | 2977 | 1.11 | 1.24 | 1.31 | Nucb1 |
| 1416912_at | Lgx only | 2816 | 2469 | 2287 | 1840 | −1.14 | −1.23 | −1.53 | 6330407G11Rik |
| 1416928_at | Res & Lgx | 295 | 289 | 197 | 136 | −1.02 | −1.50 | −2.16 | Rbm12 |
| 1416931_at | Lgx only | 695 | 726 | 597 | 533 | 1.05 | −1.16 | −1.30 | Nif3l1 |
| 1416933_at | Res & Lgx | 1482 | 1837 | 2077 | 2466 | 1.24 | 1.40 | 1.66 | Por |
| 1416940_at | Lgx only | 9984 | 10810 | 10055 | 11650 | 1.08 | 1.01 | 1.17 | Ppif |
| 1416943_at | Lgx only | 1721 | 1477 | 1227 | 872 | −1.16 | −1.40 | −1.97 | Ube2e1 |
| 1416953_at | CR only | 2136 | 3722 | 2891 | 2928 | 1.74 | 1.35 | 1.37 | Ctgf |
| 1416963_at | Res only | 4471 | 4553 | 3878 | 4212 | 1.02 | −1.15 | −1.06 | Ubac1 |
| 1416981_at | Lgx only | 926 | 912 | 1189 | 1623 | −1.02 | 1.28 | 1.75 | Foxo1 |
| 1416990_at | Lgx only | 926 | 985 | 1107 | 1231 | 1.06 | 1.19 | 1.33 | Rxrb |
| 1417000_at | Lgx only | 448 | 513 | 582 | 661 | 1.14 | 1.30 | 1.48 | Abtb1 |
| 1417006_at | Lgx only | 917 | 923 | 1024 | 1208 | 1.01 | 1.12 | 1.32 | Commd4 |
| 1417007_a_at | Lgx only | 2381 | 1901 | 1585 | 1243 | −1.25 | −1.50 | −1.92 | Vps4b |
| 1417008_at | Lgx only | 4995 | 5792 | 7236 | 8553 | 1.16 | 1.45 | 1.71 | Crat |
| 1417010_at | Lgx only | 1566 | 1338 | 1138 | 897 | −1.17 | −1.38 | −1.75 | Zfp238 |
| 1417018_at | Lgx only | 442 | 453 | 487 | 682 | 1.03 | 1.10 | 1.54 | Efemp2 |
| 1417026_at | Lgx only | 1683 | 1749 | 1357 | 1217 | 1.04 | −1.24 | −1.38 | Pfdn1 |
| 1417044_at | Lgx only | 959 | 1047 | 1076 | 1385 | 1.09 | 1.12 | 1.44 | Lcmt1 |
| 1417049_at | CR only | 693 | 515 | 650 | 556 | −1.34 | −1.07 | −1.25 | Rhd |
| 1417061_at | All | 1418 | 1003 | 1945 | 1761 | −1.41 | 1.37 | 1.24 | Slc40a1 |
| 1417065_at | CR only | 667 | 1043 | 914 | 758 | 1.56 | 1.37 | 1.14 | Egr1 |
| 1417068_a_at | Res & Lgx | 1030 | 1108 | 1271 | 1589 | 1.08 | 1.23 | 1.54 | Ptpn1 |
| 1417073_a_at | Lgx only | 2797 | 2634 | 1839 | 1346 | −1.06 | −1.52 | −2.08 | Qk |
| 1417075_at | Lgx only | 2020 | 1802 | 1853 | 1594 | −1.12 | −1.09 | −1.27 | 2010309E21Rik |
| 1417081_a_at | Lgx only | 3594 | 4012 | 4252 | 4459 | 1.12 | 1.18 | 1.24 | Syngr2 |
| 1417082_at | Lgx only | 1946 | 1607 | 1455 | 1101 | −1.21 | −1.34 | −1.77 | Anp32b |
| 1417091_at | Lgx only | 520 | 510 | 358 | 207 | −1.02 | −1.45 | −2.51 | Chuk |
| 1417105_at | Lgx only | 5064 | 4514 | 4396 | 4198 | −1.12 | −1.15 | −1.21 | Trappc2l |
| 1417109_at | Res & Lgx | 1215 | 1681 | 1773 | 1883 | 1.38 | 1.46 | 1.55 | Tinagl |
| 1417112_at | Res & Lgx | 3493 | 4015 | 4629 | 4787 | 1.15 | 1.33 | 1.37 | Arl2bp |
| 1417124_at | Lgx only | 7386 | 6930 | 5048 | 4243 | −1.07 | −1.46 | −1.74 | Dstn |
| 1417127_at | Lgx only | 53 | 106 | 136 | 186 | 2.01 | 2.58 | 3.52 | Msx1 |
| 1417142_at | Res & Lgx | 852 | 935 | 1104 | 1181 | 1.10 | 1.30 | 1.39 | 4932442K08Rik |
| 1417146_at | Res & Lgx | 605 | 807 | 893 | 1039 | 1.33 | 1.48 | 1.72 | 2410018C20Rik |
| 1417165_at | Lgx only | 2346 | 2465 | 2903 | 2874 | 1.05 | 1.24 | 1.23 | Mbd2 |
| 1417168_a_at | Lgx only | 2358 | 2585 | 3131 | 3801 | 1.10 | 1.33 | 1.61 | Usp2 |
| 1417170_at | Lgx only | 1459 | 1405 | 1174 | 1121 | −1.04 | −1.24 | −1.30 | Lztfl1 |
| 1417174_at | Res & Lgx | 1039 | 958 | 738 | 798 | −1.08 | −1.41 | −1.30 | 1810021J13Rik |
| 1417177_at | Lgx only | 462 | 447 | 437 | 628 | −1.03 | −1.06 | 1.36 | Galk1 |
| 1417180_at | Res & Lgx | 754 | 837 | 1024 | 1133 | 1.11 | 1.36 | 1.50 | Pcsk7 |
| 1417185_at | Lgx only | 9497 | 9130 | 8221 | 7632 | −1.04 | −1.16 | −1.24 | Ly6a |
| 1417190_at | Lgx only | 4733 | 4553 | 5431 | 6212 | −1.04 | 1.15 | 1.31 | Pbef1 |
| 1417191_at | Lgx only | 3776 | 3337 | 3338 | 2706 | −1.13 | −1.13 | −1.40 | Dnajb9 |
| 1417207_at | Lgx only | 80 | 144 | 149 | 248 | 1.79 | 1.86 | 3.09 | Dvl2 |
| 1417209_at | Lgx only | 1828 | 1950 | 2173 | 2377 | 1.07 | 1.19 | 1.30 | Sertad2 |
| 1417226_at | Lgx only | 812 | 921 | 967 | 1224 | 1.13 | 1.19 | 1.51 | Fbxw4 |
| 1417228_at | Lgx only | 535 | 649 | 639 | 835 | 1.21 | 1.19 | 1.56 | Capn1 |
| 1417233_at | Res & Lgx | 2926 | 2760 | 2274 | 1898 | −1.06 | −1.29 | −1.54 | Chchd4 |
| 1417238_at | Lgx only | 767 | 878 | 883 | 1108 | 1.15 | 1.15 | 1.44 | Ewsr1 |
| 1417239_at | Lgx only | 1936 | 1557 | 1139 | 998 | −1.24 | −1.70 | −1.94 | Cetn3 |
| 1417240_at | Res & Lgx | 1783 | 2528 | 2699 | 3377 | 1.42 | 1.51 | 1.89 | Zyx |
| 1417241_at | Res & Lgx | 3699 | 3394 | 2641 | 1970 | −1.09 | −1.40 | −1.88 | X83328 |
| 1417258_at | Res & Lgx | 4947 | 4979 | 3931 | 3307 | 1.01 | −1.26 | −1.50 | Cct5 |
| 1417271_a_at | Lgx only | 4442 | 4716 | 5165 | 6247 | 1.06 | 1.16 | 1.41 | Eng |
| 1417273_at | Res & Lgx | 8604 | 12748 | 23951 | 27986 | 1.48 | 2.78 | 3.25 | Pdk4 |
| 1417285_a_at | Lgx only | 32713 | 36339 | 37425 | 43067 | 1.11 | 1.14 | 1.32 | Ndufa5 |
| 1417291_at | Res & Lgx | 1425 | 1410 | 2175 | 2102 | −1.01 | 1.53 | 1.47 | Tnfrsf1a |
| 1417294_at | Lgx only | 1787 | 1927 | 2326 | 2186 | 1.08 | 1.30 | 1.22 | Akr7a5 |
| 1417297_at | All | 145 | 259 | 295 | 450 | 1.79 | 2.03 | 3.10 | Itpr3 |
| 1417304_at | Lgx only | 198 | 308 | 367 | 470 | 1.56 | 1.85 | 2.37 | Chrd |
| 1417306_at | Lgx only | 427 | 464 | 581 | 668 | 1.09 | 1.36 | 1.56 | Tyk2 |
| 1417307_at | Lgx only | 1236 | 1290 | 1344 | 1544 | 1.04 | 1.09 | 1.25 | Dmd |
| 1417308_at | Lgx only | 15382 | 20160 | 20634 | 26200 | 1.31 | 1.34 | 1.70 | Pkm2 |
| 1417311_at | Lgx only | 47051 | 55061 | 51583 | 64453 | 1.17 | 1.10 | 1.37 | Crip2 |
| 1417312_at | Lgx only | 1316 | 1798 | 2877 | 4398 | 1.37 | 2.19 | 3.34 | Dkk3 |
| 1417327_at | Lgx only | 3779 | 3050 | 2884 | 2338 | −1.24 | −1.31 | −1.62 | Cav2 |
| 1417334_at | Lgx only | 308 | 321 | 400 | 466 | 1.04 | 1.30 | 1.51 | Stk19 |
| 1417349_at | Lgx only | 3676 | 3837 | 3192 | 2742 | 1.04 | −1.15 | −1.34 | Pldn |
| 1417357_at | Lgx only | 1014 | 1076 | 1189 | 1373 | 1.06 | 1.17 | 1.35 | Emd |
| 1417367_at | Lgx only | 11894 | 11153 | 11043 | 9572 | −1.07 | −1.08 | −1.24 | Ppp2ca |
| 1417369_at | Lgx only | 2848 | 3279 | 3554 | 4089 | 1.15 | 1.25 | 1.44 | Hsd17b4 |
| 1417373_a_at | CR & Res | 57174 | 74618 | 40192 | 59746 | 1.31 | −1.42 | 1.04 | Tuba4a |
| 1417382_at | CR & Res | 5433 | 4021 | 4266 | 4657 | −1.35 | −1.27 | −1.17 | Entpd5 |
| 1417389_at | Lgx only | 4932 | 5697 | 6811 | 9117 | 1.16 | 1.38 | 1.85 | Gpc1 |
| 1417392_a_at | Lgx only | 386 | 416 | 569 | 884 | 1.08 | 1.47 | 2.29 | Slc7a7 |
| 1417394_at | Lgx only | 1516 | 1224 | 998 | 705 | −1.24 | −1.52 | −2.15 | Klf4 |
| 1417397_at | Lgx only | 364 | 447 | 480 | 576 | 1.23 | 1.32 | 1.58 | Slc9a1 |
| 1417398_at | CR & Lgx | 2047 | 1670 | 1999 | 1532 | −1.23 | −1.02 | −1.34 | Rras2 |
| 1417399_at | Lgx only | 4367 | 4901 | 4815 | 5943 | 1.12 | 1.10 | 1.36 | Gas6 |
| 1417402_at | Lgx only | 3863 | 4030 | 3439 | 3158 | 1.04 | −1.12 | −1.22 | 1190017O12Rik |
| 1417409_at | Lgx only | 1660 | 1563 | 1378 | 1058 | −1.06 | −1.20 | −1.57 | Jun |
| 1417423_at | All | 1931 | 2829 | 3104 | 4003 | 1.47 | 1.61 | 2.07 | Grina |
| 1417433_at | Res only | 1695 | 1694 | 1291 | 1499 | −1.00 | −1.31 | −1.13 | Lypla2 |
| 1417437_at | CR only | 643 | 802 | 739 | 810 | 1.25 | 1.15 | 1.26 | Xrcc6 |
| 1417441_at | Lgx only | 970 | 856 | 665 | 557 | −1.13 | −1.46 | −1.74 | Dnajc12 |
| 1417446_at | Res & Lgx | 817 | 1066 | 1009 | 1405 | 1.31 | 1.24 | 1.72 | Slc12a4 |
| 1417466_at | Lgx only | 22355 | 20101 | 19656 | 17738 | −1.11 | −1.14 | −1.26 | Rgs5 |
| 1417475_at | Lgx only | 541 | 682 | 670 | 846 | 1.26 | 1.24 | 1.57 | Atp13a1 |
| 1417476_at | Lgx only | 1158 | 1444 | 1386 | 1454 | 1.25 | 1.20 | 1.26 | Fbxw5 |
| 1417478_a_at | Res & Lgx | 2285 | 1692 | 1323 | 979 | −1.35 | −1.73 | −2.34 | Ppp2r3c |
| 1417490_at | Lgx only | 17449 | 20356 | 20005 | 21458 | 1.17 | 1.15 | 1.23 | Ctsb |
| 1417493_at | Lgx only | 1896 | 1748 | 1628 | 1370 | −1.09 | −1.17 | −1.38 | Bmi1 |
| 1417494_a_at | Lgx only | 3168 | 2535 | 2703 | 2081 | −1.25 | −1.17 | −1.52 | Cp |
| 1417503_at | Lgx only | 813 | 899 | 1044 | 1230 | 1.11 | 1.29 | 1.51 | Rfc2 |
| 1417507_at | Res only | 303 | 313 | 206 | 250 | 1.04 | −1.47 | −1.21 | Cyb561 |
| 1417516_at | Res & Lgx | 1417 | 1442 | 975 | 1093 | 1.02 | −1.45 | −1.30 | Ddit3 |
| 1417533_a_at | Res & Lgx | 3526 | 3994 | 5408 | 5733 | 1.13 | 1.53 | 1.63 | Itgb5 |
| 1417536_at | Lgx only | 1523 | 1489 | 1327 | 1156 | −1.02 | −1.15 | −1.32 | Zmat2 |
| 1417544_a_at | Lgx only | 1725 | 2092 | 2186 | 2613 | 1.21 | 1.27 | 1.52 | Flot2 |
| 1417552_at | Lgx only | 368 | 330 | 315 | 219 | −1.12 | −1.17 | −1.68 | Fap |
| 1417562_at | Res & Lgx | 5225 | 5748 | 7049 | 8132 | 1.10 | 1.35 | 1.56 | Eif4ebp1 |
| 1417564_at | Lgx only | 1045 | 942 | 849 | 682 | −1.11 | −1.23 | −1.53 | Med7 |
| 1417574_at | Lgx only | 4249 | 4227 | 4829 | 5635 | −1.01 | 1.14 | 1.33 | Cxcl12 |
| 1417578_a_at | Lgx only | 1318 | 1466 | 1614 | 1933 | 1.11 | 1.23 | 1.47 | Gmppa |
| 1417581_at | Lgx only | 327 | 311 | 206 | 118 | −1.05 | −1.59 | −2.77 | Dhodh |
| 1417588_at | CR only | 70 | 24 | 64 | 43 | −2.88 | −1.10 | −1.62 | Galnt3 |
| 1417590_at | Res & Lgx | 1193 | 1414 | 1657 | 1976 | 1.19 | 1.39 | 1.66 | Cyp27a1 |
| 1417592_at | Lgx only | 1276 | 1544 | 1813 | 1967 | 1.21 | 1.42 | 1.54 | Frap1 |
| 1417606_a_at | Lgx only | 8113 | 8165 | 6538 | 5977 | 1.01 | −1.24 | −1.36 | Calr |
| 1417611_at | Lgx only | 410 | 483 | 562 | 660 | 1.18 | 1.37 | 1.61 | Tmem37 |
| 1417614_at | Lgx only | 52366 | 62118 | 63982 | 71743 | 1.19 | 1.22 | 1.37 | Ckm |
| 1417626_at | Lgx only | 20898 | 23134 | 29205 | 31629 | 1.11 | 1.40 | 1.51 | Pde4dip |
| 1417629_at | CR only | 550 | 726 | 507 | 529 | 1.32 | −1.08 | −1.04 | Prodh |
| 1417631_at | Res only | 576 | 686 | 816 | 800 | 1.19 | 1.42 | 1.39 | Mknk1 |
| 1417636_at | Lgx only | 357 | 496 | 550 | 864 | 1.39 | 1.54 | 2.42 | Slc6a9 |
| 1417637_a_at | Lgx only | 740 | 721 | 597 | 561 | −1.03 | −1.24 | −1.32 | Hmg20b |
| 1417659_at | Res & Lgx | 2296 | 1809 | 1478 | 837 | −1.27 | −1.55 | −2.74 | Vps29 |
| 1417664_a_at | Lgx only | 413 | 416 | 589 | 585 | 1.01 | 1.43 | 1.42 | Ndrg3 |
| 1417665_a_at | Lgx only | 1641 | 1906 | 2136 | 2541 | 1.16 | 1.30 | 1.55 | Cpsf1 |
| 1417669_at | Lgx only | 1917 | 2203 | 2177 | 2365 | 1.15 | 1.14 | 1.23 | Abhd12 |
| 1417673_at | Res & Lgx | 9046 | 10264 | 7598 | 7548 | 1.13 | −1.19 | −1.20 | Grb14 |
| 1417680_at | Lgx only | 1039 | 914 | 800 | 514 | −1.14 | −1.30 | −2.02 | Kcna5 |
| 1417681_at | Lgx only | 2481 | 2417 | 2057 | 1755 | −1.03 | −1.21 | −1.41 | Nudt21 |
| 1417683_at | Lgx only | 3002 | 3170 | 2902 | 2615 | 1.06 | −1.03 | −1.15 | Diablo |
| 1417693_a_at | Lgx only | 2110 | 2468 | 2597 | 3325 | 1.17 | 1.23 | 1.58 | Gab1 |
| 1417712_at | Lgx only | 9771 | 8265 | 8035 | 6481 | −1.18 | −1.22 | −1.51 | Eif2s2 |
| 1417715_a_at | Lgx only | 37732 | 44662 | 43599 | 56266 | 1.18 | 1.16 | 1.49 | Got2 |
| 1417722_at | Lgx only | 2172 | 2787 | 2703 | 3093 | 1.28 | 1.24 | 1.42 | Pgls |
| 1417724_at | Lgx only | 963 | 827 | 712 | 620 | −1.17 | −1.35 | −1.55 | Thoc4 |
| 1417727_at | Lgx only | 595 | 659 | 764 | 845 | 1.11 | 1.28 | 1.42 | Sfrs9 |
| 1417728_at | Lgx only | 1083 | 1178 | 1375 | 1363 | 1.09 | 1.27 | 1.26 | Mbd3 |
| 1417730_at | Res & Lgx | 759 | 785 | 1047 | 1112 | 1.03 | 1.38 | 1.47 | Ext1 |
| 1417762_a_at | Lgx only | 15317 | 17412 | 16383 | 18728 | 1.14 | 1.07 | 1.22 | Rpl8 |
| 1417775_at | Lgx only | 385 | 504 | 537 | 605 | 1.31 | 1.40 | 1.57 | Rpo1-4 |
| 1417777_at | Lgx only | 300 | 380 | 359 | 417 | 1.27 | 1.20 | 1.39 | Ltb4dh |
| 1417778_at | Lgx only | 592 | 523 | 478 | 322 | −1.13 | −1.24 | −1.84 | Zfp35 |
| 1417791_a_at | Lgx only | 752 | 638 | 524 | 553 | −1.18 | −1.43 | −1.36 | Zfml |
| 1417807_at | Lgx only | 404 | 370 | 322 | 268 | −1.09 | −1.25 | −1.51 | 2700038N03Rik |
| 1417814_at | CR only | 1089 | 821 | 1021 | 931 | −1.33 | −1.07 | −1.17 | Pla2g5 |
| 1417825_at | Res & Lgx | 10020 | 8920 | 8393 | 8135 | −1.12 | −1.19 | −1.23 | Esd |
| 1417827_at | Lgx only | 1987 | 1887 | 1725 | 1334 | −1.05 | −1.15 | −1.49 | Ngly1 |
| 1417840_at | Lgx only | 655 | 635 | 537 | 493 | −1.03 | −1.22 | −1.33 | 1500031L02Rik |
| 1417842_at | CR only | 735 | 604 | 602 | 618 | −1.22 | −1.22 | −1.19 | Caml |
| 1417865_at | Res only | 1877 | 1747 | 1566 | 1714 | −1.07 | −1.20 | −1.09 | Tnfaip1 |
| 1417868_a_at | Lgx only | 1651 | 1315 | 950 | 698 | −1.26 | −1.74 | −2.36 | Ctsz |
| 1417889_at | Lgx only | 11804 | 10711 | 10232 | 8376 | −1.10 | −1.15 | −1.41 | Apobec2 |
| 1417893_at | Lgx only | 350 | 308 | 313 | 275 | −1.14 | −1.12 | −1.27 | Sfxn3 |
| 1417912_at | Lgx only | 4436 | 4290 | 4051 | 3653 | −1.03 | −1.10 | −1.21 | Tmem93 |
| 1417916_a_at | Lgx only | 2557 | 2471 | 2075 | 1814 | −1.03 | −1.23 | −1.41 | Fxc1 |
| 1417928_at | Lgx only | 930 | 1019 | 1145 | 1635 | 1.10 | 1.23 | 1.76 | Pdlim4 |
| 1417933_at | Lgx only | 1003 | 1029 | 1202 | 1834 | 1.03 | 1.20 | 1.83 | Igfbp6 |
| 1417936_at | Res & Lgx | 627 | 556 | 388 | 317 | −1.13 | −1.62 | −1.98 | Ccl9 |
| 1417951_at | Res & Lgx | 39914 | 46728 | 50392 | 54619 | 1.17 | 1.26 | 1.37 | Eno3 |
| 1417953_at | Lgx only | 721 | 903 | 916 | 1106 | 1.25 | 1.27 | 1.53 | D6Wsu176e |
| 1417963_at | CR only | 1666 | 1274 | 1799 | 1621 | −1.31 | 1.08 | −1.03 | Pltp |
| 1417970_at | Lgx only | 2618 | 2363 | 2180 | 1814 | −1.11 | −1.20 | −1.44 | Atp5s |
| 1417974_at | Lgx only | 1516 | 1151 | 1001 | 799 | −1.32 | −1.51 | −1.90 | Kpna4 |
| 1417983_a_at | Lgx only | 654 | 514 | 464 | 439 | −1.27 | −1.41 | −1.49 | Ube2v2 |
| 1417985_at | All | 980 | 658 | 657 | 521 | −1.49 | −1.49 | −1.88 | Nrarp |
| 1418000_a_at | Lgx only | 44199 | 45002 | 51725 | 57791 | 1.02 | 1.17 | 1.31 | Itm2b |
| 1418004_a_at | Lgx only | 1596 | 1844 | 2175 | 2741 | 1.16 | 1.36 | 1.72 | Tmem176b |
| 1418007_at | Lgx only | 559 | 548 | 518 | 370 | −1.02 | −1.08 | −1.51 | 1810007M14Rik |
| 1418025_at | Res & Lgx | 1814 | 1542 | 1141 | 1035 | −1.18 | −1.59 | −1.75 | Bhlhb2 |
| 1418031_at | Res & Lgx | 433 | 559 | 602 | 614 | 1.29 | 1.39 | 1.42 | Myo9b |
| 1418048_at | Lgx only | 2291 | 1894 | 1822 | 1403 | −1.21 | −1.26 | −1.63 | 1110059G10Rik |
| 1418049_at | Lgx only | 564 | 707 | 732 | 893 | 1.25 | 1.30 | 1.58 | Ltbp3 |
| 1418058_at | Lgx only | 1896 | 1822 | 1556 | 1532 | −1.04 | −1.22 | −1.24 | Eltd1 |
| 1418085_at | Lgx only | 119 | 169 | 209 | 189 | 1.42 | 1.76 | 1.59 | Prkcz |
| 1418090_at | Lgx only | 393 | 473 | 619 | 747 | 1.20 | 1.58 | 1.90 | Plvap |
| 1418093_a_at | Lgx only | 180 | 168 | 242 | 294 | −1.07 | 1.34 | 1.63 | Egf |
| 1418124_at | Res & Lgx | 3982 | 4177 | 4816 | 5241 | 1.05 | 1.21 | 1.32 | Tmem85 |
| 1418128_at | Lgx only | 2612 | 3481 | 3429 | 3842 | 1.33 | 1.31 | 1.47 | Adcy6 |
| 1418148_at | Res & Lgx | 1168 | 915 | 1700 | 1493 | −1.28 | 1.46 | 1.28 | Abhd1 |
| 1418181_at | Lgx only | 6615 | 7164 | 10513 | 10742 | 1.08 | 1.59 | 1.62 | Ptp4a3 |
| 1418183_a_at | Lgx only | 2041 | 1857 | 1603 | 1381 | −1.10 | −1.27 | −1.48 | Pscd1 |
| 1418186_at | Lgx only | 1322 | 1528 | 1723 | 2106 | 1.16 | 1.30 | 1.59 | Gstt1 |
| 1418187_at | Lgx only | 2289 | 2761 | 3034 | 3198 | 1.21 | 1.33 | 1.40 | Ramp2 |
| 1418209_a_at | Res & Lgx | 3685 | 3274 | 2816 | 2402 | −1.13 | −1.31 | −1.53 | Pfn2 |
| 1418223_at | Res & Lgx | 2693 | 2594 | 2189 | 2015 | −1.04 | −1.23 | −1.34 | Sec11a |
| 1418228_at | Lgx only | 3494 | 3467 | 2926 | 2282 | −1.01 | −1.19 | −1.53 | Nfu1 |
| 1418244_at | Res & Lgx | 3970 | 4253 | 2852 | 2563 | 1.07 | −1.39 | −155 | Nat5 |
| 1418261_at | All | 412 | 712 | 724 | 860 | 1.73 | 1.76 | 2.09 | Syk |
| 1418275_a_at | Lgx only | 977 | 937 | 845 | 806 | −1.04 | −1.16 | −1.21 | Elf2 |
| 1418277_at | Lgx only | 5601 | 5071 | 4822 | 3833 | −1.10 | −1.16 | −1.46 | rp9 |
| 1418296_at | Lgx only | 743 | 895 | 936 | 1162 | 1.20 | 1.26 | 1.56 | Fxyd5 |
| 1418302_at | Res & Lgx | 1279 | 1585 | 1792 | 2129 | 1.24 | 1.40 | 1.66 | Ppt2 |
| 1418306_at | CR only | 212 | 99 | 186 | 157 | −2.15 | −1.14 | −1.35 | Crybb1 |
| 1418308_at | Lgx only | 312 | 316 | 235 | 144 | 1.02 | −1.33 | −2.17 | Hus1 |
| 1418310_a_at | CR only | 27 | 74 | 31 | 71 | 2.71 | 1.13 | 2.59 | Rlbp1 |
| 1418325_at | Res only | 530 | 450 | 302 | 356 | −1.18 | −1.75 | −1.49 | Sephs2 |
| 1418327_at | Lgx only | 6638 | 6594 | 5327 | 4629 | −1.01 | −1.25 | −1.43 | 1110058L19Rik |
| 1418328_at | Lgx only | 9663 | 10359 | 12241 | 14054 | 1.07 | 1.27 | 1.45 | Cpt1b |
| 1418364_a_at | Res & Lgx | 6214 | 6280 | 8701 | 10097 | 1.01 | 1.40 | 1.62 | Ftl1 |
| 1418373_at | Res & Lgx | 20321 | 25840 | 28141 | 36744 | 1.27 | 1.38 | 1.81 | Pgam2 |
| 1418384_at | Lgx only | 2697 | 2580 | 2446 | 2128 | −1.05 | −1.10 | −1.27 | Apool |
| 1418394_a_at | All | 1313 | 1651 | 1851 | 2002 | 1.26 | 1.41 | 1.52 | Cd97 |
| 1418395_at | Res only | 2083 | 2050 | 2622 | 2487 | −1.02 | 1.26 | 1.19 | Slc47a1 |
| 1418421_at | Lgx only | 409 | 447 | 356 | 219 | 1.09 | −1.15 | −1.87 | Bcl6b |
| 1418427_at | Lgx only | 4160 | 3689 | 3518 | 2931 | −1.13 | −1.18 | −1.42 | Kif5b |
| 1418433_at | Lgx only | 2097 | 2223 | 2417 | 2813 | 1.06 | 1.15 | 1.34 | Cab39 |
| 1418456_a_at | Lgx only | 1322 | 1134 | 961 | 777 | −1.17 | −1.37 | −1.70 | Cxcl14 |
| 1418461_at | Lgx only | 137 | 207 | 161 | 226 | 1.52 | 1.18 | 1.65 | Sh3d19 |
| 1418462_at | Lgx only | 938 | 943 | 764 | 665 | 1.01 | −1.23 | −1.41 | Exosc9 |
| 1418464_at | Lgx only | 54 | 55 | 98 | 186 | 1.02 | 1.80 | 3.42 | Matn4 |
| 1418467_at | CR & Lgx | 1961 | 2626 | 2477 | 2773 | 1.34 | 1.26 | 1.41 | Smarcd3 |
| 1418479_at | CR & Lgx | 971 | 752 | 799 | 628 | −1.29 | −1.22 | −1.55 | Vps54 |
| 1418483_a_at | Lgx only | 801 | 694 | 755 | 588 | −1.15 | −1.06 | −1.36 | Ggta1 |
| 1418495_at | Res only | 310 | 349 | 161 | 194 | 1.13 | −1.93 | −1.60 | Zc3h8 |
| 1418506_a_at | Res only | 20857 | 23088 | 24680 | 25457 | 1.11 | 1.18 | 1.22 | Prdx2 |
| 1418518_at | Lgx only | 701 | 821 | 854 | 1036 | 1.17 | 1.22 | 1.48 | Furin |
| 1418528_a_at | Res & Lgx | 4483 | 4424 | 3749 | 3167 | −1.01 | −1.20 | −1.42 | Dad1 |
| 1418530_at | Lgx only | 282 | 250 | 196 | 154 | −1.13 | −1.44 | −1.84 | Nup160 |
| 1418532_at | Lgx only | 180 | 202 | 332 | 354 | 1.12 | 1.85 | 1.97 | Fzd2 |
| 1418551_at | Lgx only | 34184 | 47198 | 44002 | 56324 | 1.38 | 1.29 | 1.65 | Mybpc3 |
| 1418560_at | Lgx only | 42487 | 47719 | 46215 | 55194 | 1.12 | 1.09 | 1.30 | Pdha1 |
| 1418563_at | Res & Lgx | 1576 | 1497 | 1007 | 795 | −1.05 | −1.57 | −1.98 | Serbp1 |
| 1418578_at | Lgx only | 718 | 782 | 1230 | 1501 | 1.09 | 1.71 | 2.09 | Dgka |
| 1418583_at | Lgx only | 4503 | 4409 | 3800 | 2964 | −1.02 | −1.19 | −1.52 | Hint3 |
| 1418584_at | Lgx only | 2929 | 2748 | 2413 | 2257 | −1.07 | −1.21 | −1.30 | Ccnh |
| 1418589_a_at | Res & Lgx | 5966 | 5443 | 3817 | 3292 | −1.10 | −1.56 | −1.81 | Mlf1 |
| 1418593_at | Lgx only | 967 | 1039 | 1128 | 1548 | 1.08 | 1.17 | 1.60 | Taf6 |
| 1418595_at | Lgx only | 3838 | 4270 | 5239 | 5576 | 1.11 | 1.37 | 1.45 | S3-12 |
| 1418604_at | CR only | 118 | 179 | 95 | 148 | 1.51 | −1.24 | 1.25 | Avpr1a |
| 1418621_at | Lgx only | 6696 | 7127 | 7710 | 8254 | 1.06 | 1.15 | 1.23 | Rab2 |
| 1418640_at | Lgx only | 445 | 396 | 366 | 260 | −1.12 | −1.22 | −1.71 | Sirt1 |
| 1418644_a_at | Res & Lgx | 2290 | 2528 | 3079 | 3754 | 1.10 | 1.34 | 1.64 | Stk11 |
| 1418646_at | Lgx only | 576 | 557 | 801 | 953 | −1.03 | 1.39 | 1.65 | Gna-rs1 |
| 1418649_at | Res & Lgx | 1817 | 1689 | 1010 | 1064 | −1.08 | −1.80 | −1.71 | Egln3 |
| 1418658_at | Lgx only | 3201 | 2867 | 2597 | 2170 | −1.12 | −1.23 | −1.48 | 2410005O16Rik |
| 1418659_at | Lgx only | 655 | 451 | 573 | 399 | −1.45 | −1.14 | −1.64 | Clock |
| 1418665_at | Res only | 432 | 341 | 817 | 509 | −1.27 | 1.89 | 1.18 | Impa2 |
| 1418681_at | Lgx only | 167 | 97 | 90 | 44 | −1.73 | −1.85 | −3.80 | Alg13 |
| 1418700_at | Res only | 2166 | 2223 | 2693 | 2292 | 1.03 | 1.24 | 1.06 | Lias |
| 1418703_at | Res & Lgx | 1788 | 1937 | 2728 | 2973 | 1.08 | 1.53 | 1.66 | Rbms1 |
| 1418714_at | Lgx only | 176 | 149 | 306 | 402 | −1.19 | 1.74 | 2.28 | Dusp8 |
| 1418726_a_at | Lgx only | 96588 | 107808 | 105192 | 136735 | 1.12 | 1.09 | 1.42 | Tnnt2 |
| 1418739_at | CR only | 115 | 61 | 145 | 180 | −1.88 | 1.26 | 1.56 | Sgk2 |
| 1418749_at | Lgx only | 829 | 963 | 993 | 1502 | 1.16 | 1.20 | 1.81 | Psd3 |
| 1418759_at | CR only | 65 | 25 | 85 | 74 | −2.61 | 1.30 | 1.14 | Ptpn20 |
| 1418763_at | Res only | 1354 | 1349 | 1037 | 1054 | −1.00 | −1.31 | −1.28 | Nit2 |
| 1418773_at | Lgx only | 997 | 1127 | 1373 | 1342 | 1.13 | 1.38 | 1.35 | Fads3 |
| 1418775_at | Res & Lgx | 1330 | 1611 | 1775 | 2047 | 1.21 | 1.33 | 1.54 | Al837181 |
| 1418782_at | Lgx only | 1857 | 2604 | 2789 | 2699 | 1.40 | 1.50 | 1.45 | Rxrg |
| 1418817_at | Lgx only | 868 | 707 | 713 | 565 | −1.23 | −1.22 | −1.54 | Chmp1b |
| 1418835_at | Lgx only | 1187 | 1202 | 996 | 645 | 1.01 | −1.19 | −1.84 | Phlda1 |
| 1418838_at | Lgx only | 1243 | 1517 | 1333 | 1771 | 1.22 | 1.07 | 1.43 | Abcd1 |
| 1418840_at | Lgx only | 667 | 575 | 556 | 501 | −1.16 | −1.20 | −1.33 | Pdcd4 |
| 1418846_at | CR only | 103 | 199 | 99 | 162 | 1.93 | −1.04 | 1.57 | Ap4m1 |
| 1418847_at | Lgx only | 28 | 28 | 56 | 92 | −1.04 | 1.95 | 3.24 | Arg2 |
| 1418861_at | Res only | 887 | 950 | 1409 | 1371 | 1.07 | 1.59 | 1.55 | Pias4 |
| 1418863_at | Lgx only | 1860 | 2280 | 2226 | 2561 | 1.23 | 1.20 | 1.38 | Gata4 |
| 1418869_a_at | Lgx only | 678 | 738 | 805 | 954 | 1.09 | 1.19 | 1.41 | Pus1 |
| 1418874_a_at | Lgx only | 5030 | 5073 | 6091 | 6369 | 1.01 | 1.21 | 1.27 | Psmd4 |
| 1418885_a_at | Res & Lgx | 906 | 1107 | 1173 | 1863 | 1.22 | 1.30 | 2.06 | Idh3b |
| 1418899_at | Lgx only | 1067 | 834 | 819 | 715 | −1.28 | −1.30 | −1.49 | Ufm1 |
| 1418924_at | Lgx only | 89 | 173 | 226 | 307 | 1.95 | 2.54 | 3.45 | Rassf7 |
| 1418926_at | Res & Lgx | 3012 | 2659 | 2326 | 2255 | −1.13 | −1.29 | −1.34 | Zeb1 |
| 1418928_a_at | Lgx only | 958 | 1057 | 1130 | 1663 | 1.10 | 1.18 | 1.74 | 2310038H17Rik |
| 1418929_at | Lgx only | 410 | 472 | 537 | 581 | 1.15 | 1.31 | 1.42 | Ift57 |
| 1418933_at | Res only | 8 | 28 | 16 | 13 | 3.76 | 2.08 | 1.68 | Slc1a6 |
| 1418947_at | CR only | 247 | 343 | 289 | 319 | 1.39 | 1.17 | 1.29 | Nek3 |
| 1418952_at | Res & Lgx | 9461 | 9278 | 12035 | 12244 | −1.02 | 1.27 | 1.29 | Txlnb |
| 1418967_a_at | Lgx only | 302 | 261 | 219 | 209 | −1.16 | −1.38 | −1.45 | St7 |
| 1418968_at | Lgx only | 2166 | 1884 | 1630 | 1473 | −1.15 | −1.33 | −1.47 | Rb1cc1 |
| 1418986_a_at | Lgx only | 1097 | 1006 | 854 | 642 | −1.09 | −1.28 | −1.71 | Uxt |
| 1418987_at | Res only | 161 | 117 | 260 | 269 | −1.38 | 1.61 | 1.67 | Pla2g2d |
| 1418988_at | Res & Lgx | 2768 | 2378 | 2028 | 1916 | −1.16 | −1.36 | −1.44 | Pex7 |
| 1418996_a_at | Lgx only | 3436 | 3503 | 3043 | 2693 | 1.02 | −1.13 | −1.28 | Lyrm5 |
| 1419013_at | Res only | 438 | 501 | 605 | 499 | 1.14 | 1.38 | 1.14 | Gpatch1 |
| 1419026_at | Lgx only | 910 | 978 | 1211 | 1131 | 1.08 | 1.33 | 1.24 | Daxx |
| 1419037_at | Lgx only | 1248 | 1228 | 1010 | 1005 | −1.02 | −1.24 | −1.24 | Csnk2a1 |
| 1419062_at | Lgx only | 646 | 672 | 717 | 873 | 1.04 | 1.11 | 1.35 | Epb4.1l3 |
| 1419070_at | CR only | 274 | 536 | 312 | 423 | 1.96 | 1.14 | 1.54 | Cys1 |
| 1419072_at | Lgx only | 1253 | 1214 | 1154 | 1035 | −1.03 | −1.09 | −1.21 | Gstm7 |
| 1419074_at | Lgx only | 669 | 635 | 434 | 385 | −1.05 | −1.54 | −1.74 | Chac2 |
| 1419081_at | Lgx only | 620 | 617 | 514 | 392 | −1.00 | −1.21 | −1.58 | Atg10 |
| 1419109_at | Res & Lgx | 19560 | 22347 | 25933 | 31691 | 1.14 | 1.33 | 1.62 | Hrc |
| 1419131_at | CR only | 10 | 65 | 30 | 43 | 6.52 | 2.99 | 4.25 | F13b |
| 1419144_at | Lgx only | 328 | 358 | 484 | 596 | 1.09 | 1.48 | 1.81 | Cd163 |
| 1419158_a_at | CR only | 760 | 935 | 726 | 788 | 1.23 | −1.05 | 1.04 | Hars2 |
| 1419164_at | Res & Lgx | 1598 | 1612 | 1126 | 698 | 1.01 | −1.42 | −2.29 | Zfp260 |
| 1419169_at | Lgx only | 1118 | 1026 | 941 | 817 | −1.09 | −1.19 | −1.37 | Mapk6 |
| 1419170_at | Lgx only | 2926 | 2624 | 2049 | 1889 | −1.12 | −1.43 | −1.55 | Tmem157 |
| 1419174_at | Lgx only | 1433 | 1234 | 1044 | 1065 | −1.16 | −1.37 | −1.34 | 2410004B18Rik |
| 1419182_at | Lgx only | 525 | 545 | 632 | 749 | 1.04 | 1.20 | 1.43 | Svep1 |
| 1419186_a_at | Res & Lgx | 779 | 684 | 511 | 470 | −1.14 | −1.52 | −1.66 | St8sia4 |
| 1419214_at | Res only | 146 | 199 | 310 | 284 | 1.36 | 2.12 | 1.94 | Tnfrsf11a |
| 1419238_at | Lgx only | 154 | 255 | 316 | 301 | 1.66 | 2.06 | 1.96 | Abca7 |
| 1419258_at | Lgx only | 1711 | 1423 | 1294 | 1111 | −1.20 | −1.32 | −1.54 | Tcea1 |
| 1419272_at | Res only | 428 | 435 | 585 | 498 | 1.02 | 1.37 | 1.16 | Myd88 |
| 1419292_at | Lgx only | 3158 | 3740 | 4270 | 4656 | 1.18 | 1.35 | 1.47 | Htra3 |
| 1419295_at | Res only | 209 | 211 | 411 | 276 | 1.01 | 1.96 | 1.32 | Creb3l1 |
| 1419297_at | CR only | 149 | 66 | 122 | 147 | −2.25 | −1.22 | −1.01 | H2-Oa |
| 1419302_at | Res & Lgx | 794 | 919 | 1108 | 1247 | 1.16 | 1.40 | 1.57 | Heyl |
| 1419333_at | Lgx only | 392 | 461 | 414 | 572 | 1.18 | 1.06 | 1.46 | 1110008J03Rik |
| 1419352_at | Lgx only | 1989 | 1829 | 1498 | 1340 | −1.09 | −1.33 | −1.48 | I7Rn6 |
| 1419354_at | Lgx only | 1196 | 1159 | 1578 | 1790 | −1.03 | 1.32 | 1.50 | Klf7 |
| 1419358_at | Lgx only | 207 | 323 | 185 | 348 | 1.56 | −1.11 | 1.68 | Sorcs2 |
| 1419366_at | Res & Lgx | 642 | 733 | 782 | 1009 | 1.14 | 1.22 | 1.57 | Zmat5 |
| 1419375_at | Res & Lgx | 1257 | 1211 | 921 | 872 | −1.04 | −1.36 | −1.44 | Wbp4 |
| 1419398_a_at | Lgx only | 10797 | 11078 | 10041 | 8759 | 1.03 | −1.08 | −1.23 | Reep5 |
| 1419415_a_at | CR only | 1168 | 905 | 1009 | 964 | −1.29 | −1.16 | −1.21 | Rarg |
| 1419428_a_at | Lgx only | 1374 | 1692 | 1993 | 2038 | 1.23 | 1.45 | 1.48 | Gaa |
| 1419429_at | Res & Lgx | 79 | 137 | 161 | 223 | 1.74 | 2.04 | 2.83 | Cntfr |
| 1419452_at | Lgx only | 1034 | 914 | 835 | 680 | −1.13 | −1.24 | −1.52 | Uchl5 |
| 1419455_at | Lgx only | 6361 | 6701 | 5697 | 5310 | 1.05 | −1.12 | −1.20 | Il10rb |
| 1419470_at | Res & Lgx | 865 | 849 | 644 | 649 | −1.02 | −1.34 | −1.33 | Gnb4 |
| 1419477_at | Res only | 35 | 98 | 190 | 144 | 2.81 | 5.44 | 4.12 | Clec2d |
| 1419484_a_at | Lgx only | 20734 | 22346 | 11461 | 5733 | 1.08 | −1.81 | −3.62 | Gbas |
| 1419491_at | Res only | 13 | 40 | 52 | 67 | 3.14 | 4.08 | 5.30 | Defb1 |
| 1419495_at | Lgx only | 2055 | 1752 | 1771 | 1374 | −1.17 | −1.16 | −1.50 | Immp2l |
| 1419499_at | Lgx only | 7940 | 7261 | 10653 | 11012 | −1.09 | 1.34 | 1.39 | Gpam |
| 1419518_at | All | 7504 | 12289 | 3758 | 4518 | 1.64 | −2.00 | −1.66 | Tuba8 |
| 1419527_at | Lgx only | 403 | 437 | 548 | 829 | 1.08 | 1.36 | 2.06 | Comp |
| 1419550_a_at | Lgx only | 2491 | 2352 | 1855 | 1537 | −1.06 | −1.34 | −1.62 | Stk39 |
| 1419569_a_at | Res only | 681 | 700 | 1171 | 1519 | 1.03 | 1.72 | 2.23 | Isg20 |
| 1419584_at | Lgx only | 521 | 505 | 846 | 843 | −1.03 | 1.62 | 1.62 | Ttc28 |
| 1419609_at | CR only | 53 | 133 | 65 | 112 | 2.53 | 1.24 | 2.13 | Ccr1 |
| 1419630_a_at | CR & Lgx | 549 | 668 | 630 | 755 | 1.22 | 1.15 | 1.37 | Trim11 |
| 1419631_at | Res & Lgx | 119 | 146 | 218 | 228 | 1.23 | 1.84 | 1.92 | Was |
| 1419645_at | Lgx only | 463 | 390 | 335 | 288 | −1.19 | −1.38 | −1.61 | Cstf2 |
| 1419657_a_at | CR & Lgx | 1163 | 874 | 858 | 623 | −1.33 | −1.36 | −1.87 | Slc25a36 |
| 1419660_at | Lgx only | 4264 | 3723 | 4383 | 3253 | −1.15 | 1.03 | −1.31 | 1600012F09Rik |
| 1419687_at | Lgx only | 8909 | 9784 | 12046 | 13075 | 1.10 | 1.35 | 1.47 | Macrod1 |
| 1419736_a_at | Res & Lgx | 3408 | 3084 | 2455 | 1934 | −1.10 | −1.39 | −1.76 | Eif1ay |
| 1419762_at | Res & Lgx | 38 | 84 | 155 | 154 | 2.18 | 4.03 | 4.01 | Ubd |
| 1419787_a_at | Lgx only | 312 | 385 | 461 | 581 | 1.24 | 1.48 | 1.86 | Zfp628 |
| 1419824_a_at | Res & Lgx | 455 | 428 | 270 | 228 | −1.06 | −1.69 | −2.00 | A230062G08Rik |
| 1419952_at | Lgx only | 27 | 55 | 105 | 168 | 2.03 | 3.90 | 6.26 | 1700023D09Rik |
| 1420099_at | CR only | 203 | 411 | 349 | 286 | 2.02 | 1.72 | 1.40 | D13Ertd787e |
| 1420123_at | Res & Lgx | 3511 | 3401 | 5048 | 5686 | −1.03 | 1.44 | 1.62 | Tcta |
| 1420183_at | Lgx only | 234 | 252 | 360 | 451 | 1.08 | 1.54 | 1.93 | Lor |
| 1420325_at | Res only | 57 | 130 | 144 | 132 | 2.26 | 2.52 | 2.30 | Cramp1l |
| 1420329_at | Lgx only | 567 | 517 | 392 | 323 | −1.10 | −1.45 | −1.76 | 4930455C21Rik |
| 1420339_at | Res only | 309 | 242 | 217 | 225 | −1.28 | −1.43 | −1.37 | LOC100047915 |
| 1420374_at | Res & Lgx | 758 | 989 | 1238 | 1240 | 1.31 | 1.63 | 1.64 | Foxj2 |
| 1420375_at | Res & Lgx | 401 | 402 | 615 | 593 | 1.00 | 1.53 | 1.48 | Kif3a |
| 1420377_at | Lgx only | 89 | 105 | 134 | 173 | 1.18 | 1.50 | 1.94 | St8sia2 |
| 1420387_at | Res & Lgx | 2021 | 2029 | 1347 | 1414 | 1.00 | −1.50 | −1.43 | Mpv17 |
| 1420388_at | Lgx only | 160 | 221 | 219 | 350 | 1.38 | 1.37 | 2.19 | Prss12 |
| 1420405_at | Lgx only | 181 | 177 | 327 | 423 | −1.02 | 1.81 | 2.34 | Slco1a4 |
| 1420427_a_at | CR only | 1499 | 1159 | 1676 | 1564 | −1.29 | 1.12 | 1.04 | Dhx32 |
| 1420497_a_at | Res only | 446 | 471 | 707 | 611 | 1.06 | 1.58 | 1.37 | Cebpz |
| 1420502_at | Lgx only | 1203 | 971 | 967 | 740 | −1.24 | −1.24 | −1.63 | Sat1 |
| 1420507_a_at | Res & Lgx | 787 | 742 | 523 | 391 | −1.06 | −1.50 | −2.01 | 3110031B13Rik |
| 1420513_at | Res & Lgx | 4347 | 3651 | 3096 | 2640 | −1.19 | −1.40 | −1.65 | Efcab2 |
| 1420580_at | Res only | 108 | 171 | 205 | 173 | 1.59 | 1.90 | 1.60 | 4930429B21Rik |
| 1420617_at | CR & Lgx | 1660 | 2030 | 1377 | 1316 | 1.22 | −1.21 | −1.26 | Cpeb4 |
| 1420619_a_at | Lgx only | 9782 | 11497 | 12736 | 14220 | 1.18 | 1.30 | 1.45 | Aes |
| 1420654_a_at | Lgx only | 836 | 1010 | 530 | 414 | 1.21 | −1.58 | −2.02 | Gbe1 |
| 1420657_at | CR & Lgx | 978 | 1762 | 1973 | 2727 | 1.80 | 2.02 | 2.79 | Ucp3 |
| 1420684_at | Lgx only | 184 | 272 | 311 | 338 | 1.48 | 1.70 | 1.84 | Acox3 |
| 1420693_at | Lgx only | 12144 | 13502 | 14705 | 16745 | 1.11 | 1.21 | 1.38 | Myom1 |
| 1420703_at | Lgx only | 230 | 242 | 309 | 412 | 1.05 | 1.34 | 1.79 | Csf2ra |
| 1420707_a_at | Res only | 15 | 45 | 86 | 56 | 3.03 | 5.79 | 3.77 | Traip |
| 1420711_a_at | Res & Lgx | 1740 | 1664 | 1104 | 1160 | −1.05 | −1.58 | −1.50 | Pxmp3 |
| 1420715_a_at | Res only | 264 | 353 | 394 | 398 | 1.34 | 1.49 | 1.51 | Pparg |
| 1420727_a_at | Lgx only | 834 | 685 | 683 | 642 | −1.22 | −1.22 | −1.30 | Tmlhe |
| 1420770_at | Lgx only | 119 | 204 | 196 | 229 | 1.71 | 1.64 | 1.92 | Klk1b24 |
| 1420812_at | Res only | 636 | 684 | 880 | 887 | 1.08 | 1.38 | 1.39 | Hdac7a |
| 1420815_at | Res & Lgx | 6531 | 5930 | 4529 | 3754 | −1.10 | −1.44 | −1.74 | Gdi2 |
| 1420829_a_at | Res & Lgx | 1575 | 1422 | 1004 | 915 | −1.11 | −1.57 | −1.72 | Ywhaq |
| 1420850_at | Lgx only | 811 | 697 | 641 | 504 | −1.16 | −1.27 | −1.61 | Crnkl1 |
| 1420851_at | Lgx only | 742 | 595 | 613 | 560 | −1.25 | −1.21 | −1.32 | Pard6g |
| 1420858_at | Lgx only | 4637 | 3856 | 3968 | 3017 | −1.20 | −1.17 | −1.54 | Pkia |
| 1420886_a_at | Res & Lgx | 3739 | 3833 | 4963 | 5400 | 1.03 | 1.33 | 1.44 | Xbp1 |
| 1420890_at | Res & Lgx | 5214 | 4425 | 2406 | 1933 | −1.18 | −2.17 | −2.70 | Hccs |
| 1420895_at | Lgx only | 917 | 770 | 710 | 650 | −1.19 | −1.29 | −1.41 | Tgfbr1 |
| 1420909_at | Lgx only | 2678 | 2641 | 1885 | 1364 | −1.01 | −1.42 | −1.96 | Vegfa |
| 1420911_a_at | Lgx only | 3466 | 3820 | 3678 | 4193 | 1.10 | 1.06 | 1.21 | Mfge8 |
| 1420925_at | Lgx only | 72 | 124 | 151 | 175 | 1.71 | 2.08 | 2.42 | Tub |
| 1420960_at | Res only | 96 | 110 | 192 | 231 | 1.15 | 1.99 | 2.40 | Fancg |
| 1420965_a_at | Lgx only | 874 | 1154 | 1487 | 1587 | 1.32 | 1.70 | 1.82 | Enc1 |
| 1420969_at | Lgx only | 466 | 483 | 535 | 830 | 1.04 | 1.15 | 1.78 | Btbd14b |
| 1420981_a_at | Lgx only | 827 | 972 | 964 | 1187 | 1.18 | 1.17 | 1.44 | Lmo4 |
| 1420990_at | Res only | 312 | 320 | 517 | 236 | 1.02 | 1.65 | −1.32 | Chd1 |
| 1420991_at | Lgx only | 53384 | 52666 | 61602 | 72604 | −1.01 | 1.15 | 1.36 | Ankrd1 |
| 1421019_at | Lgx only | 4065 | 3950 | 3600 | 3047 | −1.03 | −1.13 | −1.33 | 1700021F05Rik |
| 1421025_at | Lgx only | 849 | 1055 | 1040 | 1273 | 1.24 | 1.22 | 1.50 | Agpat1 |
| 1421027_a_at | Res & Lgx | 1926 | 1835 | 1240 | 1194 | −1.05 | −1.55 | −1.61 | Mef2c |
| 1421042_at | Res only | 445 | 458 | 615 | 583 | 1.03 | 1.38 | 1.31 | Arhgef2 |
| 1421054_at | Lgx only | 720 | 688 | 568 | 429 | −1.05 | −1.27 | −1.68 | Xpo4 |
| 1421087_at | CR & Lgx | 659 | 948 | 669 | 1049 | 1.44 | 1.02 | 1.59 | Per3 |
| 1421096_at | Res & Lgx | 343 | 231 | 143 | 153 | −1.48 | −2.39 | −2.24 | Trpc1 |
| 1421099_at | Lgx only | 201 | 206 | 279 | 348 | 1.03 | 1.39 | 1.74 | Bhlhb3 |
| 1421140_a_at | Lgx only | 966 | 1184 | 1048 | 1522 | 1.23 | 1.09 | 1.57 | Foxp1 |
| 1421164_a_at | Lgx only | 790 | 1029 | 1101 | 1483 | 1.30 | 1.39 | 1.88 | Arhgef1 |
| 1421174_at | CR only | 170 | 74 | 105 | 131 | −2.29 | −1.62 | −1.30 | Irf4 |
| 1421254_a_at | Res & Lgx | 8190 | 8322 | 11932 | 11052 | 1.02 | 1.46 | 1.35 | Sgcg |
| 1421265_a_at | Lgx only | 2787 | 3702 | 3156 | 4067 | 1.33 | 1.13 | 1.46 | Rbm38 |
| 1421287_a_at | Res & Lgx | 1918 | 2165 | 2346 | 2394 | 1.13 | 1.22 | 1.25 | Pecam1 |
| 1421292_a_at | Lgx only | 1159 | 1309 | 1372 | 1641 | 1.13 | 1.18 | 1.42 | A730008L03Rik |
| 1421301_at | Res & Lgx | 25 | 50 | 65 | 79 | 2.01 | 2.64 | 3.20 | Zic2 |
| 1421361_at | Res only | 7 | 12 | 14 | 17 | 1.71 | 1.97 | 2.36 | Grk1 |
| 1421373_at | Lgx only | 150 | 180 | 225 | 291 | 1.20 | 1.50 | 1.94 | Cox4i2 |
| 1421374_a_at | Res & Lgx | 22933 | 26328 | 30566 | 37711 | 1.15 | 1.33 | 1.64 | Fxyd1 |
| 1421425_a_at | Lgx only | 16449 | 16822 | 17946 | 19197 | 1.02 | 1.09 | 1.17 | Rcan2 |
| 1421444_at | Res only | 74 | 60 | 18 | 28 | −1.23 | −4.13 | −2.61 | Pgr |
| 1421468_at | Lgx only | 901 | 957 | 947 | 1325 | 1.06 | 1.05 | 1.47 | Kcnj3 |
| 1421530_a_at | Lgx only | 27 | 71 | 53 | 97 | 2.66 | 1.98 | 3.62 | Grm8 |
| 1421534_at | Lgx only | 811 | 795 | 808 | 606 | −1.02 | −1.00 | −1.34 | Dfna5h |
| 1421541_a_at | Lgx only | 21 | 44 | 48 | 100 | 2.09 | 2.27 | 4.69 | Mef2b |
| 1421654_a_at | Lgx only | 1679 | 1993 | 2038 | 2187 | 1.19 | 1.21 | 1.30 | Lmna |
| 1421657_a_at | Lgx only | 586 | 697 | 816 | 882 | 1.19 | 1.39 | 1.51 | Sox17 |
| 1421712_at | Res only | 44 | 104 | 121 | 85 | 2.35 | 2.74 | 1.93 | Sele |
| 1421729_a_at | Res & Lgx | 681 | 584 | 500 | 370 | −1.17 | −1.36 | −1.84 | Fert2 |
| 1421733_a_at | Res & Lgx | 1129 | 1104 | 1432 | 1650 | −1.02 | 1.27 | 1.46 | Tpst1 |
| 1421743_a_at | Res & Lgx | 9703 | 10330 | 11559 | 11327 | 1.06 | 1.19 | 1.17 | Pcbp2 |
| 1421750_a_at | Lgx only | 2246 | 2231 | 1829 | 1446 | −1.01 | −1.23 | −1.55 | Vbp1 |
| 1421797_a_at | Lgx only | 557 | 651 | 646 | 772 | 1.17 | 1.16 | 1.39 | Snx12 |
| 1421808_at | Res only | 72 | 37 | 25 | 26 | −1.92 | −2.88 | −2.71 | Defb5 |
| 1421810_at | Lgx only | 868 | 921 | 932 | 1143 | 1.06 | 1.07 | 1.32 | Dgcr2 |
| 1421813_a_at | Lgx only | 18627 | 21854 | 22085 | 24924 | 1.17 | 1.19 | 1.34 | Psap |
| 1421820_a_at | Lgx only | 2152 | 2587 | 2644 | 3110 | 1.20 | 1.23 | 1.45 | Nf2 |
| 1421826_at | Lgx only | 702 | 623 | 580 | 445 | −1.13 | −1.21 | −1.58 | Dll4 |
| 1421861_at | Lgx only | 1285 | 1602 | 1652 | 1882 | 1.25 | 1.28 | 1.46 | Clstn1 |
| 1421871_at | Lgx only | 1104 | 837 | 695 | 549 | −1.32 | −1.59 | −2.01 | Sh3bgrl |
| 1421872_at | Lgx only | 911 | 910 | 719 | 556 | −1.00 | −1.27 | −1.64 | Rab24 |
| 1421880_at | Res only | 970 | 1061 | 1162 | 1121 | 1.09 | 1.20 | 1.16 | Mtmr1 |
| 1421887_a_at | Lgx only | 10860 | 11944 | 14384 | 16154 | 1.10 | 1.32 | 1.49 | Aplp2 |
| 1421894_a_at | Lgx only | 1285 | 1111 | 1077 | 876 | −1.16 | −1.19 | −1.47 | Tpp2 |
| 1421900_at | Lgx only | 704 | 851 | 937 | 1086 | 1.21 | 1.33 | 1.54 | Eif2ak1 |
| 1421910_at | CR & Lgx | 616 | 466 | 496 | 388 | −1.32 | −1.24 | −1.58 | Tcf20 |
| 1421929_at | Res & Lgx | 584 | 771 | 1070 | 1011 | 1.32 | 1.83 | 1.73 | Epha4 |
| 1421960_at | Lgx only | 251 | 313 | 373 | 451 | 1.25 | 1.48 | 1.79 | Adcy3 |
| 1421985_a_at | Lgx only | 1206 | 1190 | 1259 | 970 | −1.01 | 1.04 | −1.24 | Eif4e2 |
| 1422063_a_at | Res & Lgx | 303 | 432 | 514 | 657 | 1.43 | 1.70 | 2.17 | Pex5 |
| 1422085_at | Res only | 89 | 141 | 178 | 209 | 1.58 | 2.00 | 2.34 | Tbx19 |
| 1422122_at | Lgx only | 45 | 52 | 102 | 145 | 1.15 | 2.26 | 3.23 | Fcer2a |
| 1422157_a_at | Res & Lgx | 1891 | 1621 | 1425 | 1408 | −1.17 | −1.33 | −1.34 | Itgb1bp1 |
| 1422160_at | Res & Lgx | 194 | 137 | 98 | 86 | −1.42 | −1.99 | −2.24 | H2-T24 |
| 1422183_a_at | Res & Lgx | 456 | 433 | 718 | 774 | −1.05 | 1.57 | 1.70 | Adra1b |
| 1422185_a_at | Lgx only | 2280 | 2364 | 3023 | 3264 | 1.04 | 1.33 | 1.43 | Cyb5r3 |
| 1422202_at | All | 187 | 261 | 266 | 299 | 1.39 | 1.42 | 1.60 | Thrb |
| 1422250_at | Res only | 74 | 95 | 175 | 109 | 1.29 | 2.36 | 1.47 | Map3k2 |
| 1422253_at | Res only | 72 | 82 | 22 | 25 | 1.14 | −3.30 | −2.84 | Col10a1 |
| 1422273_at | CR only | 17 | 64 | 65 | 79 | 3.66 | 3.75 | 4.54 | Mmp1b |
| 1422303_a_at | Lgx only | 115 | 142 | 161 | 192 | 1.24 | 1.40 | 1.67 | Tnfrsf18 |
| 1422325_at | CR only | 99 | 54 | 92 | 79 | −1.85 | −1.08 | −1.25 | Magea5 |
| 1422349_at | Res only | 108 | 91 | 204 | 164 | −1.19 | 1.88 | 1.51 | Ccr1l1 |
| 1422368_at | CR only | 63 | 20 | 70 | 73 | −3.19 | 1.12 | 1.17 | V1ra5 |
| 1422429_at | Res & Lgx | 3906 | 3945 | 3274 | 3064 | 1.01 | −1.19 | −1.27 | Rnf14 |
| 1422431_at | Res & Lgx | 174 | 193 | 294 | 266 | 1.11 | 1.69 | 1.53 | Magee1 |
| 1422442_at | Res & Lgx | 2592 | 2482 | 1984 | 1860 | −1.04 | −1.31 | −1.39 | Smu1 |
| 1422443_at | CR only | 2188 | 2545 | 2408 | 2383 | 1.16 | 1.10 | 1.09 | Xpnpep1 |
| 1422470_at | CR & Lgx | 10633 | 8053 | 8886 | 6881 | −1.32 | −1.20 | −1.55 | Bnip3 |
| 1422476_at | Lgx only | 632 | 824 | 773 | 895 | 1.30 | 1.22 | 1.42 | Ifi30 |
| 1422479_at | CR only | 1246 | 1673 | 1146 | 1250 | 1.34 | −1.09 | 1.00 | Acss2 |
| 1422505_at | Res & Lgx | 2509 | 2529 | 2006 | 2084 | 1.01 | −1.25 | −1.20 | Chrac1 |
| 1422514_at | Lgx only | 811 | 912 | 1085 | 1253 | 1.12 | 1.34 | 1.54 | Aebp1 |
| 1422521_at | Lgx only | 1147 | 1461 | 1635 | 2058 | 1.27 | 1.43 | 1.79 | Dctn1 |
| 1422536_at | Lgx only | 92593 | 100541 | 105298 | 145101 | 1.09 | 1.14 | 1.57 | Tnni3 |
| 1422559_at | Res & Lgx | 2320 | 2134 | 1617 | 1469 | −1.09 | −1.44 | −1.58 | Ube2n |
| 1422562_at | Lgx only | 4072 | 5149 | 5749 | 7406 | 1.26 | 1.41 | 1.82 | Rrad |
| 1422568_at | Lgx only | 4621 | 4163 | 3766 | 3704 | −1.11 | −1.23 | −1.25 | Ndel1 |
| 1422579_at | Lgx only | 2795 | 2515 | 2100 | 1950 | −1.11 | −1.33 | −1.43 | Hspe1 |
| 1422580_at | Lgx only | 21473 | 23970 | 33904 | 66655 | 1.12 | 1.58 | 3.10 | Myl4 |
| 1422589_at | Lgx only | 1557 | 2101 | 1799 | 2359 | 1.35 | 1.16 | 1.51 | Rab3a |
| 1422594_at | Res only | 1164 | 1044 | 782 | 887 | −1.12 | −1.49 | −1.31 | 5730470L24Rik |
| 1422597_at | Lgx only | 1172 | 1880 | 1299 | 1581 | 1.60 | 1.11 | 1.35 | Mmp15 |
| 1422598_at | Lgx only | 376 | 482 | 558 | 667 | 1.28 | 1.48 | 1.77 | Casq1 |
| 1422601_at | Lgx only | 678 | 602 | 614 | 451 | −1.13 | −1.11 | −1.50 | Serpinb9 |
| 1422622_at | Lgx only | 1987 | 2361 | 2556 | 2886 | 1.19 | 1.29 | 1.45 | Nos3 |
| 1422624_at | Res & Lgx | 585 | 523 | 324 | 366 | −1.12 | −1.80 | −1.60 | Rev1 |
| 1422631_at | CR only | 459 | 290 | 354 | 311 | −1.58 | −1.30 | −1.47 | Ahr |
| 1422636_at | Lgx only | 484 | 476 | 356 | 311 | −1.02 | −1.36 | −1.55 | Dmtf1 |
| 1422647_at | Lgx only | 551 | 486 | 766 | 945 | −1.13 | 1.39 | 1.72 | Ring1 |
| 1422654_at | Res & Lgx | 6849 | 8679 | 9035 | 10593 | 1.27 | 1.32 | 1.55 | Sgca |
| 1422656_at | Lgx only | 348 | 337 | 280 | 194 | −1.03 | −1.24 | −1.79 | Rasl2-9 |
| 1422669_at | Res & Lgx | 1326 | 1103 | 925 | 768 | −1.20 | −1.43 | −1.72 | Ebag9 |
| 1422678_at | Lgx only | 8205 | 10614 | 8228 | 11571 | 1.29 | 1.00 | 1.41 | Dgat2 |
| 1422687_at | Res only | 342 | 422 | 540 | 502 | 1.23 | 1.58 | 1.47 | Nras |
| 1422704_at | Lgx only | 912 | 889 | 790 | 692 | −1.03 | −1.16 | −1.32 | Gyk |
| 1422710_a_at | Lgx only | 204 | 256 | 533 | 712 | 1.26 | 2.62 | 3.50 | Cacna1h |
| 1422731_at | Lgx only | 1938 | 1979 | 1826 | 1508 | 1.02 | −1.06 | −1.29 | Limd1 |
| 1422750_a_at | Lgx only | 12 | 15 | 16 | 26 | 1.27 | 1.32 | 2.13 | Zmynd10 |
| 1422754_at | Lgx only | 5302 | 6134 | 5414 | 7120 | 1.16 | 1.02 | 1.34 | Tmod1 |
| 1422759_a_at | Lgx only | 716 | 910 | 1014 | 972 | 1.27 | 1.42 | 1.36 | Xpo6 |
| 1422771_at | Lgx only | 662 | 616 | 845 | 1033 | −1.07 | 1.28 | 1.56 | Smad6 |
| 1422794_at | Lgx only | 2668 | 2498 | 2540 | 2197 | −1.07 | −1.05 | −1.21 | Cul3 |
| 1422797_at | Lgx only | 2497 | 2556 | 2816 | 3053 | 1.02 | 1.13 | 1.22 | Mapbpip |
| 1422799_at | Lgx only | 1417 | 2232 | 2331 | 3007 | 1.58 | 1.64 | 2.12 | Bat2 |
| 1422801_at | Lgx only | 1868 | 2054 | 2333 | 2401 | 1.10 | 1.25 | 1.29 | G3bp1 |
| 1422811_at | Lgx only | 1842 | 1949 | 3745 | 4459 | 1.06 | 2.03 | 2.42 | Slc27a1 |
| 1422819_at | Lgx only | 1868 | 1541 | 1669 | 1324 | −1.21 | −1.12 | −1.41 | Mrpl36 |
| 1422820_at | Res & Lgx | 1160 | 1556 | 2287 | 3124 | 1.34 | 1.97 | 2.69 | Lipe |
| 1422845_at | Res & Lgx | 2601 | 2532 | 1767 | 1768 | −1.03 | −1.47 | −1.47 | Canx |
| 1422855_at | Lgx only | 1072 | 1244 | 1287 | 1505 | 1.16 | 1.20 | 1.40 | Cpsf3 |
| 1422858_at | Res & Lgx | 647 | 600 | 471 | 385 | −1.08 | −1.37 | −1.68 | Trip4 |
| 1422869_at | CR only | 322 | 486 | 356 | 429 | 1.51 | 1.11 | 1.33 | Mertk |
| 1422880_at | Res only | 3821 | 4166 | 5178 | 4697 | 1.09 | 1.36 | 1.23 | Sypl |
| 1422884_at | Res & Lgx | 2609 | 2458 | 1699 | 1360 | −1.06 | −1.54 | −1.92 | Snrpd3 |
| 1422888_at | Res & Lgx | 2451 | 2326 | 1824 | 1740 | −1.05 | −1.34 | −1.41 | Rnf5 |
| 1422895_at | Lgx only | 1484 | 1345 | 1128 | 1085 | −1.10 | −1.32 | −1.37 | Vamp4 |
| 1422904_at | Lgx only | 1644 | 2057 | 2434 | 3071 | 1.25 | 1.48 | 1.87 | Fmo2 |
| 1422919_at | Lgx only | 1447 | 1561 | 1174 | 1081 | 1.08 | −1.23 | −1.34 | Hrasls |
| 1422927_at | Lgx only | 9559 | 8873 | 9036 | 7917 | −1.08 | −1.06 | −1.21 | Yipf7 |
| 1422975_at | Lgx only | 270 | 215 | 239 | 143 | −1.25 | −1.13 | −1.88 | Mme |
| 1423025_a_at | Res & Lgx | 4676 | 4201 | 3674 | 3128 | −1.11 | −1.27 | −1.50 | Schip1 |
| 1423038_at | CR only | 739 | 613 | 619 | 676 | −1.21 | −1.19 | −1.09 | Stx6 |
| 1423044_at | Lgx only | 3560 | 3888 | 3058 | 2626 | 1.09 | −1.16 | −1.36 | Prosc |
| 1423047_at | Lgx only | 1895 | 2061 | 2077 | 2332 | 1.09 | 1.10 | 1.23 | Tollip |
| 1423049_a_at | Lgx only | 87808 | 100652 | 95761 | 125022 | 1.15 | 1.09 | 1.42 | Tpm1 |
| 1423067_at | Lgx only | 1021 | 1177 | 1166 | 1322 | 1.15 | 1.14 | 1.29 | Cdk5rap3 |
| 1423072_at | Res & Lgx | 9143 | 7426 | 14144 | 14099 | −1.23 | 1.55 | 1.54 | 6720475J19Rik |
| 1423073_at | Res & Lgx | 2730 | 2611 | 1976 | 1859 | −1.05 | −1.38 | −1.47 | Cmpk |
| 1423078_a_at | Res only | 281 | 268 | 138 | 193 | −1.05 | −2.03 | −1.45 | Sc4mol |
| 1423083_at | Lgx only | 829 | 634 | 640 | 576 | −1.31 | −1.30 | −1.44 | Rab33b |
| 1423085_at | Lgx only | 2081 | 2337 | 2335 | 2878 | 1.12 | 1.12 | 1.38 | Efnb3 |
| 1423086_at | Lgx only | 581 | 574 | 944 | 865 | −1.01 | 1.62 | 1.49 | Npc1 |
| 1423104_at | Lgx only | 1861 | 2159 | 1551 | 1285 | 1.16 | −1.20 | −1.45 | Irs1 |
| 1423107_at | Res & Lgx | 13435 | 12780 | 18773 | 18511 | −1.05 | 1.40 | 1.38 | Ube2b |
| 1423115_at | Lgx only | 1874 | 2293 | 2021 | 2497 | 1.22 | 1.08 | 1.33 | St6galnac6 |
| 1423116_at | Lgx only | 367 | 369 | 418 | 514 | 1.01 | 1.14 | 1.40 | Dom3z |
| 1423117_at | Lgx only | 1641 | 1974 | 2060 | 2325 | 1.20 | 1.26 | 1.42 | Pum1 |
| 1423120_at | Lgx only | 1872 | 1820 | 2582 | 2866 | −1.03 | 1.38 | 1.53 | Ide |
| 1423145_a_at | Lgx only | 67091 | 70639 | 67884 | 94069 | 1.05 | 1.01 | 1.40 | Tcap |
| 1423159_at | Lgx only | 8820 | 7770 | 8104 | 7185 | −1.14 | −1.09 | −1.23 | Dld |
| 1423167_at | Lgx only | 2048 | 1950 | 1592 | 1289 | −1.05 | −1.29 | −1.59 | Mobkl3 |
| 1423185_a_at | Lgx only | 1046 | 1030 | 1125 | 1433 | −1.01 | 1.08 | 1.37 | Ubap1 |
| 1423195_at | Lgx only | 770 | 772 | 644 | 545 | 1.00 | −1.20 | −1.41 | Hiat1 |
| 1423210_a_at | Lgx only | 15838 | 13912 | 13292 | 11593 | −1.14 | −1.19 | −1.37 | Nola3 |
| 1423238_at | CR only | 10141 | 11875 | 10734 | 10997 | 1.17 | 1.06 | 1.08 | Itgb1bp2 |
| 1423245_at | Lgx only | 2212 | 2559 | 2650 | 2839 | 1.16 | 1.20 | 1.28 | Cops7a |
| 1423247_at | Res & Lgx | 1654 | 1535 | 1377 | 1412 | −1.08 | −1.20 | −1.17 | Txndc4 |
| 1423283_at | Res only | 2245 | 2516 | 2704 | 2323 | 1.12 | 1.20 | 1.03 | Pitpna |
| 1423289_a_at | Lgx only | 590 | 520 | 490 | 411 | −1.14 | −1.21 | −1.44 | 1810029B16Rik |
| 1423296_at | Res & Lgx | 4260 | 3917 | 3455 | 3149 | −1.09 | −1.23 | −1.35 | Psmd8 |
| 1423315_at | Res only | 239 | 239 | 393 | 245 | −1.00 | 1.64 | 1.03 | Bbc3 |
| 1423332_at | Lgx only | 7044 | 6555 | 6145 | 5521 | −1.07 | −1.15 | −1.28 | Sdcbp |
| 1423347_at | Lgx only | 1717 | 1446 | 1257 | 946 | −1.19 | −1.37 | −1.81 | Sec23a |
| 1423362_at | Lgx only | 1116 | 1401 | 1546 | 2127 | 1.26 | 1.38 | 1.91 | Sort1 |
| 1423365_at | Lgx only | 220 | 341 | 321 | 487 | 1.55 | 1.46 | 2.22 | Cacna1g |
| 1423368_at | Res only | 26862 | 24889 | 23668 | 24004 | −1.08 | −1.13 | −1.12 | Laptm4a |
| 1423369_at | Lgx only | 512 | 450 | 400 | 277 | −1.14 | −1.28 | −1.84 | Fmr1 |
| 1423373_at | Lgx only | 1662 | 1543 | 1231 | 1041 | −1.08 | −1.35 | −1.60 | Rpp30 |
| 1423383_a_at | Lgx only | 3010 | 3451 | 3134 | 3991 | 1.15 | 1.04 | 1.33 | Osbpl9 |
| 1423393_at | Res & Lgx | 9550 | 11356 | 12552 | 12982 | 1.19 | 1.31 | 1.36 | Clic4 |
| 1423407_a_at | Res & Lgx | 1285 | 1527 | 1976 | 2493 | 1.19 | 1.54 | 1.94 | Fbln2 |
| 1423423_at | Res & Lgx | 3996 | 3941 | 3047 | 3212 | −1.01 | −1.31 | −1.24 | Pdia3 |
| 1423425_at | Lgx only | 639 | 706 | 761 | 947 | 1.10 | 1.19 | 1.48 | 1300012G16Rik |
| 1423431_a_at | Res & Lgx | 1593 | 1952 | 2320 | 2336 | 1.23 | 1.46 | 1.47 | Mybbp1a |
| 1423440_at | Lgx only | 631 | 546 | 529 | 467 | −1.15 | −1.19 | −1.35 | 1110001A07Rik |
| 1423441_at | Lgx only | 4060 | 3903 | 3850 | 3452 | −1.04 | −1.05 | −1.18 | Tfb2m |
| 1423448_at | Res & Lgx | 4469 | 4669 | 6840 | 6850 | 1.04 | 1.53 | 1.53 | Rab11b |
| 1423449_a_at | Lgx only | 1823 | 2337 | 2121 | 2533 | 1.28 | 1.16 | 1.39 | Actn4 |
| 1423459_at | Lgx only | 3896 | 3424 | 3126 | 2505 | −1.14 | −1.25 | −1.56 | Cops2 |
| 1423474_at | Res & Lgx | 2213 | 2036 | 1686 | 1390 | −1.09 | −1.31 | −1.59 | Top1 |
| 1423485_at | Lgx only | 523 | 580 | 631 | 930 | 1.11 | 1.21 | 1.78 | Rad54l2 |
| 1423486_at | Res & Lgx | 4116 | 3309 | 2762 | 2491 | −1.24 | −1.49 | −1.65 | Cript |
| 1423490_at | Lgx only | 3791 | 3965 | 3044 | 2767 | 1.05 | −1.25 | −1.37 | Fbxo3 |
| 1423494_at | CR only | 38 | 105 | 93 | 45 | 2.79 | 2.46 | 1.20 | 2310042E22Rik |
| 1423512_at | Lgx only | 664 | 554 | 571 | 494 | −1.20 | −1.16 | −1.35 | AW209491 |
| 1423529_at | Lgx only | 76 | 62 | 55 | 20 | −1.22 | −1.38 | −3.78 | G6pc2 |
| 1423535_at | Lgx only | 4603 | 3572 | 3482 | 2393 | −1.29 | −1.32 | −1.92 | LOC100047794 |
| 1423557_at | Res & Lgx | 4123 | 4659 | 2949 | 2995 | 1.13 | −1.40 | −1.38 | Ifngr2 |
| 1423565_at | Lgx only | 6741 | 7261 | 5935 | 5619 | 1.08 | −1.14 | −1.20 | Paics |
| 1423577_at | CR only | 866 | 691 | 713 | 759 | −1.25 | −1.22 | −1.14 | Ankrd32 |
| 1423588_at | Res & Lgx | 1204 | 1118 | 984 | 929 | −1.08 | −1.22 | −1.30 | Arpc4 |
| 1423599_a_at | Lgx only | 741 | 579 | 555 | 467 | −1.28 | −1.33 | −1.59 | Pdcl |
| 1423609_a_at | Lgx only | 2102 | 2341 | 2391 | 3062 | 1.11 | 1.14 | 1.46 | Mgat1 |
| 1423611_at | CR only | 736 | 949 | 973 | 1010 | 1.29 | 1.32 | 1.37 | Akp2 |
| 1423620_at | Lgx only | 428 | 413 | 295 | 207 | −1.04 | −1.45 | −2.07 | Cenpq |
| 1423629_at | Lgx only | 842 | 1015 | 1126 | 1457 | 1.21 | 1.34 | 1.73 | Dnm2 |
| 1423642_at | CR & Res | 11579 | 15048 | 8041 | 9198 | 1.30 | −1.44 | −1.26 | Tubb2c |
| 1423643_at | Lgx only | 1071 | 1196 | 843 | 821 | 1.12 | −1.27 | −1.30 | Ddx39 |
| 1423647_a_at | Res only | 344 | 396 | 605 | 457 | 1.15 | 1.76 | 1.33 | Zdhhc3 |
| 1423648_at | CR only | 1771 | 2210 | 1681 | 1872 | 1.25 | −1.05 | 1.06 | Pdia6 |
| 1423657_at | Res & Lgx | 1515 | 1485 | 1236 | 1166 | −1.02 | −1.23 | −1.30 | Cdipt |
| 1423662_at | Res & Lgx | 1443 | 1423 | 991 | 902 | −1.01 | −1.46 | −1.60 | Atp6ap2 |
| 1423663_at | CR only | 1057 | 1384 | 1080 | 1178 | 1.31 | 1.02 | 1.12 | Flcn |
| 1423667_at | All | 8373 | 7112 | 6577 | 5613 | −1.18 | −1.27 | −1.49 | Mat2a |
| 1423669_at | Lgx only | 1075 | 1350 | 1555 | 2209 | 1.26 | 1.45 | 2.05 | Col1a1 |
| 1423670_a_at | CR & Lgx | 3169 | 3700 | 3314 | 3615 | 1.17 | 1.05 | 1.14 | Srpr |
| 1423676_at | Lgx only | 58661 | 62536 | 61203 | 80373 | 1.07 | 1.04 | 1.37 | Atp5h |
| 1423685_at | Lgx only | 1108 | 1452 | 1375 | 1574 | 1.31 | 1.24 | 1.42 | Aars |
| 1423694_at | CR only | 1790 | 2077 | 2089 | 1818 | 1.16 | 1.17 | 1.02 | Kctd10 |
| 1423697_at | Lgx only | 6981 | 6288 | 6277 | 5248 | −1.11 | −1.11 | −1.33 | Psmd6 |
| 1423710_at | Lgx only | 9222 | 11179 | 10001 | 11429 | 1.21 | 1.08 | 1.24 | Dlst |
| 1423711_at | Res & Lgx | 2316 | 2307 | 1924 | 1627 | −1.00 | −1.20 | −1.42 | Ndufaf1 |
| 1423725_at | Lgx only | 985 | 705 | 674 | 762 | −1.40 | −1.46 | −1.29 | Pls3 |
| 1423734_at | Lgx only | 5006 | 5612 | 5707 | 6496 | 1.12 | 1.14 | 1.30 | Rac1 |
| 1423737_at | Lgx only | 24235 | 26450 | 27218 | 29730 | 1.09 | 1.12 | 1.23 | Ndufs3 |
| 1423753_at | CR only | 1118 | 734 | 898 | 856 | −1.52 | −1.24 | −1.31 | Bambi |
| 1423759_a_at | Res & Lgx | 4031 | 3513 | 2876 | 2736 | −1.15 | −1.40 | −1.47 | Tmco1 |
| 1423766_at | Res & Lgx | 1610 | 1380 | 1025 | 987 | −1.17 | −1.57 | −1.63 | Pak1ip1 |
| 1423767_at | Lgx only | 2146 | 2025 | 1774 | 1497 | −1.06 | −1.21 | −1.43 | 2810410M20Rik |
| 1423771_at | Lgx only | 815 | 750 | 811 | 1094 | −1.09 | −1.00 | 1.34 | Prkcdbp |
| 1423773_at | Lgx only | 4627 | 3985 | 3629 | 2816 | −1.16 | −1.28 | −1.64 | Gpbp1 |
| 1423780_at | Lgx only | 10898 | 11906 | 12709 | 12518 | 1.09 | 1.17 | 1.15 | Hibadh |
| 1423785_at | Lgx only | 11606 | 13426 | 11821 | 15052 | 1.16 | 1.02 | 1.30 | Egln1 |
| 1423790_at | Lgx only | 1487 | 1751 | 2032 | 2310 | 1.18 | 1.37 | 1.55 | Dap |
| 1423793_at | Res & Lgx | 936 | 1073 | 1319 | 1580 | 1.15 | 1.41 | 1.69 | D2Ertd391e |
| 1423810_at | CR only | 1783 | 2180 | 1746 | 1816 | 1.22 | −1.02 | 1.02 | Ppme1 |
| 1423822_a_at | Lgx only | 730 | 653 | 569 | 558 | −1.12 | −1.28 | −1.31 | Tmem168 |
| 1423845_at | Lgx only | 460 | 477 | 805 | 739 | 1.04 | 1.75 | 1.61 | Csdc2 |
| 1423847_at | Res & Lgx | 231 | 270 | 401 | 391 | 1.17 | 1.73 | 1.69 | Ncapd2 |
| 1423849_a_at | Lgx only | 616 | 713 | 916 | 1030 | 1.16 | 1.49 | 1.67 | Clk3 |
| 1423852_at | Lgx only | 50 | 52 | 68 | 154 | 1.03 | 1.36 | 3.07 | Tmem46 |
| 1423857_at | Res & Lgx | 13121 | 11472 | 8712 | 6533 | −1.14 | −1.51 | −2.01 | Mrpl30 |
| 1423881_at | Lgx only | 1929 | 2007 | 2122 | 2477 | 1.04 | 1.10 | 1.28 | Saps3 |
| 1423882_at | Lgx only | 933 | 840 | 739 | 618 | −1.11 | −1.26 | −1.51 | Rfwd3 |
| 1423883_at | Lgx only | 21573 | 22608 | 26023 | 29695 | 1.05 | 1.21 | 1.38 | Acsl1 |
| 1423892_at | CR only | 3733 | 2926 | 4320 | 4066 | −1.28 | 1.16 | 1.09 | Apbb1 |
| 1423896_a_at | Lgx only | 7953 | 9503 | 10177 | 13002 | 1.19 | 1.28 | 1.63 | Rnf187 |
| 1423907_a_at | Lgx only | 21309 | 20866 | 21974 | 17327 | −1.02 | 1.03 | −1.23 | Ndufs8 |
| 1423909_at | Lgx only | 670 | 657 | 729 | 1002 | −1.02 | 1.09 | 1.50 | Tmem176a |
| 1423919_at | Lgx only | 445 | 348 | 353 | 351 | −1.28 | −1.26 | −1.27 | BC023882 |
| 1423927_at | Res & Lgx | 510 | 626 | 737 | 770 | 1.23 | 1.45 | 1.51 | Slc35b2 |
| 1423939_a_at | Lgx only | 6662 | 5968 | 5430 | 4027 | −1.12 | −1.23 | −1.65 | Yif1a |
| 1423947_at | Res & Lgx | 1479 | 1457 | 2188 | 2164 | −1.01 | 1.48 | 1.46 | 1110008P14Rik |
| 1423951_at | Lgx only | 936 | 768 | 718 | 654 | −1.22 | −1.30 | −1.43 | Tm2d3 |
| 1423958_a_at | Lgx only | 1601 | 1398 | 1213 | 998 | −1.15 | −1.32 | −1.60 | Ttc33 |
| 1423960_at | Res & Lgx | 1368 | 1371 | 1946 | 2010 | 1.00 | 1.42 | 1.47 | Mboat5 |
| 1423961_at | Res & Lgx | 1414 | 1142 | 839 | 602 | −1.24 | −1.69 | −2.35 | LOC100045629 |
| 1423967_at | Lgx only | 903 | 1044 | 1073 | 1359 | 1.16 | 1.19 | 1.51 | Palm |
| 1423969_at | All | 723 | 538 | 512 | 442 | −1.34 | −1.41 | −1.64 | Nup37 |
| 1423972_at | Lgx only | 31981 | 34252 | 35126 | 43175 | 1.07 | 1.10 | 1.35 | Etfa |
| 1423973_a_at | Lgx only | 644 | 716 | 819 | 1000 | 1.11 | 1.27 | 1.55 | Arf3 |
| 1423978_at | Lgx only | 920 | 1504 | 1209 | 1467 | 1.64 | 1.31 | 1.59 | Sbk1 |
| 1423991_at | Lgx only | 392 | 425 | 448 | 564 | 1.08 | 1.14 | 1.44 | Nol14 |
| 1423993_at | Lgx only | 4392 | 3844 | 3630 | 3056 | −1.14 | −1.21 | −1.44 | Atp6v1f |
| 1424000_a_at | Lgx only | 20066 | 22495 | 23422 | 27778 | 1.12 | 1.17 | 1.38 | Rps11 |
| 1424002_at | Res & Lgx | 2495 | 2175 | 1827 | 1785 | −1.15 | −1.37 | −1.40 | Pdcl3 |
| 1424005_at | Lgx only | 3828 | 3557 | 3275 | 3040 | −1.08 | −1.17 | −1.26 | B230219D22Rik |
| 1424010_at | Lgx only | 168 | 233 | 382 | 732 | 1.38 | 2.27 | 4.36 | Mfap4 |
| 1424025_at | Res & Lgx | 5858 | 5688 | 4312 | 4383 | −1.03 | −1.36 | −1.34 | BC013529 |
| 1424027_at | Res only | 619 | 902 | 924 | 853 | 1.46 | 1.49 | 1.38 | Pxn |
| 1424028_at | Lgx only | 874 | 861 | 614 | 481 | −1.02 | −1.42 | −1.82 | 5830457O10Rik |
| 1424033_at | Res & Lgx | 935 | 735 | 479 | 332 | −1.27 | −1.95 | −2.82 | Sfrs7 |
| 1424036_at | Lgx only | 1422 | 1354 | 1874 | 1752 | −1.05 | 1.32 | 1.23 | Prpf6 |
| 1424038_a_at | Lgx only | 2801 | 3278 | 3181 | 3602 | 1.17 | 1.14 | 1.29 | 2310044H10Rik |
| 1424040_at | Lgx only | 5989 | 7521 | 7112 | 8135 | 1.26 | 1.19 | 1.36 | Mtap7d1 |
| 1424053_a_at | Res & Lgx | 5291 | 5313 | 6583 | 6840 | 1.00 | 1.24 | 1.29 | Tcf25 |
| 1424054_at | Res & Lgx | 1063 | 1467 | 1588 | 1882 | 1.38 | 1.49 | 1.77 | Btbd2 |
| 1424058_at | Lgx only | 640 | 771 | 767 | 944 | 1.21 | 1.20 | 1.48 | Prrc1 |
| 1424066_at | Lgx only | 990 | 1038 | 1084 | 1324 | 1.05 | 1.09 | 1.34 | Dus3l |
| 1424077_at | Res only | 654 | 544 | 493 | 479 | −1.20 | −1.33 | −1.37 | Gdpd1 |
| 1424081_at | Res & Lgx | 380 | 271 | 291 | 277 | −1.40 | −1.31 | −1.37 | Pcgf6 |
| 1424099_at | Lgx only | 2023 | 1663 | 1332 | 1265 | −1.22 | −1.52 | −1.60 | 2310016C16Rik |
| 1424101_at | Res & Lgx | 874 | 934 | 533 | 583 | 1.07 | −1.64 | −1.50 | Hnrpl |
| 1424105_a_at | Lgx only | 3516 | 2620 | 3158 | 2273 | −1.34 | −1.11 | −1.55 | Pttg1 |
| 1424109_a_at | Res & Lgx | 6904 | 7061 | 6174 | 6463 | 1.02 | −1.12 | −1.07 | Glo1 |
| 1424115_at | CR & Lgx | 1970 | 2313 | 2291 | 2496 | 1.17 | 1.16 | 1.27 | Ppp5c |
| 1424121_at | Lgx only | 986 | 890 | 677 | 523 | −1.11 | −1.46 | −1.88 | Commd1 |
| 1424126_at | CR & Res | 6092 | 9782 | 4437 | 5967 | 1.61 | −1.37 | −1.02 | Alas1 |
| 1424134_at | Lgx only | 646 | 529 | 499 | 436 | −1.22 | −1.29 | −1.48 | Rspry1 |
| 1424138_at | Lgx only | 1703 | 1927 | 1979 | 2218 | 1.13 | 1.16 | 1.30 | Rhbdf1 |
| 1424139_at | Lgx only | 5935 | 4960 | 4416 | 3478 | −1.20 | −1.34 | −1.71 | Rap1a |
| 1424140_at | Res only | 146 | 192 | 243 | 196 | 1.32 | 1.67 | 1.35 | Gale |
| 1424141_at | Lgx only | 2745 | 3324 | 3767 | 4195 | 1.21 | 1.37 | 1.53 | Hectd1 |
| 1424147_at | Res & Lgx | 3868 | 4234 | 2946 | 2570 | 1.09 | −1.31 | −1.50 | Ahsa1 |
| 1424149_at | CR only | 435 | 531 | 523 | 573 | 1.22 | 1.20 | 1.32 | Nsmce2 |
| 1424150_at | Lgx only | 438 | 540 | 561 | 688 | 1.23 | 1.28 | 1.57 | Gdpd5 |
| 1424151_at | Res & Lgx | 2327 | 2380 | 1795 | 1679 | 1.02 | −1.30 | −1.39 | Jtv1 |
| 1424154_a_at | Lgx only | 4155 | 3735 | 3761 | 3368 | −1.11 | −1.10 | −1.23 | Isca2 |
| 1424159_at | Res & Lgx | 618 | 578 | 459 | 454 | −1.07 | −1.34 | −1.36 | 1300010M03Rik |
| 1424160_at | Lgx only | 563 | 486 | 499 | 420 | −1.16 | −1.13 | −1.34 | Alg5 |
| 1424162_at | CR only | 115 | 37 | 144 | 125 | −3.15 | 1.25 | 1.08 | Trim29 |
| 1424163_at | Lgx only | 615 | 642 | 714 | 803 | 1.04 | 1.16 | 1.31 | Rmnd5b |
| 1424167_a_at | Lgx only | 367 | 456 | 568 | 734 | 1.24 | 1.55 | 2.00 | Pmm1 |
| 1424175_at | CR & Lgx | 4138 | 5751 | 4597 | 7356 | 1.39 | 1.11 | 1.78 | Tef |
| 1424178_at | Lgx only | 8368 | 8313 | 7872 | 6995 | −1.01 | −1.06 | −1.20 | Tmem38a |
| 1424179_at | Lgx only | 642 | 594 | 541 | 431 | −1.08 | −1.19 | −1.49 | Plekhj1 |
| 1424184_at | Lgx only | 33037 | 35163 | 37503 | 45711 | 1.06 | 1.14 | 1.38 | Acadvl |
| 1424191_a_at | Lgx only | 613 | 688 | 706 | 837 | 1.12 | 1.15 | 1.36 | Tmem41a |
| 1424209_at | Lgx only | 1349 | 1298 | 1166 | 988 | −1.04 | −1.16 | −1.37 | Rars2 |
| 1424210_at | Res & Lgx | 892 | 764 | 657 | 670 | −1.17 | −1.36 | −1.33 | Erlin1 |
| 1424211_at | CR & Lgx | 1496 | 2374 | 1415 | 1975 | 1.59 | −1.06 | 1.32 | Slc25a33 |
| 1424216_a_at | Lgx only | 1633 | 1442 | 1293 | 1181 | −1.13 | −1.26 | −1.38 | Papola |
| 1424223_at | Lgx only | 7099 | 8501 | 6040 | 6025 | 1.20 | −1.18 | −1.18 | 1700020C11Rik |
| 1424236_at | Lgx only | 741 | 789 | 838 | 950 | 1.06 | 1.13 | 1.28 | Tbc1d10b |
| 1424237_at | Res only | 1047 | 1010 | 1262 | 1184 | −1.04 | 1.21 | 1.13 | Zfp639 |
| 1424247_at | Lgx only | 543 | 591 | 600 | 802 | 1.09 | 1.10 | 1.48 | Erc1 |
| 1424249_a_at | All | 434 | 271 | 226 | 252 | −1.60 | −1.92 | −1.72 | Arhgap9 |
| 1424255_at | Res & Lgx | 2458 | 2688 | 3230 | 3339 | 1.09 | 1.31 | 1.36 | Supt5h |
| 1424258_at | Lgx only | 955 | 696 | 690 | 604 | −1.37 | −1.38 | −1.58 | Polr2d |
| 1424261_at | Res & Lgx | 1144 | 871 | 714 | 580 | −1.31 | −1.60 | −1.97 | Zfp672 |
| 1424274_at | Res & Lgx | 1795 | 1574 | 1426 | 1084 | −1.14 | −1.26 | −1.66 | Vdp |
| 1424276_at | Lgx only | 410 | 338 | 308 | 258 | −1.22 | −1.33 | −1.59 | Snx16 |
| 1424280_at | Res & Lgx | 2125 | 1767 | 1658 | 1182 | −1.20 | −1.28 | −1.80 | Mospd1 |
| 1424303_at | Lgx only | 90 | 76 | 59 | 26 | −1.19 | −1.53 | −3.47 | Depdc7 |
| 1424309_a_at | Res & Lgx | 2945 | 2961 | 2220 | 1975 | 1.01 | −1.33 | −1.49 | Mocs2 |
| 1424318_at | Lgx only | 2148 | 2040 | 1851 | 1802 | −1.05 | −1.16 | −1.19 | 1110067D22Rik |
| 1424321_at | Lgx only | 351 | 263 | 228 | 169 | −1.34 | −1.54 | −2.07 | Rfc4 |
| 1424324_at | Lgx only | 956 | 712 | 539 | 403 | −1.34 | −1.77 | −2.37 | Esco1 |
| 1424346_at | Lgx only | 2287 | 1920 | 1820 | 1658 | −1.19 | −1.26 | −1.38 | Ppp6c |
| 1424349_a_at | Res & Lgx | 2326 | 1877 | 1516 | 1263 | −1.24 | −1.53 | −1.84 | Lpgat1 |
| 1424356_a_at | Lgx only | 770 | 697 | 694 | 603 | −1.10 | −1.11 | −1.28 | Metrnl |
| 1424359_at | Lgx only | 1036 | 1229 | 2242 | 2519 | 1.19 | 2.16 | 2.43 | Oplah |
| 1424361_at | Lgx only | 294 | 408 | 530 | 653 | 1.39 | 1.80 | 2.22 | BC019943 |
| 1424372_at | Res & Lgx | 3282 | 2988 | 2215 | 2091 | −1.10 | −1.48 | −1.57 | Mrpl32 |
| 1424374_at | Lgx only | 1936 | 1845 | 1535 | 1363 | −1.05 | −1.26 | −1.42 | Gimap4 |
| 1424377_at | Lgx only | 1007 | 971 | 895 | 801 | −1.04 | −1.12 | −1.26 | BC003885 |
| 1424380_at | Lgx only | 434 | 432 | 512 | 559 | −1.01 | 1.18 | 1.29 | Vps37b |
| 1424384_a_at | Lgx only | 726 | 981 | 1007 | 1092 | 1.35 | 1.39 | 1.50 | Znrf1 |
| 1424390_at | Lgx only | 597 | 493 | 481 | 472 | −1.21 | −1.24 | −1.26 | Nupl1 |
| 1424391_at | CR & Lgx | 3544 | 3195 | 3213 | 2673 | −1.11 | −1.10 | −1.33 | Nrd1 |
| 1424403_a_at | Lgx only | 254 | 221 | 348 | 376 | −1.15 | 1.37 | 1.48 | Rufy3 |
| 1424406_at | Lgx only | 1180 | 1319 | 1489 | 1520 | 1.12 | 1.26 | 1.29 | Bcl2l13 |
| 1424408_at | Lgx only | 3655 | 3925 | 4168 | 4751 | 1.07 | 1.14 | 1.30 | Lims2 |
| 1424416_at | Lgx only | 2199 | 2535 | 2429 | 2712 | 1.15 | 1.10 | 1.23 | Nkiras2 |
| 1424424_at | Lgx only | 1819 | 1642 | 1309 | 892 | −1.11 | −1.39 | −2.04 | Slc39a1 |
| 1424430_at | Res only | 647 | 598 | 475 | 466 | −1.08 | −1.36 | −1.39 | Mterfd2 |
| 1424433_at | Res & Lgx | 3043 | 3150 | 2599 | 2241 | 1.04 | −1.17 | −1.36 | Msrb2 |
| 1424434_at | Res & Lgx | 2817 | 2667 | 2219 | 2042 | −1.06 | −1.27 | −1.38 | BC024814 |
| 1424447_at | Lgx only | 404 | 418 | 462 | 566 | 1.03 | 1.14 | 1.40 | 1700030K09Rik |
| 1424461_at | Res & Lgx | 4865 | 4886 | 3994 | 3716 | 1.00 | −1.22 | −1.31 | Dctn2 |
| 1424463_at | Res & Lgx | 1299 | 1043 | 945 | 877 | −1.24 | −1.37 | −1.48 | 2210010L05Rik |
| 1424465_at | Lgx only | 957 | 881 | 710 | 583 | −1.09 | −1.35 | −1.64 | Ccdc58 |
| 1424467_at | Lgx only | 1725 | 2308 | 2228 | 2625 | 1.34 | 1.29 | 1.52 | Phldb1 |
| 1424473_at | Lgx only | 911 | 823 | 725 | 502 | −1.11 | −1.26 | −1.81 | Polr2h |
| 1424479_at | Lgx only | 39 | 46 | 64 | 119 | 1.18 | 1.64 | 3.02 | Cst8 |
| 1424500_at | Lgx only | 1020 | 947 | 744 | 637 | −1.08 | −1.37 | −1.60 | Utp6 |
| 1424505_at | Lgx only | 1309 | 1213 | 1172 | 895 | −1.08 | −1.12 | −1.46 | Rmnd1 |
| 1424510_at | Lgx only | 783 | 733 | 627 | 574 | −1.07 | −1.25 | −1.36 | Nudt6 |
| 1424517_at | CR only | 403 | 529 | 450 | 495 | 1.31 | 1.12 | 1.23 | Ccdc12 |
| 1424520_at | Res only | 687 | 597 | 550 | 610 | −1.15 | −1.25 | −1.13 | 2010305A19Rik |
| 1424526_a_at | Lgx only | 630 | 568 | 533 | 439 | −1.11 | −1.18 | −1.43 | Tgds |
| 1424527_at | Lgx only | 1547 | 1612 | 1940 | 2049 | 1.04 | 1.25 | 1.32 | Ppp2r2d |
| 1424531_a_at | Lgx only | 1693 | 2053 | 2103 | 2408 | 1.21 | 1.24 | 1.42 | Tcea3 |
| 1424539_at | Res & Lgx | 4074 | 4359 | 3294 | 2993 | 1.07 | −1.24 | −1.36 | Ubl4 |
| 1424541_at | Res & Lgx | 5299 | 4888 | 3970 | 2986 | −1.08 | −1.33 | −1.77 | Tmem70 |
| 1424545_at | Res & Lgx | 4029 | 3665 | 2845 | 2585 | −1.10 | −1.42 | −1.56 | BC003965 |
| 1424553_at | Lgx only | 3262 | 3958 | 4239 | 4357 | 1.21 | 1.30 | 1.34 | Hhatl |
| 1424559_at | Lgx only | 301 | 268 | 241 | 157 | −1.13 | −1.25 | −1.92 | Rpap2 |
| 1424562_a_at | Lgx only | ##### | 112473 | 113888 | 167622 | 1.08 | 1.10 | 1.61 | Slc25a4 |
| 1424564_at | Lgx only | 1274 | 1360 | 1557 | 1708 | 1.07 | 1.22 | 1.34 | 2410001C21Rik |
| 1424572_a_at | Lgx only | 1146 | 1213 | 1654 | 1775 | 1.06 | 1.44 | 1.55 | H2afy |
| 1424585_at | Lgx only | 895 | 932 | 1128 | 1160 | 1.04 | 1.26 | 1.30 | Ranbp10 |
| 1424594_at | Lgx only | 3378 | 3499 | 3621 | 4257 | 1.04 | 1.07 | 1.26 | Lgals7 |
| 1424595_at | Lgx only | 1220 | 1320 | 1490 | 2039 | 1.08 | 1.22 | 1.67 | F11r |
| 1424598_at | Lgx only | 2389 | 1604 | 1838 | 1438 | −1.49 | −1.30 | −1.66 | Ddx6 |
| 1424614_at | Lgx only | 2262 | 2581 | 3033 | 2935 | 1.14 | 1.34 | 1.30 | Frag1 |
| 1424635_at | Lgx only | 40976 | 41854 | 46791 | 54473 | 1.02 | 1.14 | 1.33 | Eef1a1 |
| 1424639_a_at | Lgx only | 1837 | 2131 | 2120 | 2408 | 1.16 | 1.15 | 1.31 | Hmgcl |
| 1424642_at | Res & Lgx | 839 | 793 | 585 | 514 | −1.06 | −1.43 | −1.63 | Thoc1 |
| 1424643_at | Lgx only | 361 | 461 | 466 | 524 | 1.28 | 1.29 | 1.45 | Tcof1 |
| 1424644_at | Lgx only | 751 | 910 | 920 | 970 | 1.21 | 1.22 | 1.29 | Tbcc |
| 1424669_at | Res & Lgx | 1167 | 1216 | 852 | 883 | 1.04 | −1.37 | −1.32 | Zfyve21 |
| 1424682_at | Lgx only | 844 | 922 | 1107 | 1316 | 1.09 | 1.31 | 1.56 | Atpbd1c |
| 1424683_at | Lgx only | 15130 | 13625 | 13449 | 10806 | −1.11 | −1.12 | −1.40 | 1810015C04Rik |
| 1424686_at | Lgx only | 397 | 485 | 579 | 584 | 1.22 | 1.46 | 1.47 | Heatr6 |
| 1424700_at | Lgx only | 1348 | 1564 | 1695 | 1953 | 1.16 | 1.26 | 1.45 | Tmem38b |
| 1424715_at | All | 1674 | 1255 | 2440 | 2317 | −1.33 | 1.46 | 1.38 | Retsat |
| 1424720_at | Lgx only | 1275 | 1563 | 1533 | 1790 | 1.23 | 1.20 | 1.40 | Mgat4b |
| 1424727_at | Lgx only | 73 | 105 | 145 | 185 | 1.43 | 1.99 | 2.54 | Ccr5 |
| 1424728_at | CR & Lgx | 991 | 816 | 861 | 760 | −1.21 | −1.15 | −1.30 | BC011248 |
| 1424736_at | Res & Lgx | 18836 | 21343 | 24570 | 28063 | 1.13 | 1.30 | 1.49 | Eef2 |
| 1424744_at | Lgx only | 318 | 193 | 275 | 639 | −1.65 | −1.16 | 2.01 | Sds |
| 1424745_at | Lgx only | 533 | 595 | 714 | 779 | 1.11 | 1.34 | 1.46 | Agxt2l2 |
| 1424746_at | Res & Lgx | 5066 | 5452 | 6046 | 7004 | 1.08 | 1.19 | 1.38 | Kif1c |
| 1424749_at | Res & Lgx | 491 | 453 | 785 | 669 | −1.08 | 1.60 | 1.36 | Wdfy1 |
| 1424776_a_at | Lgx only | 1323 | 1302 | 1746 | 1740 | −1.02 | 1.32 | 1.32 | Slc25a28 |
| 1424777_at | Lgx only | 572 | 536 | 703 | 792 | −1.07 | 1.23 | 1.38 | Wdr21 |
| 1424790_at | Lgx only | 1894 | 2211 | 3370 | 4182 | 1.17 | 1.78 | 2.21 | Slc25a42 |
| 1424791_a_at | Lgx only | 2967 | 3682 | 3547 | 3765 | 1.24 | 1.20 | 1.27 | Bcam |
| 1424795_a_at | Res only | 62 | 70 | 170 | 154 | 1.13 | 2.74 | 2.48 | 1700001O22Rik |
| 1424819_a_at | Lgx only | 557 | 580 | 605 | 719 | 1.04 | 1.09 | 1.29 | Ric8 |
| 1424827_a_at | Lgx only | 5109 | 5113 | 4471 | 3838 | 1.00 | −1.14 | −1.33 | Csnk1a1 |
| 1424842_a_at | Lgx only | 554 | 516 | 684 | 759 | −1.07 | 1.24 | 1.37 | Arhgap24 |
| 1424850_at | Lgx only | 392 | 341 | 315 | 258 | −1.15 | −1.24 | −1.52 | Map3k1 |
| 1424873_at | Res & Lgx | 906 | 801 | 637 | 641 | −1.13 | −1.42 | −1.41 | Rnf2 |
| 1424878_at | CR & Lgx | 255 | 508 | 463 | 632 | 1.99 | 1.82 | 2.48 | Lrch4 |
| 1424898_at | CR only | 144 | 51 | 82 | 114 | −2.84 | −1.76 | −1.27 | Slc10a1 |
| 1424912_at | Lgx only | 417 | 508 | 500 | 569 | 1.22 | 1.20 | 1.36 | Slc25a17 |
| 1424918_at | Res & Lgx | 893 | 857 | 631 | 559 | −1.04 | −1.42 | −1.60 | Tbc1d19 |
| 1424929_a_at | Lgx only | 239 | 284 | 385 | 449 | 1.19 | 1.61 | 1.88 | Trim26 |
| 1424942_a_at | CR only | 55 | 147 | 154 | 55 | 2.69 | 2.81 | 1.00 | Myc |
| 1424954_a_at | Lgx only | 657 | 753 | 904 | 1030 | 1.15 | 1.38 | 1.57 | Pip5k1c |
| 1424956_at | Lgx only | 246 | 311 | 380 | 513 | 1.26 | 1.55 | 2.09 | Ahdc1 |
| 1424978_at | Res only | 23 | 79 | 87 | 88 | 3.44 | 3.76 | 3.82 | Odf4 |
| 1424988_at | Lgx only | 1652 | 1684 | 1806 | 2065 | 1.02 | 1.09 | 1.25 | Mylip |
| 1424990_at | Lgx only | 890 | 960 | 1037 | 1205 | 1.08 | 1.17 | 1.35 | Tmem142a |
| 1424996_at | Lgx only | 650 | 688 | 746 | 875 | 1.06 | 1.15 | 1.35 | Cflar |
| 1425024_at | CR only | 104 | 28 | 94 | 50 | −3.79 | −1.11 | −2.09 | E430018J23Rik |
| 1425057_at | Res only | 88 | 164 | 237 | 176 | 1.85 | 2.68 | 1.98 | Pbld |
| 1425079_at | CR only | 40 | 118 | 72 | 60 | 2.96 | 1.79 | 1.51 | Tm6sf2 |
| 1425114_at | Res & Lgx | 795 | 765 | 561 | 544 | −1.04 | −1.42 | −1.46 | Rbbp6 |
| 1425143_a_at | Lgx only | 25337 | 27260 | 28070 | 34606 | 1.08 | 1.11 | 1.37 | Ndufs1 |
| 1425158_at | Res & Lgx | 1320 | 1302 | 1751 | 1907 | −1.01 | 1.33 | 1.44 | Tbx20 |
| 1425164_a_at | Lgx only | 265 | 328 | 383 | 483 | 1.24 | 1.44 | 1.82 | Phkg1 |
| 1425189_a_at | Res & Lgx | 3583 | 3540 | 2889 | 2383 | −1.01 | −1.24 | −1.50 | Mrpl15 |
| 1425214_at | Lgx only | 324 | 237 | 207 | 133 | −1.37 | −1.57 | −2.44 | P2ry6 |
| 1425225_at | Lgx only | 94 | 142 | 190 | 249 | 1.51 | 2.02 | 2.64 | Fcgr4 |
| 1425228_a_at | Lgx only | 1536 | 1652 | 1255 | 1078 | 1.08 | −1.22 | −1.43 | Dguok |
| 1425243_at | Lgx only | 115 | 120 | 196 | 325 | 1.04 | 1.70 | 2.82 | Cd207 |
| 1425257_at | Lgx only | 7 | 19 | 41 | 16 | 2.89 | 6.34 | 2.39 | Acot5 |
| 1425270_at | Res & Lgx | 3017 | 2790 | 2111 | 1975 | −1.08 | −1.43 | −1.53 | Kif1b |
| 1425274_at | Res & Lgx | 4607 | 4976 | 3462 | 3212 | 1.08 | −1.33 | −1.43 | Asph |
| 1425314_at | Res only | 27 | 57 | 96 | 118 | 2.10 | 3.57 | 4.36 | Gpr98 |
| 1425332_at | Lgx only | 11194 | 12729 | 12529 | 16323 | 1.14 | 1.12 | 1.46 | Zfp106 |
| 1425333_at | Lgx only | 410 | 538 | 624 | 856 | 1.31 | 1.52 | 2.09 | Rab43 |
| 1425340_a_at | CR & Lgx | 1281 | 1481 | 1447 | 1573 | 1.16 | 1.13 | 1.23 | Ptpra |
| 1425341_at | Lgx only | 2691 | 3341 | 3786 | 4027 | 1.24 | 1.41 | 1.50 | Kcnk3 |
| 1425350_a_at | Res & Lgx | 106 | 147 | 186 | 212 | 1.38 | 1.75 | 2.00 | Myef2 |
| 1425455_a_at | Lgx only | 4248 | 4028 | 3479 | 3392 | −1.05 | −1.22 | −1.25 | Churc1 |
| 1425480_at | Res & Lgx | 2113 | 2091 | 1616 | 1298 | −1.01 | −1.31 | −1.63 | Cnot6l |
| 1425492_at | Res & Lgx | 2530 | 2180 | 1751 | 1792 | −1.16 | −1.44 | −1.41 | Bmpr1a |
| 1425519_a_at | Res & Lgx | 1769 | 1610 | 2963 | 2821 | −1.10 | 1.67 | 1.59 | Cd74 |
| 1425521_at | Lgx only | 337 | 279 | 205 | 188 | −1.21 | −1.64 | −1.79 | Paip1 |
| 1425558_at | Res only | 136 | 59 | 28 | 101 | −2.32 | −4.94 | −1.34 | Klc3 |
| 1425589_at | Lgx only | 148 | 111 | 123 | 38 | −1.34 | −1.21 | −3.88 | Hsd17b13 |
| 1425617_at | Lgx only | 243 | 296 | 355 | 486 | 1.22 | 1.46 | 1.99 | Dhx9 |
| 1425639_at | Lgx only | 209 | 211 | 342 | 342 | 1.01 | 1.63 | 1.64 | Centa2 |
| 1425646_at | Lgx only | 141 | 96 | 96 | 68 | −1.46 | −1.47 | −2.08 | BC016495 |
| 1425674_a_at | Lgx only | 2104 | 1965 | 1606 | 1495 | −1.07 | −1.31 | −1.41 | Ssu72 |
| 1425677_a_at | Lgx only | 11130 | 10594 | 9470 | 8378 | −1.05 | −1.18 | −1.33 | Ank1 |
| 1425682_a_at | Lgx only | 518 | 419 | 373 | 288 | −1.24 | −1.39 | −1.80 | Tprkb |
| 1425702_a_at | Lgx only | 574 | 571 | 734 | 946 | −1.01 | 1.28 | 1.65 | Enpp5 |
| 1425706_a_at | Lgx only | 371 | 337 | 391 | 239 | −1.10 | 1.05 | −1.55 | Ddb2 |
| 1425718_a_at | Lgx only | 33649 | 29835 | 25700 | 23101 | −1.13 | −1.31 | −1.46 | Ivns1abp |
| 1425742_a_at | Lgx only | 11487 | 11684 | 12973 | 13668 | 1.02 | 1.13 | 1.19 | Tsc22d1 |
| 1425753_a_at | Res only | 701 | 793 | 434 | 484 | 1.13 | −1.61 | −1.45 | Ung |
| 1425760_a_at | Lgx only | 378 | 450 | 540 | 627 | 1.19 | 1.43 | 1.66 | Pitpnm1 |
| 1425764_a_at | Lgx only | 2958 | 3344 | 3747 | 4049 | 1.13 | 1.27 | 1.37 | Bcat2 |
| 1425780_a_at | Lgx only | 1362 | 1035 | 1202 | 963 | −1.32 | −1.13 | −1.41 | Tmem167 |
| 1425792_a_at | Res & Lgx | 599 | 793 | 1003 | 995 | 1.32 | 1.67 | 1.66 | Rorc |
| 1425795_a_at | Res & Lgx | 2655 | 2581 | 1359 | 1207 | −1.03 | −1.95 | −2.20 | Map3k7 |
| 1425826_a_at | Lgx only | 6893 | 8508 | 10213 | 13158 | 1.23 | 1.48 | 1.91 | Sorbs1 |
| 1425894_at | CR only | 315 | 164 | 315 | 398 | −1.92 | 1.00 | 1.26 | Mrgprf |
| 1425895_a_at | Lgx only | 2855 | 3457 | 3880 | 4028 | 1.21 | 1.36 | 1.41 | ld1 |
| 1425904_at | Res only | 34 | 52 | 104 | 62 | 1.53 | 3.06 | 1.82 | Satb2 |
| 1425930_a_at | Lgx only | 698 | 638 | 680 | 563 | −1.09 | −1.03 | −1.24 | Mlx |
| 1425933_a_at | Res & Lgx | 1038 | 1091 | 859 | 854 | 1.05 | −1.21 | −1.21 | Nt5c2 |
| 1425940_a_at | Lgx only | 184 | 208 | 306 | 319 | 1.13 | 1.66 | 1.73 | Ssbp3 |
| 1425978_at | Res & Lgx | 1850 | 1652 | 1140 | 1026 | −1.12 | −1.62 | −1.80 | Myocd |
| 1425993_a_at | Res & Lgx | 1379 | 1079 | 897 | 848 | −1.28 | −1.54 | −1.63 | Hsp110 |
| 1426000_at | Lgx only | 164 | 196 | 167 | 317 | 1.19 | 1.02 | 1.93 | Oxtr |
| 1426016_a_at | Lgx only | 55 | 65 | 130 | 164 | 1.18 | 2.39 | 3.00 | Tro |
| 1426068_at | Lgx only | 935 | 868 | 767 | 692 | −1.08 | −1.22 | −1.35 | Slc7a4 |
| 1426089_a_at | Lgx only | 1823 | 1590 | 1565 | 1314 | −1.15 | −1.17 | −1.39 | BC003331 |
| 1426100_a_at | Lgx only | 218 | 225 | 310 | 338 | 1.03 | 1.42 | 1.55 | Tk2 |
| 1426114_at | Res & Lgx | 5491 | 5041 | 29756 | 25956 | −1.09 | 5.42 | 4.73 | Hnrpab |
| 1426118_a_at | Lgx only | 1454 | 1702 | 1714 | 1820 | 1.17 | 1.18 | 1.25 | Tomm40 |
| 1426179_a_at | Lgx only | 910 | 1026 | 1204 | 1351 | 1.13 | 1.32 | 1.48 | Twsg1 |
| 1426187_a_at | Lgx only | 1603 | 1568 | 1266 | 1056 | −1.02 | −1.27 | −1.52 | Hax1 |
| 1426195_a_at | Lgx only | 29737 | 32186 | 35405 | 39796 | 1.08 | 1.19 | 1.34 | Cst3 |
| 1426235_a_at | Res & Lgx | 2374 | 2866 | 5732 | 6147 | 1.21 | 2.41 | 2.59 | Glul |
| 1426241_a_at | Lgx only | 771 | 982 | 908 | 1062 | 1.27 | 1.18 | 1.38 | Scmh1 |
| 1426249_at | Res & Lgx | 1302 | 1459 | 1804 | 2108 | 1.12 | 1.39 | 1.62 | Adrbk1 |
| 1426254_at | Lgx only | 1276 | 1145 | 917 | 731 | −1.12 | −1.39 | −1.75 | Tm2d1 |
| 1426257_a_at | Res & Lgx | 1948 | 2192 | 2318 | 2595 | 1.13 | 1.19 | 1.33 | Sars |
| 1426263_at | Lgx only | 1203 | 1175 | 1108 | 911 | −1.02 | −1.09 | −1.32 | Cadm4 |
| 1426269_at | Res & Lgx | 684 | 560 | 450 | 371 | −1.22 | −1.52 | −1.85 | Sybl1 |
| 1426277_at | Lgx only | 386 | 336 | 445 | 519 | −1.15 | 1.15 | 1.34 | C730025P13Rik |
| 1426279_at | Lgx only | 770 | 726 | 591 | 516 | −1.06 | −1.30 | −1.49 | 5830415L20Rik |
| 1426285_at | Lgx only | 2453 | 2580 | 2755 | 3223 | 1.05 | 1.12 | 1.31 | Lama2 |
| 1426286_at | Lgx only | 335 | 275 | 211 | 119 | −1.22 | −1.59 | −2.81 | Noc3l |
| 1426297_at | Lgx only | 466 | 452 | 754 | 680 | −1.03 | 1.62 | 1.46 | Tcfe2a |
| 1426307_at | Res & Lgx | 1936 | 1817 | 1474 | 1311 | −1.07 | −1.31 | −1.48 | Cyb5r4 |
| 1426337_a_at | CR & Lgx | 32 | 84 | 49 | 110 | 2.64 | 1.55 | 3.45 | Tead4 |
| 1426344_at | Lgx only | 475 | 506 | 511 | 653 | 1.07 | 1.08 | 1.38 | Gle1l |
| 1426347_at | CR only | 783 | 635 | 743 | 686 | −1.23 | −1.05 | −1.14 | 2010321M09Rik |
| 1426353_at | Lgx only | 1157 | 1203 | 1210 | 1377 | 1.04 | 1.05 | 1.19 | Stat6 |
| 1426380_at | Lgx only | 4215 | 4149 | 4647 | 5122 | −1.02 | 1.10 | 1.22 | Eif4b |
| 1426386_at | Res & Lgx | 1464 | 1483 | 1190 | 993 | 1.01 | −1.23 | −1.47 | Rpl7l1 |
| 1426390_a_at | Lgx only | 22906 | 23329 | 19114 | 18319 | 1.02 | −1.20 | −1.25 | Arf1 |
| 1426398_at | Lgx only | 989 | 893 | 750 | 661 | −1.11 | −1.32 | −1.50 | Ube2w |
| 1426400_a_at | Res & Lgx | 9253 | 10079 | 10609 | 11708 | 1.09 | 1.15 | 1.27 | Capns1 |
| 1426406_at | Res & Lgx | 2553 | 2947 | 3706 | 4584 | 1.15 | 1.45 | 1.80 | Setd8 |
| 1426416_a_at | Res & Lgx | 1595 | 1370 | 1005 | 953 | −1.16 | −1.59 | −1.67 | Yipf4 |
| 1426423_at | Res & Lgx | 820 | 1004 | 1130 | 1251 | 1.22 | 1.38 | 1.53 | Shmt2 |
| 1426436_at | Lgx only | 760 | 764 | 675 | 568 | 1.00 | −1.13 | −1.34 | Tmem159 |
| 1426440_at | Lgx only | 2254 | 2820 | 2177 | 2947 | 1.25 | −1.04 | 1.31 | Dhrs7 |
| 1426444_at | Res only | 314 | 254 | 467 | 460 | −1.24 | 1.48 | 1.46 | Rhbdd2 |
| 1426445_at | Lgx only | 1978 | 2219 | 2502 | 2536 | 1.12 | 1.26 | 1.28 | Ctage5 |
| 1426446_at | Lgx only | 312 | 312 | 438 | 458 | 1.00 | 1.40 | 1.47 | 6430548M08Rik |
| 1426452_a_at | Lgx only | 202 | 256 | 129 | 77 | 1.26 | −1.57 | −2.64 | Rab30 |
| 1426455_at | Lgx only | 402 | 394 | 369 | 274 | −1.02 | −1.09 | −1.47 | Sdccag10 |
| 1426457_at | Lgx only | 9329 | 9212 | 7693 | 6876 | −1.01 | −1.21 | −1.36 | Slmap |
| 1426468_at | Res only | 1223 | 1032 | 910 | 976 | −1.18 | −1.34 | −1.25 | 0610037L13Rik |
| 1426477_at | Lgx only | 1012 | 893 | 905 | 676 | −1.13 | −1.12 | −1.50 | Rasa1 |
| 1426480_at | Lgx only | 2278 | 2147 | 2786 | 3030 | −1.06 | 1.22 | 1.33 | Sbds |
| 1426481_at | Res & Lgx | 1112 | 1028 | 1505 | 1624 | −1.08 | 1.35 | 1.46 | Klhl22 |
| 1426482_at | Lgx only | 3505 | 2977 | 2762 | 2410 | −1.18 | −1.27 | −1.45 | Prkrir |
| 1426495_at | Lgx only | 348 | 251 | 218 | 208 | −1.39 | −1.59 | −1.68 | 2410042D21Rik |
| 1426539_at | Lgx only | 295 | 365 | 390 | 465 | 1.24 | 1.32 | 1.58 | Usp11 |
| 1426567_a_at | Res only | 434 | 445 | 254 | 264 | 1.02 | −1.71 | −1.64 | Pqlc1 |
| 1426586_at | Lgx only | 17315 | 21329 | 19691 | 21472 | 1.23 | 1.14 | 1.24 | Slc25a11 |
| 1426596_a_at | All | 539 | 412 | 417 | 219 | −1.31 | −1.29 | −2.46 | Smn1 |
| 1426607_at | Lgx only | 304 | 267 | 198 | 175 | −1.14 | −1.54 | −1.73 | EG633640 |
| 1426613_a_at | Lgx only | 1480 | 1201 | 1246 | 838 | −1.23 | −1.19 | −1.77 | Snrpb2 |
| 1426614_at | Lgx only | 741 | 922 | 870 | 1015 | 1.25 | 1.18 | 1.37 | Prkcbp1 |
| 1426624_a_at | Res & Lgx | 2908 | 3005 | 4008 | 4108 | 1.03 | 1.38 | 1.41 | Ypel3 |
| 1426629_at | Lgx only | 450 | 528 | 534 | 613 | 1.17 | 1.19 | 1.36 | Dhx8 |
| 1426643_at | Lgx only | 959 | 1059 | 1176 | 1265 | 1.10 | 1.23 | 1.32 | Elp3 |
| 1426646_at | Lgx only | 3289 | 3191 | 2689 | 2507 | −1.03 | −1.22 | −1.31 | 9130011J15Rik |
| 1426648_at | Lgx only | 3467 | 4510 | 4856 | 5396 | 1.30 | 1.40 | 1.56 | Mapkapk2 |
| 1426670_at | Res & Lgx | 701 | 789 | 1130 | 1113 | 1.13 | 1.61 | 1.59 | Agrn |
| 1426671_a_at | Lgx only | 1307 | 1327 | 1062 | 766 | 1.02 | −1.23 | −1.71 | Rbm39 |
| 1426675_at | Lgx only | 1376 | 1180 | 884 | 626 | −1.17 | −1.56 | −2.20 | Tomm70a |
| 1426681_at | Lgx only | 284 | 309 | 369 | 440 | 1.09 | 1.30 | 1.55 | Unk |
| 1426682_at | Lgx only | 1328 | 1222 | 1113 | 852 | −1.09 | −1.19 | −1.56 | LOC100046343 |
| 1426688_at | Lgx only | 30012 | 35064 | 33818 | 42859 | 1.17 | 1.13 | 1.43 | Sdha |
| 1426690_a_at | Res & Lgx | 1211 | 1395 | 1784 | 2490 | 1.15 | 1.47 | 2.06 | Srebf1 |
| 1426691_at | Lgx only | 818 | 960 | 944 | 1189 | 1.17 | 1.15 | 1.45 | Tjap1 |
| 1426700_a_at | Lgx only | 310 | 327 | 417 | 406 | 1.06 | 1.35 | 1.31 | Usp52 |
| 1426717_at | Res & Lgx | 1834 | 1766 | 1219 | 1092 | −1.04 | −1.50 | −1.68 | Nipa2 |
| 1426718_at | Res & Lgx | 972 | 829 | 713 | 726 | −1.17 | −1.36 | −1.34 | Skiv2l2 |
| 1426731_at | Lgx only | 15714 | 18402 | 19327 | 23750 | 1.17 | 1.23 | 1.51 | Des |
| 1426741_a_at | Lgx only | 1254 | 1098 | 1044 | 860 | −1.14 | −1.20 | −1.46 | Fastkd2 |
| 1426743_at | Lgx only | 3106 | 3174 | 2664 | 2485 | 1.02 | −1.17 | −1.25 | Appl2 |
| 1426752_at | Res only | 1165 | 945 | 826 | 990 | −1.23 | −1.41 | −1.18 | Phf17 |
| 1426760_at | Res & Lgx | 1194 | 1065 | 823 | 781 | −1.12 | −1.45 | −1.53 | Ipo8 |
| 1426773_at | Lgx only | 6830 | 8123 | 9064 | 9354 | 1.19 | 1.33 | 1.37 | Mfn1 |
| 1426774_at | Res & Lgx | 607 | 595 | 485 | 347 | −1.02 | −1.25 | −1.75 | Parp12 |
| 1426794_at | Lgx only | 934 | 1249 | 1207 | 1600 | 1.34 | 1.29 | 1.71 | Ptprs |
| 1426799_at | Lgx only | 826 | 683 | 610 | 489 | −1.21 | −1.35 | −1.69 | Rab8b |
| 1426819_at | Lgx only | 9939 | 6798 | 6585 | 3448 | −1.46 | −1.51 | −2.88 | LOC100048439 |
| 1426820_at | Lgx only | 5084 | 5617 | 5406 | 6361 | 1.10 | 1.06 | 1.25 | 2610507B11Rik |
| 1426830_a_at | Lgx only | 3633 | 3395 | 3097 | 2585 | −1.07 | −1.17 | −1.41 | Ahcyl1 |
| 1426833_at | Lgx only | 846 | 870 | 1103 | 1089 | 1.03 | 1.30 | 1.29 | Eif4g3 |
| 1426854_a_at | Lgx only | 4159 | 4032 | 3462 | 3261 | −1.03 | −1.20 | −1.28 | Set |
| 1426857_a_at | Lgx only | 5854 | 4850 | 5610 | 4740 | −1.21 | −1.04 | −1.23 | Hsdl2 |
| 1426866_at | Lgx only | 571 | 766 | 674 | 802 | 1.34 | 1.18 | 1.41 | Chst14 |
| 1426886_at | Res & Lgx | 872 | 844 | 723 | 633 | −1.03 | −1.21 | −1.38 | Cln5 |
| 1426895_at | Res & Lgx | 897 | 707 | 604 | 450 | −1.27 | −1.48 | −1.99 | Zfp191 |
| 1426898_at | Res only | 391 | 401 | 533 | 528 | 1.03 | 1.36 | 1.35 | Map3k7ip1 |
| 1426900_at | Lgx only | 986 | 807 | 644 | 600 | −1.22 | −1.53 | −1.64 | Jmjd1c |
| 1426948_at | Res & Lgx | 3583 | 4241 | 4782 | 5859 | 1.18 | 1.33 | 1.64 | Tpr |
| 1426952_at | Lgx only | 524 | 503 | 478 | 259 | −1.04 | −1.10 | −2.02 | Arhgap18 |
| 1426964_at | Lgx only | 1856 | 1813 | 1314 | 1265 | −1.02 | −1.41 | −1.47 | 3110003A17Rik |
| 1426965_at | Lgx only | 402 | 405 | 499 | 568 | 1.01 | 1.24 | 1.41 | Rap2a |
| 1426969_at | Res & Lgx | 551 | 378 | 376 | 287 | −1.46 | −1.46 | −1.92 | Trim23 |
| 1426976_at | Lgx only | 3711 | 3721 | 4100 | 4747 | 1.00 | 1.10 | 1.28 | Usp47 |
| 1426979_at | Lgx only | 993 | 1192 | 1354 | 1332 | 1.20 | 1.36 | 1.34 | Mlxip |
| 1426982_at | Res & Lgx | 535 | 607 | 880 | 853 | 1.13 | 1.65 | 1.59 | Flywch1 |
| 1426984_at | Res & Lgx | 291 | 540 | 554 | 854 | 1.86 | 1.91 | 2.94 | 2310067B10Rik |
| 1426992_at | Res & Lgx | 1745 | 1757 | 1076 | 1052 | 1.01 | −1.62 | −1.66 | Xpr1 |
| 1427028_at | Lgx only | 556 | 635 | 615 | 891 | 1.14 | 1.11 | 1.60 | Lgr6 |
| 1427039_at | Lgx only | 1834 | 2329 | 2407 | 2983 | 1.27 | 1.31 | 1.63 | Epn1 |
| 1427040_at | Lgx only | 1252 | 987 | 952 | 855 | −1.27 | −1.31 | −1.46 | Mdfic |
| 1427045_at | Lgx only | 778 | 817 | 1248 | 1424 | 1.05 | 1.60 | 1.83 | Synpo |
| 1427051_at | Res & Lgx | 454 | 548 | 673 | 770 | 1.21 | 1.48 | 1.70 | Tnks1bp1 |
| 1427058_at | Res only | 8484 | 8530 | 6619 | 7155 | 1.01 | −1.28 | −1.19 | Eif4a1 |
| 1427073_at | Res only | 1956 | 2206 | 1536 | 1648 | 1.13 | −1.27 | −1.19 | Lace1 |
| 1427084_a_at | Lgx only | 686 | 606 | 508 | 463 | −1.13 | −1.35 | −1.48 | Map4k5 |
| 1427099_at | Res only | 320 | 486 | 646 | 461 | 1.52 | 2.02 | 1.44 | Maz |
| 1427117_at | Lgx only | 619 | 616 | 790 | 979 | −1.01 | 1.28 | 1.58 | Mtmr3 |
| 1427120_at | Lgx only | 835 | 688 | 573 | 468 | −1.21 | −1.46 | −1.78 | Zfp26 |
| 1427129_a_at | Lgx only | 1545 | 1252 | 1132 | 930 | −1.23 | −1.36 | −1.66 | Hnrpr |
| 1427132_at | Lgx only | 348 | 463 | 524 | 564 | 1.33 | 1.51 | 1.62 | Sbf2 |
| 1427139_at | Lgx only | 795 | 940 | 925 | 1054 | 1.18 | 1.16 | 1.33 | Adamts10 |
| 1427144_at | Res & Lgx | 1131 | 1047 | 680 | 637 | −1.08 | −1.66 | −1.78 | Hnrpll |
| 1427146_at | Res & Lgx | 673 | 839 | 1204 | 1613 | 1.25 | 1.79 | 2.40 | AI790298 |
| 1427165_at | Res & Lgx | 1308 | 1233 | 814 | 792 | −1.06 | −1.61 | −1.65 | Il13ra1 |
| 1427166_a_at | Lgx only | 1401 | 1747 | 2035 | 2399 | 1.25 | 1.45 | 1.71 | Spg7 |
| 1427170_at | All | 74 | 25 | 24 | 13 | −2.94 | −3.10 | −5.66 | Psma8 |
| 1427197_at | Lgx only | 496 | 530 | 403 | 303 | 1.07 | −1.23 | −1.64 | Atr |
| 1427201_at | Lgx only | 551 | 457 | 565 | 769 | −1.20 | 1.03 | 1.40 | Mustn1 |
| 1427228_at | Lgx only | 2099 | 2379 | 3387 | 3964 | 1.13 | 1.61 | 1.89 | Palld |
| 1427239_at | CR & Lgx | 552 | 808 | 637 | 680 | 1.46 | 1.15 | 1.23 | Ift122 |
| 1427240_at | Lgx only | 815 | 1013 | 1033 | 1520 | 1.24 | 1.27 | 1.86 | Dock6 |
| 1427241_at | Lgx only | 610 | 547 | 387 | 384 | −1.12 | −1.58 | −1.59 | Papolg |
| 1427260_a_at | Res & Lgx | 2978 | 2624 | 2355 | 1823 | −1.13 | −1.26 | −1.63 | Tpm3 |
| 1427266_at | All | 1181 | 848 | 784 | 732 | −1.39 | −1.51 | −1.61 | Pbrm1 |
| 1427296_at | Res & Lgx | 4758 | 5054 | 3684 | 3677 | 1.06 | −1.29 | −1.29 | BC010304 |
| 1427312_at | Lgx only | 12654 | 14384 | 15939 | 18697 | 1.14 | 1.26 | 1.48 | Cmya5 |
| 1427314_at | Res & Lgx | 2995 | 2439 | 2188 | 1689 | −1.23 | −1.37 | −1.77 | Tmed7 |
| 1427319_at | CR & Lgx | 694 | 468 | 551 | 427 | −1.48 | −1.26 | −1.63 | A230046K03Rik |
| 1427342_at | Res & Lgx | 802 | 683 | 597 | 499 | −1.18 | −1.35 | −1.61 | Fastkd1 |
| 1427395_a_at | Lgx only | 74 | 37 | 30 | 33 | −2.00 | −2.51 | −2.27 | Aldh1a3 |
| 1427418_a_at | Lgx only | 1864 | 1555 | 1250 | 796 | −1.20 | −1.49 | −2.34 | Hif1a |
| 1427432_a_at | Lgx only | 2372 | 2225 | 1569 | 1292 | −1.07 | −1.51 | −1.84 | Sfrs10 |
| 1427447_a_at | Res & Lgx | 1080 | 1415 | 1561 | 1854 | 1.31 | 1.44 | 1.72 | Triobp |
| 1427490_at | Lgx only | 356 | 348 | 274 | 236 | −1.02 | −1.30 | −1.51 | Abcb7 |
| 1427529_at | Res only | 151 | 90 | 67 | 82 | −1.68 | −2.25 | −1.84 | Fzd9 |
| 1427555_at | Lgx only | 35 | 64 | 76 | 96 | 1.85 | 2.18 | 2.76 | Mll2 |
| 1427557_at | Lgx only | 180 | 201 | 256 | 281 | 1.11 | 1.42 | 1.56 | Alg12 |
| 1427604_a_at | Lgx only | 871 | 1023 | 1176 | 1356 | 1.17 | 1.35 | 1.56 | Atp9a |
| 1427661_a_at | Res & Lgx | 382 | 474 | 594 | 597 | 1.24 | 1.56 | 1.56 | Tssc4 |
| 1427689_a_at | Lgx only | 1261 | 1266 | 1376 | 1652 | 1.00 | 1.09 | 1.31 | Tnip1 |
| 1427720_a_at | Lgx only | 2747 | 2851 | 3040 | 3415 | 1.04 | 1.11 | 1.24 | Rrp1 |
| 1427728_at | CR only | 34 | 97 | 56 | 57 | 2.88 | 1.65 | 1.68 | Chrng |
| 1427735_a_at | CR & Res | 8359 | 12700 | 12493 | 12108 | 1.52 | 1.49 | 1.45 | Acta1 |
| 1427873_at | Lgx only | 17 | 54 | 56 | 110 | 3.19 | 3.31 | 6.50 | Defcr15 |
| 1427874_at | Lgx only | 1399 | 1574 | 1815 | 1899 | 1.13 | 1.30 | 1.36 | Zfp313 |
| 1427876_at | Lgx only | 1630 | 1676 | 1431 | 1226 | 1.03 | −1.14 | −1.33 | Zc3h15 |
| 1427886_at | Lgx only | 969 | 755 | 581 | 374 | −1.28 | −1.67 | −2.59 | Pom121 |
| 1427888_a_at | CR & Lgx | 5357 | 6838 | 6596 | 7987 | 1.28 | 1.23 | 1.49 | Spna2 |
| 1427894_at | Lgx only | 472 | 396 | 538 | 682 | −1.19 | 1.14 | 1.44 | Vasn |
| 1427898_at | Lgx only | 822 | 714 | 616 | 515 | −1.15 | −1.33 | −1.60 | Rnf6 |
| 1427901_at | Lgx only | 3381 | 3126 | 2782 | 2128 | −1.08 | −1.22 | −1.59 | Mrps18c |
| 1427903_at | Res & Lgx | 5849 | 5774 | 4732 | 4224 | −1.01 | −1.24 | −1.38 | Phpt1 |
| 1427913_at | Lgx only | 2402 | 2230 | 1819 | 1452 | −1.08 | −1.32 | −1.65 | Rwdd1 |
| 1427918_a_at | Lgx only | 7544 | 7921 | 8074 | 9445 | 1.05 | 1.07 | 1.25 | Rhoq |
| 1427929_a_at | Lgx only | 610 | 746 | 507 | 763 | 1.22 | −1.20 | 1.25 | Pdxk |
| 1427943_at | Lgx only | 6234 | 5441 | 5157 | 3929 | −1.15 | −1.21 | −1.59 | Acyp2 |
| 1427947_at | CR only | 493 | 623 | 501 | 547 | 1.26 | 1.02 | 1.11 | BC028440 |
| 1427955_a_at | Lgx only | 6984 | 7396 | 5948 | 4819 | 1.06 | −1.17 | −1.45 | Deb1 |
| 1427971_at | Lgx only | 957 | 802 | 781 | 608 | −1.19 | −1.22 | −1.57 | Cdc73 |
| 1427983_at | Lgx only | 514 | 395 | 447 | 362 | −1.30 | −1.15 | −1.42 | Suhw3 |
| 1427990_at | Lgx only | 355 | 290 | 266 | 214 | −1.22 | −1.34 | −1.66 | Usp45 |
| 1427996_at | Lgx only | 850 | 752 | 733 | 605 | −1.13 | −1.16 | −1.41 | BC028528 |
| 1427997_at | Res & Lgx | 3311 | 2612 | 2104 | 1880 | −1.27 | −1.57 | −1.76 | 1110007M04Rik |
| 1428029_a_at | Res & Lgx | 4750 | 5138 | 6149 | 6341 | 1.08 | 1.29 | 1.33 | H2afv |
| 1428061_at | Res & Lgx | 945 | 762 | 578 | 474 | −1.24 | −1.64 | −1.99 | Hat1 |
| 1428064_at | Lgx only | 545 | 548 | 561 | 729 | 1.01 | 1.03 | 1.34 | Centd2 |
| 1428071_at | Lgx only | 1332 | 1392 | 1595 | 1813 | 1.04 | 1.20 | 1.36 | 1110038D17Rik |
| 1428083_at | CR & Lgx | 11028 | 17747 | 12934 | 18091 | 1.61 | 1.17 | 1.64 | 2310043N10Rik |
| 1428084_at | Lgx only | 1028 | 928 | 751 | 646 | −1.11 | −1.37 | −1.59 | Krr1 |
| 1428100_at | Lgx only | 710 | 679 | 484 | 450 | −1.05 | −1.47 | −1.58 | Sfrs1 |
| 1428103_at | CR & Lgx | 1072 | 830 | 813 | 700 | −1.29 | −1.32 | −1.53 | Adam10 |
| 1428124_at | Lgx only | 422 | 302 | 322 | 263 | −1.40 | −1.31 | −1.60 | Gtf2e1 |
| 1428128_at | Res & Lgx | 1502 | 1659 | 2529 | 2521 | 1.10 | 1.68 | 1.68 | 4921506J03Rik |
| 1428134_at | Lgx only | 16660 | 18474 | 18163 | 20707 | 1.11 | 1.09 | 1.24 | Coq9 |
| 1428143_a_at | Res & Lgx | 6354 | 8097 | 11246 | 12003 | 1.27 | 1.77 | 1.89 | Pnpla2 |
| 1428158_at | Res & Lgx | 1535 | 1812 | 2043 | 2286 | 1.18 | 1.33 | 1.49 | Akt1s1 |
| 1428160_at | Lgx only | 2716 | 2030 | 1757 | 1025 | −1.34 | −1.55 | −2.65 | Ndufab1 |
| 1428173_at | Lgx only | 437 | 537 | 595 | 762 | 1.23 | 1.36 | 1.74 | Eml2 |
| 1428179_at | Lgx only | 43162 | 47906 | 48747 | 57849 | 1.11 | 1.13 | 1.34 | Ndufv2 |
| 1428182_at | Lgx only | 840 | 966 | 980 | 1096 | 1.15 | 1.17 | 1.30 | Prpsap1 |
| 1428198_at | Lgx only | 360 | 322 | 266 | 217 | −1.12 | −1.36 | −1.66 | Adal |
| 1428201_at | CR only | 2131 | 2525 | 2452 | 2729 | 1.19 | 1.15 | 1.28 | 2310036O22Rik |
| 1428206_at | Res only | 104 | 76 | 36 | 108 | −1.38 | −2.90 | 1.04 | Ccdc69 |
| 1428213_at | Res & Lgx | 2496 | 2367 | 1629 | 1372 | −1.05 | −1.53 | −1.82 | Nsmce4a |
| 1428230_at | CR only | 963 | 819 | 985 | 833 | −1.18 | 1.02 | −1.16 | Prkcn |
| 1428235_at | Lgx only | 19980 | 23234 | 20415 | 24655 | 1.16 | 1.02 | 1.23 | Sdhd |
| 1428236_at | Lgx only | 556 | 408 | 377 | 306 | −1.36 | −1.48 | −1.81 | Acbd5 |
| 1428244_at | Lgx only | 821 | 1001 | 1063 | 1344 | 1.22 | 1.29 | 1.64 | Larp1 |
| 1428250_at | Lgx only | 150 | 122 | 155 | 259 | −1.23 | 1.03 | 1.73 | Gper |
| 1428251_at | Lgx only | 464 | 397 | 316 | 330 | −1.17 | −1.47 | −1.40 | Smchd1 |
| 1428253_at | Res & Lgx | 1966 | 1803 | 1418 | 1374 | −1.09 | −1.39 | −1.43 | Chmp2b |
| 1428256_at | Res & Lgx | 2199 | 1822 | 1588 | 1576 | −1.21 | −1.39 | −1.40 | 2310047H23Rik |
| 1428260_at | Res & Lgx | 200 | 180 | 123 | 107 | −1.11 | −1.62 | −1.88 | Spg3a |
| 1428277_at | Lgx only | 2436 | 2410 | 1959 | 1529 | −1.01 | −1.24 | −1.59 | Otud6b |
| 1428282_at | Lgx only | 1073 | 922 | 1060 | 757 | −1.16 | −1.01 | −1.42 | Tbce |
| 1428299_at | Res & Lgx | 1060 | 1010 | 682 | 653 | −1.05 | −1.55 | −1.62 | Dyrk1a |
| 1428307_at | Lgx only | 1160 | 1032 | 931 | 745 | −1.12 | −1.24 | −1.56 | Zdhhc13 |
| 1428316_a_at | Res & Lgx | 6077 | 4954 | 4136 | 3051 | −1.23 | −1.47 | −1.99 | Fundc2 |
| 1428332_at | CR & Lgx | 2444 | 3279 | 2758 | 3376 | 1.34 | 1.13 | 1.38 | Pik3ip1 |
| 1428335_a_at | Lgx only | 810 | 785 | 691 | 580 | −1.03 | −1.17 | −1.40 | Scfd1 |
| 1428357_at | Res only | 190 | 206 | 336 | 219 | 1.08 | 1.77 | 1.15 | 2610019F03Rik |
| 1428365_a_at | Lgx only | 2043 | 2396 | 2896 | 3413 | 1.17 | 1.42 | 1.67 | Lonp1 |
| 1428366_at | All | 798 | 646 | 577 | 485 | −1.23 | −1.38 | −1.65 | 1600027N09Rik |
| 1428370_at | CR only | 128 | 228 | 179 | 125 | 1.78 | 1.40 | −1.03 | 1500011B03Rik |
| 1428380_at | Res & Lgx | 2352 | 2151 | 1639 | 1394 | −1.09 | −1.44 | −1.69 | 0610007C21Rik |
| 1428382_at | Lgx only | 964 | 1129 | 1473 | 1531 | 1.17 | 1.53 | 1.59 | Smarcc2 |
| 1428391_at | Lgx only | 267 | 295 | 348 | 432 | 1.10 | 1.30 | 1.62 | Rab3il1 |
| 1428395_at | Lgx only | 487 | 508 | 647 | 727 | 1.04 | 1.33 | 1.49 | Smurf1 |
| 1428405_at | Lgx only | 9641 | 10670 | 11324 | 12596 | 1.11 | 1.17 | 1.31 | Hcfc1r1 |
| 1428412_at | Lgx only | 2036 | 2091 | 2325 | 2629 | 1.03 | 1.14 | 1.29 | Tm9sf3 |
| 1428421_a_at | Lgx only | 2913 | 2410 | 2394 | 2019 | −1.21 | −1.22 | −1.44 | Glod4 |
| 1428423_at | Lgx only | 1714 | 1643 | 1657 | 1494 | −1.04 | −1.03 | −1.15 | Pcgf3 |
| 1428427_at | CR only | 92 | 36 | 39 | 101 | −2.53 | −2.37 | 1.10 | Fbxl2 |
| 1428431_at | Res & Lgx | 1390 | 1453 | 1755 | 1963 | 1.04 | 1.26 | 1.41 | 2310047A01Rik |
| 1428436_at | Res & Lgx | 1165 | 1047 | 815 | 839 | −1.11 | −1.43 | −1.39 | Lsm14a |
| 1428440_at | Lgx only | 8819 | 10649 | 10766 | 12943 | 1.21 | 1.22 | 1.47 | Slc25a12 |
| 1428441_at | Lgx only | 2992 | 2477 | 1975 | 1655 | −1.21 | −1.51 | −1.81 | Cisd2 |
| 1428443_a_at | Lgx only | 732 | 902 | 816 | 1006 | 1.23 | 1.11 | 1.37 | Rap1gap |
| 1428465_at | Lgx only | 6696 | 7010 | 5690 | 4717 | 1.05 | −1.18 | −1.42 | Tmem147 |
| 1428468_at | All | 950 | 768 | 805 | 688 | −1.24 | −1.18 | −1.38 | 3110043O21Rik |
| 1428476_a_at | Lgx only | 441 | 575 | 672 | 824 | 1.30 | 1.52 | 1.87 | Elac2 |
| 1428494_a_at | Lgx only | 3176 | 3065 | 2853 | 2679 | −1.04 | −1.11 | −1.19 | Polr2i |
| 1428495_at | Res & Lgx | 2251 | 1796 | 1512 | 1278 | −1.25 | −1.49 | −1.76 | 2410003K15Rik |
| 1428503_a_at | Lgx only | 2042 | 1913 | 1596 | 1490 | −1.07 | −1.28 | −1.37 | Nkiras1 |
| 1428505_at | Lgx only | 4503 | 4374 | 3908 | 2889 | −1.03 | −1.15 | −1.56 | Ccdc90b |
| 1428507_at | Lgx only | 1620 | 1610 | 1433 | 1331 | −1.01 | −1.13 | −1.22 | Hdhd2 |
| 1428508_at | Lgx only | 415 | 458 | 547 | 572 | 1.10 | 1.32 | 1.38 | Tbc1d2b |
| 1428510_at | Lgx only | 331 | 473 | 485 | 525 | 1.43 | 1.46 | 1.58 | Lphn1 |
| 1428515_at | Lgx only | 845 | 790 | 736 | 569 | −1.07 | −1.15 | −1.48 | 2410012H22Rik |
| 1428519_at | Lgx only | 3006 | 3200 | 2041 | 1928 | 1.06 | −1.47 | −1.56 | 2610528E23Rik |
| 1428540_at | Lgx only | 162 | 219 | 253 | 370 | 1.35 | 1.56 | 2.28 | 3321401G04Rik |
| 1428544_at | Res & Lgx | 1458 | 1331 | 1205 | 1043 | −1.10 | −1.21 | −1.40 | 0610007L01Rik |
| 1428549_at | Lgx only | 404 | 587 | 708 | 973 | 1.45 | 1.75 | 2.41 | Ccdc3 |
| 1428551_at | All | 518 | 361 | 300 | 223 | −1.44 | −1.72 | −2.32 | Trmt11 |
| 1428552_at | Res & Lgx | 2497 | 2020 | 1544 | 1257 | −1.24 | −1.62 | −1.99 | 2610001J05Rik |
| 1428554_a_at | Lgx only | 2768 | 2661 | 2626 | 2233 | −1.04 | −1.05 | −1.24 | 1810035L17Rik |
| 1428564_at | Lgx only | 417 | 459 | 555 | 738 | 1.10 | 1.33 | 1.77 | Zfp579 |
| 1428580_at | Lgx only | 1441 | 1418 | 1326 | 1067 | −1.02 | −1.09 | −1.35 | Blvra |
| 1428585_at | Lgx only | 720 | 831 | 840 | 1309 | 1.15 | 1.17 | 1.82 | Actn1 |
| 1428587_at | Res & Lgx | 2171 | 2060 | 1437 | 1374 | −1.05 | −1.51 | −1.58 | Tmem41b |
| 1428589_at | Lgx only | 7082 | 6392 | 5436 | 4990 | −1.11 | −1.30 | −1.42 | Mrpl41 |
| 1428593_at | Res only | 436 | 336 | 287 | 244 | −1.30 | −1.52 | −1.79 | 1700029F09Rik |
| 1428594_at | Lgx only | 1864 | 1998 | 1599 | 1255 | 1.07 | −1.17 | −1.48 | Garnl1 |
| 1428598_at | All | 663 | 946 | 941 | 976 | 1.43 | 1.42 | 1.47 | Tbc1d7 |
| 1428613_at | Res only | 548 | 583 | 786 | 754 | 1.06 | 1.43 | 1.38 | Ldhd |
| 1428615_at | CR only | 1077 | 892 | 1088 | 1153 | −1.21 | 1.01 | 1.07 | P2ry5 |
| 1428617_at | Res & Lgx | 1207 | 1018 | 969 | 859 | −1.19 | −1.25 | −1.41 | Hcfc2 |
| 1428626_at | Lgx only | 750 | 726 | 685 | 550 | −1.03 | −1.09 | −1.36 | Lysmd2 |
| 1428651_at | CR & Lgx | 5284 | 4538 | 5141 | 4573 | −1.16 | −1.03 | −1.16 | Klhl24 |
| 1428652_at | Res & Lgx | 341 | 312 | 224 | 199 | −1.09 | −1.53 | −1.72 | 0610010F05Rik |
| 1428668_at | Lgx only | 921 | 942 | 1096 | 1235 | 1.02 | 1.19 | 1.34 | Acbd3 |
| 1428682_at | CR only | 181 | 79 | 159 | 142 | −2.29 | −1.14 | −1.27 | Zc3h6 |
| 1428691_at | Lgx only | 1373 | 1505 | 1521 | 1856 | 1.10 | 1.11 | 1.35 | Chd2 |
| 1428697_at | Lgx only | 1082 | 1076 | 1176 | 1398 | −1.01 | 1.09 | 1.29 | Dpp8 |
| 1428707_at | Lgx only | 2825 | 3531 | 3687 | 4608 | 1.25 | 1.31 | 1.63 | Ptms |
| 1428715_at | Res & Lgx | 561 | 444 | 367 | 278 | −1.26 | −1.53 | −2.02 | 2810423A18Rik |
| 1428722_at | Lgx only | 59642 | 62216 | 62442 | 77221 | 1.04 | 1.05 | 1.29 | Ckmt2 |
| 1428723_at | Lgx only | 404 | 425 | 515 | 657 | 1.05 | 1.27 | 1.62 | 2310047M10Rik |
| 1428728_at | CR only | 232 | 387 | 266 | 290 | 1.67 | 1.15 | 1.25 | Ddx51 |
| 1428731_at | Lgx only | 946 | 1052 | 960 | 1147 | 1.11 | 1.01 | 1.21 | Usp54 |
| 1428748_at | Lgx only | 426 | 369 | 313 | 221 | −1.16 | −1.36 | −1.93 | 5830428H23Rik |
| 1428749_at | Res & Lgx | 631 | 573 | 372 | 387 | −1.10 | −1.69 | −1.63 | Dmxl2 |
| 1428758_at | CR & Res | 2386 | 1678 | 1803 | 1967 | −1.42 | −1.32 | −1.21 | Tmem86a |
| 1428767_at | Lgx only | 347 | 345 | 449 | 482 | −1.00 | 1.30 | 1.39 | Gsdmdc1 |
| 1428769_at | Res only | 568 | 531 | 411 | 491 | −1.07 | −1.38 | −1.16 | Tatdn3 |
| 1428782_a_at | Res & Lgx | 40904 | 44865 | 48798 | 54806 | 1.10 | 1.19 | 1.34 | Uqcrc1 |
| 1428786_at | Res only | 261 | 274 | 406 | 363 | 1.05 | 1.55 | 1.39 | Nckap1l |
| 1428789_at | CR only | 425 | 281 | 383 | 383 | −1.51 | −1.11 | −1.11 | Ralgps2 |
| 1428791_at | Lgx only | 2024 | 2245 | 2339 | 2646 | 1.11 | 1.16 | 1.31 | Ube2h |
| 1428807_at | Lgx only | 24 | 19 | 27 | 66 | −1.30 | 1.10 | 2.72 | Pabpc3 |
| 1428810_at | Lgx only | 1298 | 1336 | 1428 | 1643 | 1.03 | 1.10 | 1.27 | 2700097O09Rik |
| 1428812_at | Res only | 600 | 608 | 802 | 795 | 1.01 | 1.34 | 1.33 | 1700040L02Rik |
| 1428829_at | CR & Lgx | 827 | 624 | 677 | 551 | −1.32 | −1.22 | −1.50 | 6820401H01Rik |
| 1428831_at | Lgx only | 626 | 553 | 446 | 354 | −1.13 | −1.40 | −1.77 | 6230429P13Rik |
| 1428835_at | Lgx only | 1354 | 1406 | 2031 | 2409 | 1.04 | 1.50 | 1.78 | Myh14 |
| 1428845_at | Res & Lgx | 2313 | 2038 | 1791 | 1813 | −1.14 | −1.29 | −1.28 | Bclaf1 |
| 1428848_a_at | Lgx only | 2317 | 2709 | 2961 | 3010 | 1.17 | 1.28 | 1.30 | Macf1 |
| 1428884_at | Lgx only | 1347 | 1481 | 1302 | 1707 | 1.10 | −1.03 | 1.27 | Tmem57 |
| 1428890_at | Lgx only | 1103 | 993 | 869 | 687 | −1.11 | −1.27 | −1.61 | Fem1c |
| 1428897_at | Res & Lgx | 1192 | 1158 | 601 | 686 | −1.03 | −1.98 | −1.74 | 2610029I01Rik |
| 1428899_at | Res & Lgx | 6189 | 6125 | 4734 | 4774 | −1.01 | −1.31 | −1.30 | Tmem182 |
| 1428914_at | Res & Lgx | 1933 | 2302 | 2457 | 2903 | 1.19 | 1.27 | 1.50 | 2310014D11Rik |
| 1428919_at | Res & Lgx | 753 | 645 | 488 | 360 | −1.17 | −1.54 | −2.09 | Fgfr1op |
| 1428926_at | Lgx only | 1791 | 2110 | 2525 | 2734 | 1.18 | 1.41 | 1.53 | 1110003O08Rik |
| 1428945_at | Res only | 511 | 424 | 286 | 365 | −1.21 | −1.79 | −1.40 | Ube1l2 |
| 1428949_at | Lgx only | 1079 | 913 | 908 | 601 | −1.18 | −1.19 | −1.80 | Xpot |
| 1428982_at | Res & Lgx | 465 | 367 | 261 | 211 | −1.27 | −1.78 | −2.20 | Atad2b |
| 1428998_at | Lgx only | 1201 | 1404 | 1356 | 1719 | 1.17 | 1.13 | 1.43 | Phf3 |
| 1429001_at | Res only | 370 | 340 | 478 | 410 | −1.09 | 1.29 | 1.11 | Pir |
| 1429024_at | Lgx only | 3812 | 4605 | 4484 | 5982 | 1.21 | 1.18 | 1.57 | Rbm20 |
| 1429028_at | Lgx only | 460 | 366 | 348 | 309 | −1.26 | −1.32 | −1.49 | Dock11 |
| 1429034_at | Lgx only | 277 | 217 | 197 | 144 | −1.28 | −1.41 | −1.93 | Eme2 |
| 1429042_at | Res & Lgx | 583 | 493 | 381 | 355 | −1.18 | −1.53 | −1.64 | 2010200O16Rik |
| 1429057_at | Res & Lgx | 412 | 390 | 249 | 239 | −1.06 | −1.65 | −1.73 | Narg1l |
| 1429070_at | Lgx only | 166 | 214 | 221 | 297 | 1.29 | 1.33 | 1.79 | 4933440H19Rik |
| 1429085_at | Lgx only | 3041 | 3379 | 3563 | 3775 | 1.11 | 1.17 | 1.24 | Vezf1 |
| 1429103_at | Res & Lgx | 4218 | 4035 | 2877 | 2623 | −1.05 | −1.47 | −1.61 | Tomm22 |
| 1429107_at | Lgx only | 2462 | 2746 | 3278 | 3325 | 1.12 | 1.33 | 1.35 | Zfp650 |
| 1429119_at | Lgx only | 962 | 917 | 756 | 744 | −1.05 | −1.27 | −1.29 | Iah1 |
| 1429121_at | Lgx only | 375 | 263 | 322 | 170 | −1.43 | −1.16 | −2.20 | 4921517N04Rik |
| 1429137_at | Lgx only | 193 | 143 | 138 | 92 | −1.36 | −1.40 | −2.09 | 2810422O20Rik |
| 1429144_at | Res only | 14307 | 13184 | 10505 | 13791 | −1.09 | −1.36 | −1.04 | Prei4 |
| 1429146_at | Lgx only | 1179 | 1070 | 980 | 860 | −1.10 | −1.20 | −1.37 | 6620401M08Rik |
| 1429155_at | Res only | 996 | 805 | 760 | 789 | −1.24 | −1.31 | −1.26 | 4933411K20Rik |
| 1429160_at | Res & Lgx | 684 | 523 | 484 | 470 | −1.31 | −1.41 | −1.45 | Mtif3 |
| 1429183_at | Lgx only | 4351 | 3822 | 4249 | 3307 | −1.14 | −1.02 | −1.32 | Pkp2 |
| 1429186_a_at | Lgx only | 1223 | 1135 | 973 | 934 | −1.08 | −1.26 | −1.31 | Cdadc1 |
| 1429188_at | Lgx only | 1122 | 1148 | 907 | 855 | 1.02 | −1.24 | −1.31 | Cox11 |
| 1429194_at | Lgx only | 546 | 487 | 456 | 394 | −1.12 | −1.20 | −1.39 | Tigd2 |
| 1429196_at | Lgx only | 1381 | 1472 | 2063 | 2670 | 1.07 | 1.49 | 1.93 | Rabgap1l |
| 1429206_at | Lgx only | 3457 | 4642 | 4046 | 4930 | 1.34 | 1.17 | 1.43 | Rhobtb1 |
| 1429209_at | CR only | 179 | 101 | 118 | 196 | −1.78 | −1.52 | 1.09 | Col23a1 |
| 1429223_a_at | Res & Lgx | 2792 | 2374 | 2100 | 1610 | −1.18 | −1.33 | −1.73 | Hfe2 |
| 1429243_at | Res & Lgx | 597 | 537 | 380 | 369 | −1.11 | −1.57 | −1.62 | 1110054O05Rik |
| 1429253_at | Lgx only | 562 | 445 | 513 | 371 | −1.26 | −1.10 | −1.52 | Zmym4 |
| 1429264_at | Lgx only | 496 | 426 | 359 | 315 | −1.16 | −1.38 | −1.58 | C030044B11Rik |
| 1429278_at | Res only | 1094 | 1016 | 863 | 869 | −1.08 | −1.27 | −1.26 | Nubpl |
| 1429281_at | Res & Lgx | 323 | 264 | 201 | 185 | −1.22 | −1.60 | −1.75 | 2610008E11Rik |
| 1429300_at | Lgx only | 742 | 943 | 806 | 1172 | 1.27 | 1.09 | 1.58 | Ankrd9 |
| 1429321_at | Lgx only | 284 | 215 | 514 | 719 | −1.32 | 1.81 | 2.53 | Rnf149 |
| 1429328_at | Lgx only | 5245 | 4514 | 4643 | 3961 | −1.16 | −1.13 | −1.32 | Nsfl1c |
| 1429335_at | Lgx only | 320 | 284 | 287 | 200 | −1.13 | −1.12 | −1.60 | Snapc1 |
| 1429352_at | CR only | 291 | 116 | 140 | 150 | −2.52 | −2.08 | −1.95 | Mocos |
| 1429362_a_at | Res & Lgx | 1201 | 1528 | 1807 | 2347 | 1.27 | 1.50 | 1.95 | Sf3b2 |
| 1429364_at | Lgx only | 108 | 72 | 103 | 47 | −1.51 | −1.05 | −2.30 | 4930579G24Rik |
| 1429367_at | Lgx only | 693 | 717 | 798 | 936 | 1.03 | 1.15 | 1.35 | Wipi2 |
| 1429375_at | Lgx only | 1484 | 1372 | 1276 | 913 | −1.08 | −1.16 | −1.63 | Anapc10 |
| 1429395_at | Lgx only | 138 | 142 | 96 | 54 | 1.02 | −1.45 | −2.55 | Gstcd |
| 1429400_at | Lgx only | 189 | 176 | 206 | 314 | −1.07 | 1.09 | 1.66 | Clcn5 |
| 1429407_at | Lgx only | 188 | 211 | 333 | 337 | 1.12 | 1.77 | 1.79 | Pex11c |
| 1429413_at | Res & Lgx | 292 | 246 | 187 | 156 | −1.18 | −1.56 | −1.88 | Cpm |
| 1429425_at | Res & Lgx | 3510 | 3354 | 2840 | 2532 | −1.05 | −1.24 | −1.39 | Rnf139 |
| 1429454_at | Res & Lgx | 1848 | 1764 | 1340 | 1334 | −1.05 | −1.38 | −1.38 | Gapvd1 |
| 1429460_at | CR only | 15 | 60 | 60 | 28 | 4.09 | 4.13 | 1.89 | Gpr115 |
| 1429486_at | CR only | 1047 | 1648 | 912 | 1004 | 1.57 | −1.15 | −1.04 | Pfkfb2 |
| 1429487_at | Lgx only | 1134 | 1266 | 1668 | 1601 | 1.12 | 1.47 | 1.41 | Ppp1r12a |
| 1429505_at | CR & Lgx | 2791 | 1949 | 2450 | 1714 | −1.43 | −1.14 | −1.63 | 2310076G13Rik |
| 1429514_at | Lgx only | 4969 | 4087 | 4128 | 2951 | −1.22 | −1.20 | −1.68 | Ppap2b |
| 1429521_at | Lgx only | 323 | 250 | 235 | 153 | −1.29 | −1.37 | −2.12 | Alkbh8 |
| 1429532_at | Res only | 865 | 998 | 1089 | 938 | 1.15 | 1.26 | 1.08 | Morc2a |
| 1429534_a_at | Lgx only | 10910 | 12210 | 12327 | 12968 | 1.12 | 1.13 | 1.19 | Immt |
| 1429553_at | Lgx only | 151 | 181 | 260 | 574 | 1.20 | 1.73 | 3.81 | Cilp2 |
| 1429556_at | Lgx only | 2326 | 2174 | 1662 | 1363 | −1.07 | −1.40 | −1.71 | 2610024B07Rik |
| 1429570_at | Res only | 126 | 63 | 36 | 102 | −2.00 | −3.45 | −1.24 | Mlkl |
| 1429621_at | Lgx only | 839 | 1173 | 1240 | 1343 | 1.40 | 1.48 | 1.60 | Cand2 |
| 1429648_at | Lgx only | 637 | 552 | 475 | 461 | −1.15 | −1.34 | −1.38 | Slc35a3 |
| 1429681_a_at | Lgx only | 21171 | 22951 | 25817 | 28204 | 1.08 | 1.22 | 1.33 | Gpsn2 |
| 1429698_at | Res & Lgx | 163 | 155 | 82 | 89 | −1.05 | −1.99 | −1.83 | Mterf |
| 1429710_at | Res & Lgx | 839 | 763 | 558 | 459 | −1.10 | −1.51 | −1.83 | Styx |
| 1429723_at | Res & Lgx | 2893 | 2604 | 1673 | 1495 | −1.11 | −1.73 | −1.94 | 6330409N04Rik |
| 1429764_at | CR only | 2988 | 2220 | 3256 | 2552 | −1.35 | 1.09 | −1.17 | 1500005K14Rik |
| 1429771_at | Lgx only | 927 | 823 | 776 | 635 | −1.13 | −1.19 | −1.46 | 3110073H01Rik |
| 1429783_at | Res & Lgx | 13193 | 13213 | 10275 | 9645 | 1.00 | −1.28 | −1.37 | Pdlim5 |
| 1429819_at | Lgx only | 393 | 378 | 337 | 275 | −1.04 | −1.17 | −1.43 | Nmnat1 |
| 1429836_at | CR only | 142 | 251 | 216 | 269 | 1.78 | 1.53 | 1.90 | Ugcgl2 |
| 1429860_at | CR only | 43 | 117 | 154 | 113 | 2.69 | 3.56 | 2.61 | LOC677447 |
| 1429888_a_at | Lgx only | 4996 | 5288 | 5443 | 6090 | 1.06 | 1.09 | 1.22 | Hspb2 |
| 1429915_at | Lgx only | 56 | 63 | 79 | 116 | 1.12 | 1.42 | 2.08 | 4930426L09Rik |
| 1429918_at | CR only | 433 | 297 | 409 | 416 | −1.46 | −1.06 | −1.04 | Arhgap20 |
| 1429961_at | CR & Lgx | 308 | 198 | 313 | 200 | −1.55 | 1.02 | −1.54 | 1700021C14Rik |
| 1429990_at | Lgx only | 114 | 138 | 174 | 186 | 1.21 | 1.52 | 1.63 | Hyal4 |
| 1430000_at | CR only | 71 | 24 | 62 | 56 | −2.92 | −1.16 | −1.27 | B230117O15Rik |
| 1430045_at | CR & Res | 416 | 259 | 269 | 292 | −1.61 | −1.55 | −1.42 | Tsnax |
| 1430078_a_at | Lgx only | 284 | 264 | 259 | 154 | −1.07 | −1.09 | −1.85 | Ogg1 |
| 1430089_at | Lgx only | 355 | 291 | 227 | 142 | −1.22 | −1.57 | −2.50 | 5830469G19Rik |
| 1430095_at | Lgx only | 90 | 95 | 135 | 240 | 1.06 | 1.50 | 2.66 | D930020B18Rik |
| 1430123_a_at | Lgx only | 13843 | 13323 | 16691 | 18357 | −1.04 | 1.21 | 1.33 | Akr1a4 |
| 1430137_at | Res only | 148 | 166 | 220 | 222 | 1.12 | 1.49 | 1.51 | LOC100043489 |
| 1430170_at | Res only | 162 | 131 | 69 | 89 | −1.24 | −2.35 | −1.82 | Bbs10 |
| 1430224_at | Res only | 101 | 97 | 238 | 112 | −1.03 | 2.36 | 1.12 | Wfdc3 |
| 1430253_at | Lgx only | 114 | 159 | 173 | 252 | 1.40 | 1.52 | 2.22 | 2900006B11Rik |
| 1430292_a_at | Res & Lgx | 3466 | 3219 | 2398 | 2125 | −1.08 | −1.45 | −1.63 | 1810030N24Rik |
| 1430309_at | Res & Lgx | 1747 | 1890 | 2535 | 2577 | 1.08 | 1.45 | 1.47 | Nipbl |
| 1430378_at | Res & Lgx | 205 | 160 | 103 | 72 | −1.28 | −2.00 | −2.85 | 2900011G08Rik |
| 1430388_a_at | Lgx only | 1818 | 2657 | 2347 | 2718 | 1.46 | 1.29 | 1.49 | Sulf2 |
| 1430474_a_at | Res & Lgx | 8337 | 8097 | 5357 | 5222 | −1.03 | −1.56 | −1.60 | Mtch2 |
| 1430518_at | Lgx only | 85 | 28 | 55 | 17 | −3.02 | −1.56 | −5.03 | 5430402E10Rik |
| 1430519_a_at | Lgx only | 939 | 787 | 784 | 667 | −1.19 | −1.20 | −1.41 | Cnot7 |
| 1430527_a_at | Res & Lgx | 2831 | 2873 | 3201 | 3440 | 1.01 | 1.13 | 1.22 | Rnf167 |
| 1430544_at | Lgx only | 259 | 358 | 390 | 468 | 1.39 | 1.51 | 1.81 | 5830404H04Rik |
| 1430656_a_at | Res & Lgx | 3948 | 3401 | 2556 | 2175 | −1.16 | −1.54 | −1.82 | Asnsd1 |
| 1430676_at | CR & Lgx | 26 | 92 | 89 | 122 | 3.52 | 3.39 | 4.65 | Col19a1 |
| 1430685_at | Res only | 17 | 41 | 35 | 64 | 2.38 | 2.07 | 3.78 | 6330503C03Rik |
| 1430736_at | Lgx only | 176 | 135 | 173 | 48 | −1.31 | −1.02 | −3.70 | 9030411M15Rik |
| 1430768_at | Lgx only | 206 | 134 | 121 | 101 | −1.54 | −1.70 | −2.03 | 9530018H14Rik |
| 1430770_at | Res & Lgx | 56 | 28 | 13 | 12 | −1.97 | −4.28 | −4.68 | 3110080E11Rik |
| 1430781_at | Lgx only | 164 | 131 | 118 | 78 | −1.26 | −1.39 | −2.11 | Ak7 |
| 1430799_at | Res & Lgx | 68 | 35 | 14 | 18 | −1.91 | −4.91 | −3.76 | 5830432E09Rik |
| 1430818_at | CR only | 37 | 121 | 118 | 66 | 3.27 | 3.20 | 1.78 | Tmc1 |
| 1430835_at | CR only | 112 | 29 | 98 | 107 | −3.85 | −1.14 | −1.05 | Ccdc125 |
| 1430883_at | CR only | 103 | 36 | 71 | 54 | −2.83 | −1.44 | −1.89 | 4933402C05Rik |
| 1430910_at | CR only | 173 | 44 | 108 | 126 | −3.91 | −1.60 | −1.37 | 4930544L04Rik |
| 1430999_a_at | Res & Lgx | 840 | 643 | 415 | 329 | −1.31 | −2.02 | −2.55 | Scoc |
| 1431043_at | Lgx only | 596 | 596 | 738 | 893 | −1.00 | 1.24 | 1.50 | Kbtbd5 |
| 1431255_at | CR only | 604 | 385 | 610 | 572 | −1.57 | 1.01 | −1.06 | Calr3 |
| 1431287_at | CR only | 71 | 140 | 68 | 62 | 1.97 | −1.05 | −1.15 | Pcm1 |
| 1431293_a_at | Res & Lgx | 2705 | 1856 | 1662 | 1146 | −1.46 | −1.63 | −2.36 | Cldnd1 |
| 1431302_a_at | CR & Lgx | 4139 | 2946 | 3637 | 2685 | −1.41 | −1.14 | −1.54 | Nudt7 |
| 1431322_at | Lgx only | 704 | 836 | 854 | 982 | 1.19 | 1.21 | 1.40 | Igsf3 |
| 1431415_a_at | Lgx only | 681 | 643 | 595 | 493 | −1.06 | −1.14 | −1.38 | Tbpl1 |
| 1431428_a_at | Lgx only | 1082 | 1202 | 1304 | 1403 | 1.11 | 1.21 | 1.30 | Nosip |
| 1431429_a_at | Lgx only | 858 | 839 | 1095 | 1129 | −1.02 | 1.28 | 1.32 | Arl4a |
| 1431473_at | Lgx only | 18 | 7 | 25 | 81 | −2.53 | 1.36 | 4.43 | 5330423I11Rik |
| 1431498_at | Res only | 94 | 120 | 187 | 171 | 1.28 | 1.99 | 1.82 | 9530097N15Rik |
| 1431551_at | Lgx only | 62 | 105 | 104 | 164 | 1.69 | 1.68 | 2.64 | 2610028D06Rik |
| 1431561_a_at | Res only | 485 | 676 | 690 | 684 | 1.39 | 1.42 | 1.41 | Dhx34 |
| 1431587_at | Res only | 70 | 55 | 24 | 53 | −1.28 | −2.90 | −1.32 | Ccdc7 |
| 1431610_at | Res only | 49 | 101 | 141 | 107 | 2.05 | 2.87 | 2.18 | 5330439A09Rik |
| 1431618_a_at | CR & Res | 325 | 230 | 221 | 179 | −1.41 | −1.47 | −1.82 | D14Ertd581e |
| 1431619_a_at | Lgx only | 3465 | 3627 | 4405 | 4627 | 1.05 | 1.27 | 1.34 | Dtnbp1 |
| 1431679_at | Res only | 49 | 89 | 165 | 104 | 1.80 | 3.35 | 2.11 | 2510042H12Rik |
| 1431746_a_at | Lgx only | 3679 | 3526 | 2924 | 2509 | −1.04 | −1.26 | −1.47 | Ube1c |
| 1431785_at | Lgx only | 175 | 194 | 145 | 90 | 1.11 | −1.20 | −1.94 | Rnaset2a |
| 1431796_at | Lgx only | 31 | 106 | 75 | 91 | 3.40 | 2.40 | 2.93 | 2810430I11Rik |
| 1431804_a_at | Res & Lgx | 690 | 548 | 337 | 279 | −1.26 | −2.04 | −2.47 | Sp3 |
| 1431822_a_at | Res & Lgx | 3622 | 3320 | 2534 | 2374 | −1.09 | −1.43 | −1.53 | Azi2 |
| 1431827_a_at | Lgx only | 896 | 797 | 717 | 590 | −1.12 | −1.25 | −1.52 | Tlk2 |
| 1431853_at | CR only | 92 | 40 | 75 | 92 | −2.33 | −1.22 | 1.00 | 4933413C19Rik |
| 1431893_a_at | Res & Lgx | 638 | 758 | 379 | 436 | 1.19 | −1.68 | −1.46 | Pdss1 |
| 1431900_a_at | CR only | 32 | 90 | 31 | 85 | 2.79 | −1.06 | 2.64 | Foxa3 |
| 1431934_at | Lgx only | 72 | 109 | 81 | 157 | 1.51 | 1.13 | 2.18 | 4930505O20Rik |
| 1431986_at | Res only | 197 | 193 | 296 | 133 | −1.02 | 1.50 | −1.48 | 4933421A08Rik |
| 1431998_at | CR only | 69 | 14 | 61 | 59 | −4.95 | −1.14 | −1.16 | 4930432L08Rik |
| 1432000_a_at | Lgx only | 342 | 378 | 448 | 482 | 1.11 | 1.31 | 1.41 | Dedd |
| 1432016_a_at | CR only | 31956 | 37162 | 31250 | 32250 | 1.16 | −1.02 | 1.01 | Idh3a |
| 1432057_a_at | Lgx only | 662 | 743 | 770 | 911 | 1.12 | 1.16 | 1.38 | Prdm5 |
| 1432073_at | Res & Lgx | 3205 | 3584 | 2249 | 2107 | 1.12 | −1.43 | −1.52 | 1700113I22Rik |
| 1432122_at | Lgx only | 128 | 89 | 121 | 44 | −1.44 | −1.05 | −2.91 | Lrrc44 |
| 1432158_a_at | Res & Lgx | 2091 | 1924 | 1569 | 1489 | −1.09 | −1.33 | −1.40 | Trappc2 |
| 1432207_a_at | Lgx only | 454 | 448 | 459 | 350 | −1.01 | 1.01 | −1.30 | Toe1 |
| 1432248_at | Lgx only | 31 | 74 | 52 | 105 | 2.36 | 1.66 | 3.36 | 5430402P08Rik |
| 1432271_a_at | Res & Lgx | 1856 | 1636 | 1172 | 967 | −1.13 | −1.58 | −1.92 | Dcun1d5 |
| 1432348_at | Lgx only | 59 | 61 | 97 | 183 | 1.03 | 1.63 | 3.09 | 4930524O07Rik |
| 1432369_at | CR & Res | 60 | 139 | 152 | 155 | 2.30 | 2.52 | 2.57 | 3010027C24Rik |
| 1432420_a_at | Lgx only | 1929 | 1605 | 1516 | 1137 | −1.20 | −1.27 | −1.70 | 2310002L09Rik |
| 1432444_a_at | Lgx only | 1027 | 964 | 956 | 784 | −1.07 | −1.08 | −1.31 | Eapp |
| 1432457_at | Res only | 99 | 147 | 183 | 188 | 1.48 | 1.85 | 1.90 | 4930448F12Rik |
| 1432466_a_at | Lgx only | 9714 | 10651 | 13047 | 18186 | 1.10 | 1.34 | 1.87 | Apoe |
| 1432533_a_at | Lgx only | 372 | 420 | 476 | 655 | 1.13 | 1.28 | 1.76 | Slc35a2 |
| 1432543_a_at | Res only | 510 | 488 | 700 | 806 | −1.04 | 1.37 | 1.58 | Klf13 |
| 1432560_at | Res only | 52 | 58 | 122 | 70 | 1.13 | 2.37 | 1.35 | 1700127D06Rik |
| 1432590_at | CR only | 156 | 51 | 107 | 119 | −3.09 | −1.46 | −1.31 | 4930573O21Rik |
| 1432625_at | Res only | 32 | 59 | 123 | 62 | 1.81 | 3.78 | 1.90 | 5830487K18Rik |
| 1432626_at | Lgx only | 132 | 227 | 247 | 311 | 1.72 | 1.87 | 2.36 | 5730507A11Rik |
| 1432648_at | Res only | 67 | 77 | 17 | 67 | 1.16 | −4.01 | 1.01 | 4930466F19Rik |
| 1432662_at | Res only | 37 | 77 | 112 | 109 | 2.11 | 3.05 | 2.97 | 0610042E11Rik |
| 1432735_at | Lgx only | 256 | 258 | 314 | 474 | 1.01 | 1.23 | 1.86 | 1700017H01Rik |
| 1432930_at | Res only | 200 | 222 | 294 | 287 | 1.11 | 1.47 | 1.43 | 4930453O09Rik |
| 1433048_at | CR only | 185 | 107 | 139 | 196 | −1.74 | −1.33 | 1.06 | 4933428L01Rik |
| 1433132_at | CR only | 86 | 27 | 80 | 127 | −3.21 | −1.07 | 1.47 | Edaradd |
| 1433148_at | CR only | 226 | 484 | 386 | 319 | 2.14 | 1.71 | 1.41 | 4930513N20Rik |
| 1433164_at | Res only | 54 | 99 | 180 | 154 | 1.82 | 3.31 | 2.83 | 4930570E01Rik |
| 1433207_at | Lgx only | 57 | 67 | 66 | 136 | 1.17 | 1.14 | 2.37 | 5033430J17Rik |
| 1433241_at | Lgx only | 142 | 102 | 87 | 51 | −1.39 | −1.63 | −2.79 | 9430013L17Rik |
| 1433253_at | Lgx only | 40 | 88 | 74 | 128 | 2.21 | 1.86 | 3.24 | 5033423K11Rik |
| 1433314_at | Lgx only | 77 | 51 | 77 | 21 | −1.52 | 1.00 | −3.65 | 4930486A15Rik |
| 1433442_at | Lgx only | 2641 | 2244 | 1952 | 1694 | −1.18 | −1.35 | −1.56 | Klhl9 |
| 1433464_at | Lgx only | 1806 | 2481 | 2178 | 2563 | 1.37 | 1.21 | 1.42 | Ipo13 |
| 1433503_at | Res & Lgx | 5903 | 5778 | 4609 | 4467 | −1.02 | −1.28 | −1.32 | Zadh1 |
| 1433504_at | Lgx only | 18431 | 20243 | 22647 | 25548 | 1.10 | 1.23 | 1.39 | Pygb |
| 1433514_at | Lgx only | 1169 | 1037 | 957 | 909 | −1.13 | −1.22 | −1.29 | Etnk1 |
| 1433518_at | Lgx only | 529 | 567 | 612 | 674 | 1.07 | 1.16 | 1.27 | Lcmt2 |
| 1433519_at | Lgx only | 2411 | 2000 | 2022 | 1774 | −1.21 | −1.19 | −1.36 | Nucks1 |
| 1433520_at | Lgx only | 1068 | 1427 | 1464 | 1816 | 1.34 | 1.37 | 1.70 | Scap |
| 1433521_at | Res & Lgx | 1059 | 954 | 812 | 613 | −1.11 | −1.31 | −1.73 | Ankrd13c |
| 1433522_at | Lgx only | 465 | 508 | 536 | 616 | 1.09 | 1.15 | 1.32 | Pskh1 |
| 1433528_at | Lgx only | 1899 | 1634 | 1420 | 1242 | −1.16 | −1.34 | −1.53 | Gtf2a2 |
| 1433537_at | Lgx only | 1183 | 1089 | 1042 | 737 | −1.09 | −1.14 | −1.60 | 4833408C14Rik |
| 1433539_at | Res & Lgx | 2660 | 2213 | 1775 | 1485 | −1.20 | −1.50 | −1.79 | Commd3 |
| 1433544_at | Lgx only | 1808 | 1547 | 1540 | 1070 | −1.17 | −1.17 | −1.69 | Als2cr2 |
| 1433555_at | Lgx only | 416 | 379 | 412 | 237 | −1.10 | −1.01 | −1.75 | Eaf1 |
| 1433556_at | Res & Lgx | 138 | 182 | 263 | 232 | 1.32 | 1.90 | 1.68 | Centa1 |
| 1433561_at | Lgx only | 660 | 543 | 419 | 307 | −1.22 | −1.58 | −2.15 | Centb2 |
| 1433575_at | Lgx only | 3214 | 2986 | 2497 | 1537 | −1.08 | −1.29 | −2.09 | Sox4 |
| 1433585_at | Lgx only | 1024 | 898 | 841 | 644 | −1.14 | −1.22 | −1.59 | Tnpo1 |
| 1433597_at | Res & Lgx | 3769 | 3787 | 2160 | 1597 | 1.00 | −1.75 | −2.36 | 9430010O03Rik |
| 1433599_at | Lgx only | 331 | 323 | 351 | 222 | −1.03 | 1.06 | −1.49 | Baz1a |
| 1433645_at | Lgx only | 1238 | 1288 | 985 | 887 | 1.04 | −1.26 | −1.40 | Slc44a1 |
| 1433648_at | Lgx only | 3984 | 3473 | 3149 | 2552 | −1.15 | −1.26 | −1.56 | Spag9 |
| 1433656_a_at | Lgx only | 1182 | 1107 | 1036 | 879 | −1.07 | −1.14 | −1.34 | Gnl3 |
| 1433664_at | Res & Lgx | 664 | 522 | 428 | 377 | −1.27 | −1.55 | −1.76 | Ube2q2 |
| 1433676_at | Res & Lgx | 12728 | 12400 | 8809 | 8342 | −1.03 | −1.44 | −1.53 | Wnk1 |
| 1433682_at | Lgx only | 618 | 849 | 914 | 1124 | 1.37 | 1.48 | 1.82 | Arhgef17 |
| 1433686_at | Lgx only | 489 | 533 | 546 | 724 | 1.09 | 1.12 | 1.48 | Cabin1 |
| 1433691_at | Res & Lgx | 10244 | 10216 | 6379 | 5292 | −1.00 | −1.61 | −1.94 | Ppp1r3c |
| 1433698_a_at | Res & Lgx | 2618 | 2633 | 2921 | 3059 | 1.01 | 1.12 | 1.17 | Txnl4 |
| 1433700_at | Res & Lgx | 1009 | 844 | 673 | 749 | −1.19 | −1.50 | −1.35 | 4933433P14Rik |
| 1433705_at | Lgx only | 300 | 269 | 253 | 170 | −1.11 | −1.19 | −1.77 | Zfp213 |
| 1433712_at | Lgx only | 362 | 437 | 443 | 538 | 1.20 | 1.22 | 1.48 | AW555464 |
| 1433717_at | All | 1355 | 1876 | 1743 | 2058 | 1.38 | 1.29 | 1.52 | D19Wsu162e |
| 1433718_a_at | Lgx only | 862 | 744 | 773 | 554 | −1.16 | −1.12 | −1.56 | LOC100047028 |
| 1433722_at | Lgx only | 1946 | 2601 | 2972 | 3714 | 1.34 | 1.53 | 1.91 | Akap13 |
| 1433725_at | Res & Lgx | 840 | 1078 | 1085 | 1123 | 1.28 | 1.29 | 1.34 | Acvr1b |
| 1433727_at | Lgx only | 79 | 101 | 90 | 160 | 1.28 | 1.15 | 2.03 | BC038479 |
| 1433733_a_at | Lgx only | 179 | 226 | 163 | 115 | 1.26 | −1.10 | −1.55 | Cry1 |
| 1433738_at | Res & Lgx | 1127 | 966 | 802 | 666 | −1.17 | −1.40 | −1.69 | Papd5 |
| 1433741_at | Lgx only | 877 | 693 | 635 | 628 | −1.26 | −1.38 | −1.40 | Cd38 |
| 1433746_at | Lgx only | 484 | 409 | 361 | 275 | −1.18 | −1.34 | −1.76 | Wdr3 |
| 1433751_at | Lgx only | 580 | 428 | 435 | 248 | −1.36 | −1.33 | −2.34 | Slc39a10 |
| 1433760_a_at | Lgx only | 732 | 943 | 926 | 1027 | 1.29 | 1.26 | 1.40 | Rhbdd3 |
| 1433765_at | Lgx only | 521 | 587 | 784 | 834 | 1.13 | 1.50 | 1.60 | Ube2o |
| 1433770_at | Lgx only | 4705 | 4826 | 3514 | 3354 | 1.03 | −1.34 | −1.40 | Dpysl2 |
| 1433772_at | Res & Lgx | 820 | 636 | 421 | 437 | −1.29 | −1.95 | −1.87 | Stch |
| 1433790_at | CR only | 350 | 155 | 281 | 350 | −2.26 | −1.25 | −1.00 | Troap |
| 1433794_at | Lgx only | 1506 | 1268 | 1106 | 881 | −1.19 | −1.36 | −1.71 | Setx |
| 1433799_at | Lgx only | 877 | 735 | 582 | 537 | −1.19 | −1.51 | −1.63 | Rdh13 |
| 1433808_at | Lgx only | 912 | 1110 | 1207 | 1392 | 1.22 | 1.32 | 1.53 | D330001F17Rik |
| 1433811_at | Res & Lgx | 1795 | 2006 | 2548 | 2534 | 1.12 | 1.42 | 1.41 | Mllt6 |
| 1433847_at | Lgx only | 747 | 626 | 504 | 348 | −1.19 | −1.48 | −2.15 | D330017J20Rik |
| 1433868_at | Lgx only | 702 | 663 | 569 | 482 | −1.06 | −1.23 | −1.46 | Btbd3 |
| 1433875_at | Lgx only | 1471 | 1304 | 1208 | 1141 | −1.13 | −1.22 | −1.29 | 4732418C07Rik |
| 1433883_at | Lgx only | 3597 | 3657 | 2717 | 2316 | 1.02 | −1.32 | −1.55 | Tpm4 |
| 1433891_at | Res & Lgx | 1002 | 916 | 685 | 639 | −1.09 | −1.46 | −1.57 | Lgr4 |
| 1433897_at | Res & Lgx | 1769 | 1483 | 1048 | 773 | −1.19 | −1.69 | −2.29 | Al597468 |
| 1433898_at | Lgx only | 236 | 213 | 201 | 140 | −1.11 | −1.18 | −1.69 | AV025504 |
| 1433910_at | Lgx only | 1523 | 1421 | 1465 | 1231 | −1.07 | −1.04 | −1.24 | Zcchc6 |
| 1433914_at | Res & Lgx | 589 | 442 | 369 | 340 | −1.33 | −1.60 | −1.73 | Al747699 |
| 1433918_at | Res & Lgx | 1665 | 1645 | 1006 | 852 | −1.01 | −1.65 | −1.95 | Atg4d |
| 1433922_at | Res & Lgx | 1341 | 1190 | 903 | 781 | −1.13 | −1.48 | −1.72 | Rab35 |
| 1433926_at | Res & Lgx | 1929 | 1830 | 1548 | 1580 | −1.05 | −1.25 | −1.22 | Dync1li2 |
| 1433931_at | Lgx only | 1207 | 1431 | 1706 | 2152 | 1.18 | 1.41 | 1.78 | C030046l01Rik |
| 1433952_at | Res & Lgx | 1764 | 2403 | 2540 | 2663 | 1.36 | 1.44 | 1.51 | Tufm |
| 1433953_at | Lgx only | 1145 | 1007 | 993 | 868 | −1.14 | −1.15 | −1.32 | Zfp277 |
| 1433979_at | Lgx only | 1885 | 2263 | 1938 | 2261 | 1.20 | 1.03 | 1.20 | Rbms2 |
| 1433986_at | Lgx only | 2432 | 2354 | 2145 | 2176 | −1.03 | −1.13 | −1.12 | BC024659 |
| 1434001_at | Res & Lgx | 591 | 503 | 394 | 398 | −1.17 | −1.50 | −1.48 | Ttc9c |
| 1434008_at | Lgx only | 1391 | 940 | 1006 | 885 | −1.48 | −1.38 | −1.57 | Scn4b |
| 1434009_at | Res & Lgx | 624 | 786 | 1028 | 1225 | 1.26 | 1.65 | 1.96 | Grlf1 |
| 1434018_at | Lgx only | 996 | 1035 | 1127 | 1368 | 1.04 | 1.13 | 1.37 | BC043098 |
| 1434059_at | Lgx only | 519 | 522 | 671 | 756 | 1.00 | 1.29 | 1.46 | B230312A22Rik |
| 1434064_at | Res & Lgx | 474 | 589 | 715 | 770 | 1.24 | 1.51 | 1.62 | Tmem142c |
| 1434066_at | Lgx only | 807 | 953 | 1127 | 1361 | 1.18 | 1.40 | 1.69 | Gtf3c1 |
| 1434067_at | Res only | 21 | 38 | 74 | 62 | 1.83 | 3.54 | 2.96 | Al662270 |
| 1434072_at | Lgx only | 893 | 774 | 728 | 646 | −1.15 | −1.23 | −1.38 | Smcr7 |
| 1434075_at | Res & Lgx | 1023 | 923 | 673 | 623 | −1.11 | −1.52 | −1.64 | BC030336 |
| 1434082_at | Lgx only | 1059 | 957 | 940 | 850 | −1.11 | −1.13 | −1.25 | Pctk2 |
| 1434084_at | Lgx only | 536 | 417 | 380 | 265 | −1.28 | −1.41 | −2.02 | 5730601F06Rik |
| 1434088_at | Lgx only | 814 | 957 | 921 | 1043 | 1.18 | 1.13 | 1.28 | Zkscan17 |
| 1434096_at | Lgx only | 760 | 778 | 873 | 1035 | 1.02 | 1.15 | 1.36 | Slc4a4 |
| 1434105_at | Lgx only | 1682 | 1795 | 2023 | 2368 | 1.07 | 1.20 | 1.41 | Epm2aip1 |
| 1434115_at | Lgx only | 3429 | 4304 | 3972 | 4713 | 1.26 | 1.16 | 1.37 | Cdh13 |
| 1434131_at | Lgx only | 1087 | 1126 | 1252 | 1367 | 1.04 | 1.15 | 1.26 | Rufy1 |
| 1434135_at | Lgx only | 634 | 615 | 511 | 451 | −1.03 | −1.24 | −1.40 | B3galnt2 |
| 1434138_at | Lgx only | 910 | 747 | 789 | 685 | −1.22 | −1.15 | −1.33 | Prune |
| 1434140_at | CR only | 1207 | 1781 | 1342 | 1311 | 1.48 | 1.11 | 1.09 | Mcf2l |
| 1434153_at | CR & Lgx | 845 | 1513 | 953 | 1127 | 1.79 | 1.13 | 1.33 | Shb |
| 1434174_at | Res only | 619 | 536 | 463 | 444 | −1.16 | −1.34 | −1.40 | Lysmd3 |
| 1434200_at | Res & Lgx | 604 | 514 | 322 | 304 | −1.17 | −1.88 | −1.99 | BC010981 |
| 1434205_at | Res & Lgx | 3650 | 3118 | 2521 | 1621 | −1.17 | −1.45 | −2.25 | Ppp2r5c |
| 1434234_at | Lgx only | 23 | 72 | 52 | 115 | 3.07 | 2.22 | 4.92 | Zfp341 |
| 1434238_at | Lgx only | 417 | 408 | 282 | 247 | −1.02 | −1.48 | −1.69 | Taf2 |
| 1434248_at | Res only | 842 | 810 | 1061 | 848 | −1.04 | 1.26 | 1.01 | Prkch |
| 1434268_at | Lgx only | 761 | 830 | 965 | 1097 | 1.09 | 1.27 | 1.44 | Adar |
| 1434271_at | Res & Lgx | 520 | 568 | 711 | 747 | 1.09 | 1.37 | 1.44 | Gba2 |
| 1434273_at | Lgx only | 11070 | 12667 | 12227 | 13559 | 1.14 | 1.10 | 1.22 | BC034069 |
| 1434277_a_at | CR only | 774 | 1200 | 842 | 881 | 1.55 | 1.09 | 1.14 | Ypel2 |
| 1434278_at | Lgx only | 1459 | 881 | 1015 | 473 | −1.66 | −1.44 | −3.09 | Mtm1 |
| 1434281_at | Lgx only | 1018 | 999 | 812 | 698 | −1.02 | −1.25 | −1.46 | 1500034J01Rik |
| 1434283_at | Res & Lgx | 893 | 1206 | 1278 | 1477 | 1.35 | 1.43 | 1.65 | LOC100044968 |
| 1434284_at | Res & Lgx | 829 | 710 | 613 | 588 | −1.17 | −1.35 | −1.41 | Bdp1 |
| 1434296_at | Lgx only | 291 | 265 | 237 | 224 | −1.10 | −1.23 | −1.30 | BC049349 |
| 1434303_at | Lgx only | 1077 | 1189 | 2323 | 2458 | 1.10 | 2.16 | 2.28 | Raph1 |
| 1434320_at | Lgx only | 512 | 413 | 369 | 377 | −1.24 | −1.39 | −1.36 | Gtf3c4 |
| 1434328_at | Lgx only | 2487 | 2492 | 2004 | 1703 | 1.00 | −1.24 | −1.46 | Rpl15 |
| 1434339_at | Res & Lgx | 966 | 835 | 676 | 499 | −1.16 | −1.43 | −1.94 | Fnbp1l |
| 1434344_at | Lgx only | 1331 | 1208 | 1085 | 944 | −1.10 | −1.23 | −1.41 | Gpkow |
| 1434354_at | Lgx only | 3473 | 3028 | 2675 | 2382 | −1.15 | −1.30 | −1.46 | Maob |
| 1434356_a_at | Lgx only | 6321 | 5829 | 5156 | 4746 | −1.08 | −1.23 | −1.33 | Psma5 |
| 1434372_at | Lgx only | 2170 | 1834 | 1771 | 1504 | −1.18 | −1.23 | −1.44 | AW112010 |
| 1434378_a_at | Lgx only | 952 | 996 | 1039 | 1163 | 1.05 | 1.09 | 1.22 | Mxd4 |
| 1434387_at | Lgx only | 2313 | 2828 | 3081 | 3362 | 1.22 | 1.33 | 1.45 | Itfg3 |
| 1434392_at | Res & Lgx | 1484 | 1245 | 1133 | 925 | −1.19 | −1.31 | −1.61 | Usp34 |
| 1434402_at | Lgx only | 683 | 631 | 619 | 432 | −1.08 | −1.10 | −1.58 | Samd8 |
| 1434405_at | Res & Lgx | 1119 | 991 | 685 | 678 | −1.13 | −1.63 | −1.65 | Fnip1 |
| 1434422_at | CR & Lgx | 232 | 140 | 152 | 120 | −1.66 | −1.53 | −1.94 | AI428479 |
| 1434441_at | Lgx only | 1752 | 1522 | 1369 | 1299 | −1.15 | −1.28 | −1.35 | 1110018J18Rik |
| 1434442_at | CR only | 391 | 549 | 429 | 449 | 1.40 | 1.10 | 1.15 | Stbd1 |
| 1434461_at | Res & Lgx | 1215 | 1185 | 926 | 822 | −1.03 | −1.31 | −1.48 | Zfp715 |
| 1434487_at | Lgx only | 1586 | 1697 | 2179 | 2499 | 1.07 | 1.37 | 1.58 | Mef2d |
| 1434493_at | Lgx only | 158 | 164 | 101 | 106 | 1.03 | −1.57 | −1.50 | 1810022K09Rik |
| 1434511_at | Lgx only | 1331 | 1195 | 1108 | 920 | −1.11 | −1.20 | −1.45 | Phkb |
| 1434516_at | Lgx only | 1502 | 1422 | 1254 | 1162 | −1.06 | −1.20 | −1.29 | Pstk |
| 1434518_at | Res & Lgx | 918 | 972 | 1104 | 1186 | 1.06 | 1.20 | 1.29 | Phka2 |
| 1434558_at | Lgx only | 280 | 256 | 227 | 155 | −1.09 | −1.23 | −1.81 | Wdr47 |
| 1434565_at | Res & Lgx | 1072 | 1078 | 773 | 729 | 1.01 | −1.39 | −1.47 | Cgrrf1 |
| 1434586_a_at | Lgx only | 2001 | 1938 | 1529 | 1209 | −1.03 | −1.31 | −1.66 | Ptdss2 |
| 1434597_at | Lgx only | 2702 | 3318 | 3044 | 3746 | 1.23 | 1.13 | 1.39 | Larp5 |
| 1434604_at | Lgx only | 1414 | 1229 | 1298 | 1039 | −1.15 | −1.09 | −1.36 | Eif5b |
| 1434610_at | Res & Lgx | 2684 | 3974 | 4970 | 6496 | 1.48 | 1.85 | 2.42 | Plec1 |
| 1434613_at | Lgx only | 2786 | 2883 | 1811 | 1538 | 1.03 | −1.54 | −1.81 | 1810013L24Rik |
| 1434625_at | Lgx only | 882 | 647 | 703 | 609 | −1.36 | −1.25 | −1.45 | 4930432O21Rik |
| 1434642_at | Res & Lgx | 3093 | 2964 | 4527 | 4450 | −1.04 | 1.46 | 1.44 | Hsd17b11 |
| 1434647_at | Lgx only | 754 | 584 | 603 | 458 | −1.29 | −1.25 | −1.65 | Egflam |
| 1434648_a_at | Lgx only | 900 | 688 | 589 | 361 | −1.31 | −1.53 | −2.50 | Ccm2 |
| 1434655_at | Lgx only | 206 | 244 | 274 | 363 | 1.18 | 1.33 | 1.76 | Foxk1 |
| 1434665_at | Lgx only | 1767 | 1845 | 1861 | 2289 | 1.04 | 1.05 | 1.30 | Aga |
| 1434671_at | Lgx only | 1246 | 959 | 1311 | 840 | −1.30 | 1.05 | −1.48 | B230337E12Rik |
| 1434672_at | Lgx only | 1613 | 1191 | 1046 | 779 | −1.35 | −1.54 | −2.07 | Gpr22 |
| 1434714_at | Lgx only | 227 | 243 | 341 | 399 | 1.07 | 1.50 | 1.76 | Ero1lb |
| 1434736_at | Lgx only | 894 | 1036 | 1010 | 1326 | 1.16 | 1.13 | 1.48 | Hlf |
| 1434765_at | Lgx only | 788 | 738 | 602 | 509 | −1.07 | −1.31 | −1.55 | Ep300 |
| 1434775_at | Lgx only | 573 | 537 | 690 | 773 | −1.07 | 1.20 | 1.35 | Pard3 |
| 1434791_at | Lgx only | 746 | 909 | 934 | 1128 | 1.22 | 1.25 | 1.51 | Atp6v0a2 |
| 1434792_at | Lgx only | 639 | 423 | 604 | 401 | −1.51 | −1.06 | −1.60 | 2010320M18Rik |
| 1434805_at | Lgx only | 439 | 529 | 521 | 670 | 1.20 | 1.19 | 1.53 | Mllt1 |
| 1434822_at | Lgx only | 1213 | 1095 | 1009 | 970 | −1.11 | −1.20 | −1.25 | Pphln1 |
| 1434824_at | Lgx only | 1740 | 2163 | 2205 | 2627 | 1.24 | 1.27 | 1.51 | Baz1b |
| 1434826_at | Lgx only | 548 | 525 | 447 | 344 | −1.04 | −1.23 | −1.59 | Rfesd |
| 1434835_at | Lgx only | 1066 | 952 | 926 | 824 | −1.12 | −1.15 | −1.29 | Wapal |
| 1434838_at | Lgx only | 4161 | 4913 | 5731 | 5398 | 1.18 | 1.38 | 1.30 | Kcng2 |
| 1434864_at | Res & Lgx | 690 | 688 | 467 | 358 | −1.00 | −1.48 | −1.93 | Nipa1 |
| 1434891_at | Lgx only | 949 | 1098 | 1092 | 1555 | 1.16 | 1.15 | 1.64 | Ptgfrn |
| 1434896_at | Res & Lgx | 968 | 863 | 618 | 562 | −1.12 | −1.57 | −1.72 | Zfp422-rs1 |
| 1434900_at | Lgx only | 265 | 301 | 367 | 406 | 1.13 | 1.38 | 1.53 | Mkl1 |
| 1434904_at | Res & Lgx | 462 | 569 | 661 | 898 | 1.23 | 1.43 | 1.94 | Hivep2 |
| 1434909_at | Res & Lgx | 2412 | 2355 | 1922 | 1743 | −1.02 | −1.26 | −1.38 | Rragd |
| 1434916_at | Lgx only | 470 | 406 | 406 | 314 | −1.16 | −1.16 | −1.50 | Vkorc1l1 |
| 1434927_at | Lgx only | 38292 | 44321 | 50306 | 62045 | 1.16 | 1.31 | 1.62 | Hspb7 |
| 1434930_at | Lgx only | 665 | 821 | 984 | 1031 | 1.23 | 1.48 | 1.55 | Tpcn1 |
| 1434934_at | Res & Lgx | 4033 | 3850 | 2890 | 2753 | −1.05 | −1.40 | −1.46 | Atpaf1 |
| 1434936_at | Lgx only | 374 | 413 | 491 | 592 | 1.10 | 1.31 | 1.58 | Hirip3 |
| 1434942_at | Res & Lgx | 901 | 696 | 666 | 528 | −1.30 | −1.35 | −1.71 | Esf1 |
| 1434944_at | Lgx only | 6233 | 7918 | 9727 | 9182 | 1.27 | 1.56 | 1.47 | Dmpk |
| 1434967_at | Res & Lgx | 535 | 436 | 358 | 324 | −1.23 | −1.49 | −1.65 | Zswim6 |
| 1434978_at | Lgx only | 1957 | 2176 | 1745 | 1353 | 1.11 | −1.12 | −1.45 | 4933403F05Rik |
| 1434981_at | Res only | 109 | 143 | 215 | 123 | 1.31 | 1.97 | 1.12 | E130303B06Rik |
| 1434997_at | Lgx only | 592 | 529 | 618 | 756 | −1.12 | 1.04 | 1.28 | Cdc2l6 |
| 1435016_at | Lgx only | 3394 | 3426 | 2781 | 2210 | 1.01 | −1.22 | −1.54 | Trak2 |
| 1435017_at | Lgx only | 3573 | 4157 | 4007 | 4259 | 1.16 | 1.12 | 1.19 | Mel13 |
| 1435018_at | Lgx only | 872 | 1011 | 949 | 1112 | 1.16 | 1.09 | 1.28 | 5930434B04Rik |
| 1435032_at | Lgx only | 747 | 869 | 992 | 1031 | 1.16 | 1.33 | 1.38 | Golgb1 |
| 1435091_at | Lgx only | 591 | 460 | 474 | 418 | −1.29 | −1.25 | −1.41 | Zfp568 |
| 1435117_a_at | Lgx only | 460 | 552 | 597 | 615 | 1.20 | 1.30 | 1.34 | Rbj |
| 1435135_at | Res & Lgx | 6817 | 7003 | 9784 | 10952 | 1.03 | 1.44 | 1.61 | Aadacl1 |
| 1435153_at | Lgx only | 764 | 784 | 688 | 591 | 1.03 | −1.11 | −1.29 | Btbd6 |
| 1435169_at | CR only | 1062 | 1377 | 1382 | 1273 | 1.30 | 1.30 | 1.20 | A930001N09Rik |
| 1435180_at | Lgx only | 460 | 729 | 770 | 1246 | 1.58 | 1.67 | 2.71 | Podn |
| 1435183_at | All | 452 | 596 | 827 | 928 | 1.32 | 1.83 | 2.05 | Tbkbp1 |
| 1435224_at | Lgx only | 1147 | 1340 | 1495 | 1677 | 1.17 | 1.30 | 1.46 | Crebbp |
| 1435242_at | Res & Lgx | 596 | 524 | 452 | 456 | −1.14 | −1.32 | −1.31 | Pds5b |
| 1435248_a_at | Lgx only | 793 | 736 | 625 | 445 | −1.08 | −1.27 | −1.78 | Btaf1 |
| 1435250_at | Res & Lgx | 509 | 498 | 329 | 263 | −1.02 | −1.55 | −1.93 | Ints8 |
| 1435261_at | Res & Lgx | 2639 | 2800 | 3398 | 4623 | 1.06 | 1.29 | 1.75 | 4732416N19Rik |
| 1435295_at | Res only | 394 | 484 | 605 | 566 | 1.23 | 1.54 | 1.44 | Dopey1 |
| 1435340_at | Lgx only | 409 | 634 | 611 | 728 | 1.55 | 1.49 | 1.78 | Jmjd2a |
| 1435349_at | Lgx only | 646 | 526 | 503 | 317 | −1.23 | −1.28 | −2.04 | Nrp2 |
| 1435351_at | CR only | 601 | 471 | 619 | 512 | −1.28 | 1.03 | −1.18 | 2310026E23Rik |
| 1435360_at | Lgx only | 1234 | 1322 | 1550 | 1828 | 1.07 | 1.26 | 1.48 | Zfp651 |
| 1435377_at | Res & Lgx | 561 | 417 | 320 | 302 | −1.34 | −1.75 | −1.86 | 2410002O22Rik |
| 1435378_at | Lgx only | 977 | 1013 | 1221 | 1341 | 1.04 | 1.25 | 1.37 | 2210020M01Rik |
| 1435386_at | Lgx only | 966 | 1325 | 1564 | 2231 | 1.37 | 1.62 | 2.31 | Vwf |
| 1435437_at | Lgx only | 1823 | 1595 | 1637 | 1411 | −1.14 | −1.11 | −1.29 | Setd7 |
| 1435441_at | Res only | 366 | 339 | 487 | 474 | −1.08 | 1.33 | 1.29 | Ablim2 |
| 1435442_at | Res & Lgx | 1660 | 1466 | 1220 | 1214 | −1.13 | −1.36 | −1.37 | Wdsof1 |
| 1435443_at | Lgx only | 715 | 866 | 940 | 998 | 1.21 | 1.31 | 1.40 | Eya3 |
| 1435461_at | Res & Lgx | 2568 | 2130 | 1753 | 1415 | −1.21 | −1.46 | −1.81 | Magi3 |
| 1435490_at | CR only | 110 | 246 | 176 | 195 | 2.23 | 1.60 | 1.77 | Hk3 |
| 1435505_at | Res only | 1051 | 1219 | 1346 | 1482 | 1.16 | 1.28 | 1.41 | Dmwd |
| 1435524_at | Lgx only | 1085 | 1246 | 1347 | 1415 | 1.15 | 1.24 | 1.30 | 2010109N14Rik |
| 1435526_at | Lgx only | 566 | 469 | 430 | 359 | −1.21 | −1.32 | −1.58 | Tor1aip2 |
| 1435527_at | Lgx only | 1442 | 1796 | 2081 | 2372 | 1.24 | 1.44 | 1.64 | Nfic |
| 1435529_at | Res & Lgx | 949 | 677 | 595 | 504 | −1.40 | −1.60 | −1.88 | OTTMUSG00000016644 |
| 1435543_at | Lgx only | 986 | 868 | 772 | 684 | −1.14 | −1.28 | −1.44 | LOC100048863 |
| 1435547_at | Lgx only | 1442 | 1468 | 1270 | 1189 | 1.02 | −1.14 | −1.21 | ENSMUSG00000075401 |
| 1435548_at | Lgx only | 726 | 703 | 596 | 466 | −1.03 | −1.22 | −1.56 | Mrs2l |
| 1435549_at | Lgx only | 434 | 440 | 504 | 549 | 1.01 | 1.16 | 1.26 | Trpm4 |
| 1435554_at | Res & Lgx | 1663 | 1372 | 1080 | 922 | −1.21 | −1.54 | −1.80 | Tmcc3 |
| 1435556_at | Lgx only | 530 | 463 | 373 | 373 | −1.15 | −1.42 | −1.42 | Zfp597 |
| 1435589_at | Res & Lgx | 510 | 650 | 831 | 1114 | 1.27 | 1.63 | 2.18 | Ccdc85b |
| 1435641_at | Res & Lgx | 437 | 400 | 308 | 300 | −1.09 | −1.42 | −1.45 | 9530018I07Rik |
| 1435655_at | Lgx only | 248 | 223 | 189 | 131 | −1.11 | −1.31 | −1.89 | Snora65 |
| 1435674_at | Lgx only | 361 | 478 | 368 | 510 | 1.33 | 1.02 | 1.41 | Rhobtb2 |
| 1435679_at | Lgx only | 1778 | 1866 | 1603 | 1228 | 1.05 | −1.11 | −1.45 | Optn |
| 1435693_at | CR only | 348 | 448 | 320 | 452 | 1.29 | −1.09 | 1.30 | Mall |
| 1435695_a_at | Lgx only | 876 | 756 | 620 | 449 | −1.16 | −1.41 | −1.95 | AO30007L17Rik |
| 1435743_at | Res & Lgx | 987 | 808 | 671 | 634 | −1.22 | −1.47 | −1.56 | Klhl23 |
| 1435754_at | Res & Lgx | 1015 | 818 | 741 | 714 | −1.24 | −1.37 | −1.42 | Zyg11b |
| 1435768_at | Lgx only | 919 | 909 | 725 | 632 | −1.01 | −1.27 | −1.46 | Arid4b |
| 1435774_at | Lgx only | 487 | 394 | 360 | 292 | −1.24 | −1.35 | −1.67 | AV024533 |
| 1435777_at | Lgx only | 1189 | 995 | 853 | 715 | −1.19 | −1.39 | −1.66 | E030018N11Rik |
| 1435782_at | Lgx only | 166 | 244 | 291 | 295 | 1.47 | 1.75 | 1.78 | LOC668206 |
| 1435808_at | Res & Lgx | 579 | 704 | 924 | 1015 | 1.22 | 1.60 | 1.75 | A230051G13Rik |
| 1435813_at | Lgx only | 2373 | 3193 | 3181 | 3356 | 1.35 | 1.34 | 1.41 | Mypn |
| 1435864_a_at | Res & Lgx | 2251 | 2148 | 1526 | 1411 | −1.05 | −1.48 | −1.59 | 1810063B05Rik |
| 1435874_at | Lgx only | 1005 | 1045 | 939 | 727 | 1.04 | −1.07 | −1.38 | Prkab2 |
| 1435900_at | Res & Lgx | 775 | 768 | 643 | 518 | −1.01 | −1.20 | −1.49 | Zbtb43 |
| 1435912_at | Lgx only | 670 | 587 | 579 | 475 | −1.14 | −1.16 | −1.41 | Ubxd7 |
| 1435947_at | Lgx only | 647 | 572 | 495 | 456 | −1.13 | −1.31 | −1.42 | 2810455D13Rik |
| 1436014_a_at | CR only | 449 | 349 | 494 | 518 | −1.28 | 1.10 | 1.16 | Rusc1 |
| 1436026_at | Lgx only | 476 | 418 | 620 | 736 | −1.14 | 1.30 | 1.54 | Zfp703 |
| 1436033_at | Lgx only | 879 | 984 | 1031 | 1238 | 1.12 | 1.17 | 1.41 | BC031353 |
| 1436041_at | CR & Lgx | 1831 | 1425 | 1788 | 1414 | −1.28 | −1.02 | −1.29 | LOC100046086 |
| 1436045_at | Lgx only | 381 | 321 | 282 | 216 | −1.19 | −1.35 | −1.76 | Tsga10 |
| 1436059_at | Res & Lgx | 254 | 366 | 397 | 446 | 1.44 | 1.56 | 1.75 | Rfx1 |
| 1436075_at | Lgx only | 280 | 256 | 422 | 622 | −1.09 | 1.51 | 2.22 | Sfrp5 |
| 1436081_a_at | Lgx only | 1053 | 1205 | 1255 | 1434 | 1.14 | 1.19 | 1.36 | Zfp414 |
| 1436112_at | Res only | 158 | 196 | 325 | 246 | 1.24 | 2.06 | 1.55 | AI118078 |
| 1436113_a_at | Res only | 2773 | 2845 | 2148 | 2176 | 1.03 | −1.29 | −1.27 | St13 |
| 1436121_a_at | Lgx only | 1870 | 1812 | 1483 | 1122 | −1.03 | −1.26 | −1.67 | Nsmce1 |
| 1436122_at | Res only | 242 | 299 | 396 | 310 | 1.24 | 1.64 | 1.28 | Zfp667 |
| 1436157_at | Lgx only | 1826 | 1496 | 1478 | 1265 | −1.22 | −1.24 | −1.44 | Ccar1 |
| 1436188_a_at | CR only | 7033 | 11508 | 7677 | 9091 | 1.64 | 1.09 | 1.29 | Ndrg4 |
| 1436208_at | CR & Lgx | 551 | 716 | 663 | 794 | 1.30 | 1.20 | 1.44 | Asb1 |
| 1436214_at | Lgx only | 567 | 465 | 395 | 293 | −1.22 | −1.44 | −1.93 | 1110028C15Rik |
| 1436215_at | Lgx only | 642 | 508 | 549 | 433 | −1.26 | −1.17 | −1.48 | Ipmk |
| 1436233_at | Lgx only | 1039 | 871 | 997 | 736 | −1.19 | −1.04 | −1.41 | Btnl9 |
| 1436240_at | Lgx only | 382 | 383 | 308 | 256 | 1.00 | −1.24 | −1.49 | Sost |
| 1436243_at | Lgx only | 2945 | 3472 | 2166 | 1917 | 1.18 | −1.36 | −1.54 | Frmd5 |
| 1436275_at | Lgx only | 5589 | 6926 | 4236 | 4096 | 1.24 | −1.32 | −1.36 | Kcnip2 |
| 1436299_at | Res & Lgx | 1490 | 1306 | 1113 | 877 | −1.14 | −1.34 | −1.70 | Gls |
| 1436310_at | Lgx only | 199 | 301 | 245 | 313 | 1.51 | 1.23 | 1.57 | Gemin5 |
| 1436332_at | Lgx only | 32378 | 38509 | 38078 | 46348 | 1.19 | 1.18 | 1.43 | Hspb6 |
| 1436339_at | Lgx only | 6840 | 6976 | 10138 | 9417 | 1.02 | 1.48 | 1.38 | 1810058I24Rik |
| 1436342_a_at | Lgx only | 1693 | 1473 | 1337 | 1170 | −1.15 | −1.27 | −1.45 | D19Ertd721e |
| 1436367_at | Lgx only | 3325 | 3749 | 4038 | 4164 | 1.13 | 1.21 | 1.25 | C130094E24 |
| 1436377_at | Res only | 595 | 716 | 830 | 810 | 1.20 | 1.40 | 1.36 | Gpr137 |
| 1436408_at | Lgx only | 88 | 45 | 76 | 29 | −1.95 | −1.16 | −3.02 | Rprml |
| 1436425_at | Lgx only | 182 | 121 | 125 | 126 | −1.50 | −1.45 | −1.45 | Ankrd38 |
| 1436446_at | Res & Lgx | 675 | 568 | 441 | 416 | −1.19 | −1.53 | −1.62 | 2310007O11Rik |
| 1436505_at | CR only | 722 | 511 | 816 | 739 | −1.41 | 1.13 | 1.02 | Ppig |
| 1436511_at | Res & Lgx | 675 | 634 | 510 | 460 | −1.06 | −1.32 | −1.47 | BC031781 |
| 1436521_at | Lgx only | 995 | 1371 | 1104 | 1496 | 1.38 | 1.11 | 1.50 | Slc36a2 |
| 1436537_at | CR only | 624 | 518 | 554 | 573 | −1.20 | −1.13 | −1.09 | Zfp629 |
| 1436538_at | Res & Lgx | 425 | 320 | 201 | 116 | −1.33 | −2.12 | −3.68 | Ankrd37 |
| 1436546_at | Res & Lgx | 1262 | 1105 | 994 | 881 | −1.14 | −1.27 | −1.43 | Lix1l |
| 1436547_at | CR only | 380 | 285 | 282 | 352 | −1.33 | −1.34 | −1.08 | Dgke |
| 1436562_at | Lgx only | 579 | 545 | 498 | 363 | −1.06 | −1.16 | −1.59 | Ddx58 |
| 1436584_at | Res & Lgx | 825 | 721 | 541 | 545 | −1.15 | −1.53 | −1.51 | Spry2 |
| 1436594_at | Lgx only | 180 | 124 | 152 | 103 | −1.45 | −1.18 | −1.74 | Zfp719 |
| 1436609_a_at | Lgx only | 4537 | 4960 | 5297 | 5655 | 1.09 | 1.17 | 1.25 | Lrpap1 |
| 1436650_at | CR & Lgx | 729 | 1049 | 944 | 1072 | 1.44 | 1.30 | 1.47 | Filip1 |
| 1436665_a_at | Lgx only | 3944 | 5547 | 5624 | 7673 | 1.41 | 1.43 | 1.95 | Ltbp4 |
| 1436739_at | Lgx only | 2627 | 2725 | 2561 | 2047 | 1.04 | −1.03 | −1.28 | Agtr1a |
| 1436747_at | Lgx only | 2419 | 2508 | 2836 | 3129 | 1.04 | 1.17 | 1.29 | 1110014K08Rik |
| 1436797_a_at | Lgx only | 1519 | 1814 | 1790 | 2289 | 1.19 | 1.18 | 1.51 | Surf4 |
| 1436809_a_at | All | 1520 | 2518 | 2464 | 2994 | 1.66 | 1.62 | 1.97 | Spin1 |
| 1436817_at | Lgx only | 1182 | 1030 | 929 | 752 | −1.15 | −1.27 | −1.57 | Exoc5 |
| 1436842_at | Res & Lgx | 3911 | 3696 | 2482 | 2369 | −1.06 | −1.58 | −1.65 | B230380D07Rik |
| 1436844_at | Lgx only | 536 | 431 | 451 | 371 | −1.24 | −1.19 | −1.44 | AW046287 |
| 1436865_at | CR only | 308 | 173 | 311 | 209 | −1.78 | 1.01 | −1.47 | Slc26a11 |
| 1436867_at | CR only | 23230 | 28014 | 23888 | 25409 | 1.21 | 1.03 | 1.09 | Srl |
| 1436883_at | Lgx only | 558 | 425 | 421 | 399 | −1.31 | −1.32 | −1.40 | Mbtps2 |
| 1436918_at | Lgx only | 857 | 823 | 801 | 562 | −1.04 | −1.07 | −1.53 | LOC100044376 |
| 1436947_a_at | Lgx only | 4903 | 4267 | 3399 | 2703 | −1.15 | −1.44 | −1.81 | Txnl1 |
| 1436984_at | Lgx only | 752 | 734 | 574 | 497 | −1.02 | −1.31 | −1.51 | Abi2 |
| 1436985_at | Res & Lgx | 1800 | 1660 | 1487 | 1422 | −1.08 | −1.21 | −1.27 | Zfp644 |
| 1436999_at | Res only | 619 | 512 | 464 | 480 | −1.21 | −1.33 | −1.29 | 5033414K04Rik |
| 1437026_at | Lgx only | 472 | 457 | 287 | 194 | −1.03 | −1.65 | −2.44 | BC057893 |
| 1437069_at | Lgx only | 1632 | 1471 | 1282 | 972 | −1.11 | −1.27 | −1.68 | Osbpl8 |
| 1437077_at | Res & Lgx | 2086 | 1987 | 1493 | 1649 | −1.05 | −1.40 | −1.26 | Dcun1d2 |
| 1437092_at | Res & Lgx | 535 | 440 | 282 | 286 | −1.22 | −1.90 | −1.87 | LOC100048376 |
| 1437111_at | Lgx only | 474 | 441 | 430 | 320 | −1.08 | −1.10 | −1.48 | Zc3h12c |
| 1437136_at | Res only | 60 | 130 | 240 | 141 | 2.17 | 4.00 | 2.35 | 5830436I19Rik |
| 1437143_a_at | All | 4498 | 3397 | 2547 | 1894 | −1.32 | −1.77 | −2.38 | Txndc1 |
| 1437148_at | CR & Lgx | 7652 | 9004 | 8835 | 9409 | 1.18 | 1.15 | 1.23 | Arpc2 |
| 1437149_at | Lgx only | 2370 | 2826 | 3182 | 3347 | 1.19 | 1.34 | 1.41 | Slc6a6 |
| 1437151_at | Lgx only | 1757 | 1972 | 2085 | 2237 | 1.12 | 1.19 | 1.27 | Usp22 |
| 1437216_at | Lgx only | 363 | 289 | 295 | 210 | −1.25 | −1.23 | −1.73 | Ccdc88a |
| 1437236_a_at | Lgx only | 449 | 348 | 371 | 302 | −1.29 | −1.21 | −1.49 | Zfp110 |
| 1437241_at | CR only | 760 | 1025 | 755 | 926 | 1.35 | −1.01 | 1.22 | Klf11 |
| 1437283_at | Lgx only | 376 | 462 | 480 | 662 | 1.23 | 1.28 | 1.76 | Tnpo2 |
| 1437287_at | Res & Lgx | 2217 | 1647 | 1611 | 1014 | −1.35 | −1.38 | −2.19 | 1110020G09Rik |
| 1437290_at | Lgx only | 1027 | 946 | 711 | 722 | −1.09 | −1.44 | −1.42 | Impad1 |
| 1437354_at | Res & Lgx | 1278 | 1069 | 925 | 811 | −1.20 | −1.38 | −1.58 | C230091D08Rik |
| 1437382_at | Lgx only | 1211 | 1154 | 1069 | 922 | −1.05 | −1.13 | −1.31 | Acvr2a |
| 1437394_at | Lgx only | 343 | 383 | 446 | 567 | 1.12 | 1.30 | 1.65 | Centg2 |
| 1437397_at | Res & Lgx | 277 | 210 | 181 | 160 | −1.32 | −1.53 | −1.73 | Prlr |
| 1437398_a_at | All | 781 | 1052 | 1289 | 1566 | 1.35 | 1.65 | 2.01 | Aldh9a1 |
| 1437403_at | Lgx only | 41 | 70 | 69 | 98 | 1.72 | 1.69 | 2.41 | Samd5 |
| 1437405_a_at | Lgx only | 5956 | 7647 | 8743 | 11879 | 1.28 | 1.47 | 1.99 | Igfbp4 |
| 1437426_at | Lgx only | 275 | 200 | 273 | 162 | −1.37 | −1.01 | −1.70 | Wac |
| 1437432_a_at | Res only | 220 | 153 | 98 | 165 | −1.44 | −2.25 | −1.33 | Trim12 |
| 1437442_at | Res & Lgx | 1223 | 963 | 774 | 621 | −1.27 | −1.58 | −1.97 | Pcdh7 |
| 1437449_at | Lgx only | 727 | 595 | 602 | 560 | −1.22 | −1.21 | −1.30 | Rsad1 |
| 1437482_at | Res & Lgx | 7128 | 6639 | 4396 | 3270 | −1.07 | −1.62 | −2.18 | Srd5a2l2 |
| 1437484_at | Res & Lgx | 321 | 372 | 527 | 541 | 1.16 | 1.64 | 1.68 | Zbtb5 |
| 1437513_a_at | Lgx only | 5779 | 5896 | 6781 | 7125 | 1.02 | 1.17 | 1.23 | Serinc1 |
| 1437533_at | Res & Lgx | 2000 | 1894 | 1502 | 1237 | −1.06 | −1.33 | −1.62 | Birc4 |
| 1437537_at | Res & Lgx | 249 | 339 | 340 | 398 | 1.36 | 1.36 | 1.60 | Casp9 |
| 1437704_at | Res & Lgx | 194 | 157 | 376 | 378 | −1.23 | 1.93 | 1.94 | 2900024O10Rik |
| 1437729_at | Lgx only | 1474 | 1094 | 793 | 412 | −1.35 | −1.86 | −3.58 | Rpl27a |
| 1437740_at | Lgx only | 763 | 1122 | 1005 | 1343 | 1.47 | 1.32 | 1.76 | Plekhm2 |
| 1437741_at | Lgx only | 2327 | 2390 | 1778 | 1560 | 1.03 | −1.31 | −1.49 | Rab21 |
| 1437785_at | Lgx only | 474 | 397 | 463 | 337 | −1.19 | −1.03 | −1.41 | Adamts9 |
| 1437869_at | Res & Lgx | 14570 | 13664 | 11826 | 11731 | −1.07 | −1.23 | −1.24 | 3222402P14Rik |
| 1437875_at | Lgx only | 2753 | 2607 | 2485 | 2181 | −1.06 | −1.11 | −1.26 | Bicd2 |
| 1437900_at | Lgx only | 566 | 564 | 505 | 437 | −1.00 | −1.12 | −1.30 | 4930523C07Rik |
| 1437917_at | Lgx only | 370 | 334 | 293 | 223 | −1.11 | −1.26 | −1.65 | D530037H12Rik |
| 1438024_at | Res & Lgx | 3610 | 3226 | 2484 | 2237 | −1.12 | −1.45 | −1.61 | Ccdc90a |
| 1438026_at | Lgx only | 623 | 498 | 498 | 393 | −1.25 | −1.25 | −1.58 | Zfp560 |
| 1438045_at | Lgx only | 737 | 678 | 702 | 495 | −1.09 | −1.05 | −1.49 | Eea1 |
| 1438047_at | Res & Lgx | 1053 | 1176 | 1296 | 1256 | 1.12 | 1.23 | 1.19 | Zfp384 |
| 1438062_at | CR only | 588 | 372 | 440 | 434 | −1.58 | −1.34 | −1.36 | 4832420A03Rik |
| 1438077_at | Lgx only | 18 | 38 | 17 | 62 | 2.11 | −1.09 | 3.42 | Nlrp4a |
| 1438097_at | Lgx only | 611 | 574 | 490 | 391 | −1.06 | −1.25 | −1.56 | Rab20 |
| 1438169_a_at | Lgx only | 747 | 494 | 424 | 347 | −1.51 | −1.76 | −2.15 | Frmd4b |
| 1438195_at | Lgx only | 2915 | 3104 | 2321 | 2279 | 1.06 | −1.26 | −1.28 | Gpd1l |
| 1438208_at | Res only | 286 | 277 | 424 | 361 | −1.03 | 1.48 | 1.26 | Taok2 |
| 1438213_at | Lgx only | 15 | 30 | 39 | 79 | 2.00 | 2.61 | 5.30 | A830018L16Rik |
| 1438229_at | Res only | 843 | 887 | 609 | 701 | 1.05 | −1.38 | −1.20 | Pggt1b |
| 1438238_at | Lgx only | 231 | 217 | 198 | 106 | −1.07 | −1.17 | −2.18 | 2010315B03Rik |
| 1438241_at | Lgx only | 525 | 593 | 734 | 837 | 1.13 | 1.40 | 1.59 | Rgma |
| 1438258_at | CR & Res | 3530 | 2388 | 4783 | 3703 | −1.48 | 1.35 | 1.05 | Vldlr |
| 1438340_at | Lgx only | 219 | 117 | 157 | 125 | −1.86 | −1.40 | −1.75 | A930006D11Rik |
| 1438349_at | Lgx only | 195 | 145 | 121 | 75 | −1.34 | −1.61 | −2.60 | BC043476 |
| 1438400_at | Lgx only | 1226 | 1403 | 1426 | 1614 | 1.14 | 1.16 | 1.32 | 4632411B12Rik |
| 1438416_at | Lgx only | 1855 | 2525 | 2386 | 2992 | 1.36 | 1.29 | 1.61 | Med16 |
| 1438422_at | Res & Lgx | 1011 | 996 | 736 | 664 | −1.01 | −1.37 | −1.52 | Lrrc20 |
| 1438444_at | Lgx only | 9 | 29 | 35 | 26 | 3.33 | 3.99 | 3.02 | Spink10 |
| 1438510_a_at | Lgx only | 910 | 841 | 730 | 535 | −1.08 | −1.25 | −1.70 | Hars |
| 1438512_at | Res only | 68 | 151 | 177 | 104 | 2.21 | 2.59 | 1.52 | BC048679 |
| 1438532_at | Lgx only | 292 | 307 | 310 | 176 | 1.05 | 1.06 | −1.66 | Hmcn1 |
| 1438610_a_at | Lgx only | 887 | 695 | 489 | 435 | −1.28 | −1.82 | −2.04 | Cryz |
| 1438658_a_at | All | 1738 | 1228 | 1017 | 715 | −1.42 | −1.71 | −2.43 | Edg3 |
| 1438673_at | Lgx only | 759 | 586 | 535 | 411 | −1.29 | −1.42 | −1.85 | Slc4a7 |
| 1438676_at | CR & Lgx | 499 | 296 | 432 | 285 | −1.68 | −1.16 | −1.75 | Mpa2l |
| 1438678_at | Lgx only | 3261 | 2863 | 2608 | 2178 | −1.14 | −1.25 | −1.50 | 1500011K16Rik |
| 1438691_at | Res only | 664 | 655 | 437 | 488 | −1.01 | −1.52 | −1.36 | Zzef1 |
| 1438693_at | Res & Lgx | 311 | 353 | 461 | 515 | 1.14 | 1.48 | 1.66 | Tmem110 |
| 1438895_at | Res only | 571 | 519 | 762 | 647 | −1.10 | 1.33 | 1.13 | A430102J17Rik |
| 1438908_at | Lgx only | 246 | 204 | 303 | 415 | −1.21 | 1.23 | 1.69 | Map3k12 |
| 1438909_at | Lgx only | 51 | 62 | 76 | 173 | 1.23 | 1.49 | 3.41 | Sccpdh |
| 1438910_a_at | Lgx only | 3956 | 3657 | 4466 | 5068 | −1.08 | 1.13 | 1.28 | Stom |
| 1439008_at | Lgx only | 251 | 272 | 389 | 478 | 1.09 | 1.55 | 1.91 | Zfp319 |
| 1439010_at | Lgx only | 1107 | 987 | 790 | 675 | −1.12 | −1.40 | −1.64 | Larp4 |
| 1439024_at | CR only | 297 | 211 | 222 | 198 | −1.41 | −1.34 | −1.50 | Bag4 |
| 1439029_at | Lgx only | 629 | 843 | 709 | 1038 | 1.34 | 1.13 | 1.65 | Gpt2 |
| 1439055_at | Lgx only | 13 | 23 | 23 | 25 | 1.76 | 1.80 | 1.89 | OTTMUSG- |
| | | | | | | | | | 00000001305 |
| 1439078_at | Lgx only | 188 | 135 | 137 | 90 | −1.39 | −1.37 | −2.08 | Klhl4 |
| 1439143_at | CR & Lgx | 411 | 604 | 540 | 582 | 1.47 | 1.32 | 1.42 | A930018M24Rik |
| 1439153_at | Lgx only | 917 | 1123 | 739 | 645 | 1.22 | −1.24 | −1.42 | Ibrdc2 |
| 1439189_at | CR only | 592 | 915 | 521 | 472 | 1.54 | −1.14 | −1.25 | D630023B12Rik |
| 1439244_a_at | Lgx only | 2004 | 1777 | 2537 | 3175 | −1.13 | 1.27 | 1.58 | Tnrc6a |
| 1439266_a_at | Res & Lgx | 524 | 404 | 272 | 229 | −1.30 | −1.93 | −2.29 | Polr3k |
| 1439364_a_at | Lgx only | 1881 | 2097 | 2565 | 2782 | 1.11 | 1.36 | 1.48 | Mmp2 |
| 1439397_at | All | 322 | 190 | 197 | 172 | −1.69 | −1.63 | −1.88 | Fmn1 |
| 1439460_a_at | All | 2411 | 1816 | 1398 | 1125 | −1.33 | −1.73 | −2.14 | Zfp289 |
| 1439483_at | Res only | 47 | 63 | 129 | 96 | 1.33 | 2.71 | 2.02 | AI506816 |
| 1439488_at | Lgx only | 427 | 660 | 693 | 889 | 1.55 | 1.62 | 2.08 | Dot1l |
| 1439509_at | Res only | 161 | 198 | 257 | 226 | 1.22 | 1.59 | 1.40 | 2900008C10Rik |
| 1439529_at | Res & Lgx | 293 | 347 | 451 | 554 | 1.18 | 1.54 | 1.89 | A430110N23Rik |
| 1439675_at | Res only | 1299 | 1589 | 964 | 977 | 1.22 | −1.35 | −1.33 | 4933429D07Rik |
| 1439793_at | Lgx only | 539 | 391 | 382 | 304 | −1.38 | −1.41 | −1.77 | Gja3 |
| 1439797_at | Lgx only | 324 | 388 | 353 | 514 | 1.20 | 1.09 | 1.59 | Ppard |
| 1439815_at | Lgx only | 1354 | 1602 | 1962 | 2028 | 1.18 | 1.45 | 1.50 | Heatr5b |
| 1439821_at | Res only | 147 | 79 | 51 | 75 | −1.86 | −2.91 | −1.96 | Lrp2bp |
| 1440018_at | Lgx only | 32 | 47 | 70 | 136 | 1.48 | 2.21 | 4.30 | A330043J11Rik |
| 1440096_at | Lgx only | 360 | 318 | 281 | 177 | −1.13 | −1.28 | −2.03 | Ecm2 |
| 1440143_at | Lgx only | 141 | 181 | 180 | 383 | 1.28 | 1.28 | 2.71 | Pigz |
| 1440232_at | Res only | 47 | 67 | 145 | 121 | 1.41 | 3.07 | 2.55 | EG622645 |
| 1440234_at | Lgx only | 1187 | 1250 | 1054 | 915 | 1.05 | −1.13 | −1.30 | 1810012P15Rik |
| 1440279_at | Lgx only | 326 | 267 | 247 | 189 | −1.22 | −1.32 | −1.73 | Txndc10 |
| 1440335_at | Lgx only | 2118 | 2238 | 2120 | 1677 | 1.06 | 1.00 | −1.26 | LOC100046468 |
| 1440478_at | Res only | 176 | 92 | 68 | 123 | −1.90 | −2.57 | −1.43 | LOC100047601 |
| 1440480_at | Lgx only | 60 | 174 | 203 | 293 | 2.89 | 3.38 | 4.87 | AB182283 |
| 1440520_a_at | CR only | 147 | 330 | 264 | 161 | 2.24 | 1.79 | 1.09 | 1700051A21Rik |
| 1440537_at | Res only | 313 | 264 | 495 | 395 | −1.19 | 1.58 | 1.26 | Kcnv2 |
| 1440542_at | Res only | 82 | 53 | 18 | 48 | −1.54 | −4.45 | −1.72 | 7420416P09Rik |
| 1440561_at | Lgx only | 65 | 85 | 111 | 158 | 1.30 | 1.70 | 2.43 | C87259 |
| 1440739_at | CR only | 524 | 357 | 538 | 443 | −1.47 | 1.03 | −1.18 | Vegfc |
| 1440781_at | CR only | 133 | 48 | 101 | 60 | −2.75 | −1.32 | −2.21 | B830007D08Rik |
| 1440831_at | Res only | 1257 | 1218 | 923 | 944 | −1.03 | −1.36 | −1.33 | Bach1 |
| 1440838_at | Lgx only | 431 | 523 | 579 | 624 | 1.21 | 1.34 | 1.45 | AI852064 |
| 1440841_at | Lgx only | 745 | 669 | 622 | 529 | −1.11 | −1.20 | −1.41 | BB217526 |
| 1440849_at | CR only | 660 | 558 | 616 | 573 | −1.18 | −1.07 | −1.15 | 6330417G04Rik |
| 1440874_at | CR only | 311 | 151 | 297 | 337 | −2.06 | −1.05 | 1.08 | Slco5a1 |
| 1440908_at | Lgx only | 346 | 365 | 317 | 547 | 1.06 | −1.09 | 1.58 | D030063E12 |
| 1440934_at | Lgx only | 102 | 101 | 173 | 224 | −1.00 | 1.70 | 2.20 | AW742931 |
| 1440969_at | All | 199 | 103 | 102 | 67 | −1.93 | −1.94 | −2.95 | BC030308 |
| 1441001_at | Lgx only | 52 | 82 | 140 | 150 | 1.59 | 2.70 | 2.91 | AI225934 |
| 1441075_at | Lgx only | 317 | 274 | 260 | 171 | −1.16 | −1.22 | −1.86 | Nostrin |
| 1441081_a_at | Lgx only | 202 | 199 | 256 | 320 | −1.02 | 1.27 | 1.58 | 1110038B12Rik |
| 1441139_at | CR only | 2413 | 2791 | 2076 | 2479 | 1.16 | −1.16 | 1.03 | ENSMUSG- |
| | | | | | | | | | 00000071543 |
| 1441174_a_at | Res & Lgx | 123 | 85 | 72 | 87 | −1.44 | −1.71 | −1.41 | Lmln |
| 1441229_at | Res & Lgx | 174 | 114 | 73 | 70 | −1.52 | −2.36 | −2.48 | D230019N24Rik |
| 1441266_at | Res & Lgx | 812 | 636 | 542 | 457 | −1.28 | −1.50 | −1.78 | Strn3 |
| 1441320_a_at | Res & Lgx | 602 | 634 | 812 | 873 | 1.05 | 1.35 | 1.45 | AI413194 |
| 1441438_at | CR only | 269 | 183 | 277 | 225 | −1.47 | 1.03 | −1.20 | Gpc6 |
| 1441536_at | Lgx only | 18 | 44 | 32 | 86 | 2.39 | 1.74 | 4.68 | Hmgcs1 |
| 1441590_at | Lgx only | 1067 | 1051 | 1373 | 1614 | −1.01 | 1.29 | 1.51 | Kcnj5 |
| 1441970_at | Lgx only | 419 | 391 | 340 | 302 | −1.07 | −1.23 | −1.39 | E430010N07Rik |
| 1441987_at | Res & Lgx | 585 | 488 | 465 | 395 | −1.20 | −1.26 | −1.48 | Mbd5 |
| 1442002_at | Lgx only | 348 | 254 | 303 | 186 | −1.37 | −1.15 | −1.87 | 7030402D04Rik |
| 1442027_at | Lgx only | 1531 | 1126 | 1055 | 790 | −1.36 | −1.45 | −1.94 | Nbeal1 |
| 1442050_at | Res & Lgx | 328 | 298 | 521 | 618 | −1.10 | 1.59 | 1.88 | Zfp608 |
| 1442064_at | Res & Lgx | 84 | 144 | 242 | 284 | 1.71 | 2.88 | 3.37 | AW556556 |
| 1442090_at | Res only | 33 | 77 | 108 | 83 | 2.30 | 3.24 | 2.47 | 8030463A06Rik |
| 1442174_at | Lgx only | 360 | 314 | 499 | 581 | −1.15 | 1.39 | 1.61 | Tspan18 |
| 1442186_at | Res & Lgx | 215 | 323 | 448 | 443 | 1.50 | 2.09 | 2.06 | LOC100045002 |
| 1442362_at | Res & Lgx | 165 | 113 | 51 | 64 | −1.45 | −3.22 | −2.59 | Gm104 |
| 1442434_at | Res only | 466 | 588 | 765 | 673 | 1.26 | 1.64 | 1.44 | D8Ertd82e |
| 1442659_at | Lgx only | 332 | 320 | 312 | 473 | −1.04 | −1.07 | 1.42 | Pcdh9 |
| 1442695_at | Lgx only | 49 | 67 | 48 | 156 | 1.35 | −1.03 | 3.15 | C030007I01Rik |
| 1442732_at | CR only | 380 | 683 | 596 | 574 | 1.80 | 1.57 | 1.51 | Hadhb |
| 1442757_at | CR & Res | 432 | 288 | 287 | 372 | −1.50 | −1.50 | −1.16 | Lrch1 |
| 1442867_at | Res only | 135 | 148 | 262 | 224 | 1.10 | 1.94 | 1.65 | Megf11 |
| 1442926_at | Lgx only | 10 | 49 | 42 | 75 | 5.07 | 4.40 | 7.80 | 1700011B04Rik |
| 1443103_at | CR only | 69 | 23 | 45 | 45 | −2.98 | −1.53 | −1.53 | D830046C22Rik |
| 1443416_at | Lgx only | 197 | 253 | 192 | 328 | 1.28 | −1.03 | 1.66 | C79741 |
| 1443483_at | Lgx only | 59 | 59 | 64 | 17 | 1.01 | 1.08 | −3.39 | Xlr5a |
| 1443632_at | CR only | 10172 | 12920 | 10060 | 11150 | 1.27 | −1.01 | 1.10 | Obscn |
| 1443682_at | Res only | 259 | 192 | 424 | 371 | −1.35 | 1.64 | 1.43 | AI662476 |
| 1443854_at | Lgx only | 405 | 288 | 380 | 192 | −1.41 | −1.07 | −2.11 | Hand2 |
| 1443856_at | Lgx only | 726 | 687 | 651 | 538 | −1.06 | −1.11 | −1.35 | Rabep1 |
| 1443896_at | Lgx only | 748 | 888 | 896 | 1033 | 1.19 | 1.20 | 1.38 | Tbc1d5 |
| 1443932_at | Res & Lgx | 2283 | 1935 | 1827 | 1552 | −1.18 | −1.25 | −1.47 | Klhdc1 |
| 1443935_at | Res only | 401 | 429 | 494 | 464 | 1.07 | 1.23 | 1.16 | BC032203 |
| 1444041_at | Lgx only | 146 | 127 | 94 | 62 | −1.15 | −1.55 | −2.35 | AU041133 |
| 1444112_at | Lgx only | 1457 | 1326 | 1343 | 1080 | −1.10 | −1.09 | −1.35 | ENSMUSG- |
| | | | | | | | | | 00000074466 |
| 1444217_at | Lgx only | 212 | 168 | 299 | 382 | −1.26 | 1.41 | 1.80 | Mrpl38 |
| 1444254_at | CR only | 84 | 28 | 74 | 105 | −2.95 | −1.12 | 1.26 | Tns4 |
| 1444294_at | Lgx only | 127 | 204 | 249 | 265 | 1.61 | 1.97 | 2.10 | 4930551A22Rik |
| 1444370_at | CR only | 46 | 16 | 60 | 24 | −2.80 | 1.31 | −1.91 | C77058 |
| 1444456_at | Lgx only | 245 | 268 | 240 | 176 | 1.09 | −1.02 | −1.39 | 9030425P06Rik |
| 1444494_at | All | 2200 | 1830 | 1616 | 1601 | −1.20 | −1.36 | −1.37 | Kbtbd10 |
| 1444537_at | Lgx only | 219 | 296 | 347 | 346 | 1.35 | 1.59 | 1.58 | AI429363 |
| 1444684_at | Lgx only | 50 | 23 | 43 | 16 | −2.18 | −1.15 | −3.18 | 8030475D13Rik |
| 1444766_at | Lgx only | 107 | 102 | 101 | 31 | −1.04 | −1.06 | −3.48 | Atxn7I1 |
| 1445191_at | CR only | 187 | 125 | 201 | 197 | −1.50 | 1.08 | 1.05 | Exdl1 |
| 1445459_at | Lgx only | 50 | 100 | 93 | 186 | 2.00 | 1.87 | 3.72 | Sstr5 |
| 1446062_at | Res only | 58 | 100 | 121 | 102 | 1.74 | 2.10 | 1.77 | B830028B13Rik |
| 1446214_at | CR only | 18 | 68 | 77 | 45 | 3.67 | 4.16 | 2.45 | D430018E03Rik |
| 1446678_at | Lgx only | 37 | 60 | 62 | 121 | 1.62 | 1.68 | 3.26 | D14Ertd16e |
| 1446781_at | CR only | 95 | 21 | 55 | 68 | −4.54 | −1.73 | −1.39 | D8Ertd54e |
| 1446812_at | Res & Lgx | 890 | 716 | 584 | 552 | −1.24 | −1.52 | −1.61 | LOC100040515 |
| 1446842_at | CR only | 68 | 13 | 26 | 27 | −5.38 | −2.60 | −2.47 | D4Ertd571e |
| 1447035_at | Lgx only | 41 | 91 | 109 | 139 | 2.21 | 2.63 | 3.36 | A230091C14Rik |
| 1447095_at | Lgx only | 109 | 130 | 166 | 195 | 1.19 | 1.52 | 1.79 | C86942 |
| 1447923_at | Lgx only | 781 | 678 | 544 | 448 | −1.15 | −1.44 | −1.74 | 1810026B05Rik |
| 1447967_at | Res only | 256 | 260 | 440 | 360 | 1.01 | 1.72 | 1.41 | Tmem69 |
| 1447981_at | CR only | 68 | 13 | 66 | 56 | −5.40 | −1.03 | −1.21 | C78441 |
| 1448091_at | CR & Lgx | 18 | 85 | 41 | 75 | 4.74 | 2.28 | 4.20 | D15Ertd50e |
| 1448100_at | Res & Lgx | 4461 | 4342 | 3733 | 3467 | −1.03 | −1.20 | −1.29 | 4833439L19Rik |
| 1448116_at | Res & Lgx | 3337 | 3991 | 4522 | 5277 | 1.20 | 1.35 | 1.58 | Ube1x |
| 1448122_at | CR & Lgx | 8342 | 9257 | 8220 | 9546 | 1.11 | −1.01 | 1.14 | Tcp1 |
| 1448143_at | Lgx only | 6719 | 7951 | 7830 | 8811 | 1.18 | 1.17 | 1.31 | Aldh2 |
| 1448145_at | Lgx only | 304 | 323 | 454 | 535 | 1.06 | 1.50 | 1.76 | Wwp2 |
| 1448148_at | Res & Lgx | 1666 | 2147 | 2587 | 3224 | 1.29 | 1.55 | 1.94 | Grn |
| 1448153_at | Lgx only | 60488 | 68353 | 63338 | 84246 | 1.13 | 1.05 | 1.39 | Cox5a |
| 1448154_at | Lgx only | 19344 | 22511 | 19920 | 25912 | 1.16 | 1.03 | 1.34 | Ndrg2 |
| 1448163_at | Lgx only | 798 | 1050 | 981 | 1240 | 1.32 | 1.23 | 1.55 | Gnpda1 |
| 1448167_at | Lgx only | 684 | 808 | 806 | 1130 | 1.18 | 1.18 | 1.65 | Ifngr1 |
| 1448174_at | Lgx only | 1875 | 1928 | 2181 | 2232 | 1.03 | 1.16 | 1.19 | Cul1 |
| 1448181_at | CR only | 1549 | 2211 | 1745 | 1742 | 1.43 | 1.13 | 1.12 | Klf15 |
| 1448185_at | Lgx only | 5495 | 7055 | 5636 | 8302 | 1.28 | 1.03 | 1.51 | Herpud1 |
| 1448188_at | Lgx only | 5526 | 6650 | 8466 | 12288 | 1.20 | 1.53 | 2.22 | Ucp2 |
| 1448189_a_at | Lgx only | 2277 | 2853 | 3180 | 3351 | 1.25 | 1.40 | 1.47 | Flii |
| 1448196_at | Res & Lgx | 3368 | 2651 | 2008 | 1537 | −1.27 | −1.68 | −2.19 | Mat2b |
| 1448198_a_at | Lgx only | 38229 | 43229 | 40539 | 48367 | 1.13 | 1.06 | 1.27 | Ndufb8 |
| 1448199_at | Res only | 679 | 631 | 507 | 543 | −1.08 | −1.34 | −1.25 | Ankrd10 |
| 1448206_at | Lgx only | 6419 | 7041 | 7577 | 10844 | 1.10 | 1.18 | 1.69 | Psma2 |
| 1448208_at | Lgx only | 632 | 474 | 499 | 348 | −1.34 | −1.27 | −1.82 | Smad1 |
| 1448209_a_at | Lgx only | 246 | 316 | 363 | 502 | 1.29 | 1.48 | 2.04 | Slc22a17 |
| 1448212_at | Lgx only | 1004 | 973 | 746 | 707 | −1.03 | −1.35 | −1.42 | Tnfsf5ip1 |
| 1448221_at | CR only | 4042 | 4996 | 4086 | 4247 | 1.24 | 1.01 | 1.05 | Bat1a |
| 1448224_at | Lgx only | 1460 | 1359 | 1117 | 995 | −1.07 | −1.31 | −1.47 | Tfam |
| 1448225_at | Lgx only | 498 | 691 | 692 | 796 | 1.39 | 1.39 | 1.60 | Gpaa1 |
| 1448240_at | Lgx only | 1199 | 1319 | 1410 | 1668 | 1.10 | 1.18 | 1.39 | Mbtps1 |
| 1448242_at | Lgx only | 1162 | 1336 | 1617 | 1835 | 1.15 | 1.39 | 1.58 | Sec61a1 |
| 1448244_at | Lgx only | 1573 | 1505 | 1192 | 813 | −1.05 | −1.32 | −1.93 | Lypla1 |
| 1448252_a_at | Lgx only | 7588 | 7012 | 6014 | 5451 | −1.08 | −1.26 | −1.39 | Eef1b2 |
| 1448258_a_at | Lgx only | 6413 | 6297 | 5305 | 4246 | −1.02 | −1.21 | −1.51 | Spcs1 |
| 1448269_a_at | Res & Lgx | 613 | 517 | 374 | 350 | −1.19 | −1.64 | −1.75 | Klhl13 |
| 1448276_at | Lgx only | 1808 | 2156 | 1862 | 2730 | 1.19 | 1.03 | 1.51 | Tspan4 |
| 1448284_a_at | Lgx only | 37586 | 40364 | 44938 | 56730 | 1.07 | 1.20 | 1.51 | Ndufc1 |
| 1448287_at | Lgx only | 4436 | 4040 | 3639 | 2830 | −1.10 | −1.22 | −1.57 | Rpo1-3 |
| 1448304_a_at | Res & Lgx | 3053 | 2867 | 2183 | 1770 | −1.06 | −1.40 | −1.72 | Rab6 |
| 1448313_at | Lgx only | 2609 | 2807 | 3061 | 3302 | 1.08 | 1.17 | 1.27 | Tpp1 |
| 1448325_at | Res & Lgx | 757 | 893 | 1112 | 1080 | 1.18 | 1.47 | 1.43 | Myd116 |
| 1448327_at | Lgx only | 30728 | 36837 | 37776 | 43889 | 1.20 | 1.23 | 1.43 | Actn2 |
| 1448330_at | Res & Lgx | 7365 | 8521 | 9720 | 11395 | 1.16 | 1.32 | 1.55 | Gstm1 |
| 1448336_at | Lgx only | 1711 | 1622 | 1611 | 1433 | −1.05 | −1.06 | −1.19 | Drg1 |
| 1448339_at | Res & Lgx | 4104 | 3995 | 2913 | 2634 | −1.03 | −1.41 | −1.56 | Tmem30a |
| 1448341_a_at | Lgx only | 321 | 317 | 503 | 653 | −1.01 | 1.57 | 2.04 | Stxbp2 |
| 1448345_at | Lgx only | 1081 | 1303 | 1204 | 1435 | 1.21 | 1.11 | 1.33 | Tomm34 |
| 1448346_at | CR only | 2920 | 2207 | 2287 | 2450 | −1.32 | −1.28 | −1.19 | Cfl1 |
| 1448351_at | Lgx only | 1035 | 1248 | 1300 | 1665 | 1.21 | 1.26 | 1.61 | Coro1b |
| 1448356_at | Lgx only | 2083 | 1903 | 1705 | 1572 | −1.09 | −1.22 | −1.32 | Ube2d2 |
| 1448362_at | Lgx only | 1772 | 2097 | 2189 | 2519 | 1.18 | 1.24 | 1.42 | Dnajc7 |
| 1448363_at | Res & Lgx | 2486 | 2740 | 3232 | 3785 | 1.10 | 1.30 | 1.52 | Yap1 |
| 1448365_at | Res & Lgx | 1790 | 1723 | 1184 | 1227 | −1.04 | −1.51 | −1.46 | Exosc7 |
| 1448379_at | Res only | 647 | 563 | 413 | 518 | −1.15 | −1.57 | −1.25 | Pot1a |
| 1448380_at | Res & Lgx | 1020 | 1208 | 1439 | 1659 | 1.19 | 1.41 | 1.63 | Lgals3bp |
| 1448388_a_at | Lgx only | 2505 | 2405 | 1802 | 1158 | −1.04 | −1.39 | −2.16 | 1110002B05Rik |
| 1448402_at | Lgx only | 1415 | 1869 | 2287 | 2869 | 1.32 | 1.62 | 2.03 | Tln1 |
| 1448412_a_at | Lgx only | 3472 | 4076 | 4244 | 4721 | 1.17 | 1.22 | 1.36 | Tsc22d4 |
| 1448415_a_at | Lgx only | 387 | 438 | 593 | 653 | 1.13 | 1.53 | 1.69 | Sema3b |
| 1448416_at | Lgx only | 16601 | 20394 | 19345 | 31156 | 1.23 | 1.17 | 1.88 | Mgp |
| 1448417_at | Lgx only | 1000 | 1181 | 1301 | 1299 | 1.18 | 1.30 | 1.30 | Ninj1 |
| 1448429_at | Res & Lgx | 3339 | 3198 | 2421 | 1974 | −1.04 | −1.38 | −1.69 | Gyg |
| 1448430_a_at | CR & Lgx | 8516 | 10720 | 9639 | 10638 | 1.26 | 1.13 | 1.25 | Naca |
| 1448432_at | Lgx only | 369 | 514 | 544 | 661 | 1.40 | 1.48 | 1.79 | Plcd1 |
| 1448438_at | Lgx only | 2204 | 1814 | 1971 | 1577 | −1.22 | −1.12 | −1.40 | Derl2 |
| 1448463_at | Lgx only | 4645 | 4072 | 3480 | 3091 | −1.14 | −1.33 | −1.50 | 4933434E20Rik |
| 1448467_a_at | Lgx only | 1218 | 1447 | 1612 | 1817 | 1.19 | 1.32 | 1.49 | Ehbp1l1 |
| 1448476_at | CR & Lgx | 5612 | 6609 | 6127 | 7217 | 1.18 | 1.09 | 1.29 | Nap1l4 |
| 1448480_at | Res & Lgx | 1000 | 728 | 556 | 360 | −1.37 | −1.80 | −2.78 | Nip7 |
| 1448484_at | Res & Lgx | 5744 | 5110 | 3530 | 3419 | −1.12 | −1.63 | −1.68 | Amd1 |
| 1448488_at | CR & Lgx | 831 | 1036 | 919 | 1013 | 1.25 | 1.11 | 1.22 | Mrps5 |
| 1448492_a_at | Res & Lgx | 4733 | 4441 | 3842 | 3020 | −1.07 | −1.23 | −1.57 | Psmd12 |
| 1448493_at | CR & Lgx | 9202 | 6905 | 8442 | 6228 | −1.33 | −1.09 | −1.48 | Paip2 |
| 1448495_at | Lgx only | 723 | 911 | 890 | 1046 | 1.26 | 1.23 | 1.45 | Tsta3 |
| 1448498_at | Lgx only | 793 | 1061 | 951 | 1228 | 1.34 | 1.20 | 1.55 | Rps6ka4 |
| 1448505_at | Res & Lgx | 1738 | 1400 | 1033 | 860 | −1.24 | −1.68 | −2.02 | C1d |
| 1448508_at | Lgx only | 237 | 284 | 322 | 442 | 1.20 | 1.36 | 1.86 | Traf3ip2 |
| 1448517_at | Lgx only | 2047 | 2144 | 1562 | 1376 | 1.05 | −1.31 | −1.49 | Timm22 |
| 1448527_at | Lgx only | 1955 | 1753 | 1345 | 1338 | −1.12 | −1.45 | −1.46 | Pdcd10 |
| 1448533_at | Res & Lgx | 3076 | 2967 | 2613 | 2263 | −1.04 | −1.18 | −1.36 | Tbcb |
| 1448535_at | Lgx only | 306 | 250 | 237 | 154 | −1.22 | −1.29 | −1.98 | Elp4 |
| 1448536_at | Lgx only | 4579 | 4812 | 3261 | 2727 | 1.05 | −1.40 | −1.68 | Lsm3 |
| 1448537_at | Res & Lgx | 2417 | 2026 | 1762 | 1591 | −1.19 | −1.37 | −1.52 | Ttc1 |
| 1448543_at | Res & Lgx | 1715 | 1494 | 908 | 822 | −1.15 | −1.89 | −2.09 | Slmo2 |
| 1448548_at | Lgx only | 1328 | 1385 | 1288 | 1631 | 1.04 | −1.03 | 1.23 | Tulp4 |
| 1448549_a_at | Res only | 1179 | 1068 | 850 | 1106 | −1.10 | −1.39 | −1.07 | Dpagt1 |
| 1448559_at | Lgx only | 5131 | 5547 | 6227 | 7143 | 1.08 | 1.21 | 1.39 | Flot1 |
| 1448564_at | Res only | 511 | 609 | 713 | 573 | 1.19 | 1.40 | 1.12 | Cib1 |
| 1448565_at | Lgx only | 2831 | 2386 | 2740 | 1942 | −1.19 | −1.03 | −1.46 | Ppp1r11 |
| 1448567_at | Lgx only | 590 | 668 | 763 | 806 | 1.13 | 1.29 | 1.37 | Tmem115 |
| 1448568_a_at | CR only | 688 | 524 | 629 | 533 | −1.31 | −1.09 | −1.29 | Slc20a1 |
| 1448570_at | Lgx only | 1666 | 1576 | 1258 | 948 | −1.06 | −1.32 | −1.76 | Gmfb |
| 1448579_at | Lgx only | 2689 | 2976 | 3392 | 3692 | 1.11 | 1.26 | 1.37 | Glg1 |
| 1448585_at | Res only | 751 | 809 | 578 | 655 | 1.08 | −1.30 | −1.15 | Gtf2h4 |
| 1448591_at | CR only | 1010 | 720 | 1099 | 736 | −1.40 | 1.09 | −1.37 | Ctss |
| 1448613_at | Lgx only | 1206 | 1308 | 1426 | 1969 | 1.08 | 1.18 | 1.63 | Ecm1 |
| 1448615_at | Res & Lgx | 769 | 858 | 1028 | 1113 | 1.12 | 1.34 | 1.45 | Ccs |
| 1448621_a_at | Res & Lgx | 7092 | 7555 | 5213 | 5291 | 1.07 | −1.36 | −1.34 | Smpd1 |
| 1448623_at | Lgx only | 1699 | 1287 | 1178 | 807 | −1.32 | −1.44 | −2.11 | Tmem123 |
| 1448625_at | Res & Lgx | 1612 | 1957 | 2268 | 2558 | 1.21 | 1.41 | 1.59 | Golga2 |
| 1448637_at | Lgx only | 1033 | 1248 | 1507 | 1924 | 1.21 | 1.46 | 1.86 | Med25 |
| 1448638_at | Res only | 68 | 79 | 140 | 150 | 1.17 | 2.06 | 2.21 | Mtbp |
| 1448644_at | Lgx only | 1286 | 1471 | 1597 | 1684 | 1.14 | 1.24 | 1.31 | Pef1 |
| 1448645_at | Lgx only | 2239 | 2328 | 1993 | 1844 | 1.04 | −1.12 | −1.21 | Msl31 |
| 1448649_at | Lgx only | 540 | 500 | 484 | 392 | −1.08 | −1.12 | −1.38 | Enpep |
| 1448684_at | Res & Lgx | 4396 | 3940 | 3485 | 3517 | −1.12 | −1.26 | −1.25 | Ppp1r2 |
| 1448696_at | Res only | 95 | 115 | 47 | 40 | 1.20 | −2.03 | −2.37 | Heph |
| 1448700_at | Lgx only | 4037 | 3143 | 2765 | 2331 | −1.28 | −1.46 | −1.73 | G0s2 |
| 1448717_at | CR only | 889 | 671 | 980 | 830 | −1.32 | 1.10 | −1.07 | Gcdh |
| 1448720_at | Lgx only | 342 | 284 | 279 | 229 | −1.21 | −1.23 | −1.49 | Lrrc40 |
| 1448724_at | Res only | 4162 | 4532 | 2682 | 2820 | 1.09 | −1.55 | −1.48 | Cish |
| 1448727_at | Lgx only | 269 | 370 | 398 | 484 | 1.37 | 1.48 | 1.80 | Tle6 |
| 1448729_a_at | CR & Lgx | 1559 | 1304 | 1446 | 1143 | −1.20 | −1.08 | −1.36 | 39329 |
| 1448737_at | Lgx only | 4695 | 4240 | 4264 | 3613 | −1.11 | −1.10 | −1.30 | Tspan7 |
| 1448760_at | Lgx only | 643 | 554 | 539 | 457 | −1.16 | −1.19 | −1.41 | Zfp68 |
| 1448762_at | Res & Lgx | 1357 | 1116 | 910 | 676 | −1.22 | −1.49 | −2.01 | Rad17 |
| 1448769_at | Lgx only | 2415 | 2426 | 2058 | 1875 | 1.00 | −1.17 | −1.29 | Slc35b1 |
| 1448770_a_at | Lgx only | 6679 | 6835 | 5272 | 3909 | 1.02 | −1.27 | −1.71 | Atpif1 |
| 1448771_a_at | Lgx only | 49028 | 51355 | 54395 | 64994 | 1.05 | 1.11 | 1.33 | Fth1 |
| 1448788_at | Res & Lgx | 1474 | 1445 | 1194 | 1065 | −1.02 | −1.24 | −1.38 | Cd200 |
| 1448792_a_at | Res only | 137 | 80 | 49 | 1448 | −1.71 | −2.79 | 10.55 | Cyp2f2 |
| 1448797_at | Res & Lgx | 945 | 842 | 674 | 584 | −1.12 | −1.40 | −1.62 | Elk3 |
| 1448809_at | Res & Lgx | 2605 | 2313 | 1681 | 1641 | −1.13 | −1.55 | −1.59 | Cse1l |
| 1448810_at | Res & Lgx | 397 | 445 | 638 | 701 | 1.12 | 1.61 | 1.77 | Gne |
| 1448826_at | Lgx only | 95590 | 115497 | 118388 | 173815 | 1.21 | 1.24 | 1.82 | Myh6 |
| 1448830_at | Lgx only | 5138 | 4291 | 4152 | 3187 | −1.20 | −1.24 | −1.61 | Dusp1 |
| 1448835_at | Res & Lgx | 5346 | 5044 | 3924 | 3656 | −1.06 | −1.36 | −1.46 | E2f6 |
| 1448838_at | Lgx only | 386 | 350 | 281 | 214 | −1.10 | −1.37 | −1.81 | Topors |
| 1448840_at | Lgx only | 461 | 474 | 456 | 753 | 1.03 | −1.01 | 1.63 | Tmub1 |
| 1448844_at | Lgx only | 1373 | 1278 | 1179 | 983 | −1.07 | −1.17 | −1.40 | Cyb5b |
| 1448853_at | Lgx only | 5831 | 5559 | 4659 | 4095 | −1.05 | −1.25 | −1.42 | Synj2bp |
| 1448856_a_at | Res & Lgx | 2552 | 2477 | 2140 | 2204 | −1.03 | −1.19 | −1.16 | Msra |
| 1448860_at | Res only | 17 | 35 | 50 | 64 | 2.14 | 3.02 | 3.84 | Rem2 |
| 1448864_at | Lgx only | 3508 | 4978 | 4574 | 4515 | 1.42 | 1.30 | 1.29 | Snrk |
| 1448867_at | Lgx only | 2465 | 2370 | 1926 | 1977 | −1.04 | −1.28 | −1.25 | Tmem9b |
| 1448883_at | Lgx only | 986 | 1264 | 880 | 1339 | 1.28 | −1.12 | 1.36 | Lgmn |
| 1448884_at | Lgx only | 742 | 688 | 554 | 408 | −1.08 | −1.34 | −1.82 | Gtf2e2 |
| 1448885_at | CR only | 299 | 214 | 340 | 415 | −1.40 | 1.14 | 1.39 | Rap2b |
| 1448893_at | Lgx only | 851 | 1373 | 1459 | 1947 | 1.61 | 1.71 | 2.29 | Ncor2 |
| 1448894_at | Lgx only | 832 | 852 | 961 | 1170 | 1.02 | 1.15 | 1.41 | Akr1b8 |
| 1448900_at | Lgx only | 4895 | 4713 | 4330 | 3890 | −1.04 | −1.13 | −1.26 | D16H22S680E |
| 1448903_at | Lgx only | 7279 | 5973 | 5176 | 3901 | −1.22 | −1.41 | −1.87 | 39340 |
| 1448917_at | Lgx only | 1574 | 1229 | 1326 | 1221 | −1.28 | −1.19 | −1.29 | Med30 |
| 1448918_at | Lgx only | 1286 | 1478 | 1757 | 1991 | 1.15 | 1.37 | 1.55 | Slco3a1 |
| 1448943_at | Lgx only | 4428 | 4185 | 3289 | 2623 | −1.06 | −1.35 | −1.69 | Nrp1 |
| 1448947_at | Lgx only | 2361 | 2309 | 1900 | 1544 | −1.02 | −1.24 | −1.53 | 2810004N23Rik |
| 1448948_at | Lgx only | 1247 | 1146 | 1163 | 1058 | −1.09 | −1.07 | −1.18 | Rag1ap1 |
| 1448956_at | Lgx only | 3964 | 4452 | 6177 | 9601 | 1.12 | 1.56 | 2.42 | Stard10 |
| 1448960_at | Lgx only | 1075 | 1032 | 1164 | 1411 | −1.04 | 1.08 | 1.31 | Cxxc5 |
| 1448970_at | Lgx only | 3836 | 3551 | 3364 | 2960 | −1.08 | −1.14 | −1.30 | Slc25a46 |
| 1448971_at | Lgx only | 389 | 353 | 307 | 236 | −1.10 | −1.26 | −1.65 | 2410022L05Rik |
| 1448987_at | Lgx only | 43424 | 51619 | 52391 | 80140 | 1.19 | 1.21 | 1.85 | Acadl |
| 1448993_at | Res & Lgx | 2740 | 2404 | 1834 | 1517 | −1.14 | −1.49 | −1.81 | Atg3 |
| 1449014_at | Res only | 881 | 843 | 642 | 705 | −1.05 | −1.37 | −1.25 | Lactb |
| 1449025_at | Lgx only | 548 | 576 | 657 | 774 | 1.05 | 1.20 | 1.41 | Ifit3 |
| 1449026_at | Res & Lgx | 503 | 552 | 779 | 792 | 1.10 | 1.55 | 1.57 | Ifnar1 |
| 1449042_at | Lgx only | 936 | 870 | 1132 | 1241 | −1.08 | 1.21 | 1.33 | Ctcf |
| 1449044_at | Lgx only | 1437 | 1195 | 953 | 801 | −1.20 | −1.51 | −1.79 | Eef1e1 |
| 1449045_at | Lgx only | 952 | 1152 | 1135 | 1427 | 1.21 | 1.19 | 1.50 | Afg3l1 |
| 1449046_a_at | Lgx only | 1537 | 1426 | 1431 | 1340 | −1.08 | −1.07 | −1.15 | Josd2 |
| 1449058_at | Lgx only | 143 | 148 | 92 | 232 | 1.04 | −1.55 | 1.63 | Gli1 |
| 1449062_at | Lgx only | 347 | 452 | 475 | 608 | 1.30 | 1.37 | 1.75 | Khk |
| 1449063_at | Res only | 1262 | 1084 | 930 | 1007 | −1.16 | −1.36 | −1.25 | Sec22b |
| 1449071_at | Lgx only | 25977 | 29211 | 40937 | 73543 | 1.12 | 1.58 | 2.83 | Myl7 |
| 1449072_a_at | Lgx only | 1737 | 1759 | 1601 | 1044 | 1.01 | −1.09 | −1.66 | N6amt2 |
| 1449078_at | Res & Lgx | 1395 | 1028 | 1025 | 751 | −1.36 | −1.36 | −1.86 | St3gal6 |
| 1449080_at | All | 447 | 289 | 283 | 232 | −1.55 | −1.58 | −1.93 | Hdac2 |
| 1449088_at | Lgx only | 1611 | 1389 | 2284 | 2373 | −1.16 | 1.42 | 1.47 | Fbp2 |
| 1449096_at | Lgx only | 1028 | 1107 | 863 | 714 | 1.08 | −1.19 | −1.44 | Ccdc127 |
| 1449106_at | Lgx only | 16622 | 17727 | 24198 | 34324 | 1.07 | 1.46 | 2.07 | Gpx3 |
| 1449108_at | Lgx only | 7575 | 7320 | 6088 | 4914 | −1.03 | −1.24 | −1.54 | Fdx1 |
| 1449113_at | Res only | 848 | 830 | 647 | 687 | −1.02 | −1.31 | −1.23 | Gpbp1l1 |
| 1449123_at | Lgx only | 84 | 43 | 111 | 29 | −1.96 | 1.32 | −2.93 | Itih3 |
| 1449124_at | Res & Lgx | 310 | 370 | 480 | 455 | 1.19 | 1.55 | 1.47 | Rgl1 |
| 1449125_at | Lgx only | 202 | 143 | 142 | 96 | −1.42 | −1.43 | −2.11 | Tnfaip8l1 |
| 1449138_at | Lgx only | 3517 | 3750 | 4329 | 4360 | 1.07 | 1.23 | 1.24 | Sf3b1 |
| 1449140_at | Lgx only | 273 | 266 | 217 | 136 | −1.03 | −1.26 | −2.00 | Nudcd2 |
| 1449145_a_at | Lgx only | 11453 | 13083 | 13559 | 16570 | 1.14 | 1.18 | 1.45 | Cav1 |
| 1449151_at | Lgx only | 508 | 569 | 665 | 675 | 1.12 | 1.31 | 1.33 | Pctk3 |
| 1449187_at | Lgx only | 529 | 457 | 392 | 333 | −1.16 | −1.35 | −1.59 | Pdgfa |
| 1449217_at | Lgx only | 230 | 198 | 132 | 111 | −1.16 | −1.74 | −2.07 | Casp8ap2 |
| 1449256_a_at | Lgx only | 2956 | 2919 | 3638 | 3430 | −1.01 | 1.23 | 1.16 | Rab11a |
| 1449269_at | Lgx only | 168 | 147 | 139 | 61 | −1.15 | −1.21 | −2.75 | F5 |
| 1449281_at | Lgx only | 1873 | 2466 | 2400 | 2905 | 1.32 | 1.28 | 1.55 | Nrtn |
| 1449298_a_at | Lgx only | 237 | 268 | 246 | 322 | 1.13 | 1.04 | 1.36 | Pde1a |
| 1449300_at | Lgx only | 553 | 544 | 493 | 380 | −1.02 | −1.12 | −1.46 | Cttnbp2nl |
| 1449304_at | Lgx only | 463 | 404 | 649 | 649 | −1.15 | 1.40 | 1.40 | 2310061J03Rik |
| 1449333_at | Lgx only | 549 | 662 | 682 | 852 | 1.21 | 1.24 | 1.55 | Sf3a1 |
| 1449335_at | Lgx only | 5926 | 8089 | 8111 | 11301 | 1.37 | 1.37 | 1.91 | Timp3 |
| 1449338_at | Lgx only | 3319 | 3175 | 3019 | 2290 | −1.05 | −1.10 | −1.45 | D10Ertd641e |
| 1449345_at | Res only | 139 | 99 | 60 | 69 | −1.40 | −2.32 | −2.02 | Ccdc34 |
| 1449348_at | Lgx only | 2011 | 1876 | 1690 | 1475 | −1.07 | −1.19 | −1.36 | Mpp6 |
| 1449354_at | Res & Lgx | 959 | 1115 | 1378 | 1597 | 1.16 | 1.44 | 1.67 | Zrsr1 |
| 1449355_a_at | Lgx only | 676 | 967 | 976 | 975 | 1.43 | 1.44 | 1.44 | Eps15l1 |
| 1449356_at | Lgx only | 1478 | 999 | 1176 | 608 | −1.48 | −1.26 | −2.43 | Asb5 |
| 1449357_at | Res & Lgx | 286 | 192 | 172 | 175 | −1.49 | −1.67 | −1.64 | 2310030G06Rik |
| 1449363_at | Res only | 306 | 165 | 208 | 182 | −1.86 | −1.47 | −1.68 | Atf3 |
| 1449368_at | Lgx only | 25201 | 21347 | 33485 | 36603 | −1.18 | 1.33 | 1.45 | Dcn |
| 1449372_at | Res only | 1022 | 867 | 1303 | 1225 | −1.18 | 1.27 | 1.20 | Dnajc3a |
| 1449388_at | Lgx only | 707 | 620 | 583 | 439 | −1.14 | −1.21 | −1.61 | Thbs4 |
| 1449396_at | Lgx only | 250 | 389 | 302 | 405 | 1.56 | 1.21 | 1.62 | Aoc3 |
| 1449398_at | Lgx only | 8560 | 9487 | 11462 | 12020 | 1.11 | 1.34 | 1.40 | Rpl3l |
| 1449400_at | Lgx only | 22 | 43 | 45 | 102 | 1.93 | 2.04 | 4.58 | Csl |
| 1449408_at | Lgx only | 3014 | 3550 | 3724 | 3878 | 1.18 | 1.24 | 1.29 | Jam2 |
| 1449491_at | Lgx only | 1509 | 1650 | 1845 | 1973 | 1.09 | 1.22 | 1.31 | Card10 |
| 1449505_at | Res & Lgx | 2070 | 2142 | 1758 | 1736 | 1.03 | −1.18 | −1.19 | Kpna1 |
| 1449511_a_at | Lgx only | 661 | 691 | 781 | 854 | 1.05 | 1.18 | 1.29 | Ssbp4 |
| 1449514_at | CR only | 2473 | 1904 | 2807 | 2301 | −1.30 | 1.14 | −1.07 | Grk5 |
| 1449547_at | Res & Lgx | 2990 | 2491 | 1986 | 1510 | −1.20 | −1.51 | −1.98 | Asb14 |
| 1449551_at | Lgx only | 1623 | 1886 | 1869 | 1988 | 1.16 | 1.15 | 1.22 | Myo1c |
| 1449553_at | CR & Lgx | 868 | 640 | 1034 | 1201 | −1.36 | 1.19 | 1.38 | 2610200G18Rik |
| 1449566_at | Lgx only | 2519 | 2746 | 3461 | 3832 | 1.09 | 1.37 | 1.52 | Nkx2-5 |
| 1449575_a_at | Res & Lgx | 13795 | 15659 | 17129 | 18543 | 1.14 | 1.24 | 1.34 | Gstp1 |
| 1449588_at | Lgx only | 588 | 491 | 438 | 385 | −1.20 | −1.34 | −1.53 | Abca4 |
| 1449592_at | CR only | 1101 | 791 | 1255 | 1092 | −1.39 | 1.14 | −1.01 | Tcf15 |
| 1449813_at | Res only | 276 | 352 | 86 | 217 | 1.27 | −3.22 | −1.27 | Zfp30 |
| 1449818_at | CR only | 1563 | 2002 | 1765 | 1942 | 1.28 | 1.13 | 1.24 | Abcb4 |
| 1449842_at | Res & Lgx | 718 | 590 | 533 | 420 | −1.22 | −1.35 | −1.71 | 1810059G22Rik |
| 1449845_a_at | Lgx only | 745 | 860 | 974 | 1211 | 1.16 | 1.31 | 1.63 | Ephb4 |
| 1449849_a_at | Lgx only | 371 | 465 | 477 | 508 | 1.26 | 1.29 | 1.37 | Fbxl6 |
| 1449851_at | Lgx only | 442 | 679 | 726 | 810 | 1.54 | 1.64 | 1.83 | Per1 |
| 1449852_a_at | CR only | 6223 | 8480 | 5751 | 5735 | 1.36 | −1.08 | −1.09 | Ehd4 |
| 1449860_at | Lgx only | 730 | 574 | 585 | 447 | −1.27 | −1.25 | −1.63 | Higd1b |
| 1449872_at | Lgx only | 1222 | 1194 | 1026 | 765 | −1.02 | −1.19 | −1.60 | Hspb3 |
| 1449935_a_at | CR only | 3719 | 4445 | 4158 | 4213 | 1.20 | 1.12 | 1.13 | Dnaja3 |
| 1449942_a_at | Lgx only | 2010 | 2379 | 2324 | 2472 | 1.18 | 1.16 | 1.23 | Ilk |
| 1449944_a_at | Res only | 767 | 785 | 647 | 741 | 1.02 | −1.19 | −1.03 | Sec61a2 |
| 1449964_a_at | Lgx only | 2099 | 2072 | 3263 | 3012 | −1.01 | 1.55 | 1.43 | Mlycd |
| 1449969_at | Lgx only | 1482 | 1463 | 1356 | 1042 | −1.01 | −1.09 | −1.42 | Tmod4 |
| 1450016_at | Lgx only | 26432 | 25470 | 23049 | 19945 | −1.04 | −1.15 | −1.33 | Ccng1 |
| 1450023_at | Lgx only | 732 | 879 | 873 | 1230 | 1.20 | 1.19 | 1.68 | Gtpbp1 |
| 1450031_at | Lgx only | 1227 | 1176 | 1239 | 982 | −1.04 | 1.01 | −1.25 | Aff4 |
| 1450054_at | Lgx only | 2952 | 3635 | 3914 | 4173 | 1.23 | 1.33 | 1.41 | Add1 |
| 1450067_a_at | Res & Lgx | 2875 | 2957 | 2406 | 2199 | 1.03 | −1.19 | −1.31 | 1810034K20Rik |
| 1450086_at | Lgx only | 172 | 236 | 254 | 316 | 1.37 | 1.48 | 1.84 | Gmeb1 |
| 1450095_a_at | Lgx only | 387 | 339 | 395 | 207 | −1.14 | 1.02 | −1.87 | Acyp1 |
| 1450122_at | Lgx only | 539 | 488 | 473 | 337 | −1.11 | −1.14 | −1.60 | Ptprg |
| 1450123_at | Lgx only | 11629 | 13643 | 15609 | 19737 | 1.17 | 1.34 | 1.70 | Ryr2 |
| 1450138_a_at | Lgx only | 3319 | 3219 | 2766 | 2714 | −1.03 | −1.20 | −1.22 | Serpinb6a |
| 1450180_a_at | Lgx only | 38 | 71 | 80 | 181 | 1.85 | 2.09 | 4.70 | Rara |
| 1450195_at | Lgx only | 35 | 102 | 106 | 144 | 2.91 | 3.04 | 4.11 | Ndst4 |
| 1450199_a_at | Lgx only | 795 | 979 | 986 | 1080 | 1.23 | 1.24 | 1.36 | Stab1 |
| 1450203_at | Lgx only | 5141 | 5970 | 4705 | 4331 | 1.16 | −1.09 | −1.19 | Smyd1 |
| 1450308_a_at | Res & Lgx | 373 | 389 | 657 | 764 | 1.04 | 1.76 | 2.05 | Xrn1 |
| 1450355_a_at | Res only | 432 | 329 | 314 | 351 | −1.31 | −1.38 | −1.23 | Capg |
| 1450361_at | CR only | 129 | 52 | 132 | 65 | −2.47 | 1.02 | −1.99 | Prop1 |
| 1450376_at | Lgx only | 2003 | 2127 | 2276 | 2577 | 1.06 | 1.14 | 1.29 | Mxi1 |
| 1450377_at | Lgx only | 165 | 219 | 246 | 321 | 1.33 | 1.49 | 1.94 | LOC640441 |
| 1450395_at | Lgx only | 1580 | 1719 | 1822 | 2403 | 1.09 | 1.15 | 1.52 | Slc22a5 |
| 1450405_at | Res & Lgx | 725 | 643 | 354 | 320 | −1.13 | −2.05 | −2.27 | Mrpl19 |
| 1450409_a_at | Lgx only | 1748 | 1899 | 2253 | 2570 | 1.09 | 1.29 | 1.47 | 4930570C03Rik |
| 1450415_at | CR only | 56 | 17 | 56 | 44 | −3.33 | −1.01 | −1.29 | Pde6a |
| 1450424_a_at | CR only | 188 | 296 | 252 | 253 | 1.58 | 1.34 | 1.34 | Il18bp |
| 1450431_a_at | Res & Lgx | 11524 | 12897 | 16998 | 18939 | 1.12 | 1.48 | 1.64 | Nedd4 |
| 1450435_at | Lgx only | 277 | 284 | 353 | 397 | 1.03 | 1.27 | 1.44 | L1cam |
| 1450449_a_at | Lgx only | 3983 | 4570 | 4434 | 5830 | 1.15 | 1.11 | 1.46 | 2900002H16Rik |
| 1450490_at | Lgx only | 417 | 575 | 570 | 646 | 1.38 | 1.37 | 1.55 | Kcna7 |
| 1450519_a_at | Lgx only | 4324 | 5666 | 5682 | 6518 | 1.31 | 1.31 | 1.51 | Prkaca |
| 1450531_at | CR only | 102 | 24 | 54 | 78 | −4.30 | −1.90 | −1.30 | H2-Bl |
| 1450584_at | Res only | 25 | 69 | 107 | 61 | 2.76 | 4.32 | 2.45 | Hoxd11 |
| 1450623_at | Lgx only | 3269 | 3884 | 4094 | 4247 | 1.19 | 1.25 | 1.30 | Gnb2 |
| 1450627_at | Res & Lgx | 10383 | 11098 | 8410 | 8645 | 1.07 | −1.23 | −1.20 | Ank |
| 1450649_at | Lgx only | 1489 | 966 | 883 | 760 | −1.54 | −1.69 | −1.96 | Gng10 |
| 1450650_at | Lgx only | 987 | 1096 | 1732 | 1739 | 1.11 | 1.76 | 1.76 | Myo10 |
| 1450662_at | Lgx only | 584 | 632 | 750 | 830 | 1.08 | 1.28 | 1.42 | Tesk1 |
| 1450664_at | CR & Lgx | 1309 | 969 | 977 | 814 | −1.35 | −1.34 | −1.61 | Gabpa |
| 1450670_at | Lgx only | 884 | 1083 | 1043 | 1276 | 1.22 | 1.18 | 1.44 | Dbh |
| 1450672_a_at | Lgx only | 892 | 966 | 977 | 1154 | 1.08 | 1.10 | 1.29 | Trex1 |
| 1450678_at | CR only | 384 | 292 | 365 | 338 | −1.32 | −1.05 | −1.14 | Itgb2 |
| 1450690_at | Lgx only | 1537 | 1178 | 953 | 774 | −1.30 | −1.61 | −1.98 | Ranbp2 |
| 1450691_at | Res only | 621 | 793 | 919 | 894 | 1.28 | 1.48 | 1.44 | Caskin2 |
| 1450700_at | Res & Lgx | 1859 | 1642 | 1543 | 1404 | −1.13 | −1.20 | −1.32 | Cdc42ep3 |
| 1450706_a_at | Lgx only | 2959 | 2743 | 2744 | 2114 | −1.08 | −1.08 | −1.40 | Arl3 |
| 1450714_at | Res & Lgx | 2451 | 2021 | 1702 | 1453 | −1.21 | −1.44 | −1.69 | Azin1 |
| 1450729_at | Lgx only | 637 | 561 | 625 | 508 | −1.14 | −1.02 | −1.25 | Hs2st1 |
| 1450735_at | Res & Lgx | 2114 | 2019 | 1724 | 1463 | −1.05 | −1.23 | −1.45 | Pno1 |
| 1450738_at | Lgx only | 938 | 886 | 1089 | 1210 | −1.06 | 1.16 | 1.29 | Kif21a |
| 1450740_a_at | Res & Lgx | 4235 | 4039 | 3546 | 3025 | −1.05 | −1.19 | −1.40 | Mapre1 |
| 1450744_at | Lgx only | 488 | 355 | 297 | 213 | −1.38 | −1.65 | −2.29 | Ell2 |
| 1450759_at | Res only | 852 | 857 | 1146 | 1021 | 1.01 | 1.35 | 1.20 | Bmp6 |
| 1450791_at | Lgx only | 15042 | 15107 | 14450 | 8913 | 1.00 | −1.04 | −1.69 | Nppb |
| 1450798_at | Lgx only | 819 | 1305 | 1266 | 1651 | 1.59 | 1.54 | 2.01 | Tnxb |
| 1450801_at | Res & Lgx | 37 | 93 | 146 | 227 | 2.53 | 3.98 | 6.20 | Adam21 |
| 1450816_at | Res only | 384 | 341 | 301 | 362 | −1.13 | −1.27 | −1.06 | Polg2 |
| 1450839_at | Res only | 1123 | 1002 | 829 | 1015 | −1.12 | −1.35 | −1.11 | D0H4S114 |
| 1450840_a_at | Lgx only | 50799 | 56471 | 65223 | 75285 | 1.11 | 1.28 | 1.48 | Rpl39 |
| 1450842_a_at | CR & Lgx | 2062 | 1704 | 1758 | 1347 | −1.21 | −1.17 | −1.53 | Cenpa |
| 1450857_a_at | Lgx only | 2960 | 3430 | 3914 | 4745 | 1.16 | 1.32 | 1.60 | Col1a2 |
| 1450866_a_at | Lgx only | 3015 | 3253 | 3652 | 3857 | 1.08 | 1.21 | 1.28 | Mrpl17 |
| 1450878_at | Lgx only | 2049 | 1928 | 1635 | 1368 | −1.06 | −1.25 | −1.50 | Sri |
| 1450879_at | Lgx only | 610 | 710 | 716 | 993 | 1.16 | 1.17 | 1.63 | Atp9b |
| 1450883_a_at | CR only | 31708 | 23985 | 32717 | 30704 | −1.32 | 1.03 | −1.03 | Cd36 |
| 1450890_a_at | Lgx only | 948 | 726 | 729 | 585 | −1.31 | −1.30 | −1.62 | Abi1 |
| 1450891_at | Lgx only | 5718 | 5089 | 4197 | 3750 | −1.12 | −1.36 | −1.53 | Srp19 |
| 1450894_a_at | Res & Lgx | 2906 | 3585 | 3746 | 3815 | 1.23 | 1.29 | 1.31 | Ap2m1 |
| 1450897_at | Lgx only | 1688 | 1363 | 1427 | 1165 | −1.24 | −1.18 | −1.45 | Arhgap5 |
| 1450903_at | Lgx only | 1020 | 958 | 834 | 627 | −1.06 | −1.22 | −1.63 | Rad23b |
| 1450927_at | Lgx only | 514 | 591 | 685 | 712 | 1.15 | 1.33 | 1.39 | Lztr1 |
| 1450934_at | Res & Lgx | 22353 | 18933 | 16228 | 15963 | −1.18 | −1.38 | −1.40 | Eif4a2 |
| 1450948_a_at | Lgx only | 827 | 736 | 541 | 458 | −1.12 | −1.53 | −1.81 | Mrpl1 |
| 1450953_at | Res & Lgx | 2794 | 2661 | 2247 | 2084 | −1.05 | −1.24 | −1.34 | Ciao1 |
| 1450957_a_at | Lgx only | 14361 | 13299 | 20795 | 20648 | −1.08 | 1.45 | 1.44 | Sqstm1 |
| 1450958_at | Lgx only | 5954 | 4641 | 4404 | 4138 | −1.28 | −1.35 | −1.44 | Tm4sf1 |
| 1450965_at | Res & Lgx | 1995 | 2296 | 2538 | 3135 | 1.15 | 1.27 | 1.57 | Tex261 |
| 1450966_at | Lgx only | 927 | 836 | 863 | 679 | −1.11 | −1.07 | −1.37 | Crot |
| 1450968_at | Lgx only | 39927 | 41899 | 42555 | 55205 | 1.05 | 1.07 | 1.38 | Uqcrfs1 |
| 1450970_at | Lgx only | 25949 | 32719 | 31690 | 36332 | 1.26 | 1.22 | 1.40 | Got1 |
| 1450971_at | Res & Lgx | 551 | 827 | 965 | 1069 | 1.50 | 1.75 | 1.94 | Gadd45b |
| 1450974_at | Res & Lgx | 776 | 1077 | 1180 | 1212 | 1.39 | 1.52 | 1.56 | Timp4 |
| 1450994_at | Lgx only | 2773 | 2136 | 2256 | 1821 | −1.30 | −1.23 | −1.52 | Rock1 |
| 1451002_at | Lgx only | 54266 | 62734 | 61354 | 75692 | 1.16 | 1.13 | 1.39 | Aco2 |
| 1451006_at | Lgx only | 1143 | 1483 | 1634 | 1746 | 1.30 | 1.43 | 1.53 | Xdh |
| 1451010_at | Lgx only | 656 | 786 | 728 | 858 | 1.20 | 1.11 | 1.31 | Nol11 |
| 1451017_at | Lgx only | 1586 | 1728 | 1871 | 2173 | 1.09 | 1.18 | 1.37 | Ergic3 |
| 1451019_at | Lgx only | 1338 | 1281 | 1678 | 2014 | −1.04 | 1.25 | 1.51 | Ctsf |
| 1451022_at | Lgx only | 545 | 506 | 713 | 852 | −1.08 | 1.31 | 1.56 | Lrp6 |
| 1451025_at | Lgx only | 2443 | 2161 | 1929 | 1347 | −1.13 | −1.27 | −1.81 | Arl1 |
| 1451050_at | Lgx only | 3147 | 2948 | 2847 | 2290 | −1.07 | −1.11 | −1.37 | Nt5c3 |
| 1451051_a_at | Lgx only | 837 | 1040 | 1137 | 1455 | 1.24 | 1.36 | 1.74 | Scyl1 |
| 1451067_at | Lgx only | 3259 | 4112 | 4448 | 4416 | 1.26 | 1.37 | 1.36 | Sgta |
| 1451070_at | Lgx only | 2031 | 2545 | 2472 | 3265 | 1.25 | 1.22 | 1.61 | Gdi1 |
| 1451074_at | Res & Lgx | 4210 | 3824 | 3180 | 2816 | −1.10 | −1.32 | −1.49 | Rnf13 |
| 1451096_at | Lgx only | 29178 | 32946 | 32150 | 36689 | 1.13 | 1.10 | 1.26 | Ndufs2 |
| 1451099_at | Lgx only | 535 | 685 | 718 | 838 | 1.28 | 1.34 | 1.57 | Mbc2 |
| 1451104_a_at | CR & Lgx | 1056 | 1430 | 1492 | 1790 | 1.35 | 1.41 | 1.69 | Snrp70 |
| 1451118_a_at | Lgx only | 790 | 918 | 855 | 1042 | 1.16 | 1.08 | 1.32 | 2410018C17Rik |
| 1451119_a_at | Lgx only | 2418 | 2685 | 3222 | 3877 | 1.11 | 1.33 | 1.60 | Fbln1 |
| 1451121_a_at | Res & Lgx | 1112 | 1325 | 1636 | 1783 | 1.19 | 1.47 | 1.60 | Gltscr2 |
| 1451126_at | Lgx only | 4627 | 4444 | 5283 | 5589 | −1.04 | 1.14 | 1.21 | Maf1 |
| 1451134_a_at | Res & Lgx | 1207 | 953 | 612 | 601 | −1.27 | −1.97 | −2.01 | Tm2d2 |
| 1451144_at | All | 1442 | 1150 | 986 | 886 | −1.25 | −1.46 | −1.63 | Bxdc2 |
| 1451159_at | Res & Lgx | 4282 | 5561 | 6247 | 8339 | 1.30 | 1.46 | 1.95 | Arhgef12 |
| 1451168_a_at | Res & Lgx | 4732 | 5809 | 6829 | 7722 | 1.23 | 1.44 | 1.63 | Arhgdia |
| 1451177_at | Res & Lgx | 6681 | 6017 | 4465 | 4080 | −1.11 | −1.50 | −1.64 | Dnajb4 |
| 1451187_at | Lgx only | 4203 | 4110 | 3516 | 3225 | −1.02 | −1.20 | −1.30 | 0610037P05Rik |
| 1451204_at | Res only | 2057 | 1633 | 1312 | 1410 | −1.26 | −1.57 | −1.46 | Scara5 |
| 1451217_a_at | Lgx only | 6459 | 5713 | 5401 | 4388 | −1.13 | −1.20 | −1.47 | Immp1l |
| 1451219_at | Lgx only | 915 | 777 | 738 | 662 | −1.18 | −1.24 | −1.38 | Ormdl1 |
| 1451223_a_at | Lgx only | 2697 | 2588 | 2232 | 1739 | −1.04 | −1.21 | −1.55 | Btf3l4 |
| 1451225_at | Lgx only | 6229 | 6428 | 6944 | 7210 | 1.03 | 1.11 | 1.16 | Ptpn11 |
| 1451226_at | Lgx only | 1149 | 1523 | 1685 | 2053 | 1.32 | 1.47 | 1.79 | Pex6 |
| 1451232_at | Lgx only | 6475 | 6003 | 4860 | 4346 | −1.08 | −1.33 | −1.49 | Cd151 |
| 1451244_a_at | Lgx only | 1155 | 1068 | 902 | 915 | −1.08 | −1.28 | −1.26 | Zfp422 |
| 1451245_at | Lgx only | 1838 | 1659 | 1561 | 1417 | −1.11 | −1.18 | −1.30 | Lrrc3b |
| 1451248_at | Res & Lgx | 1477 | 1443 | 1184 | 1086 | −1.02 | −1.25 | −1.36 | Prmt7 |
| 1451254_at | Res & Lgx | 1081 | 1005 | 719 | 681 | −1.08 | −1.50 | −1.59 | Ikbkap |
| 1451269_at | Lgx only | 1025 | 970 | 886 | 831 | −1.06 | −1.16 | −1.23 | Pdzd11 |
| 1451272_a_at | Res & Lgx | 1518 | 1300 | 972 | 856 | −1.17 | −1.56 | −1.77 | Ube2f |
| 1451274_at | Lgx only | 18853 | 24930 | 23212 | 26074 | 1.32 | 1.23 | 1.38 | Ogdh |
| 1451281_at | Res only | 182 | 134 | 126 | 176 | −1.36 | −1.44 | −1.03 | Zscan12 |
| 1451284_at | Lgx only | 824 | 1042 | 937 | 1011 | 1.26 | 1.14 | 1.23 | Yipf3 |
| 1451285_at | Lgx only | 1415 | 1654 | 1921 | 2284 | 1.17 | 1.36 | 1.61 | Fus |
| 1451290_at | Lgx only | 19210 | 20375 | 21862 | 26463 | 1.06 | 1.14 | 1.38 | Map1lc3a |
| 1451291_at | CR only | 985 | 1221 | 1225 | 1080 | 1.24 | 1.24 | 1.10 | Obfc2b |
| 1451293_at | CR only | 334 | 573 | 472 | 317 | 1.71 | 1.41 | −1.05 | Rrp9 |
| 1451295_a_at | Res & Lgx | 645 | 582 | 1919 | 1477 | −1.11 | 2.98 | 2.29 | Chd4 |
| 1451297_at | Res only | 17 | 25 | 80 | 44 | 1.45 | 4.66 | 2.55 | Gulo |
| 1451298_at | Lgx only | 536 | 562 | 684 | 976 | 1.05 | 1.28 | 1.82 | Plekhh3 |
| 1451312_at | Lgx only | 31762 | 35863 | 37350 | 45671 | 1.13 | 1.18 | 1.44 | Ndufs7 |
| 1451316_a_at | Res & Lgx | 5859 | 5369 | 3411 | 3242 | −1.09 | −1.72 | −1.81 | Picalm |
| 1451343_at | Lgx only | 832 | 752 | 752 | 595 | −1.11 | −1.11 | −1.40 | Vps36 |
| 1451344_at | Lgx only | 284 | 314 | 447 | 534 | 1.11 | 1.57 | 1.88 | Tmem119 |
| 1451349_at | Lgx only | 129 | 111 | 75 | 50 | −1.16 | −1.73 | −2.56 | BC020077 |
| 1451364_at | Res & Lgx | 1068 | 1040 | 814 | 549 | −1.03 | −1.31 | −1.94 | Polr3gl |
| 1451369_at | Lgx only | 1208 | 1284 | 1405 | 1759 | 1.06 | 1.16 | 1.46 | Commd5 |
| 1451381_at | Lgx only | 3059 | 2545 | 2352 | 2042 | −1.20 | −1.30 | −1.50 | 1810020D17Rik |
| 1451382_at | Res & Lgx | 247 | 447 | 470 | 562 | 1.81 | 1.90 | 2.28 | Chac1 |
| 1451388_a_at | Lgx only | 1122 | 1092 | 1224 | 1351 | −1.03 | 1.09 | 1.20 | Atp11b |
| 1451405_at | Lgx only | 3973 | 4289 | 5228 | 5041 | 1.08 | 1.32 | 1.27 | Pcca |
| 1451415_at | CR only | 1529 | 2052 | 1641 | 1570 | 1.34 | 1.07 | 1.03 | 1810011O10Rik |
| 1451420_at | Res & Lgx | 2878 | 2478 | 2242 | 2325 | −1.16 | −1.28 | −1.24 | Ccdc47 |
| 1451427_a_at | Lgx only | 1907 | 2012 | 2224 | 2426 | 1.05 | 1.17 | 1.27 | Egfl7 |
| 1451448_a_at | Lgx only | 1765 | 1548 | 1206 | 984 | −1.14 | −1.46 | −1.79 | 1110005A03Rik |
| 1451453_at | Lgx only | 246 | 248 | 265 | 336 | 1.01 | 1.08 | 1.36 | Dapk2 |
| 1451455_at | Res only | 273 | 220 | 478 | 374 | −1.24 | 1.75 | 1.37 | Thnsl2 |
| 1451462_a_at | Lgx only | 1261 | 1287 | 1200 | 958 | 1.02 | −1.05 | −1.32 | Ifnar2 |
| 1451465_at | Lgx only | 1407 | 1531 | 1643 | 1908 | 1.09 | 1.17 | 1.36 | Ubl7 |
| 1451471_at | Lgx only | 421 | 391 | 360 | 290 | −1.08 | −1.17 | −1.45 | Ears2 |
| 1451488_at | Lgx only | 7929 | 9254 | 9001 | 10760 | 1.17 | 1.14 | 1.36 | 1110028A07Rik |
| 1451502_at | CR only | 160 | 305 | 224 | 282 | 1.90 | 1.40 | 1.76 | Pla2g10 |
| 1451508_at | Lgx only | 1347 | 1314 | 1107 | 988 | −1.02 | −1.22 | −1.36 | Larp2 |
| 1451523_a_at | Lgx only | 1126 | 879 | 1016 | 827 | −1.28 | −1.11 | −1.36 | Mif4gd |
| 1451538_at | Lgx only | 135 | 123 | 153 | 242 | −1.10 | 1.13 | 1.78 | Sox9 |
| 1451553_at | Lgx only | 713 | 650 | 619 | 510 | −1.10 | −1.15 | −1.40 | Art5 |
| 1451561_at | Res & Lgx | 450 | 622 | 809 | 978 | 1.38 | 1.80 | 2.18 | Prr12 |
| 1451583_a_at | Res & Lgx | 69 | 119 | 158 | 261 | 1.72 | 2.29 | 3.78 | BC025076 |
| 1451604_a_at | Lgx only | 658 | 716 | 707 | 849 | 1.09 | 1.07 | 1.29 | Acvrl1 |
| 1451622_at | Lgx only | 2006 | 2187 | 2476 | 2643 | 1.09 | 1.23 | 1.32 | Lmbrd1 |
| 1451663_a_at | Lgx only | 743 | 755 | 847 | 912 | 1.02 | 1.14 | 1.23 | Trim3 |
| 1451665_a_at | Res & Lgx | 5435 | 4923 | 4504 | 4166 | −1.10 | −1.21 | −1.30 | Ap4s1 |
| 1451674_at | Lgx only | 114 | 131 | 180 | 195 | 1.15 | 1.57 | 1.70 | Slc12a5 |
| 1451678_at | Res & Lgx | 2482 | 2409 | 1553 | 1507 | −1.03 | −1.60 | −1.65 | Narf |
| 1451700_a_at | Res & Lgx | 2076 | 2271 | 2807 | 2740 | 1.09 | 1.35 | 1.32 | 1110007L15Rik |
| 1451728_at | Lgx only | 792 | 823 | 931 | 1122 | 1.04 | 1.17 | 1.42 | Wdr13 |
| 1451741_a_at | Lgx only | 732 | 614 | 585 | 518 | −1.19 | −1.25 | −1.41 | Cdk7 |
| 1451742_a_at | Lgx only | 14534 | 14261 | 12397 | 11426 | −1.02 | −1.17 | −1.27 | Ugp2 |
| 1451782_a_at | Lgx only | 2095 | 2186 | 2027 | 2552 | 1.04 | −1.03 | 1.22 | Slc29a1 |
| 1451789_a_at | CR only | 651 | 1037 | 851 | 972 | 1.59 | 1.31 | 1.49 | Ryk |
| 1451803_a_at | Lgx only | 6764 | 8556 | 8339 | 9104 | 1.26 | 1.23 | 1.35 | Vegfb |
| 1451820_at | Lgx only | 131 | 214 | 241 | 536 | 1.64 | 1.84 | 4.10 | Diras1 |
| 1451839_a_at | CR only | 890 | 1444 | 1029 | 1071 | 1.62 | 1.16 | 1.20 | Pde7a |
| 1451854_a_at | Lgx only | 383 | 572 | 575 | 694 | 1.49 | 1.50 | 1.81 | Shroom3 |
| 1451857_a_at | Lgx only | 194 | 239 | 319 | 373 | 1.23 | 1.64 | 1.92 | Notum |
| 1451883_at | Res & Lgx | 401 | 497 | 932 | 825 | 1.24 | 2.32 | 2.06 | ENSMUSG- |
| | | | | | | | | | 00000074670 |
| 1451902_at | Lgx only | 572 | 427 | 446 | 344 | −1.34 | −1.28 | −1.66 | Zfp758 |
| 1451911_a_at | Lgx only | 784 | 928 | 1055 | 1218 | 1.18 | 1.35 | 1.55 | Ace |
| 1451974_at | Lgx only | 3494 | 3897 | 3875 | 4394 | 1.12 | 1.11 | 1.26 | Osbpl2 |
| 1451984_at | Res & Lgx | 1996 | 1833 | 1480 | 1574 | −1.09 | −1.35 | −1.27 | Hnrpul1 |
| 1452012_a_at | Lgx only | 688 | 561 | 589 | 433 | −1.23 | −1.17 | −1.59 | Exosc1 |
| 1452024_a_at | Lgx only | 972 | 977 | 1305 | 1295 | 1.00 | 1.34 | 1.33 | Ldb1 |
| 1452043_at | Lgx only | 638 | 593 | 728 | 510 | −1.07 | 1.14 | −1.25 | 2310011J03Rik |
| 1452047_at | Res & Lgx | 2343 | 2094 | 1524 | 1383 | −1.12 | −1.54 | −1.69 | Cacybp |
| 1452057_at | Lgx only | 1040 | 1024 | 1176 | 1469 | −1.02 | 1.13 | 1.41 | Actr1b |
| 1452058_a_at | Res & Lgx | 8691 | 8360 | 5764 | 5728 | −1.04 | −1.51 | −1.52 | Rnf11 |
| 1452072_at | Res & Lgx | 1185 | 937 | 765 | 822 | −1.27 | −1.55 | −1.44 | Myct1 |
| 1452080_a_at | Lgx only | 1213 | 1057 | 971 | 775 | −1.15 | −1.25 | −1.57 | Dcun1d1 |
| 1452088_at | Res only | 153 | 231 | 81 | 127 | 1.51 | −1.89 | −1.21 | Zbed3 |
| 1452091_a_at | CR & Lgx | 852 | 1127 | 962 | 1169 | 1.32 | 1.13 | 1.37 | Rbm28 |
| 1452110_at | Lgx only | 459 | 361 | 351 | 327 | −1.27 | −1.31 | −1.41 | Mtrr |
| 1452130_at | Res & Lgx | 1764 | 1802 | 966 | 869 | 1.02 | −1.83 | −2.03 | Txndc14 |
| 1452140_at | Res & Lgx | 867 | 934 | 1137 | 1294 | 1.08 | 1.31 | 1.49 | Tbc1d20 |
| 1452141_a_at | Lgx only | 25419 | 26171 | 28265 | 32953 | 1.03 | 1.11 | 1.30 | Sepp1 |
| 1452143_at | All | 21772 | 24259 | 24942 | 27902 | 1.11 | 1.15 | 1.28 | Spnb2 |
| 1452145_at | Lgx only | 524 | 698 | 748 | 1076 | 1.33 | 1.43 | 2.06 | H6pd |
| 1452152_at | Lgx only | 2710 | 2333 | 2165 | 1785 | −1.16 | −1.25 | −1.52 | Clint1 |
| 1452155_a_at | Res & Lgx | 3205 | 3897 | 4271 | 4549 | 1.22 | 1.33 | 1.42 | Ddx17 |
| 1452156_a_at | Lgx only | 4703 | 5715 | 5941 | 7648 | 1.22 | 1.26 | 1.63 | Nisch |
| 1452159_at | Res & Lgx | 1379 | 1311 | 1056 | 977 | −1.05 | −1.31 | −1.41 | 2310001A20Rik |
| 1452173_at | Lgx only | 26826 | 30831 | 34012 | 38315 | 1.15 | 1.27 | 1.43 | Hadha |
| 1452174_at | Res & Lgx | 500 | 634 | 880 | 1000 | 1.27 | 1.76 | 2.00 | Srebf2 |
| 1452202_at | Lgx only | 1135 | 1145 | 1336 | 1475 | 1.01 | 1.18 | 1.30 | Pde2a |
| 1452203_at | All | 2414 | 1686 | 1491 | 1035 | −1.43 | −1.62 | −2.33 | Obfc2a |
| 1452208_at | Lgx only | 485 | 620 | 638 | 696 | 1.28 | 1.31 | 1.43 | Prdm4 |
| 1452213_at | Res & Lgx | 1320 | 1264 | 1036 | 845 | −1.04 | −1.27 | −1.56 | Tex2 |
| 1452214_at | Lgx only | 1513 | 1185 | 1126 | 850 | −1.28 | −1.34 | −1.78 | Skil |
| 1452221_a_at | Lgx only | 1063 | 1125 | 1281 | 1393 | 1.06 | 1.20 | 1.31 | Cxxc1 |
| 1452222_at | Lgx only | 1656 | 1643 | 1732 | 2090 | −1.01 | 1.05 | 1.26 | Utrn |
| 1452225_at | Lgx only | 1335 | 1061 | 1047 | 1000 | −1.26 | −1.28 | −1.33 | 2010106G01Rik |
| 1452250_a_at | Lgx only | 2260 | 2785 | 2667 | 3592 | 1.23 | 1.18 | 1.59 | Col6a2 |
| 1452262_at | Lgx only | 708 | 642 | 550 | 557 | −1.10 | −1.29 | −1.27 | Grpel2 |
| 1452286_at | Res & Lgx | 6581 | 4659 | 2979 | 2444 | −1.41 | −2.21 | −2.69 | Slain2 |
| 1452291_at | Lgx only | 564 | 461 | 566 | 360 | −1.22 | 1.00 | −1.57 | Centd1 |
| 1452292_at | Lgx only | 1094 | 1334 | 1435 | 1847 | 1.22 | 1.31 | 1.69 | Ap2b1 |
| 1452296_at | Lgx only | 237 | 266 | 412 | 579 | 1.12 | 1.74 | 2.45 | Slit3 |
| 1452308_a_at | Lgx only | 4905 | 5839 | 6466 | 6497 | 1.19 | 1.32 | 1.32 | Atp1a2 |
| 1452309_at | Lgx only | 721 | 839 | 763 | 1132 | 1.16 | 1.06 | 1.57 | Cgnl1 |
| 1452318_a_at | Res & Lgx | 536 | 385 | 278 | 289 | −1.39 | −1.93 | −1.86 | Hspa1b |
| 1452319_at | CR only | 283 | 194 | 251 | 220 | −1.46 | −1.13 | −1.28 | Zfp82 |
| 1452327_at | Lgx only | 904 | 1190 | 1305 | 1397 | 1.32 | 1.44 | 1.55 | Iqsec1 |
| 1452329_at | Res & Lgx | 265 | 311 | 451 | 486 | 1.17 | 1.71 | 1.84 | Plekhn1 |
| 1452330_a_at | Lgx only | 2664 | 2991 | 3519 | 4501 | 1.12 | 1.32 | 1.69 | Mxra8 |
| 1452333_at | Res & Lgx | 475 | 512 | 815 | 822 | 1.08 | 1.71 | 1.73 | smarca2 |
| 1452335_at | Res & Lgx | 485 | 467 | 382 | 319 | −1.04 | −1.27 | −1.52 | Mfsd8 |
| 1452339_at | Lgx only | 320 | 425 | 415 | 535 | 1.33 | 1.30 | 1.67 | Adamts7 |
| 1452374_at | Lgx only | 756 | 605 | 602 | 553 | −1.25 | −1.26 | −1.37 | Zfp322a |
| 1452375_at | Lgx only | 1059 | 1280 | 1336 | 1441 | 1.21 | 1.26 | 1.36 | Aldh4a1 |
| 1452395_at | Res & Lgx | 550 | 421 | 386 | 295 | −1.31 | −1.42 | −1.87 | Med19 |
| 1452398_at | Lgx only | 1052 | 998 | 837 | 640 | −1.05 | −1.26 | −1.64 | Plce1 |
| 1452401_at | Lgx only | 1940 | 1839 | 1632 | 1573 | −1.05 | −1.19 | −1.23 | Wtap |
| 1452411_at | Lgx only | 261 | 364 | 308 | 474 | 1.40 | 1.18 | 1.82 | Lrrc1 |
| 1452432_at | Lgx only | 2342 | 2445 | 2339 | 1802 | 1.04 | −1.00 | −1.30 | Tfpi |
| 1452446_a_at | Lgx only | 1260 | 1260 | 1436 | 1569 | 1.00 | 1.14 | 1.25 | Tmub2 |
| 1452462_a_at | Lgx only | 342 | 469 | 538 | 591 | 1.37 | 1.58 | 1.73 | Banp |
| 1452469_a_at | Res & Lgx | 1833 | 2687 | 2690 | 3184 | 1.47 | 1.47 | 1.74 | Smtn |
| 1452472_at | Lgx only | 128 | 142 | 189 | 242 | 1.11 | 1.47 | 1.89 | Rtp3 |
| 1452499_a_at | Res only | 705 | 659 | 530 | 573 | −1.07 | −1.33 | −1.23 | Kif2a |
| 1452502_at | Lgx only | 190 | 121 | 131 | 88 | −1.56 | −1.45 | −2.15 | ENSMUSG- |
| | | | | | | | | | 00000050599 |
| 1452509_at | CR only | 114 | 33 | 80 | 89 | −3.44 | −1.44 | −1.29 | Usp9y |
| 1452587_at | Lgx only | 3962 | 3990 | 3352 | 3268 | 1.01 | −1.18 | −1.21 | Actr2 |
| 1452596_at | Lgx only | 4654 | 3854 | 3014 | 2105 | −1.21 | −1.54 | −2.21 | Polr2k |
| 1452601_a_at | Lgx only | 978 | 969 | 1221 | 1242 | −1.01 | 1.25 | 1.27 | Acbd6 |
| 1452607_at | Lgx only | 978 | 917 | 707 | 587 | −1.07 | −1.38 | −1.67 | 2610030H06Rik |
| 1452608_at | Res & Lgx | 727 | 527 | 412 | 452 | −1.38 | −1.76 | −1.61 | Mycbp |
| 1452625_at | Lgx only | 772 | 766 | 950 | 1078 | −1.01 | 1.23 | 1.40 | Kctd2 |
| 1452653_at | Lgx only | 1121 | 1286 | 1501 | 1804 | 1.15 | 1.34 | 1.61 | Slc25a22 |
| 1452657_at | Lgx only | 1898 | 1692 | 1530 | 1326 | −1.12 | −1.24 | −1.43 | Ap1s2 |
| 1452661_at | Res & Lgx | 4325 | 6173 | 1466 | 1666 | 1.43 | −2.95 | −2.60 | Tfrc |
| 1452689_at | Res only | 1202 | 1315 | 1457 | 1353 | 1.09 | 1.21 | 1.13 | Zfp512 |
| 1452694_at | Lgx only | 1776 | 1941 | 2134 | 2382 | 1.09 | 1.20 | 1.34 | Ihpk1 |
| 1452698_at | Lgx only | 1101 | 1252 | 1139 | 1500 | 1.14 | 1.04 | 1.36 | Tsfm |
| 1452709_at | Res & Lgx | 590 | 680 | 819 | 833 | 1.15 | 1.39 | 1.41 | Poldip3 |
| 1452714_at | Lgx only | 872 | 1040 | 976 | 1284 | 1.19 | 1.12 | 1.47 | Tanc1 |
| 1452737_at | Lgx only | 1487 | 1388 | 1103 | 899 | −1.07 | −1.35 | −1.65 | 2810008M24Rik |
| 1452745_at | Lgx only | 788 | 1066 | 1236 | 1408 | 1.35 | 1.57 | 1.79 | 1810044A24Rik |
| 1452749_at | Lgx only | 1136 | 1094 | 893 | 738 | −1.04 | −1.27 | −1.54 | Papd1 |
| 1452753_at | Res only | 837 | 908 | 529 | 625 | 1.08 | −1.58 | −1.34 | Foxk2 |
| 1452761_a_at | Lgx only | 485 | 513 | 599 | 1042 | 1.06 | 1.24 | 2.15 | 8430436O14Rik |
| 1452767_at | Lgx only | 2638 | 2966 | 3499 | 3821 | 1.12 | 1.33 | 1.45 | Rrbp1 |
| 1452776_a_at | Res only | 663 | 841 | 1073 | 970 | 1.27 | 1.62 | 1.46 | Nub1 |
| 1452813_a_at | Res & Lgx | 2436 | 2172 | 1960 | 1741 | −1.12 | −1.24 | −1.40 | Tmem188 |
| 1452843_at | Res & Lgx | 3451 | 4038 | 4593 | 5346 | 1.17 | 1.33 | 1.55 | Il6st |
| 1452844_at | Res only | 751 | 823 | 1059 | 1074 | 1.10 | 1.41 | 1.43 | Pou6f1 |
| 1452856_at | Res only | 739 | 644 | 536 | 657 | −1.15 | −1.38 | −1.13 | Crebzf |
| 1452866_at | CR & Res | 2752 | 2260 | 2177 | 2385 | −1.22 | −1.26 | −1.15 | Nars |
| 1452867_at | Lgx only | 2212 | 1946 | 1780 | 1556 | −1.14 | −1.24 | −1.42 | Col4a3bp |
| 1452871_at | Lgx only | 416 | 352 | 302 | 259 | −1.18 | −1.38 | −1.61 | Neil1 |
| 1452874_at | Lgx only | 2271 | 2311 | 2091 | 1836 | 1.02 | −1.09 | −1.24 | 2510003E04Rik |
| 1452877_at | Lgx only | 2296 | 2197 | 1472 | 1361 | −1.05 | −1.56 | −1.69 | 2700029M09Rik |
| 1452885_at | Lgx only | 1428 | 1377 | 2182 | 2172 | −1.04 | 1.53 | 1.52 | Sfrs2ip |
| 1452897_at | CR & Lgx | 696 | 543 | 615 | 531 | −1.28 | −1.13 | −1.31 | Cdc2l5 |
| 1452901_at | Lgx only | 1170 | 1145 | 777 | 693 | −1.02 | −1.50 | −1.69 | Creb1 |
| 1452913_at | Lgx only | 2974 | 3092 | 3677 | 4408 | 1.04 | 1.24 | 1.48 | Pcp4l1 |
| 1452918_at | Res & Lgx | 829 | 733 | 480 | 371 | −1.13 | −1.73 | −2.23 | D19Ertd737e |
| 1452920_a_at | Lgx only | 1190 | 1193 | 1012 | 760 | 1.00 | −1.18 | −1.57 | Ppil2 |
| 1452952_at | Res & Lgx | 922 | 773 | 689 | 632 | −1.19 | −1.34 | −1.46 | 9030418K01Rik |
| 1452953_at | Res & Lgx | 992 | 827 | 679 | 580 | −1.20 | −1.46 | −1.71 | Fam18b |
| 1452960_at | Lgx only | 245 | 207 | 208 | 163 | −1.18 | −1.18 | −1.51 | Scyl3 |
| 1452965_at | Res only | 29 | 40 | 111 | 84 | 1.36 | 3.79 | 2.86 | Ankrd13d |
| 1452972_at | Lgx only | 1013 | 814 | 912 | 729 | −1.25 | −1.11 | −1.39 | Ttc32 |
| 1452977_at | Lgx only | 531 | 595 | 622 | 751 | 1.12 | 1.17 | 1.42 | Zhx3 |
| 1452985_at | Lgx only | 878 | 899 | 1344 | 1287 | 1.02 | 1.53 | 1.47 | Uaca |
| 1452999_at | Lgx only | 842 | 889 | 680 | 565 | 1.06 | −1.24 | −1.49 | Smndc1 |
| 1453007_at | CR only | 283 | 386 | 348 | 337 | 1.36 | 1.23 | 1.19 | 3110082I17Rik |
| 1453013_at | Res & Lgx | 382 | 472 | 620 | 612 | 1.23 | 1.62 | 1.60 | Zfp740 |
| 1453014_a_at | Lgx only | 1817 | 2542 | 2796 | 3042 | 1.40 | 1.54 | 1.67 | Sec31a |
| 1453023_at | Lgx only | 617 | 600 | 801 | 808 | −1.03 | 1.30 | 1.31 | Ankhd1 |
| 1453028_at | Lgx only | 215 | 210 | 184 | 94 | −1.03 | −1.17 | −2.29 | 4631424J17Rik |
| 1453039_at | Lgx only | 590 | 726 | 780 | 855 | 1.23 | 1.32 | 1.45 | Zfp335 |
| 1453058_at | Lgx only | 174 | 168 | 136 | 55 | −1.04 | −1.28 | −3.18 | Wdr5b |
| 1453059_at | Lgx only | 8551 | 8336 | 7741 | 7324 | −1.03 | −1.10 | −1.17 | 2310046A06Rik |
| 1453062_at | CR only | 223 | 400 | 295 | 433 | 1.80 | 1.33 | 1.95 | A930026I22Rik |
| 1453097_a_at | Lgx only | 1577 | 1678 | 1749 | 2148 | 1.06 | 1.11 | 1.36 | Ubtf |
| 1453119_at | Res & Lgx | 559 | 481 | 365 | 290 | −1.16 | −1.53 | −1.92 | Otud1 |
| 1453129_a_at | Lgx only | 557 | 617 | 773 | 837 | 1.11 | 1.39 | 1.50 | Rgs12 |
| 1453137_at | Lgx only | 1023 | 1022 | 916 | 767 | −1.00 | −1.12 | −1.33 | Fbxo30 |
| 1453149_at | Lgx only | 290 | 191 | 219 | 111 | −1.52 | −1.33 | −2.61 | Slc25a32 |
| 1453154_at | CR only | 79 | 24 | 84 | 44 | −3.27 | 1.06 | −1.78 | 1700029M20Rik |
| 1453155_at | Lgx only | 2675 | 2692 | 2995 | 3076 | 1.01 | 1.12 | 1.15 | Tmem50a |
| 1453160_at | Lgx only | 738 | 596 | 734 | 458 | −1.24 | −1.01 | −1.61 | Med13 |
| 1453180_at | Res only | 782 | 784 | 635 | 639 | 1.00 | −1.23 | −1.23 | 6530404N21Rik |
| 1453187_at | All | 605 | 410 | 365 | 248 | −1.48 | −1.66 | −2.44 | Ociad2 |
| 1453191_at | Lgx only | 374 | 503 | 391 | 738 | 1.34 | 1.04 | 1.97 | Col27a1 |
| 1453196_a_at | Res & Lgx | 254 | 340 | 389 | 411 | 1.34 | 1.53 | 1.62 | Oasl2 |
| 1453206_at | Res & Lgx | 890 | 1145 | 1255 | 1633 | 1.29 | 1.41 | 1.84 | Acad9 |
| 1453212_at | Lgx only | 319 | 237 | 226 | 141 | −1.34 | −1.41 | −2.27 | Zfp383 |
| 1453224_at | Res & Lgx | 809 | 940 | 1461 | 1631 | 1.16 | 1.81 | 2.02 | Zfand5 |
| 1453257_at | Lgx only | 853 | 753 | 660 | 637 | −1.13 | −1.29 | −1.34 | Agpat5 |
| 1453271_at | Res & Lgx | 1296 | 1098 | 939 | 900 | −1.18 | −1.38 | −1.44 | Phf14 |
| 1453296_at | CR only | 113 | 32 | 90 | 134 | −3.51 | −1.25 | 1.19 | Tmem103 |
| 1453312_at | Lgx only | 790 | 818 | 632 | 503 | 1.04 | −1.25 | −1.57 | Iqwd1 |
| 1453377_at | Res & Lgx | 606 | 603 | 406 | 399 | −1.00 | −1.49 | −1.52 | Sh2d4a |
| 1453391_at | CR only | 28 | 75 | 84 | 70 | 2.64 | 2.97 | 2.47 | Speer7-ps1 |
| 1453399_at | Res & Lgx | 607 | 726 | 1159 | 1232 | 1.20 | 1.91 | 2.03 | Ccnt2 |
| 1453412_a_at | CR only | 2089 | 2711 | 2591 | 2801 | 1.30 | 1.24 | 1.34 | Sec14l1 |
| 1453486_a_at | Lgx only | 1455 | 1240 | 1142 | 1034 | −1.17 | −1.27 | −1.41 | Scube2 |
| 1453494_at | Res only | 98 | 79 | 44 | 66 | −1.23 | −2.21 | −1.49 | 4921513H07Rik |
| 1453502_at | Lgx only | 77 | 79 | 94 | 27 | 1.02 | 1.22 | −2.82 | 2210408I21Rik |
| 1453552_at | CR & Lgx | 1567 | 1069 | 1454 | 980 | −1.47 | −1.08 | −1.60 | 2310014F07Rik |
| 1453572_a_at | Lgx only | 2411 | 2727 | 2793 | 3149 | 1.13 | 1.16 | 1.31 | Plp2 |
| 1453592_at | Lgx only | 3846 | 3055 | 3329 | 2431 | −1.26 | −1.16 | −1.58 | Lrrc39 |
| 1453673_at | Lgx only | 76 | 65 | 94 | 172 | −1.18 | 1.24 | 2.26 | LOC100046982 |
| 1453728_a_at | Lgx only | 3261 | 3568 | 2905 | 2627 | 1.09 | −1.12 | −1.24 | Mrps17 |
| 1453729_a_at | Lgx only | 41216 | 43763 | 46983 | 55549 | 1.06 | 1.14 | 1.35 | Rpl37 |
| 1453731_a_at | Lgx only | 3325 | 2887 | 2681 | 2049 | −1.15 | −1.24 | −1.62 | Tmem77 |
| 1453739_at | Res & Lgx | 450 | 425 | 316 | 266 | −1.06 | −1.42 | −1.69 | Tmem126b |
| 1453740_a_at | Lgx only | 2063 | 2311 | 2547 | 2926 | 1.12 | 1.24 | 1.42 | Ccnl2 |
| 1453761_at | Res only | 74 | 144 | 179 | 199 | 1.95 | 2.43 | 2.71 | Phf6 |
| 1453795_at | Lgx only | 1896 | 2038 | 2175 | 2434 | 1.08 | 1.15 | 1.28 | Fahd2a |
| 1453804_a_at | Lgx only | 804 | 758 | 637 | 604 | −1.06 | −1.26 | −1.33 | Orc4l |
| 1453821_at | Res only | 642 | 596 | 1039 | 923 | −1.08 | 1.62 | 1.44 | N6amt1 |
| 1453850_at | Lgx only | 43 | 35 | 37 | 131 | −1.24 | −1.19 | 3.01 | 1500002I01Rik |
| 1453851_a_at | Lgx only | 352 | 496 | 457 | 640 | 1.41 | 1.30 | 1.82 | Gadd45g |
| 1453865_a_at | Res & Lgx | 869 | 969 | 1199 | 1124 | 1.12 | 1.38 | 1.29 | Otud5 |
| 1453866_a_at | Lgx only | 43 | 74 | 75 | 126 | 1.71 | 1.73 | 2.91 | Xk |
| 1453898_at | CR only | 501 | 793 | 335 | 384 | 1.58 | −1.49 | −1.30 | Itgb1bp3 |
| 1453913_a_at | Lgx only | 849 | 962 | 1213 | 1299 | 1.13 | 1.43 | 1.53 | Tap2 |
| 1453976_at | CR only | 143 | 76 | 125 | 134 | −1.89 | −1.15 | −1.07 | 4432414F05Rik |
| 1453983_a_at | Lgx only | 927 | 804 | 894 | 635 | −1.15 | −1.04 | −1.46 | Mett10d |
| 1454017_at | Res & Lgx | 25 | 38 | 74 | 84 | 1.54 | 3.00 | 3.41 | 4921509O07Rik |
| 1454020_at | Lgx only | 51 | 52 | 75 | 163 | 1.04 | 1.49 | 3.21 | 4930554H23Rik |
| 1454021_a_at | CR only | 1143 | 1359 | 1194 | 1279 | 1.19 | 1.04 | 1.12 | Exosc10 |
| 1454023_a_at | Lgx only | 385 | 503 | 469 | 613 | 1.31 | 1.22 | 1.59 | D1Bwg1363e |
| 1454034_a_at | Lgx only | 569 | 709 | 792 | 894 | 1.25 | 1.39 | 1.57 | Usp21 |
| 1454047_a_at | CR only | 276 | 183 | 203 | 229 | −1.51 | −1.36 | −1.20 | 2410017P07Rik |
| 1454074_a_at | Res & Lgx | 1611 | 1425 | 1123 | 900 | −1.13 | −1.43 | −1.79 | Rsrc2 |
| 1454092_a_at | Res only | 593 | 635 | 442 | 463 | 1.07 | −1.34 | −1.28 | Gtf2h3 |
| 1454109_a_at | Res & Lgx | 591 | 481 | 406 | 366 | −1.23 | −1.46 | −1.61 | Jmjd6 |
| 1454116_a_at | Lgx only | 1801 | 1594 | 1238 | 1086 | −1.13 | −1.46 | −1.66 | Mterfd1 |
| 1454189_at | Lgx only | 73 | 110 | 141 | 142 | 1.51 | 1.93 | 1.94 | 4930557J02Rik |
| 1454206_a_at | Lgx only | 392 | 464 | 580 | 728 | 1.18 | 1.48 | 1.86 | Adam15 |
| 1454214_a_at | Lgx only | 746 | 743 | 637 | 541 | −1.00 | −1.17 | −1.38 | 2410019A14Rik |
| 1454236_a_at | Res only | 269 | 286 | 159 | 248 | 1.06 | −1.69 | −1.08 | Ppp4r1l |
| 1454395_at | Lgx only | 439 | 558 | 623 | 761 | 1.27 | 1.42 | 1.73 | 4632404M16Rik |
| 1454455_at | Lgx only | 72 | 65 | 114 | 167 | −1.10 | 1.59 | 2.33 | 6530413G14Rik |
| 1454466_at | CR only | 80 | 13 | 67 | 52 | −6.28 | −1.18 | −1.54 | 4933407I18Rik |
| 1454606_at | Lgx only | 1497 | 1775 | 1754 | 1897 | 1.19 | 1.17 | 1.27 | 4933426M11Rik |
| 1454611_a_at | Res only | 4696 | 4347 | 3422 | 3420 | −1.08 | −1.37 | −1.37 | Calm1 |
| 1454612_at | Lgx only | 610 | 597 | 535 | 437 | −1.02 | −1.14 | −1.40 | Mex3c |
| 1454613_at | Lgx only | 978 | 1329 | 1415 | 1720 | 1.36 | 1.45 | 1.76 | Dpysl3 |
| 1454619_at | Lgx only | 1392 | 1555 | 1880 | 2331 | 1.12 | 1.35 | 1.67 | Tmem112b |
| 1454626_at | Lgx only | 3655 | 3568 | 2821 | 2614 | −1.02 | −1.30 | −1.40 | Cltc |
| 1454631_at | Lgx only | 639 | 592 | 552 | 433 | −1.08 | −1.16 | −1.48 | Gtf2a1 |
| 1454638_a_at | Lgx only | 1004 | 949 | 703 | 592 | −1.06 | −1.43 | −1.70 | Pah |
| 1454644_at | Lgx only | 470 | 566 | 617 | 705 | 1.20 | 1.31 | 1.50 | 6330569M22Rik |
| 1454645_at | Res & Lgx | 3953 | 5231 | 5398 | 6226 | 1.32 | 1.37 | 1.57 | Mgrn1 |
| 1454646_at | Res only | 5976 | 6134 | 4613 | 4866 | 1.03 | −1.30 | −1.23 | Tcp11l2 |
| 1454647_at | Lgx only | 3249 | 2669 | 2518 | 2112 | −1.22 | −1.29 | −1.54 | Acad11 |
| 1454654_at | Lgx only | 1244 | 1418 | 1330 | 1645 | 1.14 | 1.07 | 1.32 | Dirc2 |
| 1454658_at | Res only | 790 | 826 | 1079 | 882 | 1.05 | 1.37 | 1.12 | Ilvbl |
| 1454666_at | Lgx only | 2164 | 1836 | 1500 | 1233 | −1.18 | −1.44 | −1.76 | LOC100046855 |
| 1454670_at | Lgx only | 1041 | 1359 | 1101 | 1577 | 1.31 | 1.06 | 1.51 | Rere |
| 1454679_at | Lgx only | 374 | 407 | 335 | 481 | 1.09 | −1.12 | 1.29 | D8Ertd457e |
| 1454682_at | Lgx only | 1062 | 918 | 870 | 788 | −1.16 | −1.22 | −1.35 | A430005L14Rik |
| 1454693_at | Lgx only | 512 | 617 | 534 | 706 | 1.20 | 1.04 | 1.38 | Hdac4 |
| 1454697_at | Lgx only | 1244 | 1274 | 1567 | 1632 | 1.02 | 1.26 | 1.31 | Tloc1 |
| 1454704_at | Lgx only | 8488 | 8673 | 9500 | 10160 | 1.02 | 1.12 | 1.20 | Scarb2 |
| 1454706_at | Res & Lgx | 841 | 933 | 1147 | 1655 | 1.11 | 1.36 | 1.97 | Uvrag |
| 1454709_at | CR only | 1558 | 1256 | 1428 | 1353 | −1.24 | −1.09 | −1.15 | Tmem64 |
| 1454718_at | CR only | 405 | 601 | 518 | 565 | 1.48 | 1.28 | 1.40 | Nagpa |
| 1454723_at | Lgx only | 553 | 511 | 424 | 378 | −1.08 | −1.31 | −1.46 | 1110033M05Rik |
| 1454727_at | Lgx only | 1394 | 1215 | 977 | 798 | −1.15 | −1.43 | −1.75 | Afap1l1 |
| 1454730_at | All | 769 | 590 | 524 | 464 | −1.30 | −1.47 | −1.66 | Tapt1 |
| 1454733_at | Res only | 911 | 985 | 1221 | 1082 | 1.08 | 1.34 | 1.19 | Nod1 |
| 1454739_at | Res & Lgx | 644 | 552 | 440 | 462 | −1.17 | −1.46 | −1.39 | Cdc27 |
| 1454745_at | Lgx only | 3289 | 3105 | 2800 | 2804 | −1.06 | −1.17 | −1.17 | Arhgap29 |
| 1454749_at | Lgx only | 684 | 403 | 1251 | 1482 | −1.70 | 1.83 | 2.17 | Pcnt |
| 1454753_at | Lgx only | 1587 | 2106 | 2105 | 2294 | 1.33 | 1.33 | 1.45 | Rnpepl1 |
| 1454759_at | Lgx only | 438 | 629 | 684 | 820 | 1.43 | 1.56 | 1.87 | Git1 |
| 1454780_at | Res & Lgx | 369 | 411 | 559 | 501 | 1.12 | 1.52 | 1.36 | Galntl4 |
| 1454797_at | Res & Lgx | 1016 | 1208 | 1395 | 1607 | 1.19 | 1.37 | 1.58 | Tmem55b |
| 1454801_at | Lgx only | 432 | 318 | 409 | 291 | −1.36 | −1.06 | −1.48 | Ankrd28 |
| 1454813_at | Lgx only | 14224 | 13070 | 12391 | 10412 | −1.09 | −1.15 | −1.37 | Ccdc72 |
| 1454820_at | Res & Lgx | 41 | 115 | 186 | 231 | 2.83 | 4.59 | 5.68 | BC037034 |
| 1454834_at | Lgx only | 2775 | 2711 | 2558 | 2415 | −1.02 | −1.08 | −1.15 | Nfib |
| 1454848_at | Lgx only | 465 | 542 | 638 | 645 | 1.16 | 1.37 | 1.39 | Ppp1r12c |
| 1454861_at | Lgx only | 629 | 688 | 770 | 967 | 1.09 | 1.22 | 1.54 | Txlna |
| 1454868_at | Lgx only | 1589 | 2148 | 2178 | 2607 | 1.35 | 1.37 | 1.64 | D4Ertd429e |
| 1454887_at | Lgx only | 747 | 899 | 852 | 1049 | 1.20 | 1.14 | 1.41 | Pak2 |
| 1454888_at | Res & Lgx | 1357 | 1167 | 902 | 903 | −1.16 | −1.50 | −1.50 | Pfdn4 |
| 1454892_at | Lgx only | 2663 | 2785 | 3279 | 3697 | 1.05 | 1.23 | 1.39 | Pitpnb |
| 1454894_at | Lgx only | 1514 | 1552 | 1292 | 1165 | 1.03 | −1.17 | −1.30 | LOC100045522 |
| 1454895_at | Res & Lgx | 580 | 737 | 920 | 969 | 1.27 | 1.59 | 1.67 | Cybasc3 |
| 1454906_at | Res & Lgx | 1336 | 1248 | 925 | 804 | −1.07 | −1.44 | −1.66 | Rarb |
| 1454914_at | Lgx only | 1116 | 1031 | 928 | 845 | −1.08 | −1.20 | −1.32 | 2610101N10Rik |
| 1454915_at | Res & Lgx | 351 | 379 | 517 | 591 | 1.08 | 1.47 | 1.68 | Rab3gap2 |
| 1454921_at | Lgx only | 2860 | 2668 | 2533 | 1763 | −1.07 | −1.13 | −1.62 | Gm561 |
| 1454922_at | Res & Lgx | 927 | 893 | 748 | 601 | −1.04 | −1.24 | −1.54 | Wdr92 |
| 1454928_at | Lgx only | 431 | 638 | 710 | 848 | 1.48 | 1.65 | 1.97 | Safb |
| 1454930_at | CR only | 902 | 684 | 840 | 692 | −1.32 | −1.07 | −1.30 | Tbcel |
| 1454934_at | CR only | 783 | 941 | 754 | 809 | 1.20 | −1.04 | 1.03 | Ppm1f |
| 1454937_at | Lgx only | 411 | 351 | 295 | 228 | −1.17 | −1.39 | −1.80 | B630005N14Rik |
| 1454938_at | Lgx only | 1417 | 1321 | 1222 | 1011 | −1.07 | −1.16 | −1.40 | Snx13 |
| 1454955_at | Lgx only | 6741 | 6181 | 6145 | 5319 | −1.09 | −1.10 | −1.27 | Ipo7 |
| 1454960_at | Lgx only | 737 | 915 | 886 | 1114 | 1.24 | 1.20 | 1.51 | Smad3 |
| 1454964_at | CR only | 4312 | 5102 | 4189 | 4991 | 1.18 | −1.03 | 1.16 | BC021395 |
| 1454977_at | Lgx only | 235 | 290 | 342 | 395 | 1.23 | 1.46 | 1.68 | AU020772 |
| 1454979_at | Lgx only | 940 | 990 | 1118 | 1206 | 1.05 | 1.19 | 1.28 | Diap1 |
| 1454980_at | Lgx only | 2726 | 3305 | 3169 | 3859 | 1.21 | 1.16 | 1.42 | 4930402E16Rik |
| 1454985_at | Lgx only | 374 | 389 | 391 | 531 | 1.04 | 1.05 | 1.42 | D030051N19Rik |
| 1454991_at | Res & Lgx | 1439 | 1242 | 1003 | 894 | −1.16 | −1.43 | −1.61 | Slc7a1 |
| 1454997_at | CR only | 2397 | 3094 | 2233 | 2451 | 1.29 | −1.07 | 1.02 | Msrb3 |
| 1455014_at | Res & Lgx | 1453 | 1054 | 833 | 759 | −1.38 | −1.75 | −1.91 | AV009015 |
| 1455018_at | Lgx only | 1301 | 1669 | 1774 | 2383 | 1.28 | 1.36 | 1.83 | Lmtk2 |
| 1455025_at | Res only | 1073 | 1005 | 788 | 849 | −1.07 | −1.36 | −1.26 | Paqr9 |
| 1455051_at | Lgx only | 500 | 578 | 720 | 851 | 1.16 | 1.44 | 1.70 | Rnf31 |
| 1455061_a_at | Lgx only | 19692 | 20976 | 24991 | 27704 | 1.07 | 1.27 | 1.41 | Acaa2 |
| 1455081_at | Lgx only | 393 | 471 | 526 | 526 | 1.20 | 1.34 | 1.34 | Txnl4b |
| 1455082_at | Lgx only | 455 | 375 | 356 | 312 | −1.21 | −1.28 | −1.46 | Cblb |
| 1455090_at | Lgx only | 2902 | 3308 | 2931 | 3642 | 1.14 | 1.01 | 1.26 | Angptl2 |
| 1455105_at | Lgx only | 1072 | 1049 | 833 | 740 | −1.02 | −1.29 | −1.45 | Ptpn12 |
| 1455118_at | Res & Lgx | 1938 | 1850 | 1436 | 1478 | −1.05 | −1.35 | −1.31 | D9Ertd402e |
| 1455131_at | Lgx only | 2218 | 2140 | 2041 | 1890 | −1.04 | −1.09 | −1.17 | Opa3 |
| 1455142_at | Lgx only | 409 | 403 | 306 | 230 | −1.02 | −1.34 | −1.78 | Socs4 |
| 1455143_at | Res & Lgx | 326 | 441 | 617 | 772 | 1.35 | 1.89 | 2.36 | Nlgn2 |
| 1455152_at | Res & Lgx | 2267 | 2135 | 1838 | 1619 | −1.06 | −1.23 | −1.40 | AI462493 |
| 1455153_at | Lgx only | 505 | 453 | 471 | 342 | −1.11 | −1.07 | −1.47 | Zfp236 |
| 1455155_at | Lgx only | 1223 | 1091 | 976 | 862 | −1.12 | −1.25 | −1.42 | Lsm14b |
| 1455157_a_at | Res & Lgx | 788 | 737 | 1180 | 1481 | −1.07 | 1.50 | 1.88 | BC039210 |
| 1455159_at | Lgx only | 527 | 455 | 447 | 310 | −1.16 | −1.18 | −1.70 | Appl1 |
| 1455164_at | Lgx only | 1752 | 2160 | 2211 | 2588 | 1.23 | 1.26 | 1.48 | Cdgap |
| 1455166_at | Res & Lgx | 1049 | 897 | 812 | 763 | −1.17 | −1.29 | −1.37 | Arl5b |
| 1455188_at | Res only | 445 | 474 | 263 | 378 | 1.06 | −1.69 | −1.18 | Ephb1 |
| 1455197_at | Res only | 89 | 155 | 200 | 137 | 1.74 | 2.24 | 1.54 | Rnd1 |
| 1455204_at | Res & Lgx | 1784 | 1576 | 1400 | 1239 | −1.13 | −1.27 | −1.44 | Pitpnc1 |
| 1455205_a_at | Lgx only | 1304 | 1651 | 1627 | 1812 | 1.27 | 1.25 | 1.39 | Usp19 |
| 1455207_at | Lgx only | 687 | 606 | 578 | 507 | −1.13 | −1.19 | −1.36 | 2410017P09Rik |
| 1455210_at | Lgx only | 573 | 682 | 716 | 811 | 1.19 | 1.25 | 1.42 | Zhx2 |
| 1455226_at | Res & Lgx | 980 | 1142 | 1530 | 1869 | 1.17 | 1.56 | 1.91 | Spnb1 |
| 1455244_at | Res & Lgx | 3049 | 2993 | 2023 | 1746 | −1.02 | −1.51 | −1.75 | Daam1 |
| 1455267_at | Lgx only | 1101 | 1050 | 793 | 685 | −1.05 | −1.39 | −1.61 | Esrrg |
| 1455268_at | Res & Lgx | 575 | 477 | 313 | 321 | −1.20 | −1.84 | −1.79 | Dph3 |
| 1455285_at | Res & Lgx | 760 | 771 | 569 | 445 | 1.01 | −1.34 | −1.71 | Slc31a1 |
| 1455288_at | Res & Lgx | 1136 | 1274 | 1630 | 1957 | 1.12 | 1.43 | 1.72 | 1110036O03Rik |
| 1455293_at | Lgx only | 249 | 208 | 259 | 124 | −1.20 | 1.04 | −2.02 | Leo1 |
| 1455296_at | Lgx only | 784 | 1049 | 929 | 1119 | 1.34 | 1.19 | 1.43 | LOC100047385 |
| 1455300_at | Res & Lgx | 629 | 510 | 389 | 317 | −1.23 | −1.62 | −1.98 | E130014J05Rik |
| 1455307_at | Lgx only | 487 | 544 | 697 | 794 | 1.12 | 1.43 | 1.63 | BC037112 |
| 1455309_at | Res & Lgx | 761 | 829 | 1263 | 1387 | 1.09 | 1.66 | 1.82 | Tmem16f |
| 1455312_at | Lgx only | 785 | 972 | 944 | 1067 | 1.24 | 1.20 | 1.36 | Phc3 |
| 1455320_at | CR & Lgx | 3658 | 2918 | 2870 | 2793 | −1.25 | −1.27 | −1.31 | AI480535 |
| 1455340_at | Res & Lgx | 576 | 384 | 329 | 275 | −1.50 | −1.75 | −2.09 | D030011O10Rik |
| 1455349_at | Lgx only | 697 | 526 | 549 | 463 | −1.32 | −1.27 | −1.50 | LOC100048397 |
| 1455353_at | Lgx only | 968 | 809 | 836 | 782 | −1.20 | −1.16 | −1.24 | Tmcc1 |
| 1455356_at | Lgx only | 320 | 299 | 499 | 662 | −1.07 | 1.56 | 2.07 | Camsap1 |
| 1455387_at | Res & Lgx | 1358 | 1115 | 995 | 758 | −1.22 | −1.36 | −1.79 | Nufip2 |
| 1455390_at | Lgx only | 1575 | 1427 | 1439 | 1118 | −1.10 | −1.09 | −1.41 | Alkbh6 |
| 1455434_a_at | Res & Lgx | 6402 | 5782 | 5247 | 4407 | −1.11 | −1.22 | −1.45 | Ktn1 |
| 1455442_at | Res & Lgx | 74 | 104 | 176 | 197 | 1.41 | 2.37 | 2.67 | Slc6a19 |
| 1455450_at | Lgx only | 1232 | 1200 | 1327 | 1592 | −1.03 | 1.08 | 1.29 | Ptpn3 |
| 1455456_a_at | Lgx only | 1242 | 1516 | 1332 | 1587 | 1.22 | 1.07 | 1.28 | Timm50 |
| 1455462_at | Res & Lgx | 218 | 251 | 334 | 356 | 1.15 | 1.53 | 1.63 | Adcy2 |
| 1455479_a_at | Res & Lgx | 12338 | 12135 | 7678 | 6396 | −1.02 | −1.61 | −1.93 | Ube2d3 |
| 1455482_at | All | 516 | 724 | 797 | 1018 | 1.40 | 1.54 | 1.97 | Ap2a2 |
| 1455491_at | Res & Lgx | 1099 | 1208 | 811 | 846 | 1.10 | −1.36 | −1.30 | Hnrph3 |
| 1455506_at | Lgx only | 5998 | 5890 | 8637 | 9404 | −1.02 | 1.44 | 1.57 | Slc25a34 |
| 1455508_at | Lgx only | 354 | 367 | 312 | 217 | 1.04 | −1.14 | −1.63 | A530082C11Rik |
| 1455538_at | Lgx only | 942 | 726 | 609 | 472 | −1.30 | −1.55 | −2.00 | 6330403M23Rik |
| 1455585_at | Lgx only | 601 | 526 | 486 | 404 | −1.14 | −1.24 | −1.49 | Rnf168 |
| 1455587_at | Res only | 11 | 16 | 20 | 21 | 1.39 | 1.75 | 1.82 | BC030183 |
| 1455588_at | Res & Lgx | 1065 | 816 | 578 | 556 | −1.30 | −1.84 | −1.91 | Lyrm4 |
| 1455655_a_at | Res & Lgx | 1506 | 1115 | 925 | 831 | −1.35 | −1.63 | −1.81 | Tardbp |
| 1455688_at | Lgx only | 951 | 817 | 745 | 702 | −1.16 | −1.28 | −1.35 | Ddr2 |
| 1455689_at | Lgx only | 270 | 358 | 391 | 488 | 1.33 | 1.45 | 1.81 | Fzd10 |
| 1455700_at | Res & Lgx | 1328 | 1183 | 811 | 664 | −1.12 | −1.64 | −2.00 | Mterfd3 |
| 1455702_at | Lgx only | 584 | 734 | 962 | 803 | 1.26 | 1.65 | 1.37 | Wdr22 |
| 1455733_at | Res & Lgx | 135 | 216 | 228 | 227 | 1.60 | 1.69 | 1.68 | Taok3 |
| 1455734_at | CR & Lgx | 1289 | 881 | 1118 | 921 | −1.46 | −1.15 | −1.40 | Crbn |
| 1455741_a_at | Res & Lgx | 1206 | 1346 | 1718 | 2034 | 1.12 | 1.42 | 1.69 | Ece1 |
| 1455750_at | Res & Lgx | 1072 | 1041 | 733 | 717 | −1.03 | −1.46 | −1.50 | A230067G21Rik |
| 1455757_at | Res & Lgx | 522 | 526 | 371 | 305 | 1.01 | −1.41 | −1.71 | D3Ertd254e |
| 1455794_at | Lgx only | 566 | 625 | 809 | 1034 | 1.10 | 1.43 | 1.83 | Smtnl2 |
| 1455832_a_at | Res only | 420 | 352 | 325 | 287 | −1.19 | −1.29 | −1.46 | Umps |
| 1455854_a_at | Res & Lgx | 470 | 513 | 771 | 829 | 1.09 | 1.64 | 1.76 | Ssh1 |
| 1455870_at | Lgx only | 2176 | 2652 | 2908 | 3503 | 1.22 | 1.34 | 1.61 | Akap2 |
| 1455873_a_at | Lgx only | 271 | 383 | 402 | 483 | 1.41 | 1.48 | 1.78 | Vps18 |
| 1455884_at | Lgx only | 508 | 747 | 821 | 851 | 1.47 | 1.62 | 1.67 | Dpp9 |
| 1455914_at | Lgx only | 168 | 138 | 101 | 93 | −1.22 | −1.66 | −1.81 | AI987944 |
| 1455915_at | CR only | 191 | 132 | 150 | 138 | −1.44 | −1.27 | −1.38 | Galnt4 |
| 1455922_at | Lgx only | 1213 | 1256 | 1440 | 1560 | 1.04 | 1.19 | 1.29 | Rab3gap1 |
| 1455936_a_at | CR & Lgx | 1540 | 2056 | 1827 | 2200 | 1.33 | 1.19 | 1.43 | Rbpms |
| 1455944_at | Lgx only | 153 | 127 | 219 | 289 | −1.21 | 1.43 | 1.88 | Zfp516 |
| 1455945_at | Lgx only | 192 | 166 | 136 | 117 | −1.16 | −1.41 | −1.65 | Zfp817 |
| 1456046_at | Lgx only | 3587 | 3017 | 2786 | 2266 | −1.19 | −1.29 | −1.58 | Cd93 |
| 1456058_at | Lgx only | 807 | 745 | 690 | 598 | −1.08 | −1.17 | −1.35 | Rbm27 |
| 1456059_at | Lgx only | 4664 | 4475 | 3594 | 3214 | −1.04 | −1.30 | −1.45 | Psmd11 |
| 1456061_at | Res & Lgx | 740 | 574 | 371 | 258 | −1.29 | −1.99 | −2.87 | Gimap8 |
| 1456065_at | CR only | 128 | 49 | 171 | 119 | −2.60 | 1.33 | −1.07 | Ubash3a |
| 1456092_at | Lgx only | 86 | 52 | 58 | 26 | −1.67 | −1.49 | −3.35 | Kctd7 |
| 1456099_at | Res only | 128 | 174 | 254 | 207 | 1.35 | 1.98 | 1.61 | D930017J03Rik |
| 1456161_at | Lgx only | 403 | 283 | 297 | 279 | −1.42 | −1.36 | −1.44 | 0610040B10Rik |
| 1456169_at | Res only | 128 | 171 | 178 | 144 | 1.34 | 1.39 | 1.13 | EG226654 |
| 1456210_at | Lgx only | 150 | 178 | 101 | 68 | 1.18 | −1.48 | −2.21 | 5430407P10Rik |
| 1456241_a_at | Lgx only | 3667 | 3705 | 4635 | 4984 | 1.01 | 1.26 | 1.36 | 1810073N04Rik |
| 1456257_at | Res only | 495 | 565 | 327 | 409 | 1.14 | −1.51 | −1.21 | C130065N10Rik |
| 1456315_a_at | Res & Lgx | 5639 | 5549 | 3253 | 2716 | −1.02 | −1.73 | −2.08 | Ptpla |
| 1456398_at | Lgx only | 1951 | 1675 | 1544 | 1204 | −1.16 | −1.26 | −1.62 | Tug1 |
| 1456487_at | Lgx only | 270 | 438 | 425 | 592 | 1.62 | 1.57 | 2.19 | Adcy1 |
| 1456599_at | CR only | 106 | 165 | 118 | 109 | 1.55 | 1.11 | 1.03 | Nxt2 |
| 1456604_a_at | Lgx only | 2365 | 2051 | 1676 | 1367 | −1.15 | −1.41 | −1.73 | Pcmt1 |
| 1456611_at | Lgx only | 462 | 581 | 505 | 608 | 1.26 | 1.09 | 1.31 | D430015B01Rik |
| 1456625_at | Lgx only | 192 | 140 | 145 | 126 | −1.37 | −1.33 | −1.52 | Aasdhppt |
| 1456643_at | Lgx only | 386 | 308 | 278 | 233 | −1.25 | −1.39 | −1.65 | 9230114K14Rik |
| 1456659_at | Lgx only | 319 | 363 | 307 | 153 | 1.14 | −1.04 | −2.08 | LOC552902 |
| 1456727_a_at | Res & Lgx | 2775 | 2975 | 4693 | 5107 | 1.07 | 1.69 | 1.84 | Csnk1d |
| 1456768_a_at | Lgx only | 1333 | 1602 | 1733 | 2588 | 1.20 | 1.30 | 1.94 | Mmrn2 |
| 1456774_at | Lgx only | 324 | 594 | 806 | 961 | 1.83 | 2.49 | 2.97 | Ppp1r13l |
| 1456777_at | CR & Lgx | 38 | 14 | 75 | 13 | −2.79 | 1.95 | −3.02 | Mgam |
| 1456827_at | Lgx only | 196 | 148 | 119 | 61 | −1.33 | −1.64 | −3.22 | AA987161 |
| 1456836_at | CR only | 151 | 39 | 112 | 115 | −3.85 | −1.34 | −1.31 | Itk |
| 1456871_a_at | Res only | 1810 | 1658 | 2405 | 2210 | −1.09 | 1.33 | 1.22 | Phf20l1 |
| 1456888_at | Lgx only | 252 | 244 | 313 | 371 | −1.03 | 1.24 | 1.47 | Pfkfb4 |
| 1456896_at | Res only | 473 | 464 | 642 | 654 | −1.02 | 1.36 | 1.38 | 6720462K09Rik |
| 1456914_at | Lgx only | 239 | 197 | 190 | 142 | −1.21 | −1.26 | −1.69 | Slc16a4 |
| 1457058_at | Lgx only | 245 | 319 | 349 | 556 | 1.31 | 1.43 | 2.27 | Adamts2 |
| 1457111_at | Res only | 267 | 251 | 189 | 227 | −1.07 | −1.41 | −1.18 | AA415038 |
| 1457276_at | Res only | 149 | 225 | 240 | 200 | 1.51 | 1.61 | 1.35 | Snf1lk2 |
| 1457285_at | Lgx only | 1194 | 1178 | 764 | 664 | −1.01 | −1.56 | −1.80 | Zfp187 |
| 1457334_at | All | 286 | 516 | 618 | 643 | 1.80 | 2.16 | 2.25 | C130057M05Rik |
| 1457401_at | Lgx only | 63 | 89 | 114 | 167 | 1.41 | 1.81 | 2.64 | Dnahc9 |
| 1457448_at | CR only | 154 | 79 | 90 | 117 | −1.94 | −1.71 | −1.32 | Pnpla1 |
| 1457501_at | Res only | 20 | 78 | 111 | 63 | 3.99 | 5.68 | 3.23 | RP23-233B9.8 |
| 1457508_at | Res & Lgx | 400 | 281 | 286 | 239 | −1.42 | −1.40 | −1.67 | C430003N24Rik |
| 1457557_at | Lgx only | 81 | 31 | 72 | 23 | −2.59 | −1.13 | −3.55 | A330076H08Rik |
| 1457626_at | Res only | 58 | 84 | 135 | 152 | 1.44 | 2.31 | 2.59 | D3Wsu106e |
| 1457671_at | Lgx only | 767 | 700 | 555 | 483 | −1.10 | −1.38 | −1.59 | 9330120H11Rik |
| 1457681_at | Lgx only | 1108 | 992 | 1385 | 1651 | −1.12 | 1.25 | 1.49 | 2610301F02Rik |
| 1457707_at | Res & Lgx | 55 | 71 | 153 | 177 | 1.29 | 2.78 | 3.21 | Mctp2 |
| 1457745_at | Res only | 361 | 391 | 519 | 505 | 1.08 | 1.44 | 1.40 | Gpr4 |
| 1457747_at | Res only | 8 | 14 | 30 | 35 | 1.77 | 3.81 | 4.34 | 9330109K16Rik |
| 1457801_at | CR only | 268 | 388 | 343 | 329 | 1.45 | 1.28 | 1.23 | 9930024M15Rik |
| 1458058_at | Lgx only | 149 | 104 | 122 | 59 | −1.44 | −1.21 | −2.53 | 7030407E18Rik |
| 1458190_at | Res only | 120 | 146 | 291 | 240 | 1.21 | 2.41 | 2.00 | Arhgap4 |
| 1458311_at | Res only | 249 | 304 | 433 | 381 | 1.22 | 1.74 | 1.53 | Usp36 |
| 1458353_at | Res only | 162 | 269 | 340 | 244 | 1.66 | 2.10 | 1.51 | Nwd1 |
| 1458438_at | Lgx only | 240 | 181 | 180 | 118 | −1.33 | −1.34 | −2.03 | Ccdc122 |
| 1458455_at | Lgx only | 566 | 438 | 512 | 358 | −1.29 | −1.11 | −1.58 | Abra |
| 1458461_at | Res only | 12 | 21 | 40 | 20 | 1.66 | 3.22 | 1.57 | E330021D16Rik |
| 1458478_at | Res only | 65 | 48 | 12 | 53 | −1.36 | −5.63 | −1.23 | 9230110F11Rik |
| 1458482_at | Lgx only | 1340 | 1453 | 1156 | 1017 | 1.08 | −1.16 | −1.32 | Tnni3k |
| 1458624_at | Res & Lgx | 1590 | 1831 | 2206 | 2675 | 1.15 | 1.39 | 1.68 | Rbm24 |
| 1458780_at | Res only | 20 | 68 | 74 | 55 | 3.33 | 3.61 | 2.70 | D8Ertd620e |
| 1458863_at | Res only | 152 | 101 | 37 | 85 | −1.50 | −4.15 | −1.79 | 6330415G19Rik |
| 1459108_a_at | Res & Lgx | 219 | 259 | 328 | 445 | 1.18 | 1.50 | 2.03 | Yeats2 |
| 1459220_at | Lgx only | 138 | 217 | 222 | 276 | 1.57 | 1.61 | 2.00 | C78651 |
| 1459363_at | Lgx only | 312 | 405 | 407 | 506 | 1.30 | 1.31 | 1.62 | Atxn2 |
| 1459578_at | Res only | 54 | 61 | 155 | 88 | 1.12 | 2.85 | 1.62 | AA407175 |
| 1460033_at | CR only | 74 | 180 | 125 | 105 | 2.42 | 1.69 | 1.42 | C030002C11Rik |
| 1460053_at | CR only | 242 | 144 | 218 | 221 | −1.68 | −1.11 | −1.10 | Smyd4 |
| 1460113_at | CR only | 104 | 187 | 144 | 134 | 1.80 | 1.38 | 1.29 | B930093H17Rik |
| 1460165_at | Res & Lgx | 9064 | 9043 | 7129 | 6522 | −1.00 | −1.27 | −1.39 | Ppp1ca |
| 1460167_at | Lgx only | 988 | 1120 | 1063 | 1253 | 1.13 | 1.08 | 1.27 | Aldh7a1 |
| 1460169_a_at | Lgx only | 2453 | 3136 | 3555 | 4351 | 1.28 | 1.45 | 1.77 | Pctk1 |
| 1460177_at | Res & Lgx | 1301 | 1575 | 1640 | 1671 | 1.21 | 1.26 | 1.28 | Cndp2 |
| 1460184_at | Lgx only | 36182 | 38955 | 42606 | 49147 | 1.08 | 1.18 | 1.36 | Hadh |
| 1460189_at | Res & Lgx | 4600 | 3988 | 3323 | 2755 | −1.15 | −1.38 | −1.67 | Wdr23 |
| 1460194_at | Lgx only | 12145 | 13386 | 14000 | 14613 | 1.10 | 1.15 | 1.20 | Phyh |
| 1460196_at | Lgx only | 1079 | 1068 | 1158 | 1385 | −1.01 | 1.07 | 1.28 | Cbr1 |
| 1460210_at | Lgx only | 683 | 848 | 775 | 1026 | 1.24 | 1.13 | 1.50 | Pkd1 |
| 1460214_at | CR only | 27 | 97 | 92 | 108 | 3.67 | 3.46 | 4.07 | Pcp4 |
| 1460216_at | Lgx only | 5275 | 5712 | 6310 | 7377 | 1.08 | 1.20 | 1.40 | Acads |
| 1460230_at | Lgx only | 106 | 114 | 120 | 192 | 1.07 | 1.13 | 1.81 | Syn2 |
| 1460239_at | Lgx only | 3477 | 3308 | 3484 | 3037 | −1.05 | 1.00 | −1.15 | Tspan13 |
| 1460251_at | Lgx only | 464 | 467 | 407 | 262 | 1.01 | −1.14 | −1.77 | Fas |
| 1460254_at | Res & Lgx | 1563 | 1408 | 1168 | 924 | −1.11 | −1.34 | −1.69 | 1810049H13Rik |
| 1460271_at | CR only | 157 | 66 | 118 | 125 | −2.38 | −1.33 | −1.26 | Trem3 |
| 1460276_a_at | Lgx only | 822 | 886 | 967 | 1097 | 1.08 | 1.18 | 1.33 | Gpr175 |
| 1460321_at | CR only | 129 | 52 | 117 | 105 | −2.48 | −1.10 | −1.23 | Cntn4 |
| 1460326_at | Res & Lgx | 1395 | 1325 | 1867 | 1690 | −1.05 | 1.34 | 1.21 | Pik3ca |
| 1460328_at | Res & Lgx | 619 | 628 | 880 | 862 | 1.01 | 1.42 | 1.39 | Brd3 |
| 1460329_at | Res only | 713 | 637 | 458 | 504 | −1.12 | −1.56 | −1.42 | LOC675709 |
| 1460330_at | Lgx only | 1601 | 1521 | 1413 | 1219 | −1.05 | −1.13 | −1.31 | Anxa3 |
| 1460331_at | Res & Lgx | 5861 | 5156 | 4527 | 4030 | −1.14 | −1.29 | −1.45 | Tm9sf2 |
| 1460336_at | Lgx only | 2689 | 2686 | 4293 | 5218 | −1.00 | 1.60 | 1.94 | Ppargc1a |
| 1460337_at | CR only | 4383 | 5948 | 3663 | 4114 | 1.36 | −1.20 | −1.07 | Sh3kbp1 |
| 1460344_at | Lgx only | 1002 | 1085 | 1313 | 1398 | 1.08 | 1.31 | 1.40 | 2310033F14Rik |
| 1460396_at | Lgx only | 483 | 621 | 790 | 817 | 1.29 | 1.63 | 1.69 | Ddx54 |
| 1460409_at | Lgx only | 1345 | 1292 | 1713 | 2001 | −1.04 | 1.27 | 1.49 | Cpt1a |
| 1460412_at | CR only | 58 | 20 | 45 | 57 | −2.84 | −1.30 | −1.03 | 1600015H20Rik |
| 1460420_a_at | Lgx only | 634 | 820 | 934 | 1142 | 1.29 | 1.47 | 1.80 | Egfr |
| 1460428_at | Lgx only | 919 | 958 | 1120 | 1123 | 1.04 | 1.22 | 1.22 | Ankrd13a |
| 1460432_a_at | Lgx only | 12140 | 9937 | 10609 | 8730 | −1.22 | −1.14 | −1.39 | Eif3e |
| 1460433_at | Lgx only | 662 | 763 | 813 | 1034 | 1.15 | 1.23 | 1.56 | Entpd6 |
| 1460435_at | Lgx only | 573 | 713 | 699 | 935 | 1.25 | 1.22 | 1.63 | 1500002O20Rik |
| 1460444_at | Lgx only | 329 | 323 | 411 | 491 | −1.02 | 1.25 | 1.49 | Arrb1 |
| 1460500_at | Lgx only | 313 | 176 | 158 | 98 | −1.78 | −1.98 | −3.20 | 5033421C21Rik |
| 1460510_a_at | Res & Lgx | 3505 | 3184 | 2702 | 2908 | −1.10 | −1.30 | −1.21 | Coq10b |
| 1460539_at | Res only | 68 | 139 | 141 | 153 | 2.04 | 2.07 | 2.25 | 4933404K13Rik |
| 1460547_a_at | Res & Lgx | 4912 | 3450 | 2670 | 1733 | −1.42 | −1.84 | −2.84 | Hnrpk |
| 1460552_at | Lgx only | 2039 | 2109 | 2286 | 2886 | 1.03 | 1.12 | 1.42 | Ascc3l1 |
| 1460557_at | Lgx only | 1577 | 1866 | 1401 | 1297 | 1.18 | −1.13 | −1.22 | Supv3l1 |
| 1460559_at | Lgx only | 2459 | 2747 | 3020 | 4193 | 1.12 | 1.23 | 1.71 | Ankrd25 |
| 1460570_at | Lgx only | 90 | 123 | 168 | 176 | 1.36 | 1.87 | 1.95 | Pgbd5 |
| 1460573_at | Res & Lgx | 838 | 611 | 460 | 341 | −1.37 | −1.82 | −2.46 | AI848100 |
| 1460576_at | Res & Lgx | 1390 | 1299 | 1107 | 878 | −1.07 | −1.26 | −1.58 | LOC100047539 |
| 1460580_at | Lgx only | 950 | 1086 | 1029 | 1226 | 1.14 | 1.08 | 1.29 | Pcnx |
| 1460586_at | Lgx only | 802 | 890 | 990 | 1316 | 1.11 | 1.23 | 1.64 | Megf8 |
| 1460603_at | Res only | 703 | 632 | 490 | 590 | −1.11 | −1.43 | −1.19 | Samd9l |
| 1460607_at | CR only | 43 | 102 | 138 | 99 | 2.38 | 3.21 | 2.30 | Igsf11 |
| 1460610_at | Lgx only | 191 | 208 | 233 | 328 | 1.09 | 1.22 | 1.72 | Agbl5 |
| 1460614_at | Lgx only | 331 | 289 | 291 | 199 | −1.14 | −1.14 | −1.66 | LOC100045020 |
| 1460624_at | Res & Lgx | 363 | 266 | 217 | 198 | −1.36 | −1.67 | −1.83 | 6330564D18Rik |
| 1460643_at | Lgx only | 270 | 360 | 304 | 400 | 1.34 | 1.13 | 1.48 | Ell |
| 1460644_at | Lgx only | 2028 | 2060 | 2523 | 2936 | 1.02 | 1.24 | 1.45 | Bckdk |
| 1460645_at | Res & Lgx | 1384 | 1178 | 880 | 926 | −1.17 | −1.57 | −1.49 | chordc1 |
| 1460648_at | Lgx only | 1100 | 1245 | 1478 | 1574 | 1.13 | 1.34 | 1.43 | Nr2f6 |
| 1460674_at | Lgx only | 477 | 623 | 657 | 970 | 1.31 | 1.38 | 2.04 | Paqr7 |
| 1460675_at | Lgx only | 412 | 429 | 642 | 729 | 1.04 | 1.56 | 1.77 | Igsf8 |
| 1460695_a_at | Lgx only | 2491 | 2664 | 2132 | 1926 | 1.07 | −1.17 | −1.29 | 2010111I01Rik |
| 1460704_at | Lgx only | 664 | 756 | 815 | 930 | 1.14 | 1.23 | 1.40 | Rfng |
| 1460716_a_at | Lgx only | 2287 | 2216 | 2090 | 1679 | −1.03 | −1.09 | −1.36 | Cbfb |
| 1460720_at | Res & Lgx | 1749 | 2092 | 2554 | 2716 | 1.20 | 1.46 | 1.55 | Trpc4ap |
| 1460732_a_at | Lgx only | 495 | 527 | 571 | 770 | 1.06 | 1.15 | 1.56 | Ppl |
| AFFX-b-ActinMur/ | Res & Lgx | 37967 | 41791 | 48255 | 54572 | 1.10 | 1.27 | 1.44 | Actb |
| M12481_3_at | | | | | | | | | |
| AFFX-GapdhMur/ | Lgx only | 90974 | 104691 | 98912 | 122441 | 1.15 | 1.09 | 1.35 | Gapdh |
| M32599_M_at | | | | | | | | | |

Several genes of particular interest showed expression patterns indicating that compounds of the present invention (Longevinex®) up-regulated survival/longevity genes or down-regulate genes whose expression enhances cellular damage to a greater extent than resveratrol:
(A) The sirtuin family of genes, and in particular Sirtuin 1, are thought to be critical mediators of extended lifespans (Boily, G. et al. (2008) “SirT1 Regulates Energy Metabolism And Response To Caloric Restriction In Mice,” PLoS ONE 3(3):e1759; Huang, J. et al. (2008) “SIRT1 Overexpression Antagonizes Cellular Senescence with Activated ERK/S6k1 Signaling in Human Diploid Fibroblasts,” PLoS ONE 3(3):e1710). Whereas mice receiving resveratrol showed only a 1.22 fold decrease in expression and mice subjected to a calorie restricted diet showed only a 1.12 fold reduction in Sirtuin 1 expression, expression of Sirtuin 1 was found to be decreased 1.71 fold in mice receiving Longevinex®.
(B) Pgc-1α (peroxisome proliferative activated receptor, gamma, coactivator 1 alpha; ppargc 1a) is a transcriptional co-factor that controls energy metabolism and mitochondrial biogenesis; its expression is increased in skeletal muscle tissue upon long-term calorie restriction (Conley, K. E. et al. (2007) “Mitochondrial Dysfunction and Age,” Curr. Opin. Clin. Nutr. Metab. Care. 10(6):688-692; Wu, Z. et al. (2007) “Targeting PGC-1 Alpha To Control Energy Homeostasis,” Expert Opin. Ther. Targets 11(10):1329-1338). Whereas mice receiving resveratrol showed only a 1.6 fold increase in expression and mice subjected to a calorie restricted diet showed no increase in Pgc-1α expression, mice receiving Longevinex® showed a 1.94 fold increase in Pgc-1α expression.
(C) Uncoupling protein-3 is believed to be a target of Pgc-1α and to play a role in fatty acid metabolism; its expression is increased in cardiac tissue upon long-term calorie restriction (Bézaire, V. et al. (Epub 2007 Jan. 3) “Uncoupling Protein-3: Clues In An Ongoing Mitochondrial Mystery,” FASEB J. 21(2):312-324; Chan, C. B. et al. (2006) “Uncoupling Proteins: Role In Insulin Resistance And Insulin Insufficiency,” Curr. Diabetes Rev. 2(3):271-283). Whereas mice receiving resveratrol showed only a 2.02 fold increase in expression and mice subjected to a calorie restricted diet showed only a 1.8 fold increase in uncoupling protein-3 expression, mice receiving Longevinex® showed a 2.79 fold increase in uncoupling protein-3 expression.
(D) Pyruvate dehydrogenase kinase 4 coordinates fuel selection during fasting to promote fatty acid metabolism (Sugden, M. C. et al. (2006) “Mechanisms Underlying Regulation Of The Expression And Activities Of The Mammalian Pyruvate Dehydrogenase Kinases,” Arch. Physiol. Biochem. 112(3):139-149; Pilegaard, H. et al. (2004) “Transcriptional Regulation Of Pyruvate Dehydrogenase Kinase 4 In Skeletal Muscle During And After Exercise,” Proc. Nutr. Soc. 63(2):221-226; Sugden, M. C. (2003) “PDK4: A factor in fatness?,” Obes. Res. 11(2):167-169). It is a target of Pgc-1α and is induced in multiple tissues by long-term calorie restriction. Whereas mice receiving resveratrol showed only a 2.78 fold increase in expression and mice subjected to a calorie restricted diet showed only a 1.48 fold increase in pyruvate dehydrogenase kinase 4 expression, mice receiving Longevinex® showed a 3.25 fold increase in pyruvate dehydrogenase kinase 4 expression.

Analysis of the genes up-regulated or down-regulated by a compound of the present invention (Longevinex®) revealed that oxidative phosphorylation genes, which are involved in mitochondrial ATP production, were markedly up-regulated (Table 4).

TABLE 4

| FC CR | FC RES | FCLGX | Gene |
|---|---|---|---|
| 1.11 | 1.14 | 1.32 | Ndufa5 |
| −1.00 | −1.20 | −1.42 | Ndufaf1 |
| −1.04 | −1.13 | −1.22 | Ndufb3 |
| 1.13 | 1.06 | 1.27 | Ndufb8 |
| 1.12 | 1.18 | 1.28 | Ndufb7 |
| −1.34 | −1.55 | −2.65 | Ndufab1 |
| 1.07 | 1.20 | 1.51 | Ndufc1 |
| −1.07 | −1.30 | −1.39 | Ndufc2 |
| 1.08 | 1.11 | 1.37 | Ndufs1 |
| 1.13 | 1.10 | 1.26 | Ndufs2 |
| 1.09 | 1.12 | 1.23 | Ndufs3 |
| 1.04 | 1.19 | 1.33 | Ndufs5 |
| 1.13 | 1.18 | 1.44 | Ndufs7 |
| −1.02 | 1.03 | −1.23 | Ndufs8 |
| 1.14 | 1.18 | 1.21 | Ndufv1 |
| 1.11 | 1.13 | 1.34 | Ndufv2 |
| 1.17 | 1.13 | 1.43 | Sdha |
| 1.16 | 1.02 | 1.23 | Sdhd |
| 1.46 | 1.29 | 1.49 | Sulf2 |
| 1.01 | −1.25 | −1.33 | Uqcc |
| 1.10 | 1.19 | 1.34 | Uqcrc1 |
| 1.05 | 1.07 | 1.38 | Uqcrfs1 |
| 1.20 | 1.50 | 1.94 | Cox4i2 |
| 1.13 | 1.05 | 1.39 | Cox5a |
| 1.23 | 1.13 | 1.61 | Cox8a |

### Example 4

### Biochemical Pathways Affected by the Compositions of the Present Invention

Recent research has suggested that complex traits are emergent properties of molecular networks that are modulated by complex genetic loci and environmental factors. Chen, Y. et al. (Epub 2008 Mar. 16) “Variations In DNA Elucidate Molecular Networks That Cause Disease,” Nature 452(7186):429-435).

Indeed, research within the last decade has revealed that most chronic illnesses such as cancer, cardiovascular and pulmonary diseases, neurological diseases, diabetes, and autoimmune diseases exhibit dysregulation of multiple cell signaling pathways (Harikumar, K. B. et al. (Epub Feb. 15, 2008) “Resveratrol: A Multitargeted Agent For Age-Associated Chronic Diseases,” Cell Cycle. 2008:7(8)). The compounds of the present invention were therefore evaluated for their effect on the expression of biochemical pathways and were found to affect the expression of genes involved in 220 biological processes (P<0.05) by (Table 5).

TABLE 5

| | | | | Number | | | |
|---|---|---|---|---|---|---|---|
| | | | Changed | of Genes | | | |
| GO ID | Biological Processes | Treatment | by LT-CR | in Series | CR | RES | LGX |
| GO: 0051128 | Regulation Of Cellular Component | CR only | 0.0277 | 51 | 5 | — | — |
| | Organization And Biogenesis | | | | | | |
| GO: 0001558 | Regulation Of Cell Growth | CR only | — | 74 | 5 | — | — |
| GO: 0006820 | Anion Transport | CR only | — | 155 | 6 | — | — |
| GO: 0008361 | Regulation Of Cell Size | CR only | — | 102 | 6 | — | — |
| GO: 0016049 | Cell Growth | CR only | — | 90 | 5 | — | — |
| GO: 0030217 | T Cell Differentiation | CR only | — | 55 | 3 | — | — |
| GO: 0030595 | Leukocyte Chemotaxis | CR only | — | 18 | 2 | — | — |
| GO: 0045580 | Regulation Of T Cell Differentiation | CR only | — | 15 | 2 | — | — |
| GO: 0045792 | Negative Regulation Of Cell Size | CR only | — | 16 | 2 | — | — |
| GO: 0048705 | Skeletal Morphogenesis | CR only | — | 20 | 2 | — | — |
| GO: 0051246 | Regulation Of Protein Metabolic Process | CR only | — | 204 | 8 | — | — |
| GO: 0033554 | Cellular Response To Stress | RES only | 0.0074 | 14 | — | 3 | — |
| GO: 0006888 | ER To Golgi Vesicle-Mediated Transport | RES only | 0.0284 | 16 | — | 3 | — |
| GO: 0000723 | Telomere Maintenance | RES only | — | 17 | — | 3 | — |
| GO: 0001958 | Endochondral Ossification | RES only | — | 8 | — | 2 | — |
| GO: 0006281 | DNA Repair | RES only | — | 178 | — | 13 | — |
| GO: 0006353 | Transcription Termination | RES only | — | 6 | — | 2 | — |
| GO: 0006446 | Regulation Of Translational Initiation | RES only | — | 20 | — | 4 | — |
| GO: 0006596 | Polyamine Biosynthetic Process | RES only | — | 5 | — | 2 | — |
| GO: 0006625 | Protein Targeting To Peroxisome | RES only | — | 5 | — | 2 | — |
| GO: 0006825 | Copper Ion Transport | RES only | — | 9 | — | 3 | — |
| GO: 0006919 | Caspase Activation | RES only | — | 16 | — | 3 | — |
| GO: 0006974 | Response To DNA Damage Stimulus | RES only | — | 217 | — | 15 | — |
| GO: 0006983 | ER Overload Response | RES only | — | 5 | — | 2 | — |
| GO: 0007017 | Microtubule-Based Process | RES only | — | 155 | — | 12 | — |
| GO: 0007091 | Mitotic Metaphase/Anaphase Transition | RES only | — | 8 | — | 2 | — |
| GO: 0007143 | Female Meiosis | RES only | — | 8 | — | 2 | — |
| GO: 0008299 | Isoprenoid Biosynthetic Process | RES only | — | 19 | — | 3 | — |
| GO: 0045351 | Interferon Type I Biosynthetic Process | RES only | — | 6 | — | 2 | — |
| GO: 0045577 | Regulation Of B Cell Differentiation | RES only | — | 8 | — | 2 | — |
| GO: 0046330 | Positive Regulation Of JNK Cascade | RES only | — | 8 | — | 2 | — |
| GO: 0048193 | Golgi Vesicle Transport | RES only | — | 37 | — | 6 | — |
| GO: 0050673 | Epithelial Cell Proliferation | RES only | — | 30 | — | 4 | — |
| GO: 0006119 | Oxidative Phosphorylation | LGX only | 0.0001 | 39 | — | — | 10 |
| GO: 0042773 | ATP Synthesis Coupled Electron | LGX only | 0.0019 | 11 | — | — | 5 |
| | Transport | | | | | | |
| GO: 0030036 | Actin Cytoskeleton Organization And | LGX only | 0.0024 | 146 | — | — | 34 |
| | Biogenesis | | | | | | |
| GO: 0006629 | Lipid Metabolic Process | LGX only | 0.0146 | 535 | — | — | 89 |
| GO: 0044255 | Cellular Lipid Metabolic Process | LGX only | 0.0147 | 459 | — | — | 80 |
| GO: 0001701 | In Utero Embryonic Development | LGX only | 0.0195 | 101 | — | — | 19 |
| GO: 0040008 | Regulation Of Growth | LGX only | 0.0242 | 135 | — | — | 23 |
| GO: 0000375 | RNA Splicing, Via Transesterification | LGX only | 0.0251 | 39 | — | — | 10 |
| | Reactions | | | | | | |
| GO: 0000398 | Nuclear Mrna Splicing, Via Spliceosome | LGX only | 0.0251 | 39 | — | — | 10 |
| GO: 0006366 | Transcription From RNA Polymerase II | LGX only | 0.0264 | 392 | — | — | 72 |
| | Promoter | | | | | | |
| GO: 0006357 | Regulation Of Transcription From RNA | LGX only | 0.0276 | 351 | — | — | 59 |
| | Polymerase II Promoter | | | | | | |
| GO: 0016044 | Membrane Organization And Biogenesis | LGX only | 0.0292 | 209 | — | — | 34 |
| GO: 0006066 | Alcohol Metabolic Process | LGX only | 0.0299 | 222 | — | — | 41 |
| GO: 0065002 | Intracellular Protein Transport Across A | LGX only | 0.0372 | 59 | — | — | 16 |
| | Membrane | | | | | | |
| GO: 0006099 | Tricarboxylic Acid Cycle | LGX only | 0.0396 | 23 | — | — | 7 |
| GO: 0009060 | Aerobic Respiration | LGX only | 0.0396 | 24 | — | — | 7 |
| GO: 0044265 | Cellular Macromolecule Catabolic | LGX only | 0.0417 | 201 | — | — | 41 |
| | Process | | | | | | |
| GO: 0006006 | Glucose Metabolic Process | LGX only | 0.0441 | 83 | — | — | 16 |
| GO: 0045333 | Cellular Respiration | LGX only | 0.0484 | 28 | — | — | 8 |
| GO: 0000038 | Very-Long-Chain Fatty Acid Metabolic | LGX only | — | 5 | — | — | 3 |
| | Process | | | | | | |
| GO: 0000059 | Protein Import Into Nucleus, Docking | LGX only | — | 15 | — | — | 7 |
| GO: 0000186 | Activation Of MAPKK Activity | LGX only | — | 10 | — | — | 5 |
| GO: 0001525 | Angiogenesis | LGX only | — | 124 | — | — | 26 |
| GO: 0001568 | Blood Vessel Development | LGX only | — | 188 | — | — | 42 |
| GO: 0001570 | Vasculogenesis | LGX only | — | 27 | — | — | 8 |
| GO: 0001839 | Neural Plate Morphogenesis | LGX only | — | 40 | — | — | 9 |
| GO: 0001841 | Neural Tube Formation | LGX only | — | 39 | — | — | 9 |
| GO: 0001843 | Neural Tube Closure | LGX only | — | 29 | — | — | 7 |
| GO: 0001935 | Endothelial Cell Proliferation | LGX only | — | 8 | — | — | 4 |
| GO: 0002026 | Cardiac Inotropy | LGX only | — | 10 | — | — | 4 |
| GO: 0003007 | Heart Morphogenesis | LGX only | — | 32 | — | — | 8 |
| GO: 0005978 | Glycogen Biosynthetic Process | LGX only | — | 11 | — | — | 5 |
| GO: 0006007 | Glucose Catabolic Process | LGX only | — | 44 | — | — | 11 |
| GO: 0006098 | Pentose-Phosphate Shunt | LGX only | — | 7 | — | — | 3 |
| GO: 0006118 | Electron Transport | LGX only | — | 303 | — | — | 65 |
| GO: 0006120 | Mitochondrial Electron Transport, | LGX only | — | 6 | — | — | 5 |
| | NADH To Ubiquinone | | | | | | |
| GO: 0006171 | Camp Biosynthetic Process | LGX only | — | 14 | — | — | 5 |
| GO: 0006259 | DNA Metabolic Process | LGX only | — | 524 | — | — | 77 |
| GO: 0006323 | DNA Packaging | LGX only | — | 209 | — | — | 37 |
| GO: 0006325 | Establishment And/Or Maintenance Of | LGX only | — | 203 | — | — | 34 |
| | Chromatin Architecture | | | | | | |
| GO: 0006333 | Chromatin Assembly Or Disassembly | LGX only | — | 80 | — | — | 15 |
| GO: 0006352 | Transcription Initiation | LGX only | — | 27 | — | — | 7 |
| GO: 0006354 | RNA Elongation | LGX only | — | 5 | — | — | 3 |
| GO: 0006367 | Transcription Initiation From RNA | LGX only | — | 11 | — | — | 5 |
| | Polymerase II Promoter | | | | | | |
| GO: 0006396 | RNA Processing | LGX only | — | 319 | — | — | 63 |
| GO: 0006397 | Mrna Processing | LGX only | — | 213 | — | — | 43 |
| GO: 0006414 | Translational Elongation | LGX only | — | 19 | — | — | 6 |
| GO: 0006461 | Protein Complex Assembly | LGX only | — | 122 | — | — | 28 |
| GO: 0006468 | Protein Amino Acid Phosphorylation | LGX only | — | 545 | — | — | 83 |
| GO: 0006470 | Protein Amino Acid Dephosphorylation | LGX only | — | 99 | — | — | 18 |
| GO: 0006473 | Protein Amino Acid Acetylation | LGX only | — | 12 | — | — | 4 |
| GO: 0006508 | Proteolysis | LGX only | — | 545 | — | — | 85 |
| GO: 0006520 | Amino Acid Metabolic Process | LGX only | — | 198 | — | — | 34 |
| GO: 0006606 | Protein Import Into Nucleus | LGX only | — | 53 | — | — | 13 |
| GO: 0006612 | Protein Targeting To Membrane | LGX only | — | 15 | — | — | 5 |
| GO: 0006631 | Fatty Acid Metabolic Process | LGX only | — | 142 | — | — | 35 |
| GO: 0006635 | Fatty Acid Beta-Oxidation | LGX only | — | 13 | — | — | 6 |
| GO: 0006638 | Neutral Lipid Metabolic Process | LGX only | — | 21 | — | — | 6 |
| GO: 0006641 | Triacylglycerol Metabolic Process | LGX only | — | 17 | — | — | 6 |
| GO: 0006662 | Glycerol Ether Metabolic Process | LGX only | — | 23 | — | — | 6 |
| GO: 0006766 | Vitamin Metabolic Process | LGX only | — | 57 | — | — | 14 |
| GO: 0006807 | Nitrogen Compound Metabolic Process | LGX only | — | 315 | — | — | 49 |
| GO: 0006869 | Lipid Transport | LGX only | — | 67 | — | — | 16 |
| GO: 0006913 | Nucleocytoplasmic Transport | LGX only | — | 89 | — | — | 23 |
| GO: 0006914 | Autophagy | LGX only | — | 21 | — | — | 8 |
| GO: 0007031 | Peroxisome Organization And | LGX only | — | 22 | — | — | 7 |
| | Biogenesis | | | | | | |
| GO: 0007182 | Common-Partner SMAD Protein | LGX only | — | 8 | — | — | 4 |
| | Phosphorylation | | | | | | |
| GO: 0007190 | Adenylate Cyclase Activation | LGX only | — | 12 | — | — | 4 |
| GO: 0007242 | Intracellular Signaling Cascade | LGX only | — | 915 | — | — | 133 |
| GO: 0007369 | Gastrulation | LGX only | — | 55 | — | — | 13 |
| GO: 0007498 | Mesoderm Development | LGX only | — | 44 | — | — | 13 |
| GO: 0007507 | Heart Development | LGX only | — | 158 | — | — | 39 |
| GO: 0007512 | Adult Heart Development | LGX only | — | 9 | — | — | 4 |
| GO: 0007517 | Muscle Development | LGX only | — | 105 | — | — | 19 |
| GO: 0008016 | Regulation Of Heart Contraction | LGX only | — | 27 | — | — | 9 |
| GO: 0008286 | Insulin Receptor Signaling Pathway | LGX only | — | 24 | — | — | 7 |
| GO: 0009308 | Amine Metabolic Process | LGX only | — | 294 | — | — | 46 |
| GO: 0009653 | Anatomical Structure Morphogenesis | LGX only | — | 993 | — | — | 143 |
| GO: 0009790 | Embryonic Development | LGX only | — | 387 | — | — | 61 |
| GO: 0009792 | Embryonic Development Ending In Birth | LGX only | — | 190 | — | — | 32 |
| | Or Egg Hatching | | | | | | |
| GO: 0010003 | Gastrulation (Sensu Mammalia) | LGX only | — | 17 | — | — | 6 |
| GO: 0015804 | Neutral Amino Acid Transport | LGX only | — | 6 | — | — | 3 |
| GO: 0015908 | Fatty Acid Transport | LGX only | — | 6 | — | — | 3 |
| GO: 0016071 | Mrna Metabolic Process | LGX only | — | 240 | — | — | 45 |
| GO: 0016192 | Vesicle-Mediated Transport | LGX only | — | 365 | — | — | 61 |
| GO: 0016310 | Phosphorylation | LGX only | — | 601 | — | — | 95 |
| GO: 0016311 | Dephosphorylation | LGX only | — | 111 | — | — | 20 |
| GO: 0016481 | Negative Regulation Of Transcription | LGX only | — | 223 | — | — | 40 |
| GO: 0016485 | Protein Processing | LGX only | — | 58 | — | — | 14 |
| GO: 0016540 | Protein Autoprocessing | LGX only | — | 30 | — | — | 9 |
| GO: 0016567 | Protein Ubiquitination | LGX only | — | 36 | — | — | 9 |
| GO: 0016568 | Chromatin Modification | LGX only | — | 152 | — | — | 27 |
| GO: 0016574 | Histone Ubiquitination | LGX only | — | 5 | — | — | 3 |
| GO: 0019395 | Fatty Acid Oxidation | LGX only | — | 20 | — | — | 8 |
| GO: 0019752 | Carboxylic Acid Metabolic Process | LGX only | — | 403 | — | — | 78 |
| GO: 0030163 | Protein Catabolic Process | LGX only | — | 162 | — | — | 31 |
| GO: 0030239 | Myofibril Assembly | LGX only | — | 12 | — | — | 5 |
| GO: 0030323 | Respiratory Tube Development | LGX only | — | 58 | — | — | 12 |
| GO: 0030324 | Lung Development | LGX only | — | 57 | — | — | 12 |
| GO: 0030855 | Epithelial Cell Differentiation | LGX only | — | 34 | — | — | 8 |
| GO: 0030856 | Regulation Of Epithelial Cell | LGX only | — | 7 | — | — | 3 |
| | Differentiation | | | | | | |
| GO: 0030865 | Cortical Cytoskeleton Organization And | LGX only | — | 10 | — | — | 5 |
| | Biogenesis | | | | | | |
| GO: 0031032 | Actomyosin Structure Organization And | LGX only | — | 16 | — | — | 5 |
| | Biogenesis | | | | | | |
| GO: 0032147 | Activation Of Protein Kinase Activity | LGX only | — | 28 | — | — | 8 |
| GO: 0035051 | Cardiac Cell Differentiation | LGX only | — | 13 | — | — | 6 |
| GO: 0035239 | Tube Morphogenesis | LGX only | — | 128 | — | — | 25 |
| GO: 0035295 | Tube Development | LGX only | — | 174 | — | — | 36 |
| GO: 0042254 | Ribosome Biogenesis And Assembly | LGX only | — | 97 | — | — | 18 |
| GO: 0042692 | Muscle Cell Differentiation | LGX only | — | 58 | — | — | 14 |
| GO: 0043009 | Chordate Embryonic Development | LGX only | — | 187 | — | — | 32 |
| GO: 0043087 | Regulation Of Gtpase Activity | LGX only | — | 59 | — | — | 12 |
| GO: 0043623 | Cellular Protein Complex Assembly | LGX only | — | 39 | — | — | 11 |
| GO: 0043631 | RNA Polyadenylation | LGX only | — | 11 | — | — | 4 |
| GO: 0044257 | Cellular Protein Catabolic Process | LGX only | — | 116 | — | — | 27 |
| GO: 0045214 | Sarcomere Organization | LGX only | — | 9 | — | — | 4 |
| GO: 0045761 | Regulation Of Adenylate Cyclase | LGX only | — | 16 | — | — | 5 |
| | Activity | | | | | | |
| GO: 0045893 | Positive Regulation Of Transcription, | LGX only | — | 225 | — | — | 44 |
| | DNA-Dependent | | | | | | |
| GO: 0045944 | Positive Regulation Of Transcription | LGX only | — | 186 | — | — | 34 |
| | From RNA Polymerase II Promoter | | | | | | |
| GO: 0046058 | Camp Metabolic Process | LGX only | — | 17 | — | — | 5 |
| GO: 0046777 | Protein Amino Acid | LGX only | — | 29 | — | — | 9 |
| | Autophosphorylation | | | | | | |
| GO: 0048276 | Gastrulation (Sensu Vertebrata) | LGX only | — | 24 | — | — | 7 |
| GO: 0048514 | Blood Vessel Morphogenesis | LGX only | — | 160 | — | — | 36 |
| GO: 0048646 | Anatomical Structure Formation | LGX only | — | 171 | — | — | 34 |
| GO: 0050658 | RNA Transport | LGX only | — | 48 | — | — | 11 |
| GO: 0051028 | Mrna Transport | LGX only | — | 45 | — | — | 11 |
| GO: 0051146 | Striated Muscle Cell Differentiation | LGX only | — | 26 | — | — | 11 |
| GO: 0051170 | Nuclear Import | LGX only | — | 54 | — | — | 13 |
| GO: 0055001 | Muscle Cell Development | LGX only | — | 13 | — | — | 5 |
| GO: 0055002 | Striated Muscle Cell Development | LGX only | — | 12 | — | — | 5 |
| GO: 0055007 | Cardiac Muscle Cell Differentiation | LGX only | — | 9 | — | — | 6 |
| GO: 0055012 | Ventricular Cardiac Muscle Cell | LGX only | — | 6 | — | — | 4 |
| | Differentiation | | | | | | |
| GO: 0051016 | Barbed-End Actin Filament Capping | CR & RES | 0.0487 | 17 | 2 | 3 | — |
| GO: 0030029 | Actin Filament-Based Process | CR & LGX | 0.0049 | 157 | 6 | — | 37 |
| GO: 0006084 | Acetyl-Coa Metabolic Process | CR & LGX | 0.0068 | 33 | 3 | — | 11 |
| GO: 0045941 | Positive Regulation Of Transcription | CR & LGX | 0.0119 | 267 | 8 | — | 48 |
| GO: 0006915 | Apoptosis | CR & LGX | 0.0172 | 537 | 14 | — | 84 |
| GO: 0012501 | Programmed Cell Death | CR & LGX | 0.0198 | 544 | 14 | — | 84 |
| GO: 0046356 | Acetyl-Coa Catabolic Process | CR & LGX | 0.0396 | 24 | 2 | — | 8 |
| GO: 0008219 | Cell Death | CR & LGX | 0.0410 | 564 | 14 | — | 84 |
| GO: 0006364 | Rrna Processing | CR & LGX | — | 50 | 3 | — | 11 |
| GO: 0006519 | Amino Acid And Derivative Metabolic | CR & LGX | — | 253 | 8 | — | 41 |
| | Process | | | | | | |
| GO: 0006796 | Phosphate Metabolic Process | CR & LGX | — | 714 | 17 | — | 114 |
| GO: 0006839 | Mitochondrial Transport | CR & LGX | — | 20 | 2 | — | 9 |
| GO: 0007005 | Mitochondrion Organization And | CR & LGX | — | 58 | 4 | — | 19 |
| | Biogenesis | | | | | | |
| GO: 0007167 | Enzyme Linked Receptor Protein | CR & LGX | — | 252 | 9 | — | 46 |
| | Signaling Pathway | | | | | | |
| GO: 0007179 | Transforming Growth Factor Beta | CR & LGX | — | 42 | 3 | — | 11 |
| | Receptor Signaling Pathway | | | | | | |
| GO: 0009056 | Catabolic Process | CR & LGX | — | 474 | 13 | — | 79 |
| GO: 0016265 | Death | CR & LGX | — | 564 | 14 | — | 84 |
| GO: 0030833 | Regulation Of Actin Filament | CR & LGX | — | 10 | 2 | — | 4 |
| | Polymerization | | | | | | |
| GO: 0008104 | Protein Localization | RES & LGX | 0.0007 | 663 | — | 38 | 127 |
| GO: 0015031 | Protein Transport | RES & LGX | 0.0011 | 581 | — | 38 | 121 |
| GO: 0045184 | Establishment Of Protein Localization | RES & LGX | 0.0028 | 610 | — | 38 | 123 |
| GO: 0006886 | Intracellular Protein Transport | RES & LGX | 0.0098 | 357 | — | 26 | 75 |
| GO: 0006605 | Protein Targeting | RES & LGX | 0.0099 | 161 | — | 12 | 34 |
| GO: 0044249 | Cellular Biosynthetic Process | RES & LGX | 0.0107 | 710 | — | 45 | 117 |
| GO: 0009058 | Biosynthetic Process | RES & LGX | 0.0120 | 979 | — | 60 | 155 |
| GO: 0006412 | Translation | RES & LGX | 0.0364 | 338 | — | 25 | 62 |
| GO: 0044262 | Cellular Carbohydrate Metabolic Process | RES & LGX | 0.0471 | 221 | — | 18 | 49 |
| GO: 0005975 | Carbohydrate Metabolic Process | RES & LGX | — | 316 | — | 21 | 60 |
| GO: 0005976 | Polysaccharide Metabolic Process | RES & LGX | — | 42 | — | 9 | 15 |
| GO: 0005977 | Glycogen Metabolic Process | RES & LGX | — | 31 | — | 7 | 13 |
| GO: 0006091 | Generation Of Precursor Metabolites | RES & LGX | — | 390 | — | 24 | 88 |
| | And Energy | | | | | | |
| GO: 0006112 | Energy Reserve Metabolic Process | RES & LGX | — | 35 | — | 7 | 13 |
| GO: 0006413 | Translational Initiation | RES & LGX | — | 40 | — | 6 | 9 |
| GO: 0006511 | Ubiquitin-Dependent Protein Catabolic | RES & LGX | — | 109 | — | 9 | 27 |
| | Process | | | | | | |
| GO: 0006512 | Ubiquitin Cycle | RES & LGX | — | 356 | — | 27 | 79 |
| GO: 0007178 | Transmembrane Receptor Protein | RES & LGX | — | 75 | — | 7 | 19 |
| | Serine/Threonine Kinase Signaling | | | | | | |
| | Pathway | | | | | | |
| GO: 0007264 | Small Gtpase Mediated Signal | RES & LGX | — | 320 | — | 22 | 62 |
| | Transduction | | | | | | |
| GO: 0008380 | RNA Splicing | RES & LGX | — | 162 | — | 12 | 30 |
| GO: 0009059 | Macromolecule Biosynthetic Process | RES & LGX | — | 525 | — | 35 | 90 |
| GO: 0019941 | Modification-Dependent Protein | RES & LGX | — | 111 | — | 9 | 27 |
| | Catabolic Process | | | | | | |
| GO: 0043085 | Positive Regulation Of Enzyme Activity | RES & LGX | — | 40 | — | 5 | 11 |
| GO: 0043280 | Positive Regulation Of Caspase Activity | RES & LGX | — | 17 | — | 3 | 5 |
| GO: 0043281 | Regulation Of Caspase Activity | RES & LGX | — | 27 | — | 5 | 8 |
| GO: 0045454 | Cell Redox Homeostasis | RES & LGX | — | 40 | — | 6 | 9 |
| GO: 0050790 | Regulation Of Catalytic Activity | RES & LGX | — | 241 | — | 21 | 53 |
| GO: 0008064 | Regulation Of Actin Polymerization | All | 0.0139 | 30 | 5 | 4 | 9 |
| | And/Or Depolymerization | | | | | | |
| GO: 0030832 | Regulation Of Actin Filament Length | All | 0.0139 | 31 | 5 | 4 | 9 |
| GO: 0046907 | Intracellular Transport | All | 0.0287 | 523 | 16 | 43 | 113 |
| GO: 0008154 | Actin Polymerization And/Or | All | 0.0414 | 39 | 5 | 6 | 12 |
| | Depolymerization | | | | | | |
| GO: 0051649 | Establishment Of Cellular Localization | All | 0.0467 | 653 | 17 | 45 | 125 |
| GO: 0006457 | Protein Folding | All | — | 124 | 6 | 13 | 34 |
| GO: 0006996 | Organelle Organization And Biogenesis | All | — | 897 | 27 | 53 | 158 |
| GO: 0007010 | Cytoskeleton Organization And | All | — | 403 | 12 | 26 | 70 |
| | Biogenesis | | | | | | |
| GO: 0007018 | Microtubule-Based Movement | All | — | 72 | 4 | 9 | 14 |
| GO: 0030041 | Actin Filament Polymerization | All | — | 17 | 2 | 3 | 7 |
| GO: 0051258 | Protein Polymerization | All | — | 32 | 6 | 6 | 8 |

Calorie restriction affected genes associated with 5% of these processes, administration of resveratrol affected genes associated with 10% of these processes. Compounds of the present invention (e.g., Longevinex®) were found to affect 85% of these processes. Administration of resveratrol to calorie restricted mice failed to affect any genes in any of these processes. Administration of Longevinex® to calorie restricted mice was found to affect genes associated with 8% of these processes. Administration of both resveratrol and Longevinex® was found to affect genes associated with 12% of these processes. Table 6 shows the modulation of the genes of the oxidative phosphorylation pathway (GO:0006119) caused by calorie restriction (CR), resveratrol alone (Res), or the compositions of the present invention (LGX).

TABLE 6
Modulation Of The Genes Of The Oxidative Phosphorylation Pathway
(GO: 0006119)

| | Fold Change | | |
|---|---|---|---|
| Gene | CR | Res | LGX |
| Atp6v0d1 | 1.03 | −1.21 | −1.19 |
| Atp6v1b2 | 1.27 | −1.16 | −1.23 |
| Ndufv1 | 1.14 | 1.18 | 1.21 |
| Ndufc2 | −1.07 | −1.30 | −1.39 |
| Atp6v1d | 1.13 | −1.09 | −1.05 |
| Ndufs3 | 1.09 | 1.12 | 1.23 |
| Atp6v1f | −1.14 | −1.21 | −1.44 |
| Ndufs7 | 1.13 | 1.18 | 1.44 |
| Uqcrh | 1.05 | −1.40 | −1.34 |
| Atp5j | 1.07 | −1.00 | 1.24 |
| Cyc1 | 1.14 | 1.08 | 1.29 |
| Atp5b | 1.10 | 1.01 | 1.27 |
| Atp5k | 1.05 | 1.02 | 1.24 |
| Atp5a1 | 1.09 | 1.02 | 1.29 |
| Atp5h | 1.07 | 1.04 | 1.37 |
| 1110020P15Rik | 1.06 | 1.09 | 1.26 |
| Ndufs1 | 1.08 | 1.11 | 1.37 |
| Atp5f1 | 1.09 | 1.08 | 1.19 |
| Uqcr | 1.03 | 1.10 | 1.30 |
| Atp6v1a | −1.04 | −1.18 | −1.35 |
| Ndufb9 | 1.07 | 1.07 | 1.25 |
| Atp6v1h | −1.17 | −1.19 | −1.11 |
| Uqcrb | −1.09 | −1.00 | −1.22 |
| Msh2 | 1.05 | −1.03 | −1.15 |
| Atp6v1c1 | −1.10 | −1.07 | −1.06 |
| Atp6v1c2 | 2.17 | 1.42 | −1.00 |
| Atp6v0d2 | 1.04 | 1.65 | 1.21 |
| Atp7a | −1.13 | −1.18 | −1.29 |
| Atp5c1 | −1.16 | −1.28 | −1.16 |
| Atp5g1 | 1.26 | −1.58 | −1.17 |
| Atp6v1e1 | 1.05 | −1.42 | −1.42 |
| Atp6v1e2 | 1.11 | 1.28 | 1.30 |
| Ndufa7 | −1.07 | −1.14 | −1.07 |
| Atp5g3 | 1.06 | −1.11 | −1.01 |

Table 7 shows the modulation of the genes of the glucose metabolism pathway (GO:0006006) caused by calorie restriction (CR), resveratrol alone (Res), or the compositions of the present invention (LGX).

TABLE 7
Modulation Of The Genes Of The Glucose Metabolism Pathway
(GO: 0006006)

| | Fold Change | | |
|---|---|---|---|
| Gene | CR | Res | LGX |
| Pgam2 | 1.27 | 1.38 | 1.81 |
| Pkm2 | 1.31 | 1.34 | 1.70 |
| Pgls | 1.28 | 1.24 | 1.42 |
| Eno3 | 1.17 | 1.26 | 1.37 |
| Atf3 | −1.86 | −1.47 | −1.68 |
| Pdk4 | 1.48 | 2.78 | 3.25 |
| H6pd | 1.33 | 1.43 | 2.06 |
| Pcx | 1.40 | 1.33 | 1.60 |
| Fbp2 | −1.16 | 1.42 | 1.47 |
| Pfkp | 1.32 | 1.13 | 1.24 |
| Pik3ca | −1.05 | 1.34 | 1.21 |
| Hibadh | 1.09 | 1.17 | 1.15 |
| Sds | −1.65 | −1.16 | 2.01 |
| Hk3 | 2.23 | 1.60 | 1.77 |
| Hkdc1 | 2.64 | 2.10 | 1.68 |
| Ldhb | 1.21 | 1.35 | 1.60 |
| Mdh1 | 1.07 | 1.08 | 1.41 |
| Ogdh | 1.32 | 1.23 | 1.38 |
| Gapdh | 1.15 | 1.09 | 1.35 |
| Pdha1 | 1.12 | 1.09 | 1.30 |
| Tpi1 | 1.15 | 1.06 | 1.24 |
| Mdh2 | 1.10 | 1.01 | 1.15 |
| Pgm2l1 | 1.19 | 1.31 | 1.11 |
| Pdk1 | −1.01 | 1.01 | −1.18 |
| Hk1 | 1.12 | −1.24 | −1.34 |
| Prkaa1 | −1.08 | 1.17 | −1.38 |
| Mapk14 | −1.10 | −1.13 | −1.51 |
| Pck1 | 1.68 | 2.36 | 4.03 |
| Aldoart1 | 2.17 | 2.37 | 3.08 |
| G6pd2 | 1.93 | 1.37 | 2.90 |
| Ldhal6b | 1.15 | 1.45 | 2.73 |
| Ldhc | 1.94 | 2.25 | 2.44 |
| Gpd1 | 1.83 | 1.51 | 2.41 |
| Adipoq | 1.74 | 1.24 | 2.14 |
| Eno2 | −1.19 | 1.32 | 1.86 |
| Akt1 | 1.11 | 1.66 | 1.73 |
| Bpgm | −1.28 | 1.35 | 1.57 |
| Pdha2 | 1.78 | −1.03 | 1.57 |
| Npy1r | 1.11 | 1.37 | 1.53 |
| Gapdhs | 1.35 | 1.62 | 1.50 |
| G6pdx | 1.30 | 1.00 | 1.38 |
| Adpgk | −1.02 | 1.24 | 1.37 |
| Ins1 | 1.19 | 1.49 | 1.37 |
| Aldoc | −1.32 | 1.14 | 1.32 |
| Pdk2 | 1.11 | 1.11 | 1.24 |
| Aldob | −1.06 | 1.24 | 1.21 |
| Pdk3 | −2.02 | −1.04 | 1.21 |
| Pgm1 | 1.05 | 1.14 | 1.21 |
| Fbp1 | −1.50 | −1.25 | 1.20 |
| Slc2a8 | 1.11 | 1.30 | 1.19 |
| Gpd2 | 1.48 | −1.41 | 1.17 |
| Gck | 1.16 | 1.26 | 1.16 |
| Pgd | 1.20 | −1.05 | 1.16 |
| Dcxr | 1.05 | 1.08 | 1.14 |
| Rpia | 1.04 | 1.06 | 1.12 |
| Tnf | −1.16 | −1.73 | 1.11 |
| Atf4 | −1.06 | 1.07 | 1.10 |
| Nr3c1 | 1.14 | −1.14 | 1.10 |
| Taldo1 | 1.12 | 1.04 | 1.08 |
| Pgam1 | 1.15 | 1.01 | 1.07 |
| Aldoart2 | −1.05 | 1.16 | 1.05 |
| Ldha | 1.10 | −1.03 | 1.04 |
| Lrrc16 | 1.00 | −1.10 | 1.03 |
| Pgm2 | 1.11 | 1.09 | 1.03 |
| Pfkm | 1.13 | 1.07 | 1.02 |
| Pgk2 | −1.79 | 1.29 | −1.01 |
| 6430537H07Rik | −1.26 | −1.73 | −1.04 |
| Pck2 | 1.06 | −1.09 | −1.05 |
| Bad | 1.05 | 1.10 | −1.07 |
| Dlat | 1.05 | −1.09 | −1.07 |
| Dhtkd1 | −1.45 | −1.32 | −1.08 |
| Pfkl | 1.19 | −1.15 | −1.11 |
| Car5a | −2.59 | 1.19 | −1.14 |
| Pdx1 | −3.03 | −1.37 | −1.14 |
| Cacna1a | −1.31 | 1.03 | −1.21 |
| Acn9 | −1.04 | −1.08 | −1.22 |
| Fabp5 | 1.14 | −1.13 | −1.26 |
| Ppara | 1.14 | −1.06 | −1.28 |
| Uevld | −1.01 | −1.12 | −1.28 |
| Pgm3 | 1.13 | −1.09 | −1.29 |
| Ganc | −1.26 | 1.08 | −1.30 |
| Lep | −1.57 | −1.96 | −1.43 |
| Pklr | 1.12 | −1.08 | −1.47 |
| G6pc | 1.42 | −1.62 | −1.71 |
| Onecut1 | −1.32 | −2.13 | −2.05 |

Table 8 shows the modulation of the genes of the tricarboxylic acid metabolism pathway (GO:0006099) caused by calorie restriction (CR), resveratrol alone (Res), or the compositions of the present invention (LGX).

TABLE 8
Modulation Of The Genes Of The Tricarboxylic Acid Metabolism
Pathway (GO: 0006099)

| | Fold Change | | |
|---|---|---|---|
| Gene | CR | Res | LGX |
| Idh3b | 1.22 | 1.30 | 2.06 |
| Cs | 1.22 | 1.07 | 1.46 |
| Sdhd | 1.16 | 1.02 | 1.23 |
| Sdha | 1.17 | 1.13 | 1.43 |
| Sdhb | 1.08 | 1.13 | 1.42 |
| Mdh1 | 1.07 | 1.08 | 1.41 |
| Aco2 | 1.16 | 1.13 | 1.39 |
| Idh2 | 1.12 | 1.21 | 1.36 |
| 2610507B11Rik | 1.10 | 1.06 | 1.25 |
| Dlst | 1.21 | 1.08 | 1.24 |
| Fh1 | −1.01 | 1.05 | 1.18 |
| Mdh2 | 1.10 | 1.01 | 1.15 |
| Idh3a | 1.16 | −1.02 | 1.01 |
| Sdhc | −1.01 | −1.06 | −1.20 |
| Suclg2 | −1.03 | −1.00 | −1.25 |
| Polr3h | −1.19 | −1.20 | −1.34 |
| Sucla2 | 1.01 | −1.01 | 1.05 |
| Suclg1 | 1.08 | −1.07 | 1.00 |
| Mdh1b | −1.23 | −1.20 | −1.00 |
| Atp5g3 | 1.06 | −1.11 | −1.01 |
| Idh3g | 1.02 | −1.06 | −1.02 |
| Aco1 | 1.00 | −1.01 | −1.05 |

Table 9 shows the modulation of the genes of the fatty acid metabolism pathway (GO:0006631) caused by calorie restriction (CR), resveratrol alone (Res), or the compositions of the present invention (LGX).

TABLE 9
Modulation Of The Genes Of The Fatty Acid Metabolism Pathway
(GO: 0006631)

| | Fold Change | | |
|---|---|---|---|
| Gene | CR | Res | LGX |
| Ucp3 | 1.80 | 2.02 | 2.79 |
| Pex5 | 1.43 | 1.70 | 2.17 |
| Syk | 1.73 | 1.76 | 2.09 |
| Tnxb | 1.59 | 1.54 | 2.01 |
| Acox3 | 1.48 | 1.70 | 1.84 |
| Scap | 1.34 | 1.37 | 1.70 |
| Hadhb | 1.80 | 1.57 | 1.51 |
| Pex7 | −1.16 | −1.36 | −1.44 |
| Slc27a1 | 1.06 | 2.03 | 2.42 |
| Prkar2b | 1.95 | 2.01 | 2.24 |
| Acadl | 1.19 | 1.21 | 1.85 |
| Crat | 1.16 | 1.45 | 1.71 |
| Tnfrsf1a | −1.01 | 1.53 | 1.47 |
| Cav1 | 1.14 | 1.18 | 1.45 |
| Cpt1b | 1.07 | 1.27 | 1.45 |
| Hsd17b4 | 1.15 | 1.25 | 1.44 |
| Acaa2 | 1.07 | 1.27 | 1.41 |
| Gpam | −1.09 | 1.34 | 1.39 |
| Hadh | 1.08 | 1.18 | 1.36 |
| Fads3 | 1.13 | 1.38 | 1.35 |
| Lypla2 | −1.00 | −1.31 | −1.13 |
| Acsl6 | −1.15 | −1.23 | −1.26 |
| Elovl5 | −1.12 | −1.68 | −1.91 |
| Aasdh | −1.06 | −1.69 | −2.00 |
| Qk | −1.06 | −1.52 | −2.08 |
| Acox2 | 1.17 | 1.01 | 2.52 |
| Acot5 | 2.89 | 6.34 | 2.39 |
| Ptgis | 1.16 | 1.17 | 1.66 |
| Ppard | 1.20 | 1.09 | 1.59 |
| Acsf3 | 1.28 | 1.17 | 1.57 |
| Cpt1a | −1.04 | 1.27 | 1.49 |
| Hadha | 1.15 | 1.27 | 1.43 |
| Mlycd | −1.01 | 1.55 | 1.43 |
| Acads | 1.08 | 1.20 | 1.40 |
| Acadvl | 1.06 | 1.14 | 1.38 |
| Acsl1 | 1.05 | 1.21 | 1.38 |
| Apoa2 | 1.24 | 1.29 | 1.34 |
| Agpat6 | 1.15 | 1.15 | 1.32 |
| Echdc2 | 1.06 | 1.19 | 1.32 |
| Ech1 | 1.00 | 1.06 | 1.30 |
| Dci | 1.06 | 1.13 | 1.24 |
| Tpi1 | 1.15 | 1.06 | 1.24 |
| Mecr | −1.04 | 1.09 | 1.21 |
| Phyh | 1.10 | 1.15 | 1.20 |
| Ltc4s | 1.46 | −1.05 | 1.19 |
| Elovl1 | −1.03 | 1.15 | 1.18 |
| Scp2 | −1.02 | 1.06 | 1.16 |
| Acot7 | 1.20 | 1.03 | 1.06 |
| Ptges2 | 1.20 | 1.04 | −1.03 |
| Echs1 | 1.07 | 1.01 | −1.09 |
| Mcat | 1.04 | 1.01 | −1.15 |
| Acsl3 | −1.91 | −2.10 | −1.17 |
| 2010111I01Rik | 1.07 | −1.17 | −1.29 |
| Acsl4 | −1.34 | −1.20 | −1.35 |
| Crot | −1.11 | −1.07 | −1.37 |
| Prkaa1 | −1.08 | 1.17 | −1.38 |
| Prkab2 | 1.04 | −1.07 | −1.38 |
| Mapk14 | −1.10 | −1.13 | −1.51 |
| Lypla1 | −1.05 | −1.32 | −1.93 |
| Ndufab1 | −1.34 | −1.55 | −2.65 |
| Scd1 | 2.18 | 1.67 | 3.27 |
| Fasn | 2.54 | 1.54 | 3.10 |
| Agt | 1.42 | 1.57 | 2.50 |
| Baat | 2.33 | 2.78 | 2.42 |
| Elovl6 | 2.36 | 1.27 | 2.38 |
| Alox8 | 1.27 | 1.12 | 2.31 |
| Lcn5 | 1.58 | 1.74 | 2.30 |
| Acot4 | 1.58 | 1.65 | 2.24 |
| Acsm3 | −1.03 | 1.12 | 2.21 |
| Adipoq | 1.74 | 1.24 | 2.14 |
| Mlstd1 | −1.18 | 1.29 | 1.78 |
| Slc27a3 | 1.31 | 1.84 | 1.65 |
| Acsbg1 | 1.64 | 2.24 | 1.62 |
| Acot12 | −1.09 | −1.16 | 1.59 |
| Acot2 | −1.35 | 2.13 | 1.58 |
| Abat | −1.16 | 1.03 | 1.56 |
| Lta4h | 1.38 | 1.68 | 1.49 |
| Elovl3 | −1.12 | 1.11 | 1.47 |
| Acsm1 | 1.27 | 1.25 | 1.43 |
| Alox15 | 1.12 | 1.10 | 1.43 |
| Lypla3 | 1.31 | 1.42 | 1.41 |
| Slc27a2 | −1.52 | −1.96 | 1.41 |
| Elovl7 | 1.41 | 1.20 | 1.39 |
| Fads2 | 1.10 | 1.15 | 1.39 |
| Ptgs1 | 1.18 | 1.26 | 1.37 |
| Brca1 | −1.08 | 1.29 | 1.36 |
| Ptgs2 | 1.01 | 1.25 | 1.35 |
| Scd2 | 1.19 | −1.03 | 1.33 |
| Aacs | 1.35 | −1.12 | 1.32 |
| Alox5 | 1.18 | 1.22 | 1.32 |
| Fads1 | 1.19 | 1.27 | 1.28 |
| Ces3 | −1.16 | 1.06 | 1.23 |
| Pdpn | −1.14 | −1.04 | 1.22 |
| Aloxe3 | −1.14 | 1.00 | 1.21 |
| Ggtla1 | −1.03 | 1.15 | 1.20 |
| Slc27a4 | 1.23 | 1.25 | 1.20 |
| Acsl5 | 1.19 | 1.04 | 1.19 |
| Acot11 | 1.22 | −1.08 | 1.18 |
| Alox12e | −2.02 | −2.10 | 1.18 |
| Aldh5a1 | 1.25 | 1.15 | 1.17 |
| Adipor2 | −1.16 | 1.11 | 1.15 |
| Hao3 | −1.71 | −4.04 | 1.13 |
| Olah | 1.33 | −1.02 | 1.11 |
| Myo5a | 1.21 | 1.09 | 1.09 |
| Tyrp1 | −1.43 | −1.18 | 1.09 |
| Acot8 | 1.05 | −1.05 | 1.08 |
| Acsm5 | −1.38 | 1.14 | 1.08 |
| Hnf1a | 1.20 | 1.81 | 1.06 |
| Fcer1a | 1.55 | 1.73 | 1.05 |
| Fa2h | −1.27 | 1.18 | 1.04 |
| Prkag2 | −1.05 | −1.16 | 1.03 |
| Alox12 | 1.04 | 1.08 | 1.02 |
| Prkab1 | −1.12 | 1.04 | 1.02 |
| Ehhadh | −1.02 | 1.12 | 1.00 |
| Pecr | −1.01 | −1.03 | 1.00 |
| Degs1 | 1.02 | −1.16 | −1.00 |
| Scd3 | −1.37 | 1.36 | −1.01 |
| Acadm | −1.02 | −1.10 | −1.02 |
| Pccb | 1.01 | −1.01 | −1.02 |
| Prkag1 | 1.09 | −1.14 | −1.02 |
| Acox1 | −1.06 | 1.06 | −1.03 |
| Cpt2 | −1.14 | 1.01 | −1.03 |
| Tbxas1 | 1.00 | −1.06 | −1.03 |
| Pex13 | −1.05 | −1.03 | −1.04 |
| C1qtnf2 | −1.23 | −1.02 | −1.05 |
| Slc27a5 | −1.57 | −1.03 | −1.05 |
| Ptges3 | −1.08 | −1.03 | −1.07 |
| Acoxl | −1.85 | −1.39 | −1.08 |
| Alox5ap | 1.07 | −1.08 | −1.09 |
| Ankrd23 | 1.06 | 1.23 | −1.09 |
| Adipor1 | 1.08 | 1.03 | −1.10 |
| Cyb5 | −1.10 | −1.21 | −1.10 |
| Ptgds | −1.29 | 1.05 | −1.10 |
| Ptgds2 | −1.77 | −1.02 | −1.12 |
| Acsm2 | −1.33 | 1.06 | −1.19 |
| Cpt1c | −1.69 | 1.29 | −1.22 |
| Ncf1 | −1.16 | 1.07 | −1.25 |
| Ppara | 1.14 | −1.06 | −1.28 |
| Elovl4 | 1.00 | 1.45 | −1.29 |
| Mlstd2 | −1.20 | −1.19 | −1.29 |
| Oxsm | −1.02 | 1.04 | −1.29 |
| Plp1 | −1.03 | 1.06 | −1.29 |
| Cryl1 | −1.44 | −1.14 | −1.31 |
| Prkaa2 | −1.17 | −1.22 | −1.31 |
| Elovl2 | −1.91 | −1.31 | −1.34 |
| Rnpep | 1.13 | −1.07 | −1.36 |
| Acsbg2 | −1.55 | −1.14 | −1.37 |
| Ptges | −1.05 | −1.30 | −1.38 |
| Prkag3 | −1.24 | 1.34 | −1.45 |
| Hpgd | 1.17 | −1.14 | −2.59 |

A study of the expression of 20,341 genes in cardiac tissue revealed that 2,829 genes exhibited statistically significant differences in expression (P<0.01). Of these, 7% (approximately 189 genes) exhibited altered expression in animals subjected only to calorie reduced diets; 8% (approximately 226 genes) exhibited altered expression in animals receiving only resveratrol; no additional genes exhibited altered expression in animals that received resveratrol and which were subjected to calorie reduced diets. In contrast, 61% of the 20,341 genes (approximately 1,729 genes) exhibited altered expression in animals receiving only compounds of the present invention (e.g., Longevinex®); an additional 2% of the genes (approximately 56 genes) exhibited altered expression in animals that had received compounds of the present invention (e.g., Longevinex®) and which had been subjected to calorie reduced diets; an additional 21% of the genes (approximately 594 genes) exhibited altered expression in animals that had received compounds of the present invention (e.g., Longevinex®) and resveratrol; an additional 1% of the genes (approximately 28 genes) exhibited altered expression in animals that had received compounds of the present invention (e.g., Longevinex®), resveratrol and which had been subjected to calorie reduced diets.

The above data demonstrates that compounds of the present invention (e.g., Longevinex®) were effective in modulating gene expression in hear tissue to an extent surpassing even that of calorie restriction. Similar effects have been observed in non-heart tissue. A study of the expression of 20,341 genes in brain tissue revealed that 3,572 genes exhibited statistically significant differences in expression (P<0.01). Of these, 124 genes exhibited altered expression in animals subjected only to calorie reduced diets; 424 genes exhibited altered expression in animals receiving only resveratrol; 10 genes exhibited altered expression in animals that received resveratrol and which were subjected to calorie reduced diets. In contrast, 2,560 genes exhibited altered expression in animals receiving only compounds of the present invention (e.g., Longevinex®); 19 additional genes exhibited altered expression in animals that had received compounds of the present invention (e.g., Longevinex®) and which had been subjected to calorie reduced diets; 430 additional genes exhibited altered expression in animals that had received compounds of the present invention (e.g., Longevinex®) and resveratrol; 5 additional genes exhibited altered expression in animals that had received compounds of the present invention (e.g., Longevinex®), resveratrol and which had been subjected to calorie reduced diets.

### Example 4

### Model Mechanism of Action of the Compositions of the Present Invention

The compounds of the present invention were thus found to greatly exceed the modulation of gene expression observed upon calorie restriction and to alter the expression of genes in key pathways of lipid metabolism, glucose metabolism, oxidative phosphorylation, the Kreb's cycle, ATP synthesis and fatty acid β oxidation. In summary, the compounds of the present invention were found to have a greater specific activity than resveratrol alone, both in terms of the number of genes and the number of different biochemical pathways affected. The results are significant since calorie restriction (CR) is considered the unequivocal method of prolonging life in all forms of life. Generally, reduction of 50% of caloric intake doubles the lifespan of any organism. The above-described experiments demonstrate that the compositions of the present invention exert a more powerful influence over genome expression than resveratrol or CR, and marks the first time any technology has been shown to exceed the effects of CR. Furthermore, the compositions of the present invention were found to influence genome expression at an earlier stage of life than CR (which requires a life-long adherence to a CR diet to differentiate genes).

Without intending to be bound by any mechanism of action, the above results suggest that the compounds of the present invention act by enhancing the activity of the forkhead Foxo1 (daf-16, dFoxO) transcription factor (FIG. 5). Studies in model organisms has shown that Foxo1 mediates lifespan expression by enhancing gene expression. Insulin/IGF-1 signaling phosphorylates Foxo1, thereby causing it to be excluded from the nucleus and downregulating its actions. The compounds of the present invention decrease insulin and IGF-1 signaling thereby decreasing Foxo1 phosphorylation. Consistent with this model are the observations that the insulin receptor signaling pathway (e.g., GO:008286; genes Ide, Igfbp4, and Igfbp6) is affected by the compounds of the present invention. Expression of Foxo1 is increased by 1.75 fold. The compounds of the present invention mediate decreased glycolysis and increased gluconeogenesis (e.g., GO:0006006), enhanced Pgc-1α expression (thereby leading to stimulation of Pdk4 expression (e.g., a 1.94 fold increase in Ppargcl1α and a 3.25 fold increase in Pdk4), increased expression of lipid metabolism genes (e.g., a 2.79 fold increase in Ucp3, 1.49 fold increase in Cpt1a, and a 1.45 fold increase in Cpt1b). Lipid and fatty acid metabolism genes GO:0006629 and GO:0006635 are uniquely affected by the compounds of the present invention. The compounds of the present invention thus exert a more pronounced favorable effect on key processes affected by calorie restriction and resveratrol (e.g., chromatin remodeling, transcription from RNA polymerase II promoter, and the ubiquitin cycle. Genes GO:0006333 and GO:0006367 are uniquely affected by the compounds of the present invention; Gene GO:0006512 is affected by resveratrol and Longevinex®. Thus, in sum, a proposed mechanism of action is that the compositions of the present invention deliver resveratrol to cells, where it passes through cell walls, enters the cytoplasm, and facilitates the translocation of Foxo1 gene into the cell nucleus, which produces the longevity effects.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A resveratrol-containing composition that, upon administration to a recipient, modulates the concentration or activity, relative to resveratrol alone or calorie restriction, of the product of a survival/longevity gene or the product of a gene whose expression enhances cellular damage.

2. The resveratrol-containing composition of claim 1, wherein said modulation alters:
(A) oxidative phosphorylation;
(B) actin filament length or polymerization;
(C) intracellular transport;
(D) organelle biogenesis;
(E) insulin signaling;
(F) glycolysis;
(G) gluconeogenesis; or
(H) fatty acid metabolism
in said recipient.

3. The resveratrol-containing composition of claim 1, wherein said survival/longevity gene product is Sirtuin 1 or the forkhead Foxo1 transcription factor.

4. The resveratrol-containing composition of claim 1, wherein said gene whose expression enhances cellular damage encodes uncoupling protein 3 or pyruvate dehydrogenase kinase 4.

5. The resveratrol-containing composition of claim 1, wherein said composition comprises:
(a) trans-resveratrol; and
(b) a metal chelating agent;
wherein said trans-resveratrol is encapsulated to thereby substantially preserve the ability of said composition to modulate the concentration or activity of said product of said survival/longevity gene or said product of said gene whose expression enhances cellular damage, from loss due to exposure of said trans-resveratrol to light or oxygen.

6. The resveratrol-containing composition of claim 5, wherein said metal chelating agent is nordihydroguairetic acid.

7. The resveratrol-containing composition of claim 5, wherein said metal chelating agent is phytic acid.

8. The resveratrol-containing composition of claim 5, wherein said composition additionally comprises quercetin.

9. The resveratrol-containing composition of claim 5, wherein said composition additionally comprises hyaluronic acid.

10. The resveratrol-containing composition of claim 5, wherein said composition additionally comprises vitamin D.

11. The resveratrol-containing composition of claim 5, wherein said encapsulation of resveratrol is a microencapsulation.

12. A method for ameliorating a symptom associated with an existing disease of an individual or for preventing onset of said symptom in an individual prior to the occurrence of said disease in said individual, which comprises administering to said individual, a resveratrol-containing composition that modulates the concentration or activity, relative to resveratrol alone or calorie restriction, of the product of a survival/longevity gene or the product of a gene whose expression enhances cellular damage, wherein said resveratrol is provided in an amount effective to cause a modulation of said concentration or activity of said gene that ameliorates said symptom of said disease, and wherein said disease is selected from the group consisting of: cardiovascular disease, cancer, macular degeneration, a disease associated with aging, and inflammation.

13. The method of claim 12, wherein said modulation alters:
(A) oxidative phosphorylation;
(B) actin filament length or polymerization;
(C) intracellular transport;
(D) organelle biogenesis;
(E) insulin signaling;
(F) glycolysis;
(G) gluconeogenesis; or
(H) fatty acid metabolism
in said individual.

14. The method of claim 12, wherein said survival/longevity gene product is Sirtuin 1 or the forkhead Foxo1 transcription factor.

15. The method of claim 12, wherein said gene whose expression enhances cellular damage encodes uncoupling protein 3 or pyruvate dehydrogenase kinase 4.

16. The method of claim 12, wherein said composition comprises:
(a) trans-resveratrol; and
(b) a metal chelating agent;
wherein said trans-resveratrol is encapsulated to thereby substantially preserve the ability of said composition to modulate the concentration or activity of said product of said survival/longevity gene or said product of said gene whose expression enhances cellular damage, from loss due to exposure of said trans-resveratrol to light or oxygen.

17. The method of claim 12, wherein said disease is cancer.

18. The method of claim 12, wherein said disease is a disease associated with aging.

19. The method of claim 12, wherein said disease associated with aging is a neurodegenerative disease.

20. The method of claim 16, wherein said composition additionally comprises quercetin, hyaluronic acid and/or vitamin D.
